(19) United States
(12) Patent Application Publication
(10) Pub. No.: US 2004/0219645 A1
(43) Pub. Date: Nov. 4, 2004

(54) Polyketides and their synthesis

(76) Inventors: Peter Francis Leadley, Cambridge (GB); James Staunton, Cambridge (GB); Mark Yan Oliynyk, Cambridge (GB)

Correspondence Address: DANN, DORFMAN, HERRELL & SKILLMAN, saet, 1601 MARKET STREET, PHILADELPHIA, PA, 19103-2307, US

(21) Appl. No.: 09/980,217

(22) PCT Filed: May 30, 2001
(86) PCT No.: PCT/GB00/02072

(30) Foreign Application Priority Data
May 28, 1999 (GB) 9912563.5

(51) Int. Cl. C12P 19/62

(52) U.S. Cl. 435/76

(57) ABSTRACT

The complete sequence of the gene cluster for the monensin type I polyketide synthase, from S. cinnamonensis, is provided. Thus variant polyketides containing monensin-derived elements can be genetically engineered. Furthermore there are novel features, e.g. a regulatory protein mon RI, which are of wide utility.

The present invention relates to processes and materials (including enzyme systems, nucleic acids, vectors and cultures) for preparing polyketides, particularly polyethers but including polyenes, macrolides and other polyketides by recombinant synthesis, and to the polyketides so produced, particularly novel polyketides. (N.B the term “polyketide” is being used in its conventional sense to include structures notionally derived by the reduction and/or other processing or modification of one or more Ketide units). Furthermore the invention provides the entire nucleic acid sequence of the biosynthetic gene cluster that governs the production of the ionophoric antibiotic polyether polyketide monensin in Streptomyces cinnamonensis, and the use of all or part of the cloned DNA first, in the specific detection of other polyether biosynthetic gene clusters; secondly in the engineering of mutant strains of S. cinnamonensis and of other actinomycetes which are suitable host strains for the high level production of novel recombinant polyketides; and thirdly in the provision of recombinant biosynthetic genes which lead to such novel polyketide products.

Polyketides are a large and structurally diverse class of natural products that includes many compounds possessing antibiotic or other pharmacological properties, such as erythromycin, tetracyclines, rapamycin, avermectin, monensin, epothilones and FK506. In particular, polyketides are abundantly produced by Streptomyces and related actinomycete bacteria. They are synthesised by the repeated stepwise condensation of acylthioesters in a manner analogous to that of fatty acid biosynthesis. The greater structural diversity found among natural polyketides arises from the selection of (usually) acetate or propionate as “starter” or “extender” units; and from the differing degree of processing of the β-keto group observed after each condensation. Examples of processing steps include reduction to β-hydroxyacyl-, reduction followed by dehydration to 2-enoyl-, and complete reduction to the saturated acylthioester. The stereochemical outcome of these processing steps is also specified for each cycle of chain extension. In addition, the biosynthetic pathways to many polyketides involve additional enzyme-catalysed modifications which may include: methylation by O- and C-methyltransferases, hydroxylation by cytochrome P450 enzymes, other oxidation or reduction processes, and the biosynthesis and attachment of novel sugars and/or deoxy sugars.

The biosynthesis of polyketides is initiated by a group of chain-forming enzymes known as polyketide synthases. Two classes of polyketide synthase (PKS) have been described in actinomycetes. One class, named Type I PKSs, represented by the PKSs for the macrolides erythromycin, oleandomycin, avermectin and rapamycin, consists of a different set or “module” of enzymes for each cycle of polyketide chain extension. (For examples see Cortés, J. et al. Nature (1990) 348:176-178; Donadio, S. et al. Science (1991) 252:675-679; Swan, D. G. et al. Mol. Gen. Genet. (1994) 242:358-362; MacNeil, D. J. et al. Gene (1992) 115:119-125; Schwecke, T. et al. Proc. Natl. Acad. Sci. USA (1995) 92:7839-7843.)

The term “extension module” as used herein refers to the set of contiguous domains, from a β-ketoacyl-ACP synthase (“KS”) domain to the next acyl carrier protein (“ACP”) domain, which accomplishes one cycle of polyketide chain extension. The term “loading module” is used to refer to any group of contiguous domains which accomplishes the loading of the starter unit onto the PKS and thus renders it available to the KS domain of the first extension module. The length of polyketide formed has been altered, in the case of erythromycin biosynthesis, by specific relocation using genetic engineering of the enzymatic domain of the erythromycin-producing PKS that contains the chain releasing thioesterase/cyclase activity (Cortés J. et al. Science (1995) 268:1487-1489; Kao, C. M. et al. J. Am. Chem. Soc. (1995) 117:9105-9106).

In-frame deletion of the DNA encoding part of the ketoreductase domain in module 5 of the erythromycin-producing PKS (also known as 6-deoxyerythronolide B synthase, DEBS) has been shown to lead to the formation of erythromycin analogues 5,6-dideoxy-3-α-mycarosyl-5-oxoerythronolide B, 5,6-dideoxy-5-oxoerythronolide B and 5,6-dideoxy,6-β-epoxy-5-oxoerythronolide B (Donadio, S. et al. Science (1991) 252:675-679). Likewise, alteration of active site residues in the enoylreductase domain of module 4 in DEBS, by genetic engineering of the corresponding PKS-encoding DNA and its introduction into Saccharopolyspora erythraea, led to the production of 6,7-anhydroerythromycin C (Donadio, S. et al. Proc. Natl. Acad. Sci. USA (1993) 90:7119-7123).

International Patent Application number WO 93/13663 describes additional types of genetic manipulation of the DEBS genes that are capable of producing altered polyketides. However many such attempts are reported to have been unproductive (Hutchinson, C. R. and Fujii, I. Annu. Rev. Microbiol. (1995) 49:201-238, at p. 231). The complete DNA sequence of the genes from Streptomyces hygroscopicus that encode the modular Type I PKS governing the biosynthesis of the macrocyclic immunosuppressant polyketide rapamycin has been disclosed (Schwecke, T. et al. (1995) Proc. Natl. Acad. Sci. USA 92:7839-7843). The DNA sequence is deposited in the EMBL/Genbank Database under the accession number X86780.

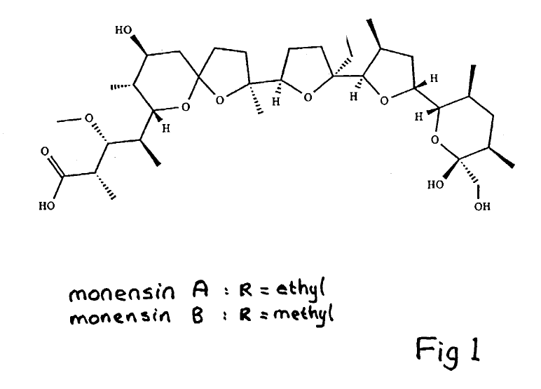

WO 98/01546 discloses that a PKS gene assembly (particularly of Type I) encodes a loading module which is followed by at least one extension module. The first open reading frame encodes the first multi-enzyme or cassette (DEBS1) which consists of three modules: the loading module (ery-load) and two extension modules (modules 1 and 2). The loading module comprises an acyltransferase and an acyl-carrier protein. This may be contrasted with FIG. 1 of WO 93/13663 (referred to above). This shows ORF1 as only two modules, the first of which is in fact both the loading module and the first extension module.

WO 98/01546 describes in general terms the production of a hybrid PKS gene assembly comprising a loading module and at least one extension module. It also describes (see also Marsden, A. F. A. et al. Science (1998) 279:199-202) construction of a hybrid PKS gene assembly by grafting the wide-specificity loading module for the avermectin-producing polyketide synthase onto the first multi-enzyme component (DEBS1) for the erythromycin PKS in place of the normal loading module. Certain novel polyketides can be prepared using the hybrid PKS gene assembly, as described for example in WO 98/01571.

WO 98/01546 further describes the construction of a hybrid PKS gene assembly by grafting the loading module for the rapamycin-producing polyketide synthase onto the first multi-enzyme component (DEBS1) for the erythromycin PKS in place of the normal loading module. The loading module of the rapamycin PKS differs from the loading modules of DEBS and the avermectin PKS in that it comprises a CoA ligase domain, an enoylreductase (“ER”) domain and an ACP, so that suitable organic acids including the natural starter unit 3,4-dihydroxycyclohexane carboxylic acid may be activated in situ on the PKS loading domain and, with or without reduction by the ER domain, transferred to the ACP for intramolecular loading of the KS of extension module 1 (Schwecke, T. et al. Proc. Natl. Acad. Sci. USA (1995) 92:7839-7843). WO 98/51695 and WO 98/49315 describe additional types of genetic manipulation of the DEBS genes that are capable of producing altered polyketides.

The second class of PKS, named Type II PKSs, is represented by the synthases for aromatic compounds. Type II PKSs contain only a single set of enzymatic activities for chain extension and these are re-used as appropriate in successive cycles (Bibb, M. J. et al. EMBO J. (1989) 8:2727-2736; Sherman, D. H. et al. EMBO J. (1989) 8:2717-2725; Fernandez-Moreno, M. A. et al. J. Biol. Chem. (1992) 267:19278-19290). The “extender” units for the Type II PKSs are usually acetate units, and the presence of specific cyclases dictates the preferred pathway for cyclisation of the completed chain into an aromatic product (Hutchinson, C. R. and Fujii, I. Ann. Rev. Microbiol. (1995) 49:201-238). Hybrid polyketides have been obtained by the introduction of cloned Type II PKS gene-containing DNA into another strain containing a different Type II PKS gene cluster, for example by introduction of DNA derived from the gene cluster for actinorhodin, a blue-pigmented polyketide from Streptomyces coelicolor, into an anthraquinone polyketide-producing strain of Streptomyces galileus (Bartel, P. L. et al. J. Bacteriol. (1990) 172:4816-4826).

The minimal number of domains required for polyketide chain extension on a Type II PKS when expressed in a Streptomyces coelicolor host cell (the “minimal PKS”) has been defined for example in WO 95/08548 as containing the following three polypeptides which are products of the actI genes: firstly KS; secondly a polypeptide termed the CLF with end-to-end amino acid sequence similarity to the KS but in which the essential active site residue of the KS, namely a cysteine residue, is substituted either by a glutamine residue or, in the case of the PKS for a spore pigment such as the whiE gene product (Davis, N. K. and Chater, K. F. Mol. Microbiol. (1990) 4:1679-1691) by a glutamic acid residue; and finally an ACP. The CLF has been stated (for example in WO 95/08548) to be a factor that determines the chain length of the polyketide chain that is produced by the minimal PKS. However it has been found (Shen, B. et al. J. Am. Chem. Soc. (1995) 117:6811-6821) that when the CLF for the octaketide actinorhodin is used to replace the CLF for the decaketide tetracenomycin in host cells of Streptomyces glaucescens, the polyketide product is not found to be altered from a decaketide to an octaketide, so the exact role of the CLF remains unclear. An alternative nomenclature has been proposed in which KS is designated KSα and CLF is designated KSβ, to reflect this lack of knowledge (Meurer, G. et al. Chemistry & Biology (1997) 4:433-443). The mechanism by which acetate starter units and acetate extender units are loaded onto the Type II PKS is not known, but it is speculated that the malonyl-CoA:ACP acyltransferase of the fatty acid synthase of the host cell can fulfil the same function for the Type II PKS (Revill, W. P. et al. J. Bacteriol. (1995) 177:3946-3952).

WO 95/08548 describes the replacement of actinorhodin PKS genes by heterologous DNA from other Type II PKS gene clusters, to obtain hybrid polyketides. It also describes the construction of a strain of Streptomyces coelicolor which substantially lacks the native gene cluster for actinorhodin, and the use in that strain of a plasmid vector pRM5 derived from the low-copy number vector SCP2* isolated from Streptomyces coelicolor (Bibb, M. J. and Hopwood, D. A. J. Gen. Microbiol. (1981) 126:427-442) and in which heterologous PKS-encoding DNA may be expressed under the control of the divergent actI/actIII promoter region of the actinorhodin gene cluster (Fernandez-Moreno, M. A. et al. J. Biol. Chem. (1992) 267:19278-19290). The plasmid pRM5 also contains DNA from the actinorhodin biosynthetic gene cluster encoding the gene for a specific activator protein, ActII-orf4. The ActII-orf4 protein is required for transcription of the genes placed under the control of the actI/actIII bidirectional promoter and activates gene expression during the transition from growth to stationary phase in the vegetative mycelium (Hallam, S. E. et al. Gene (1988) 74:305-320).

Type II clusters in Streptomyces are known to be activated by pathway-specific activator genes (Narva, K. E. and Feitelson, J. S. J. Bacteriol. (1990) 172:326-333; Stutzman-Engwall, K. J. et al. J. Bacteriol. (1992) 174:144-154; Fernandez-Moreno, M. A. et al. Cell (1991) 66:769-780; Takano, E. et al. Mol. Microbiol. (1992) 6:2797-2804; Gramajo, H. C. et al. Mol. Microbiol. (1993) 7:837-845). The DnrI gene product complements a mutation in the actII-orf4 gene of S. coelicolor, implying that DnrI and ActII-orf4 proteins act on similar targets. A gene (srmR) has been described (EP 0 524 832 A2) that is located near the Type I PKS gene cluster for the macrolide polyketide spiramycin. This gene specifically activates the production of the macrolide antibiotic spiramycin, but no other examples have been found of such a gene. Also, no homologues of the ActII-orf4/DnrI/RedD family of activators have been described that act on Type I PKS genes. WO 98/01546 describes the use of the ActII-orf4 family of activators in conjunction with their cognate promoters (e.g actII-orf4 with the actI promoter) in a heterologous actinomycete to obtain high level expression of recombinant Type I polyketide synthase genes.

Although large numbers of therapeutically important polyketides have been identified, there remains a need to obtain novel polyketides that have enhanced properties or possess completely novel bioactivity. The complex polyketides produced by Type I PKSs are particularly valuable, in that they include compounds with known utility as anthelminthics, insecticides, immunosuppressants, antifungal agents or antibacterial agents. Because of their structural complexity, such novel polyketides are not readily obtainable by total chemical synthesis, nor by chemical modifications of known polyketides.

There is also a need to develop reliable and specific ways of deploying individual genes and portions of genes in practice so that all, or a large fractions of hybrid PKS genes that are constructed, are viable and produce the desired polyketide product. This includes the development of advantageous host strains for expression of such genes. For example many polyketides are rendered bioactive by the action of further enzymes other than the polyketide synthase, and host strains that contain and are able to express the genes for such enzymes are particularly convenient for the efficient synthesis of the bioactive material. In those cases where the construction of a known or a novel polyketide requires specialised precursors, host strains containing and able to express the genes for key enzymes that enhance the production of such specialised precursors are equally valuable and desirable. There is also a need to develop rational methods of increasing the expression level of all the genes required for production of a specific polyketide. Clearly also a host cell which is advantageous for the above reasons, and/or because of other favourable characteristics including but not limited to its speed of growth, excellent handling characteristics in fermentation, and ease of transformation with DNA by various techniques, can be made even more favourable by the cloning into that cell of such auxiliary genes for polyketide modification, or gene activation, or post-translational modification, or precursor supply.

The DNA sequences have been disclosed for several Type I PKS gene clusters that govern the production of 16-membered macrolide polyketides, including the tylosin PKS from Streptomyces fradiae (application EP 0 791 655 A2), the niddamycin PKS from Streptomyces caelestis (Kavakas, S. J. et al. J. Bacteriol. (1997) 179:7515-7522) and the spiramycin PKS from Streptomyces ambofaciens (application EP 0791 655 A2). DNA sequences have also been disclosed for Type I PKS gene clusters that govern the production of further complex polyketides, for example rifamycin from Amycolatopsis mediterranei (WO 98/07868), and soraphen from Sorangium cellulosum (U.S. Pat. No. 5,716,849), but so far no DNA sequence has been disclosed for one of the most widespread and important classes of complex polyketides, the polyethers.

Polyethers form an important group of complex polyketide antibiotics (Westley, J. W. in “Antibiotics IV. Biosynthesis” (Corcoran, J. W. Ed.), Springer-Verlag, New York (1981) p. 41-73). They are polyoxygenated carboxylic acids which act as selective ionophores transporting cations across the cell membrane of target cells and thereby causing depolarisation and cell death. Certain polyethers including monensin, lasalocid and tetronasin are in widespread use in animal husbandry as coccidiostats (principally targetted against Eimeria spp.) and as growth promoters. Polyethers have also been reported to be active in vitro and in vivo against the malarial parasite Plasmodium falciparum (Gumila, C. et al. Antimicrobial Agents and Chemotherapy (1997) 41: 523-529).

Polyethers contain multiple asymmetric centres and are characterised by the presence of tetrahydrofuran and tetrahydropyran rings, producing a characteristic shape which is non-polar on its outer surface and therefore well adapted for transport of material across bacterial membranes; and provides on its inner surface polar coordinating ligands for a centrally-bound metal ion. In addition to tetrahydrofuran and tetrahydropyran rings, other groups which are often present include spiroketal, dispiroketal, and substituted benzoic acid moieties and occasionally other groups for example a tetronic acid or a 6-membered carbocyclic ring

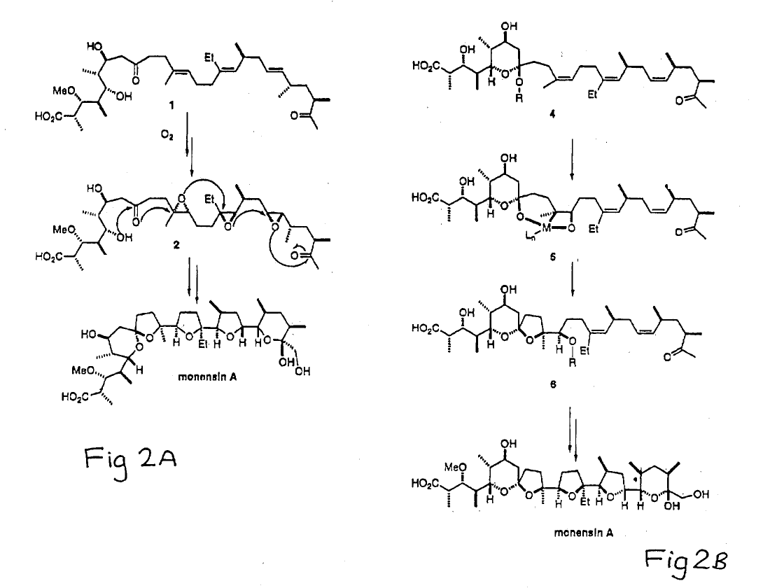

Monensins A and B are produced by the actinomycete Streptomyces cinnamonensis. Their structures are shown in FIG. 1. Monensin B differs from monensin A only in the presence of a methyl sidechain at C-16 rather than an ethyl sidechain. Monensin selectively binds and transports sodium ions. In addition to its antibacterial and antifungal properties monensin has some activity against protozoal parasites such as the malarial parasite Plasmodium falciparum. Although the structures of polyethers differ significantly from those of other complex polyketides such as the polyhydroxylated and polyene macrolides, their biosynthesis appears to take place by a metabolic pathway which has many common elements. Thus experiments using carbon 14-labelled precursors have shown that monensin A is synthesised from five acetate, one butyrate and seven propionate units (Day, L. E. et al. Antimicrob. Agents Chemother. (1973) 4:410-414). Similarly experiments using precursors doubly-labelled with carbon-13 and oxygen-18 have shown that oxygens (O)1, (O)3, (O)4, (O)5, (O)6 and (O)10 of monensin arise from the carboxylate oxygens of either propionate or acetate, while growth in the presence of oxygen-18 oxygen gas demonstrated that the three remaining ether oxygens (O)7, (O)8 and (O)9 are derived from molecular oxygen (Cane, D. E. et al., J. Am. Chem. Soc. (1981) 103:5962-5965; Cane, D. E. et al. J. Am. Chem. Soc. (1982) 104:7274-7281; Ajaz, A. A. and Robinson, J. A. J. Chem. Soc. Chem. Commun. (1983) 12:679-680). These findings have been rationalised by proposing that the biosynthesis of monensin proceeds via an acyclic triene intermediate (1) in which the geometry of all three carbon-carbon double bonds is E (entgegen) rather than Z (zusammen). The triene is then proposed to be subject to epoxidation to a tri-epoxide (2) and then ring opening is proposed to occur with concomitant sequential formation of the five ether rings as shown in FIG. 2A. Such a biosynthetic pathway, first mooted by Westley in 1974 (Westley J. W. et al., J. Antibiot. (1974) 27:597-604) accounts for the observed stereochemistry at the multiple asymmetric centres in monensin, (Cane, D. E. et al. J. Am. Chem. Soc. (1982) 104:7274-7281; Sood, G. R. et al. J. Chem. Soc. Chem. Commun. (1984) 21:1421-1424) and analogous schemes can be used to account for the biosynthesis of other known polyethers. such as lasalocid A (Hutchinson C. R. et al., J. Am. Chem. Soc. (1981) 103:5953-5956), tetronasin (ICI 139603) (Demetriadou, A. K. et al. J. Chem. Soc. Chem. Commun. (1985) 7:408-410) and narasin (Spavold, Z. et al. Tetrahedron Letters (1986) 27:3299-3302). The hydroxylation at C-26 and the introduction of an O-methyl group on oxygen 3-are proposed to occur as late steps in the biosynthesis, after formation of the polyether structure.

Unfortunately key aspects of the biosynthetic scheme shown in FIG. 2A have so far eluded experimental confirmation. No biosynthetic intermediates have been isolated from mutants of S. cinnamonensis that are blocked in early stages of monensin production. 26-deoxymonensin A has been isolated from a S. cinnamonensis mutant partially blocked in monensin production (Ashworth, D. M. et al. J. Antibiot. (1989) 42:1088-1099) and 3-0-demethylmonensins A and B have been recovered as minor components from the fermentation broth of a monensin-producing strain (Pospisil, S. et al. J. Antibiot. (1987) 40:555-557). When fed to cells of S. cinnamonensis in radio-labelled form, neither 26-deoxymonensin A, nor 3-0-demethylmonensin A, nor 3-0-demethyl, 26-deoxymonensin A were significantly incorporated into monensin A (Ashworth, D. M. et al. J. Antibiot. (1989) 42:1088-1099), either because they are actively excluded or because these modifications in fact occur earlier in the biosynthetic pathway so that these metabolites are shunt products not readily converted into the final antibiotic by the respective hydroxylase or methyltransferase. Similarly, the putative all (E)-triene precursor (1) has been synthesised and shown not to become incorporated into monensin when fed to growing cells of S. cinnamonensis (Holmes, D. S. et al. Helv. Chim. Acta (1990) 73:239-259). An alternative pathway has been proposed, as shown in FIG. 2B, based on the transition-metal-mediated oxidation of 1,5-dienes (Walba, D. M. and Edwards, P. D. Tetrahedron Lett. (1980) 21:3531-3534). The triene intermediate (4) would different from that of FIG. 2A (1) only in that each carbon-carbon double bond would have the (Z)-configuration (Townsend, C. A. and Basak, A. Tetrahedron (1991) 47:2591-2602) and not the (E)-configuration.

The genetic basis of secondary metabolite biosynthesis essentially exists in the genes which code for the individual biosynthetic enzymes and in the regulatory elements which control the expression of the biosynthetic genes. The genes encoding biosynthesis of polyketides in actinomycetes have hitherto been found as clusters of adjacent genes, ranging in size from 20 kilobasepairs (kbp) to over 100 kbp. The clusters often contain specific regulatory genes and genes conferring resistance of the producing strain to its own antibiotic.

In various of its aspects the invention provides the following:

(1) a DNA sequence encoding at least one-peptide necessary for the biosynthesis of monensin, preferably comprising one or more of the following genes: mon BI, mon BII, mon CI, mon CII, mon H, mon RI, mon RII, mon T, mon AIX and mon AX as depicted in the appended sequence data or an allele or mutation thereof;

(2) a DNA sequence according to the first aspect comprising all of the genes listed therein or an allele or mutation thereof;

(3) a DNA sequence according to the first aspect comprising the complete monensin gene cluster;

(4) a DNA sequence coding for one or more of the peptides set out below, said peptide having the amino acid sequence as set out in the appended sequence data or being a variant thereof having the specified activity:

| peptide | activity |
|---|---|
| mon CII | epoxyhydrolase/cyclase |
| mon E | S-adenosylmethionine-dependent methyltransferase |
| mon T | monensin resistance gene |
| mon RII | repressor protein |
| mon AIX | thioesterase |
| mon AI | polyketide synthase multienzyme |
| mon AII | polyketide synthase multienzyme |
| mon AIII | polyketide synthase multienzyme |
| mon AIV | polyketide synthase multienzyme |
| mon AVI | polyketide synthase multienzyme |
| mon AVII | polyketide synthase multienzyme |
| mon AVIII | polyketide synthase multienzyme |
| mon H | regulatory protein |
| mon CI | flavin-dependent epoxidase |
| mon BII | carbon-carbon double bond isomerase |
| mon BI | carbon-carbon double bond isomerase |
| mon D | cytochrome P450 hydroxylase |
| mon RI | activator protein |
| mon AX | thioesterase |

(5) a recombinant cloning or expression vector comprising a DNA sequence according to any of aspects 1-4;

(6) a transformant host cell which has been transformed to contain a DNA sequence according to any of aspects 1-4 and is capable of expressing a corresponding peptide;

(7) a hybridization probe comprising a polynucleotide which binds specifically to a region of the monensin gene cluster selected from mon BI, mon BII, mon CI, mon CII, mon H, mon RI, mon RII, mon T, mon AIX and mon AX;

(8) use of a probe according to aspect (7) in a method of detecting the presence of a gene cluster which governs the synthesis of a polyether, and optionally isolating a gene cluster detected thereby;

(9) Use of a probe comprising a polynucleotide which binds specifically to a gene responsible for levels of activity of the monensin gene cluster, preferably a regulatory gene, resistance gene or thioesterase gene, more preferably the regulatory gene mon RI, in a method of detecting an analogous gene in a gene cluster of another polyketide, preferably a polyether, and optionally manipulating the gene detected thereby to alter the level of expression of said other polyketide;

(10) a host cell, preferably Streptomyces cinnamonensis, containing a heterologous gene under the control of the mon RI gene and a monensin promoter;

(11) use of a portion of the monensin gene cluster having chain terminating activity, preferably comprising at least one of mon AIX and mon AX or a mutant or allele thereof having chain terminating activity, to effect chain release of a peptide other than one required for monensin biosynthesis;

(12) use of a portion of the monensin gene cluster having carbon-carbon double bond isomerase activity, preferably comprising at least one of mon BI and mon BII or a mutant or allele thereof having isomerase activity to provide a desired stereochemical outcome in the synthesis of a polyketide other than monensin;

(13) a polypeptide encoded by a portion of the monensin gene cluster, preferably comprising at least one of mon BI and mon BII or a mutant or allele thereof, having carbon-carbon double bond isomerase activity;

(14) an epoxidase enzyme encoded by mon CI or a derivative or variant thereof having epoxidase activity;

(15) a cyclase enzyme encoded by mon CII or a derivative or variant thereof having cyclase activity.

Some embodiments of the invention will now be described by way of example with reference to the accompanying drawings in which:

FIG. 1 shows the structure of monensins A and B;

FIG. 2 illustrates proposed biosynthetic pathways;

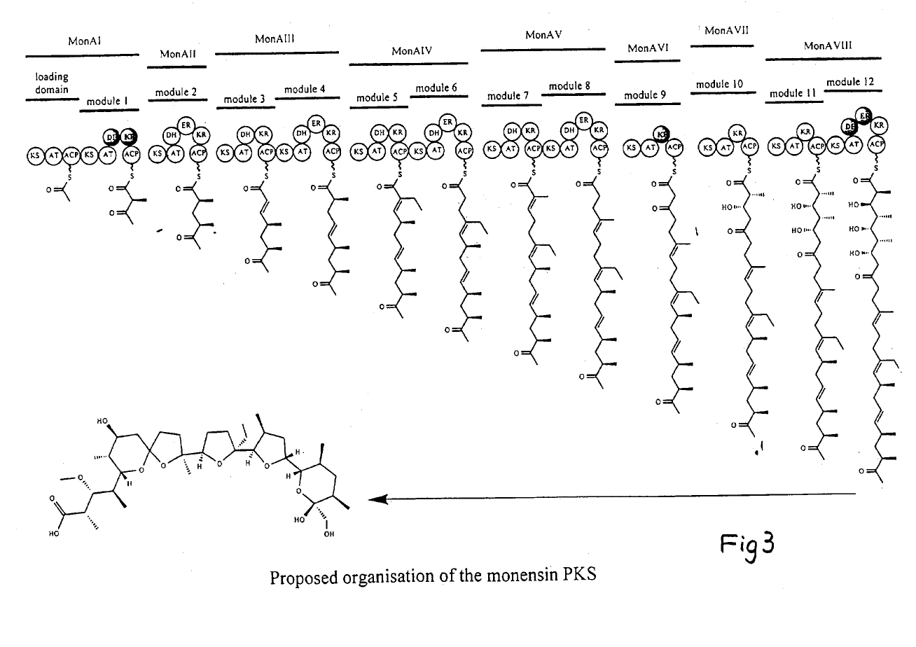

FIG. 3 illustrates the proposed organization of the monensin polyketide synthase (PKS) enzyme complex; and

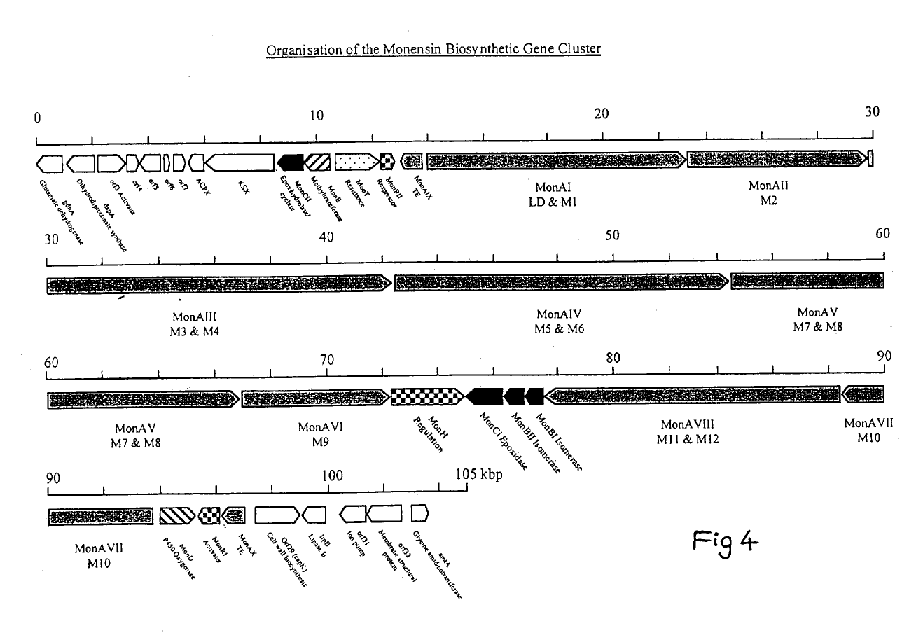

FIG. 4 illustrates the proposed organization of the monensin biosynthetic gene cluster.

The overall gene organization of the monensin biosynthetic gene cluster, as shown in FIG. 4, is similar to that previously found for many macrolide biosynthetic gene clusters, which have one or more open reading frames (ORFs) encoding large multifunctional PKSs flanked by other genes which encode functions required for the biosynthesis of the antibiotic. In the case of monensin, there is an unusually high number of distinct ORFs encoding PKS multi-enzymes (eight in total, labelled monAI to monAVIII) but there is again a separate module of enzymes for each cycle of polyketide chain extension, exactly as found for modular PKSs for macrolide biosynthesis (see FIG. 3). Thus there are 12 condensations predicted to be required for the production of the carbon skeleton of monensin, and in agreement with this there are found to be 12 extension modules of PKS enzymes distributed among the 8 PKS ORFs. However, as mentioned in detail below, the other genes in the monensin cluster include genes which have not previously been found in any other gene cluster for the biosynthesis of a complex polyketide, and which are not significantly similar to any genes in published sequence databases. The cloned DNA for these genes is useful to allow the diagnosis that a polyketide biosynthetic gene cluster in any actinomycete, uncovered previously by conventional hybridization against a PKS gene probe from (say) the DEBS or some other characterised PKS gene cluster, is one that governs the synthesis of a polyether; and these genes are also valuable either singly or in combination as specific hybridization probes for the specific detection and isolation of additional polyether biosynthetic gene clusters. Examples of these previously-unknown genes are the genes monBI, monBII, monCI and monCII. In addition the regulatory genes monH monRI, and monRII and the resistance gene monT and the thioesterase genes monAIX and monAX are all useful for the detection of analogous genes in other polyether clusters which are required for the rational manipulation of such genes in order to increase levels of the specific product.

The cloned and sequenced cluster of genes for monensin biosynthesis is useful secondly in the engineering of mutant strains of S. cinnamonensis and of other actinomycetes which are suitable strains for the high level production of either natural or novel recombinant polyketides. The sequence of the monensin cluster disclosed here shows the surprising fact, that the gene cluster contains a gene monRl whose gene product has an amino acid sequence highly similar to that of actII-orf4, the pathway-specific activator gene which activates the actI and other promoters of the actinorhodin biosynthetic gene cluster of Streptomyces coelicolor. The recognition of this aspect of the natural regulation of a Type I PKS cluster is important and valuable because first, it is possible to increase the yield of monensin by increasing the level of the activator MonRI, either by placing the gene monRI under the control of a powerful promoter or arranging for the presence within the cells of one or more additional copies of the monRI gene (as exemplified below); secondly, it will be possible to use the monRI gene as a specific hybridisation probe to locate similar genes in other complex PKS gene clusters, especially other polyether PKS gene clusters but also polyene and macrolide gene clusters and all other Type I modular PKS gene clusters; even in cases where (as for rapamycin and erythromycin) no such gene has been previously found within the currently accepted physical limits of the relevant biosynthetic gene cluster. In such cases the monRI gene probe might be expected to uncover the activator even if it resides on the chromosome at some distance from the main body of the gene cluster; and simple experiments would then show whether the activator(s) so uncovered are involved in regulation of the biosynthesis of those particular metabolites; thirdly, increasing the copy number of the monRI gene or of any of the activator genes uncovered will tend to increase the yield of a heterologous polyketide by “crosstalk” where the activator mimics the presence of the normal activator for the transcription of the genes for that heterologous polyketide synthase. It is clear from recently published work (Wietzorrek, A. and Bibb, M. Mol. Microbiol. (1997) 25:1181-1184) that the ActII-orf4 family of activators exert their effects by binding to promoter regions within the target gene cluster, so it will be possible to use the monRI gene together with monensin promoter regions to drive the high-level transcription and translation of heterologous genes in Streptomyces cinnamonensis, and perhaps in other host strains too; such genes need not be PKS genes or even involved in polyketide biosynthesis. Monensin promoter regions are found at the 5′ end of genes or groups of genes in the cluster and their location is clear from the sequence analysis disclosed here. Thus a useful vector would provide the monensin promoter and the ribosome binding site and continue up to the start of the open reading frame, after which the monensin ORF naturally found there would be replaced by the heterologous gene. The relative strength of the monensin promoters can be readily determined using any one of a number of known promoter probes, i.e. genes whose expression gives rise to readily measurable and quantifiable effects, such as Green Fluorescent Protein (GFP); or beta-galactosidase in the presence of a chromogenic substrate. It should be possible to mutate randomly the small region of the monensin promoters especially likely to interact with the MonRI activator (identified by the presence of tandem heptanucleotide repeats with a common consensus sequence between the various monensin promoters) (Wietzorrek, A. and Bibb, M. Mol. Microbiol. (1997) 25:1181-1184), and to determine the optimal DNA sequence for the maximal activation effect using either S. cinnamonensis (preferably—in case there are other unknown factors that make the activation function better in this strain than in other heterologous systems), or even in another host actinomycete strain. If the natural monensin promoters were mutated to have this optimal recognition sequence, then this would further increase the production of monensin. By extension, the use of this modified monensin promoter in conjunction with the monRI gene in heterologous systems could form the basis of further improvements in expression of polyketide synthases or other genes, either by appropriate chromosomal alterations to introduce the altered promoter and also the monRI gene; or by provision of vectors containing these optimised signals linked to specific genes and housed in suitable host cells.

The sequencing of the monensin cluster has uncovered another strategy for gene regulation in such Type I clusters. The previously-sequenced genes for the rapamycin biosynthetic pathway in Streptomyces hygroscopicus included a gene of unknown function (rapH). A closely similar gene has now been found in the monensin biosynthetic gene cluster (monH), and it is clear from this recurrence (and the comparison of the sequences with those of database proteins) that this gene is potentially an important DNA-binding sensor gene which acts to regulate the transcription of the cluster in concert with other regulatory signals. Simple experimentation is needed in order to define whether the gene is an activator, in which case putting in another copy or increasing its transcription will have the potential to increase polyketide biosynthesis; or alternatively the rapH gene product may be a negative regulator, whereupon deletion of this gene may release the biosynthetic pathway from this inhibitory effect and increase yields.

There is a continuing need to develop new methods of high-level production of bioactive metabolites and other valuable gene products in actinomycetes. Streptomyces cinnamonensis is a recognised and very valuable industrial strain for the production of very high levels of monensin, it is readily transformable with DNA by standard methods of conjugation or of protoplast transformation, it is a host for numerous known broad range plasmids including well-known expression plasmids of both high- and low-copy number, it also grows quickly relative to other actinomycete strains (for example about three times faster than wild type Saccharopolyspora erythraea the erythromycin producer, under comparable conditions) and sporulates relatively easily. Heterologous polyketides can be expressed in Streptomyces cinnamonensis using for example the low-copy number plasmid pCJR24 (which has no origin of replication active in actinomycetes so is maintained by integration into the chromosome) (Rowe, C. et al. Gene (1998) 216:215-223) or the related plasmid pCJR29 in which the polyketide synthase gene(s) are placed under the control of the acti promoter which is activated by the ActII-orf4 activator; or alternatively the monAI promoter can be substituted together with the MonRI activator; or some other pairing of activator and cognate promoter chosen from either a Type II or a Type I polyketide synthase gene cluster. As an example, the wild type strain of Streptomyces cinnamonensis has been used to express the plasmid pCJR29 (Rowe, C. et al. Gene (1998) 216:215-223) containing as insert the three ORFs for the PKS governing the production of 6-deoxyerythronolide B, the macrolide precursor of erythromycin A in Saccharopolyspora erythraea, these genes being placed under the control of the pathway-specific actI promoter from Streptomyces coelicolor together with its cognate activator gene actII-orf4. The transformed strain when cultivated in a suitable liquid medium produced 6-deoxyerythronolide B in good yield.

It is well known to the person skilled in the art that it is possible to use standard vectors unable to replicate in actinomycetes to introduce DNA into a Streptomyces cell, such DNA comprising two portions of contiguous DNA which are each identical to one of two portions of the cell's chromosome that are spaced up to 100 kbp apart; and that through recombination between the incoming DNA and the chromosome occurring in both portions of DNA the net result is that the chromosomal sequence is replaced by the defective sequence originally that of the incoming DNA. Such a procedure has been applied to the monensin-producing strain of S. cinnamonensis as described in detail below, and a strain of S. cinnamonensis has been obtained that carries a specific deletion in the monensin cluster and which is unable to produce the antibiotic. The use of such a strain facilitates the production of heterologous polyketides by removal of the background of monensin production.

The multiple uses of portions of the cloned and sequenced DNA from the monensin cluster will readily occur to the person skilled in the art. A surprising feature of the PKS of the monensin cluster is an unusual mechanism of polyketide chain initiation. We have found that the monensin PKS loading module has three domains, which from the amino-terminus of the protein are: a KSq domain, an acyltransferase domain and an ACP domain. We have uncovered this organisation in the PKS for the 14-membered macrolide oleandomycin as well as in the monensin PKS, an organisation of the loading module previously only found for the 16-membered macrolides and in which the KSq domain (which looks like a ketosynthase or condensation domain except that the active site cysteine residue is substituted by a glutamine for which the single letter notation is Q) had been previously speculated to have no function. It was realised that the acyltransferase of the loading module actually has malonyl-CoA and not acetyl-CoA as a substrate and that KSq is an active decarboxylase. It appears that a better discrimination can be achieved in the selection of the smaller acetate unit over propionate if the choice is made initially between methylmalonyl- and malonyl-CoA.

An unprecedented feature of the monensin PKS genes is that no integral chain-terminating domain is present as a C-terminal appendage of the PKS extension module that catalyzes the twelfth and final chain extension. Because the product of the monensin PKS is a carboxylic acid, it would have been firmly predicted that chain release would have been catalyzed by such a C-terminal domain containing a “thioesterase” activity. Previously sequenced PKS gene sets have been of two sorts: first, those macrolide PKSs typified by erythromycin, spiramycin, tylosin, niddamycin which have a readily recognisable C-terminal “thioesterase” domain, which in these enzymes functions as a specific cyclase rather than releasing the polyketide product as a free carboxylic acid; secondly, those macrolide PKSs typified by rapamycin, FK506, and rifamycin, where there is an alternative and recognised mode of chain termination by transfer of the polyketide chain to an acceptor moiety, catalyzed by a specific enzyme (eg pipecolate incorporating enzyme for rapamycin (Schwecke T. et al. Proc. Natl. Acad. Sci. USA (1995) 92:7839-7843) and FK506 (Mothamedi H. and Shafiee A, Eur. J. Biochemistry (1998) 256:528-534); arylamine synthetase for rifamycin (August P. R. et al. Chemistry & Biology (1998) 5:69-79).

The monensin PKS surprisingly falls into neither category, and therefore seems to be the first example of a novel mode of chain termination. It is novel and noteworthy in this connection that the monensin PKS gene cluster contains two small genes that encode discrete, monofunctional thioesterase enzymes. Although many PKS gene clusters have been previously shown to contain one such discrete thioesterase, none have been shown to have two. The role of such thioesterases is not known, although in the case of methymycin/pikromycin PKS, which has been reported to be responsible for the biosynthesis of both the 12-membered macrolide methymycin and the 14-membered macrolide pikromycin (Xue Y. Q. Proc. Natl. Acad. Sci. USA (1998) 95:12111-12116) the disruption of this thioesterase reportedly caused a ten-fold drop in the amount of both macrolides produced. A similar finding has been reported for the discrete thioesterase of the tylosin PKS gene cluster (Cundliffe E. et al. Chemistry & Biology in press). Additional copies of such thioesterases may therefore accelerate the production of specific polyketide, but this has not yet been demonstrated. However, the presence of the discrete thioesterase is not completely essential for polyketide production.

It is highly desirable to have a broadly effective method of catalysing the release of polyketide gene products from a PKS as the free acid. The well-studied integral thioesterase domain in the erythromycin PKS thioesterase has a broad specificity in cyclization to form a lactone (assuming that a hydroxy group is present in the growing polyketide chain at an appropriate position), but hydrolysis to form the free acid is very slow. The recognition of the unusual arrangement of the monensin PKS means that it is now possible to harness either the entire PKS module that catalyses the twelfth and final extension cycle in monensin biosynthesis, or the C-terminal portion of it, and graft it onto a different polyketide synthase by genetic engineering, so as to allow the release mechanism characteristic of monensin to operate in a different context. The use of this portion only of the monensin PKS suffices to allow the novel mechanism of chain release to operate successfully. The speed of the polyketide chain hydrolysis in a given case can depend on the additional presence of one or both of the discrete thioesterase genes (monAIX and monAX) from the monensin gene cluster. The use of this novel method of chain termination represents a valuable way of generating a large number of novel engineered polyketides that are currently inaccessible, and ensuring that the products have a specified chain length.

The genes monBI and monBII appear to encode very similar enzymes with significant amino acid sequence similarity to authentic ketosteroid isomerases which are known to catalyse the migration of an activated carbon-carbon double bond. The conservation of active site residues makes it very likely that these mon genes govern a reaction involving activated double bonds in the biosynthetic pathway to monensin and this surprising observation can be accommodated if the initial product of the polyketide chain growth on the monensin PKS is a linear precursor in which the double bonds were initially formed with a conventional trans or E (entgegen) geometry; but before the polyketide chain was extended by insertion of the next unit the monBI and/or the monBII gene product(s) catalyse the specific rearrangement of the newly-created double bond into the cis or Z (zusammen) geometry. This new view of the monensin biosynthetic pathway allows the deduction that the monBI and monBII genes, perhaps in combination with specific portions of the monensin modules where they normally exert their effects (namely modules 3, 5 and 7) might be used in order to achieve the extremely desirable targetted biosynthesis of novel polyketides containing double bonds with Z geometry at specified point(s) along the chain. Thus for example it should be possible to provide for the direct biosynthesis of C22-C23 cis or Z double bond in avermectins, thus avoiding tedious and expensive chemical conversion of an initial fermentation product into this important anthelminthic. Only limited experimentation is needed to see whether the monBI and/or monBII gene products are sufficient or whether the mon PKS at modules 3, 5 and 7 forms part of the specific docking site(s) for the isomerases and therefore must also be used in the creation of the hybrid PKS that will insert the cis or Z double bond at the desired position. The substrate specificity of the isomerases need not be limited to 2,3-unsaturated thioesters. The purified enzymes could also be used to effect such isomerisations in vitro, depending on the position of the equilibrium or whether further enzymes are used to achieve the further transformation of the product as it is formed (vide infra).

The product of the monCI gene is a novel oxidative enzyme with some sequence similarity to authentic examples of such enzymes in the databases; and with a clearly definable role in the monensin biosynthetic pathway, the epoxidation of the double bonds at three separate positions in the initially-formed acyclic intermediate in monensin biosynthesis. This epoxidase could therefore be used in conjunction with monBI/monBII gene products to effect oxidative reactions on suitable substrates in vitro and in vivo. Similarly the monCII gene product is a putative cyclase that opens the epoxides and causes the formation of ether rings in monensin.

Any or all of the monBI, monBII, monCI or monCII genes may be introduced into a heterologous strain containing the gene cluster for another polyether, in order to divert the biosynthetic pathway and produce a polyketide of altered structure. In these experiments the analogues of these monB genes could either be present or (once located and characterised using the mon genes as probes) they may be deleted prior to the introduction of the monB and monC genes into that strain. The converse experiment in which analogues of the monB and monC genes from other strains are introduced into S. cinnamonensis likewise has the potential to produce novel oxidised polyketides. Also, the monB and monC genes or their analogues may be introduced into a strain that normally produces a macrolide or a polyene or some other complex polyketide and expressed there, when they may effect the diversion of the growing polyketide chain on a heterologous modular PKS towards a new product, which may or may not have the structure of a polyether.

The availability of the monensin gene sequence allows the institution of domain swaps to alter the acyltransferase (AT) specificity of a given module, for example the ethylmalonyl-CoA specific extender found in one of the modules of the monensin PKS can be used to replace one of the other ATs to generate an ethyl side branch at that position in the chain, or the AT can be used to substitute in any other (e.g. macrolide) PKS, as described in WO 98/01571 and Wo 98/01546. Similarly the alteration of the level of reduction in a module, by manipulation of the reductive enzymes, can be applied to the monensin genes and here it will produce, depending on which module is affected, either an altered monensin, or a species which is only partly cyclised, or a polyether with an altered pattern of cyclisation, or even a linear polyketide.

In general the targetted alteration of the pattern of substitution of sidechains or reduction level along the polyketide chain produced by the monensin PKS will, like the disruption or deletion of the oxidative enzymes mentioned above, lead to non-polyether polyketide products. It should be possible, by introduction of the DEBS thioesterase at the C-terminus of one of the later modules of the monensin PKS, together with an appropriately placed hydroxy group earlier in the chain, to produce novel macrolide products from this polyether PKS system, or alternatively novel polyenes of defined chain length and chosen ring size.

### EXAMPLE 1

### Cloning of the Monensin A Biosynthetic Gene Cluster Using DNA Probes Derived From the Erythromycin-Producing Polyketide Synthase of Saccharopolyspora erythraea

A genomic library of the monensin A producing strain Streptomyces cinnamonensis ATCC 15413 was constructed using methods well-known in the art, namely, the production of high molecular weight genomic DNA, followed by the partial cleavage of this DNA using the frequent-cutting restriction enzyme Sau3A, fractionation of the fragments on a sucrose gradient and selection of fragments of average size 35-40 kbp, and the cloning of these fragments into the cosmid vector pWE15 (Evans, G. A. et al. Gene (1989) 79:9-20) which had been previously digested with BamHI and treated with shrimp alkaline phosphatase. The library was packaged and transfected into Escherichia coli XL-1 Blue MR cells. The library was plated out on 2×TY agar medium (10 g tryptone, 10 g yeast extract, 5 g NaCl, 15 g bactoagar per litre containing ampicillin 50 μg/ml) for cosmid selection and the colonies were allowed to grow overnight. The library was then screened by hybridisation using as a probe DNA encoding the ketosynthase domain of module 1 of the erythromycin-producing PKS (6-deoxyerythronolide B synthase, DEBS) of Saccharopolyspora erythraea. The colonies giving a positive hybridisation signal in the hybridisation were selected and the cosmid DNA from each colony was purified and mapped by restriction digestion. The presence of the target biosynthetic genes on a cosmid was verified by sequencing of the ends of the cosmid inserts using the commercially available T3 and T7 primers which hybridise specifically to the respective ends of each cosmid insert (Evans, G. A. et al. Gene (1989) 79:9-20).

### EXAMPLE 2

### Sequencing of the Biosynthetic Gene Cluster for Monensin A From Streptomyces cinnamonensis

Three cosmids obtained by screening of the genomic library of S. cinnamonensis were used to obtain the entire DNA sequence of the monensin biosynthetic gene cluster. These cosmids, MO.CN02, MO.CN11 and MO.CN33 between them contain the entire DNA sequence of the cluster and the adjacent regions of the chromosome. They have been deposited in NCIMB, 23 St Machair Drive, Aberdeen AB24 3RY, UK, under the NCIMB accession numbers 40956 (MO-CN11); 40957 (MO-CN33) and 40958 (MO-CN02) respectively.

The DNA of each cosmid was separately subjected to partial digestion with Sau3A and fragments of approximately 1.5-2.0 kbp were separated by agarose gel electrophoresis. The fragments were then ligated into the plasmid vector pUC18 (Messing, 1982), previously digested with BamHI and treated with shrimp alkaline phosphatase. The library was transformed into E. Coli strain XL1-Blue MR and plated on 2×TY agar medium containing ampicillin (100 μg/ml) to select for plasmid-containing cells. Plasmid DNA was purified from individual colonies and sequenced using the Sanger dye-terminator procedure on an ABI 377 automated sequencer (Sanger, F. Science (1981) 214:1205-1210). The sequence data obtained from single random subclones of a cosmid was assembled into a single continuous sequence and edited using GAP4.1 program of the STADEN gene analysis package (Staden, R. Molecular Biotechnology (1996) 5:233-241).

The sequence is set out in the appended sequence listing.

Tables I and II contain data about individual genes and gene products.

### EXAMPLE 3

### Inactivation of the Monensin A Biosynthetic Gene Cluster

A chromosomal gene disruption experiment was used to verify the identity of the cloned polyketide synthase gene cluster. Plasmid pMOB6314 is a pUC18 sequencing subclone of the presumed monensin A biosynthetic gene cluster prepared as described in Example 1, whose inserted DNA comprises the DNA sequence from nucleotide 9763 to nucleotide 10108 in SEQ ID 1, and which therefore contains a region of DNA wholly internal to orfE, a putative 3-O-methyltransferase. A HindIII fragment containing the thiostrepton resistance gene tsr from plasmid pIJ702 (Katz, E. et al. J. Gen. Microbiol. (1983) 129:2703-2714) was cloned into the HindIII site of plasmid pMOB6314 and the ligation mixture was used to transform E. coli cells. Transformants bearing the required plasmid pMOΔE01 were identified by isolation of plasmid DNA and analysis by restriction digestion. pMOΔE01. Plasmid pMOΔE01 was used to transform protoplasts of Streptomyces cinnamonensis as described by (Hopwood D. A. et al. (1985)). Since plasmid pMOΔE01 lacks an origin of replication that is active in Streptomyces, growth in the presence of thiostrepton (25 μg/ml) in the regeneration medium led to the isolation of stable integrants. Isolated putative integrants were tested for the presence of integrated pMOΔE01 sequences by Southern hybridisation. A clone of Streptomyces cinnamonensis identified by its restriction pattern in Southern hybridisation as bearing pMOΔE01 integrated in the region of monE of the monensin A biosynthetic gene cluster was designated S. cinnamonensis MO-DD01.

Detection of production of the monensin A related metabolites produced by S. cinnamonensis MO-DD01 was performed by GC-MS analysis of methanol extracts of the entire broth harvested in 72 hours of growth of the strain. No significant amounts of monensin A-related metabolite production were detectable.

### EXAMPLE 4

### Overproduction of Erythromycin Aglycone in Streptomyces cinnamonensis

S. cinnamonensis is a suitable system for overproduction not just of monensin A but also of other polyketide metabolites. Established techniques of genetic transformation allow fast introduction of foreign polyketide producing genes sets into this host. Fast growth of S. cinnamonensis in liquid culture and optimal precursor supply favour high yield of polyketide metabolites.

Construction of pIB061

S. erythraea NRRL2338 was transformed with pCJR30 (Rowe, C. J., et al. (1998) Gene 216:215-223) using a routine protoplast transformation technique as described by Hopwood et al. (1985). A stable integrant of S. erythraea [pCJR30] was identified and the production of 10 mg/L of the triketide lactone (delta lactone of (2S,3R,4R,5R)-2,4-dimethyl-3,5-dihydroxy-heptanoic acid) in addition to erythromycins was confirmed by MS analysis.

Total DNA of S. erythraea [pCJR30] was purified and approximately 200 ng was digested with EcoRI endonuclease. The digestion mixture was precipitated with isopropanol and the resulting DNA was treated with T4 DNA-ligase for 16 hours at 16° C. The ligation mixture was used to transform E.coli DH10B cells. The transformants were screened for the presence of the plasmid. A clone containing a 44.7kb plasmid was identified and confirmed by restriction analysis to contain three complete genes: eryAI, eryAII and eryAIII. The plasmid was named pIB061.

Transformation of S. cinnamonensis

Protoplasts of S. cinnamonensis were prepared by a modified procedure of Hopwood et al. (1985). Plasmid pIB061 was transformed into the protoplasts of S. cinnamonensis and stable thiostrepton resistant colonies were isolated. Individual colonies were checked for their plasmid content and the presence of plasmid pIB061 was confirmed by its restriction pattern. S. cinnamonensis (pIB061) was inoculated into 250 ml of M-C3 minimal production medium containing 10 μg/ml of thiostrepton and allowed to grow for 72 hours at 30° C. After this time the mycelia were removed by filtering. The broth was extracted with two volumes of ethyl acetate and the combined ethyl acetate extracts were washed with an equal volume of saturated sodium chloride, dried over anhydrous sodium sulphate, and the ethyl acetate was removed under reduced pressure to give about 200 mg of crude product. The product was analysed by LCQ and mass was confirmed to that of erythronolide B.

This example demonstrates the importance of S. cinnamonensis for production of high levels of foreign polyketide antibiotics. Introduction of the complete erythromycin gene cluster or other gene clusters into this system are likely to produce high levels of the corresponding metabolites.

### EXAMPLE 5

### Construction of Plasmid pCJW58 Containing the Monensin Activator Gene Under the ermE* Promoter

The ermE* promoter derived from the ermE resistance methyltransferase gene of S. erythraea (Bibb et al. Gene (1985) 38:215-226) was amplified by PCR as a SpeI-XbaI fragment using the following oligonucleotides 5′-CCACTAGTATGCATGCGAGTGTCCGTTCGAGT-3′ and 5′-TTGTATACACCTAGGATGGTTGGCCGTGC-3′ with pRH3 (Dhillon et al. Molecular Microbiology (1989) 3:1405-1414 as a template and cloned into SmaI-digested, phosphatase-treated pUC18, to produce plasmid pIB135. The integrative plasmid pSET152 (Bierman, M. et al. (1992) Gene 116:43-49)) was digested with XbaI and the backbone was dephosphorylated and ligated to the SpeI-XbaI fragment of pIB135 containing the ermE* promoter. The ligation mixture was used to transform E. coli DH10B and the orientation of the insert in the plasmids from individual clones was checked by using restriction analysis. A plasmid with the ermE* promoter oriented so that the NdeI and XbaI sites are adjacent to the apramycin resistance gene was selected and named pIB139.

The monR gene from the monensin biosynthetic gene cluster was amplified and NdeI and XbaI restriction sites introduced at 5′ and 3′ ends respectively, by PCR using as primers the following oligonucleotides: 5′-AGA TAC CAT ATG CTG GGC CCG CTC CGC AT-3′ and 5′-AAT GCT CTA GAC TGT CAG CGA CCG GAC AGG GCC AA-3′ and cosmid MO.CN11 as template. The PCR product was ligated into SmaI-treated and phosphatase-treated plasmid pUC18 and the ligation mixture was used to transform E. coli DH10B cells. Transformant colonies were analysed for the presence of plasmid and the identity of the plasmid inserts was verified by sequencing. A plasmid whose insert contained the monR gene flanked by NdeI and XbaI restriction sites was selected and designated pCJW57.

Plasmid pCJW57 was digested with NdeI and XbaI and the fragment containing the monR gene was ligated together with the backbone of plasmid pIB139 which had been digested with the same two restriction enzymes, and purified by gel elution. The ligation mixture was used to transform E. coli strain DH10B cells. Transformant colonies were analysed for the presence of plasmid and the identity of the plasmid inserts was verified by restriction analysis. One such recombinant was selected and named plasmid pCJW58.

Plasmid pCJW58 was used to transform the methylation-deficient E. coli strain ET 12567 (MacNeil D. J. et al. (1992) Gene 111:61-68) and the recovered, unmethylated plasmid was then used to transform the same E. coli strain ET12567 housing the plasmid pUB307, a derivative of RP4 which is mob and which contains a gene for kanamycin resistance (Piffaretti, J. C. et al. (1988) Mol. Gen. Genet. 212:215-218). Recombinants were plated on 2×TY agar medium containing apramycin and kanamycin at final concentrations of 50 micrograms per ml and 50 micrograms per ml respectively. The plasmid content of recombinants was checked isolation of plasmid DNA and checking of the identity of these plasmids by restriction analysis. One such clone which contained both pUB307 and plasmid pCJW58 was selected and used for further experiments.

Construction of Streptomyces cinnamonensis (pCJW58) and production of monensins

A single colony of E. coli ET12567 housing both pUB307 and pCJW58 was toothpicked into 3 ml of TY liquid medium, containing apramycin and kanamycin at 25 and 25 micrograms respectively, and grown overnight at 37° C. This culture was used to inoculate 25 ml of TY medium, supplemented with the same antibiotics at the same concentrations, and growth was continued until the absorbance at 600 nm (1 cm pathlength) was between 0.3-0.6. The cells were centrifuged (room temperature, 7 minutes, 2000×g), resuspended in TY liquid medium (10 ml) containing no added antibiotics, re-centrifuged as before, then resuspended in 2 ml of TSB medium and placed on ice. Meanwhile, 0.5 ml of TSB medium was added to 100 microL containing approximately 10⁸ spores of S. cinnamonensis. After a brief heat shock, at 50° C. for 10 minutes, the suspension was briefly cooled, mixed with 0.5 ml of donor E. coli cells, and plated on solid A medium, which has composition as follows:

A medium

| | |
|---|---|
| Sigma wheat starch | 5 g |
| Corn steep powder | 1.25 g |
| Yeast extract | 1.5 g |
| CaCO₃ | 1.5 g |
| FeSO₄ | 6 mg |
| DIFCO agar | 10 g |
| H₂O | to 500 ml |

pH adjusted to pH 7 with KOH.

And to which in addition was added 10 mM MgCl₂ to a final concentration of 10 mM.

The plates were allowed to dry overnight at room temperature, and were then allowed to incubate a further 18 hours at 30° C. After this time each 25 ml plate was overlaid with a solution of apramycin (final concentration 50 micrograms per ml) and nalidixic acid (final concentration 20 micrograms per ml), and the plates were allowed to incubate for four days at 30° C. At this time individual colonies were toothpicked onto solid A medium and allowed to grow. Four representative colonies from the A medium plate were grown up in liquid modified YEME medium, which has composition as follows:

Modified YEME medium

| | |
|---|---|
| Sucrose | 100 g |
| DIFCO Yeast extract | 3 g |
| Bacto peptone | 5 g |
| Oxoid Malt extract | 3 g |
| Glucose | 10 g |
| H₂O to 1 L | |

pH adjusted to pH 7.2 with NaOH.

These cultures were used to provide a 2% vol/vol inoculum for 30 ml of modified YEME which was grown for 7 days, and then transferred to SM16 medium, which has composition as follows:

SM16 medium

| | |
|---|---|
| 3-[N-Morpholino]-propane sulfonic acid | 20.9 g |
| (MOPS) buffer | |
| L-proline | 10.0 g |
| Glucose | 20 g |
| NaCl | 0.5 g |
| K₂HPO₄ | 2.1 g |
| Ethylenediaminetetraacetic acid, sodium | 0.25 g |
| salt | |
| MgSO₄.7H₂O | 0.49 g |
| CaCl₂.2H₂O | 0.029 g |
| Trace elements solution (Hopwood, D. A. | 2 ml |
| et al. (1985) Genetic Manipulation | |
| of Streptomyces - a Laboratory Manual, | |
| at p.235) | |
| 0.5 M CoCl₂ solution | 2 microliters |
| H₂0 to 1 L | |

pH adjusted to pH 7 with NaOH.

After growth for a further 7 days, mycelium was collected by centrifugation at 2000×g for 30 minutes, and the supernatant was extracted three times with 300 ml of ethyl acetate. The combined extracts were concentrated by evaporation under reduced pressure to an oil, which was mixed with 1 ml of methanol. Samples were applied to an LCQ liquid chromatograph fitted with a mass spectrometer detector unit. The column used was a C18 reversed phase column, equilibrated with a mixture of 80% 20 mM ammonium acetate/20% acetonitrile, and the column was eluted with a gradient of increasing acetonitrile, reaching 100% acetonitrile over 24 minutes. Monensins A and B emerged from the column with retention times respectively of 8.2 minutes and 9.2 minutes. The relative amounts of monensin produced by three independent clones (A-C) containing an additional copy of the monr gene were compared to a control fermentation of the wild type S. cinnamonensis strain, with the results shown in the Table below:

Table showing increased monensin production in strains
bearing additional copy of monR gene

| | monensin A | monensin B |
|---|---|---|
| | concentration | concentration |
| Strain | (arbitrary units) | (arbitrary units) |
| Control | 188 | 861 |
| A | 430 | 1 800 |
| B | 450 | 1 300 |
| C | 249 | 1 300 |

### EXAMPLE 6

### Construction of S. cinnamonensis M12AT5

A region lying immediately 5′ of the DNA encoding the acyltransferase (AT12) domain of module 12 of the monensin polyketide synthase in the mbnensin biosynthetic gene cluster was amplified with the following primers: 5′-GGTGGCCACGGAAACACCAACACCGGACCCGCGCC-3′, and 5′-CTCTCGGAGGCCCGGCGCAACGGCCACAA-3′, 3′ using casmid MO-CN11 as a template. The PCR product was ligated into SmaI digested and phosphatase-treated plasmid pUC18 and the ligation mixture was used to transform E. coli DH10B cells. Transformant colonies were analysed for the presence of plasmid and the identity of the plasmid inserts was verified by sequencing. A plasmid whose insert contained a fragment upstream of the AT12-encoding sequence from about 82.3kb to 83.2kb of the mon cluster was designated pMO81. Similarly a region lying immediately 3′ of the DNA encoding the acyltransferase (AT12) domain of module 12 of the monensin polyketide synthase in the monensin biosynthetic gene cluster was amplified with the following primers: 5′-GGCCTAGGGCTGCCTCGGGTGGTGGATCTGCCGA-3′ and 5′- TGGTCGGGCGCGGTGCGTGCGATACGT-3′, using cosmid MO-CN11 as a template. The PCR product was ligated into SmaI-treated and dephosphorylated pUC18 and the ligation mixture was used to transform DH10B E.coli cells. Transformant colonies were analysed for the presence of plasmid and the identity of the plasmid inserts was verified by sequencing. A plasmid whose insert contained a fragment downstream of the AT12-encoding sequence, from 80.5kb to 81.4kb of the mon cluster, was designated pMO82.

The DNA encoding AT of module 5 was amplified and MscI and AvrII restriction enzyme recognition sites were introduced at the ends by PCR using the following primers: 5′-CCTGGCCAGGGCGGCCAGTGGGTGGGCATG-3′ and 5′-GGCCTAGGGGTCGGCCGGGAACCAGCGCCGCCAGT-3′ and the cosmid MO-CN33 as a template. The PCR product was ligated into SmaI-treated and dephosphorylated pUC18 and the ligation mixture was used to transform DH10B E.coli cells. Transformant colonies were analysed for the presence of plasmid and the identity of the plasmid inserts was verified by sequencing. A plasmid whose insert DNA, with sequence from about 44.2kb to 45.2kb of the mon cluster, encoded the AT5 domain was designated pMO83.

pMO81 was digested with MscI and HindIII and ligated to the 0.9kb MscI-HindIII fragment of pMO82. A clone containing both fragments was designated pMO84. Plasmid pMO84 was cleaved with AvrII and HindIII, treated with phosphatase, and ligated together with the 1.0 kb AvrII-HindIII fragment of pMO83 to produce pMO85, which contains the DNA encoding the AT5 domain flanked by DNA from either side of the DNA encoding the AT12 domain of the monensin PKS. The thiostrepton resistance gene tsr, derived from plasmid pIJ702 (Katz, E. et al., J. Gen. Microbiol. 1983), was cloned into the HindIII site of pMO85. The resulting plasmid pMO86 was analysed by its restriction pattern and confirmed to contain all the desired elements.

Plasmid pMO86 was used to transform S. cinnamonensis protoplasts as described by Hopwood, D. A. (1985). Stable thiostrepton-resistant transformants were isolated and checked for the desired integration of the pMO85 into the AT12 flanking regions by Southern blot hybridisation. One such integrant, S. cinnamonensis MO-08, containing pMO85 integrated upstream of the AT12, was passed through 4 cycles of sporulation on a non-selective nutrient medium. Spores obtained after the fourth cycle were replica-plated onto media with and without thiostrepton. DNA of clones that had lost thiostrepton resistance was analysed by Southern blot hybridisation. Clones in which the DNA encoding the AT12 domain had been replace by the DNA encoding the AT5 domain was designated S. cinnamonensis M12-AT5. At this time individual colonies were toothpicked onto solid A medium and allowed to grow. Four representative colonies from the A medium plate were grown up in liquid modified YEME medium, which has composition as follows:

Modified YEME medium

| | |
|---|---|
| Sucrose | 100 g |
| DIFCO Yeast extract | 3 g |
| Bacto peptone | 5 g |
| Oxoid Malt extract | 3 g |
| Glucose | 10 g |
| H₂O to 1 L | |

pH adjusted to pH 7.2 with NaOH.

These cultures were used to provide a 2% vol/vol inoculum for 30 ml of modified YEME which was grown for 7 days, and then transferred to SM16 medium, which has composition as follows:

SM16 medium

| | |
|---|---|
| 3-[N-Morpholino]-propane sulfonic | 20.9 g |
| acid (MOPS) buffer | |
| L-proline | 10.0 g |
| Glucose | 20 g |
| NaCl | 0.5 g |
| K₂HPO₄ | 2.1 g |
| Ethylenediaminetetraacetic acid, | 0.25 g |
| sodium salt | |
| MgSO₄.7H₂O | 0.49 g |
| CaCl₂.2H₂O | 0.029 g |
| Trace elements solution (Hopwood, D. A. | 2 ml |
| et al. (1985) Genetic | |
| Manipulation of Streptomyces - a | |
| Laboratory Manual, at p. 235) | |
| 0.5 M CoCl₂ solution | 2 microliters |
| H₂0 to 1 L | |

pH adjusted to pH 7 with NaOH.

After growth for a further 7 days, mycelium was collected by centrifugation at 2000×g for 30 minutes, and the supernatant was extracted three times with 300 ml of ethyl acetate. To confirm presence of the C-2-ethyl substituents of both monensin A and B the combined extracts were concentrated by evaporation under reduced pressure to an oil, which was mixed with 1 ml of methanol. Samples were applied to an LCQ liquid chromatograph fitted with a mass spectrometer detector unit. The column used was a C18 reversed phase column, equilibrated with a mixture of 80% 20 mM ammonium acetate/20% acetonitrile, and the column was eluted with a gradient of increasing acetonitrile, reaching 100% acetonitrile over 24 minutes. Mass ions 14 mass units above those expected for both monensin A and B confirmed production of the respective C-2-ethyl substituents.

### EXAMPLE 7

### Construction of pSGK005 and Its Use in the Production of C-13 Propyl-Erythromycin

Plasmid pSGK005 is a pCJR24 based plasmid containing a PKS gene comprising a loading module plus the first and second extension modules and the chain terminating thioesterase of the PKS responsible for the production of erythromycin (DEBS). The loading module comprises the KS and ethyl-malonyl CoA specific AT from module 5 of the monensin PKS linked to the DEBS loading ACP domain. In addition, the active site cysteine of this module 5 KS has been mutated to glutamine to convert an extender di-domain to a loading di-domain. Plasmid pSGK005 was constructed as follows.

A 2769 bp DNA segment of the monensin cluster of S. cinnamonensis extending from nucleotide 42438 to 45207 was amplified by PCR using the following oligonucleotide primers. 5′-GTGACGTCATATGTCGAGTGCTGAAGAGTCG-3′ and 5′-GGGGTCGCCTAGGAACCAGCGCCGCCAGTCGA-3′

The design of these primers introduced Nde I and Avr II sites at the ends of the amplifed fragment. Monensin cosmid 05 was used as a template for the reaction. The resulting 2769 bp fragment was digested with Nde I and Xho I and a 656 bp fragment (Fragment A) purified by preparative gel electrophoresis.

A second PCR reaction was used with the same template to amplify DNA from nucleotide 43098 to 45207. The primers used were

5′-CGGCCTCGAGGGCCCGTCGGTCAGTGTCGACACGGCGCAGTCCTCCT
CGC-3′
and
5′-GGGGTCGCCTAGGAACCAGCGCCGCCAGTCGA-3′

The design of the upstream oligonucleotide primer incorporated a change of the codon specifying the KS active site cysteine (nucleotides 43135-43137, TGC) to glutamine (CAG). The resulting 2109 bp DNA fragment (Fragment B) was digested with Xho I and Avr II and purified by preparative gel electrophoresis.

Plasmid pCJW80 is derived from pCJR24 and DEBS1-TE in which Msc I and Avr II sites have been introduced to flank the AT of the DEBS loading module. This plasmid was digested with Nde I and Avr II and the larger fragment (Fragment C) purified by preparative gel electrophoresis.

The three fragments (Fragments A, B, C) were ligated together using T4 DNA ligase and the ligation mixture used to transform electrocompetent E. coli DH10B cells. Individual clones were checked for the presence of the desired plasmid pSGK005. The identity of pSGK005 was confirmed by restriction pattern and sequence analysis.

Plasmid pSGK005 was used to transform S. erythraea NRRL2338 using a routine protoplast transformation technique. Thiostrepton resistant colonies were selected on R2T20 media containing g/ml thiostrepton. Further analysis confirmed that pSGK005 had integrated into the S. erythraea NRRL2338 chromosome by Southern blot hybridisation of their genomic DNA with DIG-labelled DNA containing the actII orf4 promoter. The culture S. erythraea NRRL2338 (pSGK005) was inoculated into 5 ml tap water medium in a 30 ml flask. After three days incubation at 29° C. this flask was used to inoculate 30 ml of Ery-P medium in a 300 ml flask. The broth was incubated at 29° C. at 200rpm for 6 days. After this time the whole broth was adjusted to pH8.5 with NaOH, and then extracted twice with an equal volume of ethyl acetate. The ethyl acetate extract was evaporated to dryness at 45° C. under a nitrogen stream using a Zymark Turbovap LV evaporator. The product identities were confirmed by LC/MS. A peak was observed with a m/z value of 734 (M+H)⁺ required for erythromycin A. A second peak was observed with a m/z value of 748 (M+H)⁺, required for 13-propyl erythromycin A.

### REFERENCES

1. Ajaz, A. A. and Robinson, J. A. (1983) The utilization of oxygen atoms from molecular oxygen during the biosynthesis of Monensin-A. Journal of the Chemical Society-Chemical Communications, 12, 679-680.

2. Ashworth, D. M., Holmes, D. S., Robinson, J. A., Oikawa, H. and Cane, D. E. (1989) Selection of a specifically blocked mutant of Streptomyces cinnamonensis—isolation and synthesis of 26-deoxymonensin-A. Journal of Antibiotics, 42, 1088-1099.

3. August, P. R., Tang, L., Yoon, Y. J., Ning, S., Muller, R., Yu, T. W., Taylor, M., Hoffmann, D., Kim, C. G., Zhang, X. H., Hutchinson, C. R. and Floss, H. G. (1998) Biosynthesis of the ansamycin antibiotic rifamycin: deductions from the molecular analysis of the rif biosynthetic gene cluster of Amycolatopsis mediterranei S699. Chemistry & Biology, 5, 69-79.

4. Bartel, P. L., Zhu, C. B., Lampel, J. S., Dosch, D. C., Connors, N. C., Strohl, W. R., Beale, J. M. and Floss, H. G. (1990) Biosynthesis of anthraquinones by interspecies cloning of actinorhodin biosynthesis genes in Streptomycetes—clarification of actinorhodin gene functions. Journal of Bacteriology, 172, 4816-4826.

5. Bibb, M. J., Biro, S., Motamedi, H., Collins, J. F. and Hutchinson, C. R. (1989) Analysis of the nucleotide sequence of the Streptomyces glaucescens Tcml genes provides key information about the enzymology of polyketide antibiotic biosynthesis. EMBO Journal, 8, 2727-2736.

6. Bibb, M. J. and Hopwood, D. A. (1981) Genetic studies of the fertility plasmid SCP2 and its SCP2* variants in Streptomyces coelicolor A3(2). Journal of General Microbiology, 126, 427-442.

6a. Bibb M. J., Janssen G. R. and Ward J. M. (1985) Cloning and analysis of the promoter region of the erythromycin resistance gene (ErmE) of Streptomyces erythraeus. Gene, 38, 215-226.

6b. Bierman, M., Logan, R., O'Brien, K., Seno, E. T., Rao R. N. and Schoner B. E. (1992) Plasmid cloning vectors for the conjugal transfer of DNA from Escherichia coli to Streptomyces spp. Gene, 116, 43-49.

7. Cane, D. E., Liang, T. C. and Hasler, H. (1981) Polyether biosynthesis—origin of the oxygen atoms of Monensin-A. Journal of the American Chemical Society, 103, 5962-5965.

8. Cane, D. E., Liang, T. C. and Hasler, H. (1982) Polyether biosynthesis 2. Origin of the oxygen atoms of monensin A. Journal of the American Chemical Society, 104, 7274-7281.

9. Cortés, J., Haydock, S. F., Roberts, G. A., Bevitt, D. J. and Leadlay, P. F. (1990) An unusually large multifunctional polypeptide in the erythromycin producing polyketide synthase of Saccharopolyspora erythraea. Nature, 348, 176-178.

10. Cortés, J., Wiesmann, K. E. H., Roberts, G. A., Brown, M. J. B., Staunton, J. and Leadlay, P. F. (1995) Repositioning of a domain in a modular polyketide synthase to promote specific chain cleavage. Science, 268, 1487-1489.

11. Davis, N. K. and Chater, K. F. (1990) Spore color in Streptomyces coelicolor A3(2) involves the developmentally regulated synthesis of a compound biosynthetically related to polyketide antibiotics. Molecular Microbiology, 4, 1679-1691.

12. Day, L. E. (1973) Antimicrobial Agents and Chemotherapy, 4, 410-414.

13. Demetriadou, A. K., Laue, E. D., Staunton, J., Ritchie, G. A. F., Davies, A. and Davies, A. B. (1985) Biosynthesis of the polyketide polyether antibiotic ICI-139603 in Streptomyces longisporoflavus from O-18-labeled acetate and propionate. Journal of the Chemical Society Chemical Communications, 7, 408-410.

13a. Dhillon, N., Hale, R. S., Cortes, J. and Leadlay P. F. (1989) Molecular characterization of a gene from Saccharopolyspora erythraea (Streptomyces erythraeus) which is involved in erythromycin biosynthesis. Molecular Microbiology 3, 1405-1414.

14. Donadio, S., McAlpine, J. B., Sheldon, P. J., Jackson, M. and Katz, L. (1993) An erythromycin analog produced by reprogramming of polyketide synthesis. Proceedings of the National Academy of Sciences of the United States of America, 90, 7119-7123.

15. Donadio, S., Staver, M. J., McAlpine, J. B., Swanson, S. J. and Katz, L. (1991) Modular organization of genes required for complex polyketide biosynthesis. Science, 252, 675-679.

16. Evans, G. A., Lewis, K. and Rothenberg, B. E. (1989) High efficiency vectors for cosmid microcloning and genomic analysis. Gene, 79, 9-20.

17. Fernandez-Moreno, M. A., Caballero, J. L., Hopwood, D. A. and Malpartida, F. (1991) The Act cluster contains regulatory and antibiotic export genes, direct targets for translational control by the blda transfer-RNA gene of Streptomyces. Cell, 66, 769-780.

18. Fernandez-Moreno, M. A., Martinez, E., Boto, L., Hopwood, D. A. and Malpartida, F. (1992) Nucleotide sequence and deduced functions of a set of cotranscribed genes of Streptomyces coelicolor A3(2) including the polyketide synthase for the antibiotic actinorhodin. Journal of Biological Chemistry, 267, 19278-19290.

19. Gramajo, H. C., Takano, E. and Bibb, M. J. (1993) Stationary phase production of the antibiotic actinorhodin in Streptomyces coelicolor A3(2) is transcriptionally regulated. Molecular Microbiology, 7, 837-845.

20. Gumila, C., Ancelin, M. L., Delort, A. M., Jeminet, G. and Vial, H. J. (1997) Characterization of the potent in vitro and in vivo antimalarial activities of ionophore compounds. Antimicrobial Agents and Chemotherapy, 41, 523-529.

21. Hallam, S. E., Malpartida, F. and Hopwood, D. A. (1988) Nucleotide sequence, transcription and deduced function of a gene involved in polyketide antibiotic synthesis in Streptomyces coelicolor. Gene, 74, 305-320.

22. Holmes, D. S., Sherringham, J. A., Dyer, U. C., Russell, S. T. and Robinson, J. A. (1990) Synthesis of putative intermediates on the monensin biosynthetic pathway and incorporation experiments with the monensin-producing organism. Helvetica Chimica Acta, 73, 239-259.

23. Hopwood, D. A., Bibb, M. J., Chater, K. F., Kieser, T., Bruton, C. J., Kieser, H. M., Lydiate, D. J., Smith, C. P., Ward, J. M. and Schrempf, H. (1985) Genetic manipulation of Streptomyces, a laboratory manual. John Innes Institution, Norwich, UK.

24. Hutchinson, C. R. and Fujii, I. (1995) Polyketide synthase gene manipulation—a structure function approach in engineering novel antibiotics. Annual Review of Microbiology, 49, 201-238.

25. Hutchinson, C. R., Sherman, M. M., Vederas, J. C. and Nakashima, T. T. (1981) Biosynthesis of macrolides.5. Regiochemistry of the labeling of lasalocid a by C-13, O-18-labeled precursors. Journal of the American Chemical Society, 103, 5953-5956.

26. Kakavas, S. J., Katz, L. and Stassi, D. (1997) Identification and characterization of the niddamycin polyketide synthase genes from Streptomyces caelestis. Journal of Bacteriology, 179, 7515-7522.

27. Kao, C. M., Luo, G. L., Katz, L., Cane, D. E. and Khosla, C. (1995) Manipulation of macrolide ring size by directed mutagenesis of a modular polyketide synthase. Journal of the American Chemical Society, 117, 9105-9106.

28. Katz, E., Thompson, C. J. and Hopwood, D. A. (1983) Cloning and expression of the tyrosinase gene from Streptomyces antibioticus in Streptomyces lividans. Journal of General Microbiology, 129, 2703-2714.

29. MacNeil, D. J., Occi, J. L., Gewain, K. M., Macneil, T., Gibbons, P. H., Ruby, C. L. and Danis, S. J. (1992) Complex organization of the Streptomyces avermitilis genes encoding the avermectin polyketide synthase. Gene, 115, 119-125.

29a. MacNeil, D. J., Gewain, K. M., Ruby, C. L., Dezeny, G., Gibbons, P. H. and MacNeil, T. (1992) Analysis of Streptomyces avermitilis genes required for avermectin biosynthesis utilizing a novel integration vector. Gene 111, 61-68.

30. Marsden, A. F. A., Wilkinson, B., Cortés, J., Dunster, N. J., Staunton, J. and Leadlay, P. F. (1998) Engineering broader specificity into an antibiotic-producing polyketide synthase. Science, 279, 199-202.

31. Meurer, G., Gerlitz, M., Wendt Pienkowski, E., Vining, L. C., Rohr, J. and Hutchinson, C. R. (1997) Iterative type II polyketide synthases, cyclases and ketoreductases exhibit context-dependent behavior in the biosynthesis of linear and angular decapolyketides. Chemistry & Biology, 4, 433-443.

32. Motamedi, H. and Shafiee, A. (1998) The biosynthetic gene cluster for the macrolactone ring of the immunosuppressant FK506. European Journal of Biochemistry, 256, 528-534.

33. Narva, K. E. and Feitelson, J. S. (1990) Nucleotide sequence and transcriptional analysis of the RedD locus of Streptomyces coelicolor A3(2). Journal of Bacteriology, 172, 326-333.

33a. Piffaretti J. C., Arini A. and Frey J. (1988) pUB307 mobilizes resistance plasmids from Escherichia coli into Neisseria gonorrhoeae. Mol Gen Genet. 212, 215-218.

34. Pospisil, S., Sedmera, P., Vokoun, J., Vanek, Z. and Budesinsky, M. (1987) 3-O-Demethylmonensin-A and 3-O-demethylmonensin-B produced by Streptomyces cinnamonensis. Journal of Antibiotics, 40, 555-557.

35. Revill, W. P., Bibb, M. J. and Hopwood, D. A. (1995) Purification of a malonyltransferase from Streptomyces coelicolor A3(2) and analysis of its genetic determinant. Journal of Bacteriology, 177, 3946-3952.

36. Rowe, C. J., Cortés, J., Gaisser, S., Staunton, J. and Leadley, P. F. (1998) Construction of new vectors for high-level expression in actinomycetes. Gene, 216, 215-223.

37. Sanger, F. (1981) Determination of nucleotide sequences in DNA. Science, 214, 1205-1210.

38. Schwecke, T., Aparicio, J. F., Molnar, I., Konig, A., Khaw, L. E., Haydock, S. F., Oliynyk, M., Caffrey, P., Cortés, J., Lester, J. B., Böhm, G. A., Staunton, J. and Leadlay, P. F. (1995) The biosynthetic gene cluster for the polyketide immunosuppressant rapamycin. Proceedings of the National Academy of Sciences of the United States of America, 92, 7839-7843.

39. Shen, B., Summers, R. G., Wendtpienkowski, E. and Hutchinson, C. R. (1995) The Streptomyces glaucescens TcmKl polyketide synthase and TcmN polyketide cyclase genes govern the size and shape of aromatic polyketides. Journal of the American Chemical Society, 117, 6811-6821.

40. Sherman, D. H., Malpartida, F., Bibb, M. J., Kieser, H. M. and Hopwood, D. A. (1989) Structure and deduced function of the granaticin-producing polyketide synthase gene cluster of Streptomyces violaceoruber Tu22. EMBO Journal, 8, 2717-2725.

41. Sood, G. R., Robinson, J. A. and Ajaz, A. A. (1984) Biosynthesis of the polyether antibiotic Monensin-A—incorporation of [2-2-2H-2]-propionate, (R)-[2-2H-1]-propionate and (S)-[2-2H-1]- propionate. Journal of the Chemical Society-Chemical Communications, 21, 1421-1423.

42. Spavold, Z., Robinson, J. A. and Turner, D. L. (1986) Biosynthesis of the polyether antibiotic narasin origins of the oxygen atoms and the mechanisms of ring formation. Tetrahedron Letters, 27, 3299-3302.

43. Staden, R. (1996) The Staden sequence analysis package. Molecular Biotechnology, 5, 233-241.

44. Stutzman-Engwall, K. J., Otten, S. L. and Hutchinson, C. R. (1992) Regulation of secondary metabolism in Streptomyces Spp and overproduction of daunorubicin in Streptomyces peucetius. Journal of Bacteriology, 174, 144-154.

45. Swan, D. G., Rodriguez, A. M., Vilches, C., Mendez, C. and Salas, J. A. (1994) Characterization of a Streptomyces antibioticus gene encoding a type I polyketide synthase which has an unusual coding sequence. Molecular & General Genetics, 242, 358-362.

46. Takano, E., Gramajo, H. C., Strauch, E., Andres, N., White, J. and Bibb, M. J. (1992) Transcriptional regulation of the redD transcriptional activator gene accounts for growth phase-dependent production of the antibiotic undecylprodigiosin in Streptomyces coelicolor A3(2). Molecular Microbiology, 6, 2797-2804.

47. Townsend, C. A. and Basak, A. (1991) Experiments and speculations on the role of oxidative cyclization chemistry in natural product biosynthesis. Tetrahedron, 47, 2591-2602.

48. Walba, D. M. and Edwards, P. D. (1980) Tetrahedron Letters, 21, 3531-3534.

49. Westley, J. W. (1974) Journal of Antibiotics, 27, 597-604.

50. Westley, J. W. (1981) Antibiotics IV. Biosynthesis. Springer-Verlag, New York.

51. Wietzorrek, A. and Bibb, M. (1997) A novel family of proteins that regulates antibiotic production in Streptomycetes appears to contain an OmpR-like DNA-binding fold. Molecular Microbiology, 25, 1181-1184.

52. Xue, Y. Q., Zhao, L. S., Liu, H. W. and Sherman, D. H. (1998) A gene cluster for macrolide an antibiotic biosynthesis in Streptomyces venezuelae: Architecture of metabolic diversity. Proceedings of the National Academy of Sciences of the United States of America, 95, 12111-12116.

TABLE I

| gene | function | start | end |
|---|---|---|---|
| gdhA | glutamate dehydrogenase (partial) | 1038 | 0 |
| dapA | dihydrodipicolinate synthase | 2140 | 1220 |
| orf3 | putative transcriptional activator | 2211 | 3152 |
| orf4 | hypothetical protein | 3264 | 3680 |
| orf5 | hypothetical protein | 4307 | 3684 |
| orf6 | hypothetical protein | 4570 | 4758 |
| orf7 | hypothetical protein | 5058 | 5612 |
| acpX | acyl carrier protein | 6010 | 5693 |
| ksX | ketoacyl synthase | 8531 | 6045 |
| monCI | probable epoxihydrolase/cyclase | 9542 | 8643 |
| monE | methyltransferase | 10426 | 9596 |
| monT | monensin resistance gene (ABC- | 10656 | 12191 |
| monRI | probable repressor | 12205 | 12780 |
| monAI | thioesterase | 13829 | 13023 |
| monAI | polyketide synthase loading & | 14121 | 23198 |
| | KS-L | 14172 | 15486 |
| | AT-L malonate specific | 15777 | 16880 |
| | ACP-L | 17019 | 17276 |
| | KS1 | 17358 | 18626 |
| | AT1 methylmalonate specific | 18960 | 19976 |
| | DH1 (potential) | 20019 | 20519 |
| | KR1 (inactive) | 21636 | 22241 |
| | ACP1 | 22536 | 22793 |
| monAI | polyketide synthase module 2 | 23205 | 29921 |
| | KS2 | 23307 | 24569 |
| | AT2 methylmalonate specific | 24891 | 25913 |
| | DH2 | 25953 | 26369 |
| | ER2 | 27600 | 28463 |
| | KR2 | 28485 | 29042 |
| | ACP2 | 29313 | 29570 |
| monAI | polyketide synthase modules 3 & 4 | 29974 | 42372 |
| | KS3 | 30076 | 31347 |
| | AT3 malonate specific | 31798 | 32838 |
| | DH3 | 32884 | 33465 |
| | KR3 | 34692 | 35181 |
| | ACP3 | 35553 | 35811 |
| | KS4 | 35899 | 37170 |
| | AT4 methylmalonate specific | 37489 | 38511 |
| | DH4 | 38557 | 38982 |
| | ER4 | 40123 | 40986 |
| | KR4 | 41005 | 41562 |
| | ACP4 | 41848 | 42105 |
| monAI | polyketide synthase modules 5 & 6 | 42448 | 54564 |
| | KS5 | 42628 | 43890 |
| | AT5 ethylmalonate specific | 44221 | 45243 |
| | DH5 | 45289 | 45744 |
| | KR5 | 46785 | 47337 |
| | ACP5 | 47593 | 47850 |
| | KS6 | 47947 | 49218 |
| | AT6 malonate specific | 49579 | 50601 |
| | DH6 | 50644 | 51075 |
| | ER6 | 52222 | 53102 |
| | KR6 | 53101 | 53661 |
| | ACP6 | 54052 | 54306 |
| monA | polyketide synthase modules 7 & 8 | 54614 | 66934 |
| | KS7 | 54716 | 55978 |
| | AT7 methylmalonate specific | 56300 | 57319 |
| | DH7 | 57358 | 57802 |
| | KR7 | 59048 | 59608 |
| | ACP7 | 59867 | 60124 |
| | KS8 | 60185 | 61453 |
| | AT8 malonate specific | 61808 | 62839 |
| | DH8 | 62882 | 63316 |
| | ER8 | 64577 | 65437 |
| | KR8 | 65456 | 66016 |
| | ACP8 | 66404 | 66661 |
| monA | polyketide synthase module 9 | 66952 | 72054 |
| | KS9 | 67075 | 68340 |
| | AT9 malonate specific | 68698 | 69729 |
| | KR9 (potential) | 70735 | 71262 |
| | ACP9 | 71536 | 71783 |
| monH | probable regulator | 72051 | 74993 |
| monCI | FAD containing epoxidase | 76541 | 75051 |
| monBI | double bond isomerase | 76960 | 76538 |
| monBI | double bond isomerase | 77450 | 77016 |
| monA | polyketide synthase modules 11 & | 88708 | 77447 |
| | KS11 | 88612 | 87344 |
| | AT11 methylmalonate specific | 87022 | 85993 |
| | KR11 | 85111 | 84562 |
| | ACP11 | 84292 | 84035 |
| | KS12 | 83962 | 82694 |
| | AT12 methylmalonate specific | 82354 | 81335 |
| | DH12 (potential) delta | 81286 | 80855 |
| | ER12 (potential) | 79618 | 78914 |
| | KR12 | 78895 | 78337 |
| | ACP12 | 78070 | 77812 |
| monA | polyketide synthase module 10 | 93741 | 88816 |
| | KS10 | 93636 | 92368 |
| | AT10 methylmalonate specific | 92040 | 91021 |
| | KR10 | 90132 | 89584 |
| | ACP10 | 89322 | 89068 |
| monD | P450 oxygenase | 94081 | 95273 |
| monRI | probable activator | 96141 | 95338 |
| monA | thioesterase | 96941 | 96138 |
| orf29 | cell wall biosynthesis capK | 97580 | 98953 |
| lipB | lipase B | 99983 | 98991 |
| orf31 | ion pump | 101433 | 100507 |
| orf32 | membrane structural protein | 102581 | 101490 |
| amtA | glycine amidinotransferase | 102924 | 103450 |

TABLE II

GdhA, glutamate dehydrogenase (partial coding
sequence) Length: 346 amino acids

| | |
|---|---|
| 1 | LTTRPDTKTA LSQKTALSQL LTEIEHRNPA QPEFHQAARE VLETLAPVIA |
| 51 | ARPEYAEAGL IERLCEPERQ IVFRVPWQDD HGRVRVNRGF RVEFNSALGP |
| 101 | YKGGLRFHPS VNLGVIKFLG FEQIFKNALT GLGIGGGKGG SDFDPRGRSD |
| 151 | AEVMRFCQSF MTELYRHIGE HTDVPAGDIG VGGREIGYLF GQYRRITNRW |
| 201 | EAGVLTGKGR NWGGSLIRPE ATGYGNVLFA AAMLRERGET LEGRTAVVSG |
| 251 | SGNVAIYTIQ KLAALGANAV TCSDSSGYVV DEKGIDLDLL KQVKEVERAR |
| 301 | VDTYAQRRGA SARFVPGRRV WEVPADIALP SATQNELDAD DATALI |

DapA, dihydrodopicolinate synthase Length: 307 amino acids

| | |
|---|---|
| 1 | MTLASSLEPT TEPLFNGLYV PLVTPFTDDL RLAPEALARL ADEALSAGAS |
| 51 | GLVALGTTAE AATLTAEERE TVIRVCSAAC RAHGAPLIVG VGTNDTATAI |
| 101 | TALRELAARG DVAAALVPAP PYIRPGEAGT LAHFAALAEH GGLPLVVYDI |
| 151 | PYRTGQTLGA GTITALGRLP EVVGIKHATG SIDPTTMELL DSPLPGFAVL |
| 201 | GGDDIVLSPL VAAGAHGGIV ASANLRTADY AEMIALWRRG SAAPARALGA |
| 251 | DLARLSAALF TEPNPTVIKG VLHAQNRIPS PAVRMPLLAA SADSVRRAAP |
| 301 | LAASRK* |

ORF3, putative transcriptional activator protein
Length: 314 amino acids

| | |
|---|---|
| 1 | MLDVRRLHLL RELDRRGTIA AVAEALTFTA SAVSQQLGVL EREAGVPLLE |
| 51 | RSGRRVVLTP AGRSLVAHAD AVLNRLEQAV AELAGARDGI GGPLRIGTFP |
| 101 | SGGHTIVPGA LAELASRHPA LEPMVREIDS ARVSDGLRAG ELDVALVHDY |
| 151 | DFVPATPDTT VDEVPLLEEP MYLVTHAADT ATDSGSGSTL AALLGPCAEV |
| 201 | PWITARDGTT GHAMAVRACQ AAGFQPRIRH QVNDFRTVLA LVAAGQGAGF |
| 251 | VPRMAAEPSP AGVVLTKLPL FRRSKVAFRA GGGAHPAIAA FVAAATTAVE |
| 301 | RMAGSRGPAG GSE* |

ORF4, hypothetical protein Length: 139 amino acids

| | |
|---|---|
| 1 | MADDAYLFLL PDRHPRLGAA LAAVGALECT ETPAVHAWLQ AHEASVSSEQ |
| 51 | VRILPADAET LIPKDAERLP VPLSEEEALK VEQECAPQTV TDMESELLAF |
| 101 | RETTQDWQAL VHRALTAGIP AQRIARLTGL DPEEIGRL* |

ORF5, hypothetical protein Length: 208 amino acids

| | |
|---|---|
| 1 | LAVAACAAVV LPIDAVVRIS AADVGVLVFF AYLLPYLAIT MTVFVSVAPE |
| 51 | QVRSWARREA RGTFLQRYVL GTAPGPGGSL FIAAAALVVA VLWLPGHLST |
| 101 | TFSALPRTLV ALALVVAAWI CVVVAFAVTF QADNLVENER ALEFPGERSP |
| 151 | AWADYVYFAL AAMTTFGTTD VDVTSRDMRR TVAANTVIAF VFNTVTVAIL |
| 201 | VSALGGR* |

ORF6, hypothetical protein Length: 63 amino acids

| | |
|---|---|
| 1 | MTVMDKLKQM LKGHEDKAGQ GIDKAGDFVD GKTQGKYSGQ VDTAQDKLRD |
| 51 | QFGSDQQEPP QR* |

ORF7, hypothetical protein Length: 185 amino acids

| | |
|---|---|
| 1 | MGTAQSQEQA AAPGACAAFV RFVLCGGGVG LASSFAVVAL ASWVPWALAN |
| 51 | ALVAVVSTVV ATELHARFTF GAGGRATWRQ HAQSAGSAAA AYAVTCVAMF |
| 101 | VLQQLVAAPG AVLEQVVYLS ASALAGVARF VVLRLVVFAR NRSLPAAAAV |
| 151 | RTARPVRRVP APVPATVAHA ASRPAGPAAL CPAA* |

AcpX, acyl carrier protein (ACP) Length: 106 amino acids

| | |
|---|---|
| 1 | MTSTDHTSGQ DATELEKQLA AATPEEREKL LTDTIRTQAG TLLNTTLSDD |
| 51 | SNFLENGLNS LTALELTKTL MTLTGMEIAM VAIVENPTPA QLAHHLGQEL |
| 101 | AHTTA* |

KsX, ketoacyL-ACP synthase Length: 829 amino acids

| | |
|---|---|
| 1 | VANEEKLVEY LKWTTAELHQ AQQQLRELKA AQHEPIAVVS MACRLPGKTR |
| 51 | TPDDLWDLVS EGRDAVTGFP DDRAWELPEE RPYAELGGFL DDAAGFDAGF |
| 101 | FDISDTEAVA TEPLQRLMLH LAWETVERGH IAPHTLRSTL TGVYVGATGH |
| 151 | DYATRLETAP DELLPYLGGG TSGSLVSGRI AYALGLEGPA ISVDTACSSS |
| 201 | LVALHLLACQA LRRGECGLAL AGGGTVMSTP HTFHAFAHQK SLAQDGRCKP |
| 251 | FAAAADGMGL GEGVGLVLLE RLGDARKNGH PVLAVIRGSA VNQDGAGYGL |
| 301 | AAPNGPSQQH VTRAALADAG LTPDQIDAVE AHGTGTPIGD AIEVQALLAT |
| 351 | YGADRSPDRP LWLGSVKSNT GHTQGAAGAA ALIKMVQAFR HGTLPPTLHV |
| 401 | DRPTPLAAWK KGAVRLLTEA VDWPRREEPR RVGISAFATS GTNAHLILEE |
| 451 | PPVDEAPVPD AARDQTSPVA PELPVAWSLS ARTPEALRAQ AKALVTHLAA |
| 501 | TDPAPSPAEV AYSLAATRSP LEHRAVLTGT DHTELLAAAR ALAAGEDHPD |
| 551 | LVRSTPGAGP KKIAWHFDGR PADGVTTGAA PGAKPGATFG ATFGAAFGGA |
| 601 | EFHSAFPLFA SAFDEARALL DTHLPTPLPT PHSELARFAV HTALARLLLE |
| 651 | TGVRPHTLTG DGVGHIAAAY AAGILTLDDA CRLAAAHAAA AQAAEGEQPA |
| 701 | PPDAYEPVLK QLTFQRATLT LTSTAPADTP IASADYWHHH LTSPAPTAPP |
| 751 | TPETHTLLHL GALSPEGTQT SAVSALLTAL ARLHTTGGTV DWTPLVRRTP |
| 801 | HPRTIDLPTY SFQATRYWLH DHTAHAAV* |

MonCII, probable epoxyhydrolase/cyclase
Length: 300 amino acids

| | |
|---|---|
| 1 | VKNLRIPVSQ TVSLNVRYRP ADGPGAPGRP FLLLHGMLSN ARMWDEVAAR |
| 51 | LAAAGHPAYA VDHRGHGESD TPPDGYDNAT VVTDLVAAVT ALDLSGALVA |
| 101 | GHSWGAHLAL RLAAEHPDLV AGLALIDGGW YEFDGPVMRA FWERTADVVR |
| 151 | RAQQGTTSAA DMRAYLRATH PDWSPTSIEA RLADYRVGPD GLLIPRLTST |
| 201 | QVMSIVAGLQ REAPADWYPK VTVPVRLLPL IPAIPQLSDQ VRAWVAAAEA |
| 251 | ALEQVSVRWY PGSDHDLHAG APDEIAADLL LLARSCEAMP GGKAGVRPA* |

MonE, S-adeonosylmethionine-dependent methyltransferase
Length: 277 amino acids

| | |
|---|---|
| 1 | VNKTVAPEPS DIGHYYDHKV FDLMTQLGDG NLHYGYWFDG GEQQATFDEA |
| 51 | MVQMTDEMIR RLDPAPGDRV LDIGCGNGTP AMQLARARDV EVVGISVSAR |
| 101 | QVERGNRRAR EAGLADRVRF EQVDAMNLPF DDGSFDHCWA LESMLHMPDK |
| 151 | QQVLTEAHRV VKPGARMPIA DMVYLNPDPS RPRTATVSDT TTYAALTDIG |
| 201 | DYPDIFRAAG WTVLELTDIT RETAKTYDGY VEWIRAHRDE YVDIIGVEGY |
| 251 | ELFLHNQAAL GKMPELGYIF ATAQRP* |

MonT, putative monensin resistance gene (ABC-transporter)
Length: 512 amino acids

| | |
|---|---|
| 1 | MSADLGARRW WAVGALVLAS MVVGFDVTIL SLALPAMADD LGANNVELQW |
| 51 | FVTSYTLVFA AGMIPAGMLG DRFGRKKVLL TALVIFGIAS LACAYATSSG |
| 101 | TFIGARAVLG LGAALIMPTT LSLLPVMFSD EERPKAIGAV AGAAMLAYPL |
| 151 | GPILGGYLLN HFWWGSVFLI NVPVVILAFL AVSAWLPESK AKEAKPFDIG |
| 201 | GLVFSSVGLA ALTYGVIQGG EKGWTDVTTL VPCIGGLLAL VLFVMWEKRV |
| 251 | ADPLVDLSLF RSARFTSGTM LGTVINFTMF GVLFTMPQYY QAVLGTDAMG |
| 301 | SGFRLLPMVG GLLVGVTVAN KVAKALGPKT AVGIGFALLA AALFYGATTD |
| 351 | VSSGTGLAAA WTAAYGLGLG IALPTAMDAA LGALSEDSAG VGSGVNQSIR |
| 401 | TLGGSFGAAI LGSILNSGYR GKLDLDGVPE QAHGAVKDSV FGGLAVARAI |
| 451 | KSNGLADSVR SAYVHALDVV LVVSGGLGLL GVVLAVVWLP RHVGQSTAKT |
| 501 | AESEHEAADA V* |

MonRII, probable repressor protein Length: 192 amino acids

| | |
|---|---|
| 1 | VPGLRERKKA RTKAAIQREA VRLFREQGYT ATTIEQIAEA AEVAPSTVFR |
| 51 | YFATKQDLVF SHDYDLPFAM MVQAQSPDLT PIQAERQAIR SMLQDISEQE |
| 101 | LALQRERFVL ILSEPELWGA SLGNIGQTMQ IMSEQVAKRA GRDPRDPAVR |
| 151 | AYTGAVFGVM LQVSMDWAND PDMDFATTLD EALHYLEDLR P* |

MonAIX, thioesterase Length: 269 amino acids

| | |
|---|---|
| 1 | MDRGTAARAP QIGDEFGAAT GNGVWLRRYH AAAEAPVRLV CFPFAGGSAS |
| 51 | YYFGLSGLLA PGVEVLAVQY PCRQDRHAEP CLASVAELAD GVVPHLPCDG |
| 101 | KPFALFGHSL GAIVAFEVAR RLRGPAGPGL PVHLFVSGGL ARPYRPAGRS |
| 151 | GAFGDADILA HLRAMGGTDE RFFRSPELQE LVLPALRADY RAVATYEAPG |
| 201 | PGRLDCPITA LIGDADERTS PEQAATWRER TGAAFDLRVL PGGHFYLDGC |
| 251 | QEQVAAVVTE ALTAGPGV* |

MonAI, polyketide synthase multi-enzyme MONS1, housing
loading module and extension module 1
Length: 3026 amino acids

| | |
|---|---|
| 1 | MAASASASPS GPSAGPDPIA VVGMACRLPG APDPDAFWRL LSEGRSAVST |
| 51 | APPERRRADS GLHGPGGYLD RIDGFDADFF HISPREAVAM DPQQRLLLEL |
| 101 | SWEALEDAGI RPPTLARSRT GVFVGAFWDD YTDVLNLRAP GAVTRHTMTG |
| 151 | VHRSILANRI SYAYHLAGPS LTVDTAQSSS LVAVHLACES IRSGDSDIAF |
| 201 | AGGVNLICSP RTTELAAARF GGLSAAGRCH TFDARADGFV RGEGGGLVVL |
| 251 | KPLAAARRDG DTVYCVIRGS AVNSDGTTDG ITLPSGQAQQ DVVRLACRRA |
| 301 | RITPDQVQYV ELHGTGTPVG DPIEAAALGA ALGQDAARAV PLAVGSAKTN |
| 351 | VGHLEAAAGI VGLLKTALSI HHRRLAPSLN FTTPNPIAPL ADLGLTVQQD |
| 401 | LADWPRPEQP LIAGVSSFGM GGTNGHVVVA AAPDSVAVPE PVGVPERVEV |
| 451 | PEPVVVSEPV VVPTPWPVSA HSASALRAQA GRLRTHLAAH RPTPDAARVG |
| 501 | HALATTRAPL AHRAVLLGGD TAELLGSLDA LAEGAETASI VRGEAYTEGR |
| 551 | TAFLFSGQGA QRLGMGRELY AVFPVFADAL DEAFAALDVH LDRPLREIVL |
| 601 | GETDSGGNVS GENVIGEGAD HQALLDQTAY TQPALFAIET SLYRLAASFG |
| 651 | LKPDYVLGHS VGEIAAAHVA GVLSLPDASA LVATRGRLMQ AVRAPGAMAA |
| 701 | WQATADEAAE QLAGHERHVT VAAVNGPDSV VVSGDRATVD ELTAAWRGRG |
| 751 | RKAHHLKVSH AFHSPHMDPI LDELRAVAAG LTFHEPVIPV VSNVTGELVT |
| 801 | ATATGSGAGQ ADPEYWARHA REPVRFLSGV RGLCERGVTT FVELGPDAPL |
| 851 | SAMARDCFPA PADRSRPRPA AIATCRRGRD EVATFLRSLA QAYVRGADVD |
| 901 | FTRAYGATAT RRFPLPTYPF QRERHWPAAA CVGQQPETPE LPESSESSEQ |
| 951 | AGHEREEGAR AWGGPEGRLA GLSVNDQERV LLGLVTKHVA VVLGDASGTV |
| 1001 | QAARTFKQLG FDSMAAAELS ERLGTETGLP LPATLTFDYP TPLAVAAHLR |
| 1051 | AELTGTPAPA GSAPATGALG AGDLGTDEDP VAIVAMSCRY PGGAGTPEDL |
| 1101 | WRLVAGGADA IGDFPTDRGW DLARLFHPDP DRSGTSCTRQ GGFLYDAADF |
| 1151 | DAEFFDISPR EALAVDPQQR LLLECAWEAF ERAGLDPRAL KGSPTGVFVG |
| 1201 | MTGQDYGPRL HEPSQATDGY LLTGSTPSVA SGRLSFSFGL EGPALTVDTA |
| 1251 | CSSSLVTLHL AAQALRRGEC DLALAGGATV LATPGMFTEF SRQRGLAPDG |
| 1301 | RCKPFAAGAD GTGWAEGVGL VLLERLSEAR RKGHAVLAVI RGSAINQDGA |
| 1351 | SNGLTAPNGP SQQRVIRAAL AAARLTADEV DVVEAHGTGT TLGDPIEAQA |
| 1401 | LLATYGQGRS AERPLWLGSV KSNIGHTQAA AGVAGVIKMV MAMRHDLLPA |
| 1451 | TLHVDEPSGH VDWSTGAVRL LTEPVVWPRG ERPRRAAVSS FGISGTNAHL |
| 1501 | VLEEAGQDEY VAGAADDAGP VDGAVLPWVV SGRTGAALRE QARRLRELVT |
| 1551 | GGSADVSVSG VGRSLVTTRA VFEHRAVVVG RDRDTLIGGL EALAAGDASP |
| 1601 | DVVCGVAGDV GPGPVLVFPG QGSQWVGMGA QLLGESAVFA ARIDACEQAL |
| 1651 | SPYVDWSLTE VLRGDGRELS RVDVVQPVLW AVMVSLAAVW ADHGVTPAAV |
| 1701 | VGHSQGEIAA VVVAGALTLE DGAKIVALRS RALRQLSGGG AMASLGVGQE |
| 1751 | QAAELVEGHP GVGIAAVNGP SSTVISGPPE QVAAVVADAE ARELRGRVID |
| 1801 | VDYASHSPQV DAITDELTHT LSGVRPTTAP VAFYSAVTGT RIDTAGLDTD |
| 1851 | YWVTNLRRPV RFADAVTALL ADGHRVFIEA SSHPVLTLGL QETFEEAGVD |
| 1901 | AVTVPTLRRE DGGRARLARS LAQAFGAGCA VRWENWFPAT GTSTVELPTY |
| 1951 | AFQRRRYWLE APTGTQDAAG LGLAAAGHPL LGAATEIAIDG DIRLLTGRIS |
| 2001 | RHSHPWLAQH TLFGAAVVPA SVLAEWALRA ADEAGCPRVD DLTLRTPLVL |
| 2051 | PETAGVQVQI VVGPADARDG HRDFHVYARP DGKDASEGEG IAEGEGASEG |
| 2101 | EGASGGTDAP WTCHADGRLV AEPTGTASED SPDTVWPPPG AEPVDLGDFY |
| 2151 | ERAAATGVGY GPVFTGLRAL WRRDGELFAE AVLPQEAPET AGFGMHPALL |
| 2201 | DAALHPALLG ERPAEEDKVW LPFTLTGVTL WATGATSVRV RLTPLDDDPD |
| 2251 | ASADGRAWRV GVSDPTGAEV LTCEALVAVA AGRRELRAAG ERVSDLYAVE |
| 2301 | WVPVPGPGPV GEGADFSGWA GLGECGERWE CVGRVERWYE DLDALGAAVE |
| 2351 | GGASVPSVVL ATAAAAPGGA GDGAADALSA VRWTGALLDQ WLADARFADA |
| 2401 | RLVVITSGAV ATGDDFLPDP AAAAVRGLVE QAQVRHPGRI LLVDTEAGAG |
| 2451 | LGVGAGVDDA LLEQAVAMAL GADEPQLALR AGRVLAPRLT APQDAAVTEA |
| 2501 | ARPLDPDGTV LITGPAGAPV ADLAEHLVRT GQCRHLLLLP GDGELEEMAE |
| 2551 | ELRGLGATVD LSTADPADPT ALAEVVAAVE GDHPLTGVIH ATGVVDAFDP |
| 2601 | GDSASDLMID SASDSFAEAW SSRAGVTAAL HTATAHLPLD LFAVLSPAGA |
| 2651 | DLGIARSAAA AGADAFSAAL ALRRHTTVTT DTTAPPRTTA PPRTTASPRT |
| 2701 | TALSSSRTTG VALAYGPPTA PRPGIKGTAP GRIPVLLDAA RAHGGGSPLL |
| 2751 | GARLAARALA AESAAEGVAG LPAPLRALAV AAAAAGAPTR RTAADRKPPA |
| 2801 | DWPARLAPLS APEQLRLLID AVRTHAAAVL GRTDPEALRG DATFKQLGLD |
| 2851 | SLTAVELRNR LVEDTGLRLP TALVFRYPTP AAIAAHLRER LTSPSETTAT |
| 2901 | QRSGGQTPAA GQASSALAPG GSAAGPPAAD TVLSDLTRME NTLSVLAAQL |
| 2951 | PHTETGEITT RLEALLTRWK TTNATANDSG DGNGGDDDAA ERLKAASADQ |
| 3001 | IFDFIDNELG VGHGTSRVTP TPKAG* |

MonAII, polyketide synthase multi-enzyme MONS2, housing
extension module 2 Length: 2239 amino acids

| | |
|---|---|
| 1 | MASEEQLVEY LRRVTTELHD TRRRLVQEED RRQEPVALVG MACRFPGGVA |
| 51 | SPEDLWDLVA AGKDAIEDFP TDRGWDLEAL YDPDPAAYGT SYVRHGGFVD |
| 101 | DAGSFDADFF GISPREALAM DPQQRLMLET SWELFERAGI EPVSLKGSRT |
| 151 | GVYAGVSSED YMSQLPRIPE GFEGHATTGS LTSVISGRVA YNYGLEGPAV |
| 201 | TVDTACSASL VAIHLASQAL RQRECDLALA GGVLVLSSPL MFTEFCRQRG |
| 251 | LAPDGRCKPF AAAADGTGFS EGIGLLLLER LSDARRNGHK VLAVIRGSAV |
| 301 | NQDGASNGLT APNDAAQEQV IRAALDNARL TPSEVDAVEA HGTGTKLGDP |
| 351 | IEAGALLATY GQHRARPLLL GSLKSNIGHT HATAGVAGVI KTVMAIRNGL |
| 401 | LPATLHVEEL SPHVDWDAGA VEVVTEPTPW PETGHPRRAG VSAFGISGTN |
| 451 | AHLILEEAPP EEDVPAPVVV ESGGVVPWVV SGRTPEALRE QARRLGEFVA |
| 501 | GDTDALPNEV GWSLATTRSV FEHRAVVVGR DRDALTAGLG ALAAGEASAG |
| 551 | VVAGVAGDVG PGPVLVFPGQ GAQWVGMGAQ LLDESAVFAA RIAECERALS |
| 601 | AHVDWSLSAV LRGDGSELSR VEVVQPVLWA VMVSLAAVWA DYGVTPAAVI |
| 651 | GHSQGEMAAA CVAGALSLED AARIVAVRSD ALRQLQGHGD MASLSTGAEQ |
| 701 | AAELIGDRPG VVVAAVNGPS STVISGPPEH VAAVVADAEA RGLRARVIDV |
| 751 | GYASHGPQID QLHDLLTERL ADIRPTNTDV AFYSTVTAER LTDTTALDTD |
| 801 | YWVTNLRQPV RFADTIEALL ADGYRLFIEA SAHPVLGLGM EETIEQADMP |
| 851 | ATVVPTLRRD HGDTTQLTRA AAHAFTAGAD VDWRRWFPAD PAPRTIDLPT |
| 901 | YAFQRRRYWL ADTVKRDSGW DPAGSGHAQL PTAVALADGG VVLNGRVSAE |
| 951 | RGGWLGGHVV AGTVLVPGAA LVEWVLRAGD EAGCPSLEEL TLQAPLVLPE |
| 1001 | SGGLQVQVVV GAADEQGGRR DVHVYSRSEQ DASAVWQCHA VGELGRASVA |
| 1051 | RPVRQAGQWP PAGAEPVEVG GFYEGVAAAG YEYGPAFRGL RAMWRHGDDL |
| 1101 | LAEVELPEEA GSPAGFGIHP ALLDAALHPL LAQRSRDGAG AGAHGGQVLL |
| 1151 | PFSWSGVSLW ASEATTVRVR LTGLGGGDDE TVSLTVTDPA GGPVVDVAEL |
| 1201 | RLRSTSARQV RGSAGPGADG LYELRWTPLP EPLPVPAPAN GRDVAADLSG |
| 1251 | CAVLGELVAE PGPGIDLEGC PCYPGVGALA DNASPPSMIL APVHSDTTGG |
| 1301 | DGLALTERVL RVIQDFLAAP SLEQKQTRLA FVTRGAADTG STTGGSAAPA |
| 1351 | EAVDPAVAAV WGLVRSAQSE NPGRFVLLDT DAPLDQASVA PLVDAVRSAV |
| 1401 | EADEPQVALR GGRLLVPRWA RAGEPVELAG PAGARAWRLV GGDSGTLEAV |
| 1451 | VAEACDDIVL RPLAPGQVRV AVHTAGVNFR DVLIALGMYP DPDALPGTEA |
| 1501 | AGVVTEVGPG VTRLSVGDRV MGMMDGAFGP WAVADARMLA PVPPGWGTRQ |
| 1551 | AAAAPAAFLT AWYGLVELAG LKAGERVLIH AATGGVGMAA VQIARHVGAE |
| 1601 | VFATASPGKH AVLEEMGIDA AHRASSRDLA FEDAFRQATD GRGVDVVLNS |
| 1651 | LTGELLDASL RLLGDGGRFV EMGKSDPRDP ELVALEHPGV SYEAFDLVAD |
| 1701 | AGPERLGLML DRLGELFAGG SLVPLPVTAW PLGRAREALR HMSQARHTGK |
| 1751 | LVLDVPAPLD PDGTVLVTGG TGTIGAAVAE HLARTGESKH LLIVSRSGPA |
| 1801 | AHGAEELVSR IAEFGAEATF VAADVSEPDA VAALIEGIDP AHPLTGVVHA |
| 1851 | AGVLDNALIG SQTTESLTRV WAAKAAAAQQ LHEATRESRL GLFVMFSSFA |
| 1901 | STMGTPGQAN YSAANAYCDA LAALRRAEGL AGLSVAWGLW EATSGLTGTL |
| 1951 | SAADRARIDR YGIRPTSAAR GCALLAAARA HGRPDLLAMD LDARVPAASD |
| 2001 | APVPAVLRTL AAAGAPATAR PTAAAAADGA TDWSGRLAGL TEEARLELLT |
| 2051 | ELVCTHAAGV LGHADAGAVQ VDAPFKELGF DSLTAVELRN RIAAATGLKL |
| 2101 | PAALVFDYPQ ARVLAAHLAE RLVPEGAGAM GGVSGAEGVR DAYGAGGPGG |
| 2151 | DMTAQVLLEV ARVEHTLSAA VPHGLDRAAV AARLEALLAR CTATTAATGA |
| 2201 | AGAAVEGDGD SDGDGAVDQL ETATAEQVLD FIDNELGV* |

MonAIII, polyketide synthase multi-enzyme MONS3, housing
extension modules 3 and 4 Length: 4133 amino acids

| | |
|---|---|
| 1 | MVSEEKLVDY LKRVSADLHA TRQRLREAEE RGQEPVAVVE AACRYPGGIR |
| 51 | TPEDLWDLVA AGGNALGAFP DNRGWDLRRL FHPDPDHPGT TYAREGGFLH |
| 101 | DADLFDPEFF GISPREAAVL DPQQRLLLEC AWEALERAGI DPRSLQGSRT |
| 151 | GVYAGAALPG FGTPHIDPAA EGHLVTGSAP SVLSGRLAYT FGLEGPAVTI |
| 201 | DTACSSSLVA VHLAAHALRQ RECDLALAGG VTVMTTPYVF TEFSRQRGLA |
| 251 | ADGRCKPFAA AADGTAFSEG AGLLVLERLS DARRAGHRVL AVIRGSAVNQ |
| 301 | DGASNGLTAP NGPAQQRVIR AALAGARLSP AEVDAVEAHG TGTRLGDPIE |
| 351 | ADALLATYGQ ERHGGRPLWL GSVKSNIGHT QGAAGAAGLI KMVQALRHET |
| 401 | LPATLYADEP TPHADWESGA VRLLSAPVAW PRGEHGEHTR RAGISSFGIS |
| 451 | GTNAHLILEE APAADAEGAG GDGDGDGGGV RPVVRVGATG PREEQGQGQG |
| 501 | QEQHQQQRQQ RQRSSMMPTP HLPWLLSARS PAALRAQADA LANHVAHADH |
| 551 | SIADIGGTLL RRTLFEHRAV VLGTDRDERA AALAALAAGR AHPALTRAAG |
| 601 | PARNGGTAFL FTGQGSQRPG MGRQLYDTFD VFAESLDETC ARLDPLLEQP |
| 651 | LKPVLFAPAD TAQAAVLHGT GMTQAALFAL EVALYRQVTS FGIAPSHLTG |
| 701 | HSVGEIAAAH VAGVFSLADA CTLVAARGRL MQALPAGGAM LAVQAAEDDV |
| 751 | LPLLAGQEER LSLAAVNGPT AVVVSGEAAA VGEVEKALRG RGLKTKRLNV |
| 801 | SHAFHSPLIE PMLDDFREVA RGLTFHAPTL PVVSNLTGRL ADAELMADAE |
| 851 | YWVRHVRRPV RFHDGLRALS EQGVVRYLEL GPDPVLATMV QDGLPAPAEG |
| 901 | EEPEPVVAAA LRSKHDEGRT LLGAVAALHT DGQPADLTAL FPADAGQVPL |
| 951 | PTYRFQRRRY WRVAPDAAAP ARAAGLQETG HPLLPAVIRQ ADGGILLAGR |
| 1001 | LSLRTHPWLA DHTIAGGVPL PATAFVELAL LAGRHAACDT IDDLTLETPL |
| 1051 | LLDDTGTGVG AAVGAGADAL VDAIEVQLAL GAPDGSGRRA LTVHSRPADD |
| 1101 | AADDGDAADA ADAAGRGGPG GSGDLGDPGD PGDLGDGGGS RGWRRHATGI |
| 1151 | LSAGPAAEPA APDAAPWPPA DATALDVDAL YARLDAQGYS YGPAFRAVHA |
| 1201 | AWRHGDDLYA DVRLADEQRA EADAFALHPA LLDAALHAVD ELYRGSEGRC |
| 1251 | QEQGQGGQEP EQGRGDADAP VRLPFSFSDI RHHATGATRL WVRLSPQGDD |
| 1301 | RLRLSLTDGE GGQVATVDAL QLRLIPADRW RAARPTTAAP LYHLDWHELP |
| 1351 | LPEPAETDPA AHSWAVLGAH DAGLAPAAHY PDLAALKAAV EAGEPVPDIV |
| 1401 | FAPFPAQGTE TDVPAQVRAH ARHALELLRD WLTTEAFAAA RLVVLTTGAV |
| 1451 | TARPEDGPAD LATAPVWGLV RAAQAEQPDH VVLVDIDKDI DKDTDEETDQ |
| 1501 | ATDAGTASRH ALPAALAAAA AQAETQLALR AGTVLVPRLA VVPPRTDTPA |
| 1551 | LHATAPESTT DTVDSTGIAG AAESGGTVLI TGGTGGLGQA VARHLAAAHG |
| 1601 | ARHLLLVSRR GDAAEGVAEL RADLADDGVD VRVAACDITD RDALAGLLAD |
| 1651 | IPAAHPLTAV VHTAGVIDDS LITAMTPERL DAVLAPKADA AWHLHELTRD |
| 1701 | KDLSAFVLFS SGASVLGNGG QANYAAANTF LNTLAEHRRA AGLAATSVAW |
| 1751 | GLWESASGGM AARLGDADA RIHRTGVTGL TDEQALALFD AALTAEIIPTV |
| 1801 | LATRFDRAVL RGQAAARTLQ PALRGLVRTP RPTASAGAIG STAATGSATD |
| 1851 | ENAPSSWAAR LARLSAADRD RALNELIREQ IATVLAHPSP DTIELGRAFQ |
| 1901 | ELGFDSLTAL ELRNRLSTAT GIRLPATLVF DHPSPTALVR HLHSHLPDEA |
| 1951 | QHTSPTAPGA SAEGTAATAT GIDDDPIAIV GMACRYPGGV TSPEQLWQLV |
| 2001 | ATGTDAIGPF PEDRGWDTAG LFDPDPDQVC HSYTREGGFL YDAARFDAGF |
| 2051 | FGISPREAAA TDPQQRLLLE TAWQAFEHAG IDPAALRGTP CGVITGIMYD |
| 2101 | DYGSRFLARK PDGFEGRIMT GSTPSVASGR VAYTFGLEGP AITVDTACSS |
| 2151 | SLVAMHLAAQ ALRQGECELA LAGGVTVMAT PNTFVEFSRQ RGLAPDGRCK |
| 2201 | PFAAAADGTG WGEGAGLVVL ERLSDARRKG HRVLALLRGS AVNQDGASNG |
| 2251 | MTAPNGPSQE RVIRTALAGA GRGPEDIDVV EAHGTGTTLG DPIEAQALLA |
| 2301 | TYGQGRPEDR PLWLGSVKSN IGHTQAAAGV AGVIKMVMAL RHEQLPTTLH |
| 2351 | ADEPTPHVQW DGGGVRLLTE PVPWSRGERT RRAGVSSFGI SGTNAHLILE |
| 2401 | EPPEEDLPEP VAAEPGGVVP WVVSGRTPDA LREQARRLGE FVVGAGDVSA |
| 2451 | AEVGWSLATT RSVFEHRAVV AGRDRDDLVA GMQALAAGET PTDVVSGAAA |
| 2501 | SSGAGPVLVF PGQGSQWVGM GAQLLDESPV FAARIAECEQ ALSAYVDWSL |
| 2551 | SDVLRGDGSE LSRVEVVQPV LWAVMVSLAA VWADYGVTPA AVVGHSQGEM |
| 2601 | AAACVAGALS LEDAARIVAV RSDALRQLQG HGDMASLGTG AEQAAELIGD |
| 2651 | RPGVVVAAVN GPSSTVISGP PEHVAAVVAE AEARGLRARV IDVGYASHGP |
| 2701 | QIDQLHDLLT EGLADIRPAN TDVAFYSTVT AERLTDTTAL DTDYWVTNLR |
| 2751 | QPVRFADTIE ALLADGYRLF IEASAHPVLG LGMEETIEQA DIPATVVPTL |
| 2801 | RRDHGDTTQL TRAAAHAFTA GADVDWRRWF PADPTPRTVD LPTYAFQHQH |
| 2851 | YWLEEPSGLT GDAADLGMVA AGHPLLGACV ELAESDSYLF TGRLSRRAPS |
| 2901 | WLAEHVVAGT VLVPGAALVE WVLRAGDEAG CPTIEELTLQ APLVLPESGG |
| 2951 | LQVQVVVGAT DEQSGRRDVH VYSRSEQDAS AVWVCHAVGV VSSEMPEAAA |
| 3001 | ELSGQWPPAG AEAVDVEDFY ARAAEAGYAY GPAFQGLRAL WRHGTELFAE |
| 3051 | VVLPEQAGGH DGFGIHPALL DAALHPLMLL DRPADGQMWL PFAWSGVSLN |
| 3101 | ADRATHVRVR LSPRGEAAER DLRVVIADAT GAPVLTVDAL TLRAADPGRL |
| 3151 | GAAARGGVDG LYTVDWTPLP LPQPLPLPRT DAGGSADWVI LSDNSSAALA |
| 3201 | DAVSSATAAG GGAPWALLAP VGGGSADDGL PVVRRTLSLV QEFLAAPELT |
| 3251 | ESRLVIVTRG AVATDADGDV AASAAAVWGL IRSAQSENPG RFVLLDVEEE |
| 3301 | HLHPDGGELP YAALRHAVEE LDEPQLALRS GKFLVPRMTP AAAPEELVPP |
| 3351 | VGTSGWRLGT SGTATLENLS VIDAPEAFAP LEPGQVRISV RAAGMNFRDV |
| 3401 | LIALGMYPDK GTFAGSEGAG HVTEVGPGVT HLSVGDRVMG LFEGAFAPLA |
| 3451 | VADARMVVPI PEGWSFQEAA AVPVVFLTAW YGLVDLGRLR AGESLLIHAG |
| 3501 | TGGVGMAATQ IARHLGAEVF ATASPAKHGV LDGMGIDAAH RASSRDLDFE |
| 3551 | ETLRAATGGR GMDVVLNSLA GEFTDASLRL LAEGGRMVDM GKTDKRDPDR |
| 3601 | VAAEHAGAWY RAFDLVPHAG PDRIGEMLAE LGELFASGAL APLPVQTWPL |
| 3651 | GRAREAFRFM SQAKHTGKLV LEIPPALDPD GTVLITGGTG VLAAAVAEHL |
| 3701 | VREWGVRHLL LAGRRGSEAP GSSELAEELT ELGAEVTFAA ADVSDPDAVA |
| 3751 | ELVGKTDPAH PLTGVIHAAG VLDDAVVTAQ TPESLARVWA AKATAAHLLH |
| 3801 | EATREARLGL FLVFSSAAAT LGSPGQANYA AANAYCDALV RQRREAEGLAG |
| 3851 | LSIGWGLWQT ASGMTGHLGE TDLARMKRTG FTPLTTEGGL ALLDAARAHG |
| 3901 | RPHVVAVDLD ARAVAAQPAP SRPALLRALA AGATPGARTA RRTAAAGSVA |
| 3951 | PAGGLADRLA GLPHPERRRL LLDLVRGNVA GVLGHSDHDA VRPDTSFKEL |
| 4001 | GFDSLTAVEL RNRLAAATGL KLPAALVFDY PESATLVDHL LERLSPDGAP |
| 4051 | PPVKDAADPV LNDLGRIESS LDALALDADA RSRVTRRLNT LLSKLNGAAT |
| 4101 | AGSPADVTDL DALDALDDVS DDEMFEFIDR EL* |

MonAIV, polyketide synthase multi-enzyme MONS4, housing
extension modules 5 and 6 Length: 4039 amino acids

| | |
|---|---|
| 1 | MSSAEESSPD VSGTGVSGTG ESATGTSSTE AKLRQYLKRV TVDLGQARRR |
| 51 | LREVEERAQE PIAIVSMACR FPGDTRTPEA LWDLVAEGGD AIDDFPTNRG |
| 101 | WDLESLYHPD PDHPGTSYVR RGGFLYDAPA FDASFFGISP REALAMDPQQ |
| 151 | RVLMETAWQL LERAGIDPAS LKLSATGVYI GAGVLGFGGA QPDKTVEGHL |
| 201 | LTGSALSVLS GRISFTLGLE GPSVSVDTAC SSSLVSMHLA AQALRQGECD |
| 251 | LALAGGVTVM STPGAFTEFS RQGALSPDGR SKAFAASADG TGFSEGAGLL |
| 301 | LLERLSDARR NGHKVLAVIR GSAVNQDGAS NGLTAPNGPS QERVIRAALA |
| 351 | NAGLGAAEVD AVEAHGTGTK LGDPIEAGAL LATYGRDRDE DRPLWLGSVK |
| 401 | SNIGHPQGAA GVAGVIKMVM ALQRELLPAT LYVDEPTPHV DWSSGSVRLL |
| 451 | TEPVPWTRGE RPRRAGVSAF GMSGTNAHVI LEEAPPEEAA AAETPAEGTG |
| 501 | AVVPWVVSGR GEEALRAQAA QLAEHVRDDD QRPASPLEVG WSLATTRSVF |
| 551 | ENRAVVVGDD RDALLDGLRS LAAGEASPDV VSGAVGPTGP GPVMVFPGQG |
| 601 | GQWVGMGARL LDESPVFAAR IAECEQALSA YVDWSLTDVL RGDGSELARI |
| 651 | DVVQPVLWAV MVALAAVWAD QGIEPAAVVG HSQGEIAAAC VVGAISLDEA |
| 701 | ARIVAVRSVL LRQLSGRGGM ASLGMGQEQA ADLIDGHPGV VVAAVNGPSS |
| 751 | TVISGPPEGI AAVVADAQER GLRARAVASD VAGHGPQLDA ILDQLTEGLA |
| 801 | GIRPAATDVA FYSTVTAGHL TDTTELDTAY WVRNVRRTVR FADTIDALLA |
| 851 | DGYRLFIEVS PHPVLNLALE GLIERAAVPA TVVPTLRRDH GDTTQLARAA |
| 901 | AHAFAAGADV DWRRWFPADP APRTVDLPTY APQRQDFWPA PAGGRSGDPA |
| 951 | GLGLAASGHP LLGASVGLAS GDVHLLSGRV SRQSAAWLDD HVVAGQALVP |
| 1001 | GAAQVEWVLR AGDDAGCSAL EELTLQTPLV LPDTGGLRIQ VVVEAADAHG |
| 1051 | RRDVRLFSRP DDDDAFASTH PWTCHATGVL APAPTDGTNG TRDAADTLDG |
| 1101 | AWPPADAEPV PADDLYAQAD RTGYGYGPAF RGVRALWRHG KDVLAEVTLP |
| 1151 | KEAGDPDGFG IHPALLDAVL QPAALLLPPT DAEQVWLPFA WNDVALHAVR |
| 1201 | ATTVRVRLTP LGERIDQGLR ITVADAVGAP VLTVRDLRSR PTDTGRLAAA |
| 1251 | ATRDRHGLFD LEWIAPENAA ENAAGPARDA SEGWVTLGED AASLADLLAS |
| 1301 | VEAGAPAPQL VAAPVEPDRT DDGLALATHV LDLVQTWLAS PLHDSRLVLV |
| 1351 | TRGAVTDADV DVAAAAVWGL VRSAQSEHPG RFTLIDLGPD DTLAAAMQAA |
| 1401 | HLEEPQLAVH GGEIRVPRLV RATTDPTAPN GTPEADRTAD PSEGLHRNGT |
| 1451 | VLITGGTGVL GRLVAEHLVT EWGVRHLLLA SRRGDQAPGS AELRARLSEL |
| 1501 | GASVEIAPAD VGDAEAVAAL IASVDPAHPL TGVIHAAGVL DDAVITAQTP |
| 1551 | ESLARVWATK ATAARHLHEA TRETPLDFFV VFSSAAASLG SPGQANYAAA |
| 1601 | NAYCDALVQH RRAQGLAGLS IAWGLWQATS GMTGQLSETD LARMKRTGFA |
| 1651 | ALTDEGGLAL LDAARAHDRA YVVAADLDPR AVTDGLSPLL RALTAPATRR |
| 1701 | RVASEGLADG ALATRLAGLD ADGRLRLLTD VVREYVAAVL GHGSAARVGV |
| 1751 | DIAFKDLGFD SLTAVELRNR LSAACDVRLP ATLIFDHPTP QALATHLVDR |
| 1801 | LAGSTSATTT VNATAPAAAH VAAGADVDAD TDDPVAIVAM TCRFPGGVAS |
| 1851 | PDDLWDLLDA RKDAMGAFPT DRGWDLERLF HPDPDHPGTS YTDQGGFLPD |
| 1901 | AGDFDAAFFG INPREALAMD PQQRLLLEAS WEVLERAGID PTTLKGTPTG |
| 1951 | TYVGLMYHDY AKSFPTADAQ LEGYSYLAST GSMVSGRVAY TLGLEGPAVT |
| 2001 | VDTACSSSLV SIHLATQALR HGECDLALAG GVTVMADPDM FAGFSRQRGL |
| 2051 | SPDGRCKAYA AAADGVGFSE GVGVLLLERL SDARRHGRRV LGVVRGSAVN |
| 2101 | QDGASNGLTA PNGPSQERVI RQALASGGLS SVDVDVVEGH GTGTTLGDPI |
| 2151 | EAQALLATYG QGRPEDRPLW LGSVKSNIGH TQAAAGVAGV IKMVMANRHG |
| 2201 | VVPASLHVDV PSPHVEWDSG AVRLAVESVP WPQVEGRPRR AGVSSFGASG |
| 2251 | TNAHVIVESV PDGLEEDSVS VGGEALETET DGRLVPWVVS ARSPQALRDQ |
| 2301 | ALRLRDFASD ASFRAPLADV GWSLLKTRAL HEHRAVVVGA ERAELIAALE |
| 2351 | ALATGEPHAA LVGPACSQAR VGGDDVVWLF SGQGSQLVGM GAGLYERFPV |
| 2401 | FAAAFDEVCG LLEGPLGVEA GGLREVVFRG PRERLDHTVW AQAGLFALQV |
| 2451 | GLARLWESVG VRPDVVLGHS IGEIAAAHVA GVFDLADACR VVGARARLMG |
| 2501 | GLPEGGAMCA VQATPAELAA DVDGSAVSVA AVNTPDSTVI SGPSDEVDRI |
| 2551 | AGVWRERGRK TKALSVSHAF HSALMEPMLA EFTEAIRGVK FRQPSIPLMS |
| 2601 | NVSGERAGEE ITDPEYWARH VRNAVLFQPA IAQVADSAGV FVELGPAPVL |
| 2651 | TTAAQHTLDE SDSQESVLVA SLAGERPEES AFVEAMARLH TAGVAVDWSV |
| 2701 | LFAGDRVPGL VELPTYAFQR ERFWLSGRSG GGDAATLGLV AAGHPLLGAA |
| 2751 | VEFADRGGCL LTGRLSRSGV SWLADHVVAG AVLVPGAALV EWALRAGDEV |
| 2801 | GCVTVEELML QAPLVVPEAS GLRVQVVVEE AGEDGRRGVQ IYSRPDADAV |
| 2851 | GGDDSWICHA TGVLSPESAR LDTELGGVWP PAGAEPLDVD GFYAQAGEAG |
| 2901 | YGYGPAFRGL RAVWRHGQDL LAEVVLPEAA GAHDGYGIHP ALLDATLHPL |
| 2951 | LAARFMDGSE DDQLYVPFGW AGVSLRAVGA TTVRVRLRPV GESVDQGLSV |
| 3001 | TVTDATGGPV LSVDSLQTRP VKPSQLAAAQ QPDVRGLFTV EWTPLPQTDA |
| 3051 | DGEADWVVLS DGVGRLADVV SAAGGEAPWA VVAPVDASVG DGREGLDGRL |
| 3101 | VVERVLSLVQ EFLALPELAE SRLLVVTRGA VATGVDGDGD VDASAAAVWG |
| 3151 | LVRSAQSENP GRFILLDVDG DGDDQGPDLN GRHLPHATLR HAAEELDEPQ |
| 3201 | LALREGTLYV PRLTQARQSA ELVVPPGEPA WRLRMVHDGS LDALAAVACP |
| 3251 | EALEPLAPGQ VRIAVHAAGI NFRDVLVALG MVPAYGANGG EGAGVVTEVG |
| 3301 | PEVTHVSVGD RVMGVFEGAF GPVVIAEARM VTPVPQGWDM REAAGIPAAF |
| 3351 | LTAWYGLVEL AGLKAGERVL VHAATGGVGM AAVQIARHVG AEVFATASPG |
| 3401 | KHAVLEEMGI DAAHRASSRD LAFEGTFREA TGGRGMDVVL NSLAGEFIDA |
| 3451 | SLRLLGDGGR FLEMGKTDVR AAEEVAAEHA DVSYTAYDLV GDAGPDRISN |
| 3501 | MLDKLVELFA SERLKPLPVR SWPLDKAQEA FRFMSQAKHT GKLVLEIPPA |
| 3551 | LDPEGTVLVT GGTGALGQVV AEHLVREWGV RHLLLASRRG PEAPGSDELA |
| 3601 | SKLTGLGAEV TIVAADVSDP ASVVELVGKT DPSHPLTGVV HAAGVLEDGV |
| 3651 | VTAQTPEGLA RVWAAKAAAA ANLHEATREM RLGLFVVFSS AAATLGSPGQ |
| 3701 | ANYAAANAYC DALMQHRRAV GQVGLSVGWG LWEAPDAKPG VAADAKASAA |
| 3751 | TVGKASALSD GTNGSAPQDT TGTAPQGMTG GLTDTDVARM ARIGVKGMSN |
| 3801 | AHGLALFDAA HRHGRPHLVG FNLDLRTLAT HPLHTRPALL RGLATPTAGG |
| 3851 | ASRPTATAGG QPADLAGRLA ALSPSDRHHT LVRLIREQAA TVLGHHPDSL |
| 3901 | TTGSTFKELG FDSLTAVELR NRLSAATGLR LPAGLVFDHP DADILAEHLG |
| 3951 | AQLAPDGDTP AGAEATDPVL RDLAKLENAL SSTLVEHLDA DAVTARLEAL |
| 4001 | LSNWKAASAA PGSGSTKEQL QVATTDQVLD FIDKELGV* |

MonAV, polyketide synthase multi-enzyme MONS5, housing
extension modules 7 and 8 Length: 4107 amino acids

| | |
|---|---|
| 1 | MASEEELVDY LKRVAAELHD TRQRLREVED RRQEPVAVVG MACRFPGGIE |
| 51 | TPEGLWELVA AGDDAIEPFP TDRGWDLEGI YHPDPDHPGT CYVREGGFLA |
| 101 | APDRFDSDFF CPSPREALAS SPQLRLLLET SWEALERAGI NPASLKGSPT |
| 151 | GVYVGAATTG NQTQGDPGGK ATEGYAGTAP SVLSGRLSFT LGLEGPAVTV |
| 201 | ETACSSSLVA MHLAANALRQ GECDLALAGG VTVMSTPEVF TGFSRQRGLA |
| 251 | PDGRCKPFAA AADGTGWGEG AGLILLERLS DARRKGHKVL AVIRGSAINQ |
| 301 | DGASNGFTAP NGPSQRRVIR QALSSAHLST SEIDVVEAHG TGTRLGDPIE |
| 351 | AEALIATYGK EREDDRPLWL GSVKSNIGHT QAAAGVAGVI KMVMALQREL |
| 401 | LPATLNVDEP TPHVQWEGGG VRLLTEPVPW SRGERPRRAG ISSFGISGTN |
| 451 | AHVVLEEAPP EEDVPGPVAA EPEGVVPWVV SARTEEALSE QARRLGEFVA |
| 501 | DTDPSTADVG WSLTTSRAIL EHRAVVVGRD RDALTAGLAA LAAGEESADV |
| 551 | VAGVAGDVGP GPVLVFPGQG SQWVGMGAQL LDESPVFAAR IAECEQALSA |
| 601 | YVDWSLSAVL RGDGSELSRV EVVQPVLWAV MVSLAAVWAD YGVTPAAVIG |
| 651 | HSQGEMAAAC VAGALSLEDA ARVVAVRSDA LRQLMGQGDM ASLGASSEQA |
| 701 | AELIGDRPGV CIAAVNGPSS TVISGPPEHV AAVVADAEER GLRARVIDVG |
| 751 | YASHGPQIDQ LHDLLTDRLA DIRPATTDVA FYSTVTAERL TDTTALDTDY |
| 801 | WVTNLRQPVR FADTIDALLA DGYRLFIEAS AHPVLGLGME ETIEQADIPA |
| 851 | TVVPTLRRDH GDTTQLTRAA AHAFTAGATV DWRRWFPADP TPRTIDLPTY |
| 901 | AFQRRSYWLP VDGVGDVRSA GLRRVEHSLL PAALGLADGA LVLTGRLAAS |
| 951 | GGGGGWLADH AVAGTTLVPG AALVEWALRA ADEAGCPSLE ELTLQAPLVL |
| 1001 | PGSGGLQVQV VVGPADGQGG RREVRVFSRV DSDDEAAGQD EGWSCHATGV |
| 1051 | LSPEPGAVPD GLSGQWPPTG AEPLEISDLY EQAASAGYEY GPSPRGLRSV |
| 1101 | WRHGHNLLAE VELPEQAGAH DDFGIHPVLL DAALHPALLL DQNAPGEEQE |
| 1151 | PAQPALRLPF VWNGVSLWAT GAATVRVRLA PHGGGETDDS AGLRVTVADA |
| 1201 | TGAPVLSVDS LALRPADPEL LRTAGRAGSC TNGLFTVEWT ALPPADVADH |
| 1251 | AAGDGWAVLG QDVPDWAGAD MPRHPDMASL SAALDEGTQA PAAVFVETTA |
| 1301 | TSHATPNTAA DVTLDASGRA VAERTLHLLR DWLAEPRLAE TRLVLITHHA |
| 1351 | VTTPADDDVN AAPLDVPAAA LWGLIRSAQA EHPDRFVLLD TDAKANTDPG |
| 1401 | PDTSTDHSTA SGTYRTVIAR ALATGEPQLA VRAGELLAPR LAPAATPTPE |
| 1451 | TPTPETQPDT GSGSEAGAGS GSGPGATLDP DGTVTLIAGGT GMMGGLVAEH |
| 1501 | LVRAWSVRHL LLVSRQGPDA PDARDLADRL VGLGATVRIV AADLTDGRAT |
| 1551 | ADLVASVDPA HPLTGVIHAA GVLDDAVVTA QTSDQLARVW AAKASVAANL |
| 1601 | DAATSELPLG LFLMFSSAAG VLGNAGQAGY AAANAFVDAL VGRRRATGLP |
| 1651 | GLSIAWGLWA RGSAMTRHLD DADLARLRAG GVKPLLDEQC LALLDAARAT |
| 1701 | AAHTSLVVAA GIDVRGLNRD DVPAILRDLA GRTRRRAAAD STVDQAALER |
| 1751 | RLTGLDEAER RAVVTDVVRE CVAAVLGHRS AADVRTEANF KDLGFDSLTA |
| 1801 | VQLPNRLSAA SGLRLPATLA FDHPTPQALA AYLGTRLSGR TATPVAPVAP |
| 1851 | SAAATDEPVA IVAMACKYPG GATSPEGLWD LVAEGVDAVG AFPTGRGWDL |
| 1901 | ERLFHPDPDH PGTSYADEGA FLPDAGDFDA AFFGINPREA LAMDPQQRLL |
| 1951 | LEASWEVLER AGIDPTTLKG TPTGTYVGVM YHDYAAGLAQ DAQLEGYSML |
| 2001 | AGSGSVVSGR VAYTLGLEGP AVTVDTACSS SLVSIHLAAQ ALRQGECTLA |
| 2051 | LAGGVTVMAT PEVFTGFSRQ RGLAPDGRCK PFAAAADGTG WGEGVGVLLL |
| 2101 | ERLSDARRHG RRVLGVVRGS AVNQDGASNG LTAPNGPSQE RVIRQALASG |
| 2151 | GLSSVDVDVV EGHGTGTTLG DPIEAQALLA TYGQGRPVDR PLWLGSVKSN |
| 2201 | IGHTQAAAGV AGVIKMVMAM RHGVVPASLH VDVPSPHVEW DSGAVRLAVE |
| 2251 | SVPWPEVEGR PRRAGVSSFG ASGTNAHVIV ESVPDGLGED SVSVSGEAPE |
| 2301 | TETDGRLVPW VVSARSPQAL RDQALRLRDA VAADSTVSVQ DVGWSLLKTR |
| 2351 | ALFEQRAVVV GRERAELLSG LAVLAAGEEH PAVTRSREDG VAASGAVVWL |
| 2401 | FSGQGSQLVG MGAGLYERFP VFAAAFDEVC GLLEGPLGVE AGGLREVVFR |
| 2451 | GPRERLDHTM WAQAGLFALQ VGLARLWESV GVRPDVVLGH SIGEIAAAHV |
| 2501 | AGVFDLADAC RVVGARARLM GGLPEGGAMC AVQATPAELA ADVDDSGVSV |
| 2551 | AAVNTPDSTV ISGPSGEVDR IAGVWRERGR KTKALSVSHA FHSALMEPML |
| 2601 | AEFTEAIREV KFTRPKVSLI SNVSGLEAGE EIASPEYWAR HVRQTVLFQP |
| 2651 | GIAQVASTAG VFVELGPGPV LTTAAQHTLD DVTDRHGPEP VLVSSLAGER |
| 2701 | PEESAFVEAM ARLHTAGVAV DWSVLFAGDR VPGLVELPTY AFQRERFWLS |
| 2751 | GRSGGGDAAT LGLVAAGHPL LGAAVEFADR GGCLLTGRLS RSGVSWLADH |
| 2801 | VVAGAVLVPG AALVEWALRA GDEVGCVTVE ELMLQAPLVV PEASGLRVQV |
| 2851 | VVEEAGEDGR RGVQIYSRPD ADAVSGDDSW ICHATGTLTP QHTDAPNDGL |
| 2901 | AGAWPAAGAV PVDLAGFYER VADAGYAYGP GFQGLRAVWR HGQDLLAEVV |
| 2951 | LPEAAGAHDG YGIHPALLDA TLHPALLLDW PGEVQDDDGK VWLPFTWNQV |
| 3001 | SLRAAGAATV RVRLSPGEHD EAEREVQVLV ADATGTDVLS VGSVTLRPAD |
| 3051 | IRQLQAVPGH DDGLFSVDWT PLPLSRTDVS QTDADGDADW VVLSDGVGSL |
| 3101 | ADVVSAAGGE APWAVVAPVG ASAGGGLAGF DRREGLDGRL VVERVLSLVQ |
| 3151 | EFLAAPELAE SRLLVLTRGA VATGGDGDGD VDASAAAVWG LVRSAQSENP |
| 3201 | GRFILLDVDM DVDVDVDMDV DVDVDVDVDV DGDGNGSDLD PDLNGRRLPH |
| 3251 | ATLRHAAEEL DEPQLALRDG QLLVPRLVRA TGGGLVVAPT DRAWRLDKGS |
| 3301 | AETLESVAPV AYPGVMEPLG PGQVRLGIHA AGINFRDVLV SLGMVPGQVG |
| 3351 | LGGEGAGVVT ETGPDVTHLS VGDRVMGVLH GSFGPTAVAD TRMVAPVPQG |
| 3401 | WDMRQAAAMP VAYLTAWYGL VELAGLKAGE RVLIHAATGG VGMAAVQIAR |
| 3451 | HLGAEVFATA SAAKHVVLEE MGIDAAHRAS SRDLAFEDTF RQATDGRGMD |
| 3501 | VVLNSLTGEF IDASLRLLGD GGRFLEMGKT DVRTPEEVAA EYPGVTYTVY |
| 3551 | DLVTDAGPDR IAVMMSELGE RFASGALDPL PVRSWPLDKA REAFRFMSQA |
| 3601 | KHTGKLVLDV PAPLDPDGTV LITGGTGALG QVVAEHLVRE WGVRELLLAS |
| 3651 | RRGLDAPGSG ELADRLSDLG AEVTVAAADV SDPASVVELV GKTDPSHPLT |
| 3701 | GVVHAAGVLE DGIVTAQTPE GLARVWAAKA AAAANLHEAT REMRLGLFVV |
| 3751 | FSSAAATLGS PGQANYAAAN AYCDALMQRR RAAGQVGLSV GWGLWEAPDA |
| 3801 | KPGVAADAKP DVAADAKTGV AADGTPQGMT GTLSGTDVAR MARIGVKAMT |
| 3851 | SAHGLALLDA AHRHGRPHLV AVDLDTRVLA HKPAPALPAL LRAFAGDQGG |
| 3901 | QGGGRGGGRG GGPARPAAAT TRQNVDWAAK LSVLTAEEQH RTLLDLVRTH |
| 3951 | AAAVLGHAGT DAVRADAAFQ DLGFDSLTAV ELRNRLSAST GLRLPATFIF |
| 4001 | RHPTPSAIAD ELRAQLAPAG ADPAAPLFGE LDKLETVITG HAHDESTRTR |
| 4051 | LAARLQNLLW RLDDTSARSD HAAGASDADG DAVENRDLES ASDDELFELI |
| 4101 | DRELPS* |

MonAVI, polyketide synthase multi-enzyme MONS6, housing
extension module 9 Length: 1701 amino acids

| | |
|---|---|
| 1 | MPGTNDMPGT EDKLRHYLKR VTADLGQTRQ RLRDVEERQR EPIAIVAMAC |
| 51 | RYPGGVASPE QLWDLVASRG DAIEEFPADR GWDVAGLYHP DPDHPGTTYV |
| 101 | REAGFLRDAA RFDADFFGIN PREALAADPQ QRVLLEVSWE LFERAGIDPA |
| 151 | TLKDTLTGVY AGVSSQDHMS GSRVPPEVEG YATTGTLSSV ISGRIAYTFG |
| 201 | LEGPAVTLDT ACSASLVAIH LACQALRQGD CGIAVAGGVT VLSTPTAFVE |
| 251 | FSRQRGLAPD GRCKPFAEAA DGTGFSEGVG LILLERLSDA RRNGHQVLGV |
| 301 | VRGSAVNQDG ASNGLTAPND VAQERVIRQA LTNARVTPDA VDAVEAHGTG |
| 351 | TTLGDPIEGN ALLATYGKDR PADRPLWLGS VKSNIGHTQA AAGVAGVIKM |
| 401 | VMAMRHGELP ASLHIDRPTP HVDWEGGGVR LLTDPVPWPR ADRPRRAGVS |
| 451 | SFGISGTNAH LIVEQAPAPP DTADDAPEGA ATPGASDGLV VPWVVSARSP |
| 501 | QALRDQALRL RDFAGDASRA PLTDVGWSLL RSRALFEQRA VVAGRERAEL |
| 551 | LAGLAALAAG EEHPAVTRSR EEAAVAASGD VVWLFSGQGS QLVGMGAGLY |
| 601 | ERFPVFAAAP DEVCGLLEGE LGVGSGGLRE VVFWGPRERL DHTVWAQAGL |
| 651 | FALQVGLARL WESVGVRPDV VLGHSIGEIA AAHVAGVFDL ADACRVVGAR |
| 701 | ARLMGGLPEG GAMCAVQATP AELAADVDGS SVSVAAVNTP DSTVISGPSG |
| 751 | EVDRIAGVWR ERGRKTKALS VSHAFHSALM EPMLGEFTEA IRGVKFRQPS |
| 801 | IPLMSNVSGE RAGEEITSPE YWARHVRQTV LFQPGVAQVA AEARAFVELG |
| 851 | PGPVLTAAAQ HTLDHITEPE GPEPVVTASL HPDRPDDVAF AHAMADLHVA |
| 901 | GISVDWSAYF PDDPAPRTVD LPTYAFQGRR FWLADIAAPE AVSSTDGEEA |
| 951 | GFWAAVEGAD FQALCDTLHL KDDEHRAALE TVFPALSAWR RERRERSIVD |
| 1001 | AWRYRVDWRR VELPTPVPGA GTGPDADTGL GAWLIVAPTH GSGTWPQACA |
| 1051 | RALEEAGAPV RIVEAGPHAD RADMADLVQA WRASCADDTT QLGGVLSLLA |
| 1101 | LAEAPATSSD TTSHTSTSCG TGSLASHGLT GTLTLLHGLL DAGVEAPLWC |
| 1151 | ATRGAVSCGD ADPLVSPSQA PVWGLGRVAA LEHPELWGGL VDLPADPESL |
| 1201 | DASALYAVLR GDGGEDQVAL RRGAVLGRRL VPDATPDVAP GSSPDVSGGA |
| 1251 | AHADATSGEW QPHGAVLVTG GVGHLADQVV RWLAASGAEH VVLLDTGPAN |
| 1301 | SRGPGRNDDL AAEAAEHGTE LTVLRSLSEL TDVSVRPIRT VIHTSLPGEL |
| 1351 | APLAEVTPDA LGAAVSAAAR LSELPGIGSV ETVLFFSSVT ASLGSREHGA |
| 1401 | YAAANAYLDA LAQRAGADAA SPRTVSVGWG IWDLPDDGDV ARGAAGLSRR |
| 1451 | QGLPPLEPQL ALGALRAALD GGKGHTLVAD IEWERFAPLF TLARPTRLLD |
| 1501 | GIPAAQRVLD ASSESAEASE NASALRRELT ALPVRERTGA LLDLVRKQVA |
| 1551 | AVLRYEPGQD VAPEKAFKDL GFDSLVVVEL RNRLRAATGL RLPATLVYDY |
| 1601 | PTPRTLAAHL LDRVLPDGGA AELPVAAHLD DLEAALTDLP ADDPRRKGLV |
| 1651 | RRLQTLLWKQ PDAMGAAGPA DEEEQAAPED LSTASADDMF ALIDREWGTR |
| 1701 | * |

MonH, probable regulatory protein Length: 981 amino acids

| | |
|---|---|
| 1 | VSGVERGVGS AGPVEQGDGL AGLVERAEAL AALRGAFDGS PGTGGSLVVL |
| 51 | SGAVGTGKTA LLRAWADRIG ADADALVLTA TACRAERDLP LGVLEQLVRS |
| 101 | PGLPPASAER ALAWWDEEAS ATPGKTDANG TSANGTDANG TGAGQTGAGQ |
| 151 | AGVGQTGVGG EPVLAASALR QLCEVLRDLL AERPVVVAVD DAHHADAASL |
| 201 | QCLLSVVRRL RSARLHVLFT EYAHQKAQNA LLSSEFLHEP ALRRIRLEPL |
| 251 | SKAGVEALLA RHLDERTAQD LTPVVHGMSA GHPLLVRALA EDHRAAGGAG |
| 301 | EAYGRAVLSF LYRHETPVTQ VARAIAALGA HAGPGQVGRL LDVDAASVER |
| 351 | AVRQLTVAEV LHEGRLCHPA FAAAVLDGMP PEERRALHGR VADLLHEEGA |
| 401 | PATEVAAHLV AADRSDAPWA VPVFQEAAQL ALDEDQVETG VDYLRAAHQR |
| 451 | CRGAAQRAAV VGALADAEWR LDPAKVLRHL PDPAAMAPQT DPAALAPHTD |
| 501 | PAPTAAPTAA PTPTPIPTTP PLPTHLLWHG RVEEGLDAIG TLTGPGPNPA |
| 551 | GAPPMNPADL DTPWLWGAYL YPGHVKERLG SGALSPQRST PPAVTPELQG |
| 601 | AGTLMNDLLH GGERDATEAA ERALNRYRLG PRTIAVQTAA LAALTYRDRP |
| 651 | HRAAAWCDGL VAQADERNSP TWRALFTAWR ALLHLRQGDP AAAEQRAETA |
| 701 | LALLGSKGWG AAIGLPLAAA VQAKAALGDV DGAAALLERP VPQAVFQTRT |
| 751 | GLHYLAARGR YHLATGCHYA ALCDFYACGT RMSSWGVDLP ALEPWRLGAA |
| 801 | EAYLALGEGL LARQLVDGQL PLPTPDDGRT WGMTLRLRAA TSPAPARAEL |
| 851 | LDEAVAVLRE SGDTFELARA VADQAVAVRE GGEAERARLL ARKAELLARR |
| 901 | WGSAPAPATV PEPPERPGPA TPDAELTSAE RRVAELAAEG FTNREISRKL |
| 951 | CVTVSTVEQH LTRIYRKLDV RRLDLQAALG * |

MonCI, flavin-dependent epoxidase Length: 496 amino acids

| | |
|---|---|
| 1 | VTTTRPAHAV VLGASMAGTL AAHVLARHVD AVTVVERDAL PEEPQHRKGV |
| 51 | PQARHAHLLW SNGARLIEEM LPGTTDRLLA AGARRLGFPE DLVTLTGQGW |
| 101 | QHRFPATQFA LVASRPLLDL TVRQQALGAD NITVRQRTEA VELTGSGGGS |
| 151 | GGRVTGVVVR DLDSGRQEQL EADLVIDATG RGSRLKQWLA ALGVPALEED |
| 201 | VVDAGVAYAT RLFKAPPGAT THFPAVNIAA DDRVREPGRF GVVYPIEGGR |
| 251 | WLATLSCTRG AQLPTHEDEF IPFAENLNHP ILADLLRDAE PLTPVFGSRS |
| 301 | GANRRLYPER LEQWPDGLLV IGDSLTAFNP IYGHGMSSAA RCATTIDREF |
| 351 | ERSVQEGTGS ARAGTRALQK AIGAAVDDPW ILAATKDIDY VNCRVSATDP |
| 401 | RLIGVDTEQR LRFAEAITAA SIRSPKASEI VTDVMSLNAP QAELGSNRFL |
| 451 | MAMRADERLP ELTAPPFLPF ELAVVGLDAA TISPTPTPTP TAAVRS |

MonBII, carbon-carbon double bond isomerase
Length: 141 amino acids

| | |
|---|---|
| 1 | MPDEAARKQM AVDYAERINA GDIEGVLDLF TDDIVFEDPV GRPPMVGKDD |
| 51 | LRRHLELAVS CGTHEVPDPP MTSMDDRFVV TPTTVTVQRP RPMTFRIVGI |
| 101 | VELDEHGLGR RVQAFWGVTD VTMDDPAGPA DTTHPEGIRA * |

MonBI, carbon-carbon double bond isomerase
Length: 144 amino acids

| | |
|---|---|
| 1 | MNEFARKKRA LEHSRRINAG DLDAIIDLYA PDAVLEDPVG LPPVTGHDAL |
| 51 | RAHYEPLLAA HLREEAAEPV AGQDATHALI QISSVMDYLP VGPLYAERGW |
| 101 | LKAPDAPGTA RIHRTAMLVI RMDASGLIRH LKSYWGTSDL TVLG |

MonAVIII, polyketide synthase multi-enzyme MONS8, housing
extension modules 11 and 12 Length: 3754 amino acids

| | |
|---|---|
| 1 | MSNEEKLLDH LKWVTAELRQ ARQRLHDKES TEPVAIVGMA CRYPGGARSA |
| 51 | EDLWELVRDG GDAVAGFPDD RGWDLESLYH PDPEHPATSY VRDGAFLYDA |
| 101 | GHFDAEFFGI SPREATAMDP QQRLLLETAW EAIEHAGMNP HALKGSDTGV |
| 151 | FTGVSAHDYL TLISQTASDV EGYIGTGNLG SVVSGRISYT VGLEGPAVTV |
| 201 | DTACSSSLVA IHLASQALRQ GECSLALAGG STVMATPGSF TEFSRQRGLA |
| 251 | PDGRCKPFAA AADGTGWGEG AGVVALELLS EARRRGHKVL AVIRGSATNQ |
| 301 | DGTSNGLAAP NGPSQERVIR AALANARLSA EDIDAVEAHG TGTTLGDPIE |
| 351 | AQALIATYGQ GRPEDRPLWL GSVKSNIGHT QAAAGVAGVI KMVMAMRNGL |
| 401 | LPTSLHIDAP SPHVQWEQGS VRLLSEPVDW PAERTRRAGI SAFGISGTNA |
| 451 | HLILEEAPPE EDAPGPVAAE PGGVVPWVVS GRTPDALREQ ARRLGEFAAG |
| 501 | LADASVSEVG WSLATTRALF DQRAVVVGRD LAQAGASLEA LAAGEASADV |
| 551 | VAGVAGDVGP GPVLVFPGQG SQWVGMGAQL LDESPVFAAR IAECEQALSA |
| 601 | HVDWSLSDVL RGDGSELSRV EVVQPVLWAV MVSLAAVWAD YGITPAAVIG |
| 651 | HSQGEMAAAC VAGALSLEDA ARIVAVRSDA LRQLQGHGDM ASLSTGAEQA |
| 701 | AELIGDRPGV VVAAVNGPSS TVISGPPEHV AAVVADAEAQ GLRARVIDVR |
| 751 | YASHGPQIDQ LHDLLTDRLA DIQPTTTDVA FYSTVTAERL DDTTALDTAY |
| 801 | WVTNLRQPVR FADTIEALLA DGYRLFIEAS PHPVLNLGIQ ETIEQQAGAA |
| 851 | GTAVTIPTLR RDHGDTTQLT RAAAHAFTAG APVDWRRWFP ADPTPRTVDL |
| 901 | PTYAFQHKHY WVEPPAAVAA VGGGHDPVEA RVWQAIEDLD IDALAGSLEI |
| 951 | EGQAESVGAL ESALPVLSAW RRRHREQSTV DSWRYQVTWK HLPDVPAPEL |
| 1001 | SGAWLLLVPA AHADHPAVLA TAQTLTAHGG EVRRHVVDAR AMERTELAQE |
| 1051 | LRVLMDGAAF AGVVNLLALD EEPHPEHSAV PAGLAATTAL VQALADNGAD |
| 1101 | IAVRTLTQGA VSTSAGDALT HPVQAQVWGL GRVAALEYPR LWGGLVDLPA |
| 1151 | RIDHQTLARL AAALVPQDED QISIRPSGVH ARRLAHAPAN TVGSGLGWRP |
| 1201 | DGTTLITGGT GGIGAVLARW LARAGAPHLL LTSRRGPDAP GAQELAAELT |
| 1251 | ELGAAVTVTA CDVGDREQVR RLIDDVPAEH PLTAVIHAAG VPNYIGLGDV |
| 1301 | SGAELDEVLR PKALAAHHLH ELTRELPLSA FVMFSSGAGV WGSGQQGAYG |
| 1351 | AANHFLDALA EHRRAEGLPA TSIAWGPWAE AGMAADQAAL TFFSRFGLHP |
| 1401 | LSPELCVKAL QQALDAGETT LTVANFDWAQ FTSTFTAQRP SPLLADLPEN |
| 1451 | RRASAPAAQQ EDATEASSLQ QELTEAKPAQ QRQLLLQHVR SQAAATLGHS |
| 1501 | DVDAVPATKP FQELGFDSLT AVELRNRLNK STGLTLPTTV VPDHPTPDAL |
| 1551 | TDVLRAELSG DAAASADPVR AAGASRGAAD DEPIAIVGMA CRYPGDVRSA |
| 1601 | EELWDLVAAG KDAMGAFPDD RGWDLETLYD PDPESRGTSY VREGGFLYDA |
| 1651 | GDFDAGFFGI SPREAVAMDP QQRLLLETAW EAIERAGLDR ETLKGSDAGV |
| 1701 | FTGLTIFDYL ALVGEQPTEV EGYIGTGNLG CVASGRVSYV LGLEGPAMTI |
| 1751 | DTGCSSSLVA IHQAAHALRQ GECSLALAGG ATVMATPGSF VEFSLQRGLA |
| 1801 | KDGRCKPFAA AADGTGWAEG VGLVVLERLS EARRNGHNVL AVIRCSAINQ |
| 1851 | DGTSNGLTAP NGQAQQRVIR QALANARLSA EDVDAVEAHG TGTMLGDPIE |
| 1901 | ASALVATYGK ERPADRPLWL GSIKSNIGHA QASAGVAGVI KMVMALRNEQ |
| 1951 | LPASLHIDAP TPHVDWDGSG VRLLSEPVSW PRGERPRRAG VSAFGISGTN |
| 2001 | AHLILEQAPD APEPVTAPAE DAAAPAGVVP WVVSARGEEA LRAQARLLAD |
| 2051 | RATADPRLAS PLDVGWSLVK TRSVFENPAV VVGKDRQTLL AGLRSLAAGE |
| 2101 | PSPDVVEGAV QGASGAGPVL VFPGQGSQWV GMGAQLLDES PVFAARIAEC |
| 2151 | ERALSAHVDW SLSAVLRGDG SELSRVEVVQ PVLWAVMVSL ASVWADYGIT |
| 2201 | PAAVIGHSQG EMAAACVAGA LSLEDAARIV AVRSDALRQL MGQGDMASLG |
| 2251 | AGSEQVAELI GDRPGVCVAA VNGPSSTVIS GPPEHVAAVV ADAEARGLRA |
| 2301 | RVIDVGYASH GPQIDQLHDL LTERLADIRP TTTDVAFYST VTAERLDDTT |
| 2351 | TLDTDYWVTN LRQPVRFADT IEALLADGYR LFIEASPHPV LNLGMEETIE |
| 2401 | RADMPATVVP TLRRDHGDAA QLTRAAAQAF GAGAEVDWTG WFPAVPLPRV |
| 2451 | VDLPTYAFQR ERFWLEGRRG LAGDPAGLGL ASAGHPLLGA AVELADGGSH |
| 2501 | LLTGRISPRD QAWLAEHRVM DTVLLPGSAF VELALQAAVR AGCAELAELT |
| 2551 | LHTPLAFGDE GAGAVDVQVV VGSVAEDGRR PVTVHSRPTG EGEEAVWTRH |
| 2601 | AAGVVAPPGP DAGDASFGGT WPPPGATPVG EQDPYGELAS YGYDFGPGSQ |
| 2651 | GLVSAWRLGD DLFAEVALPE AESGRADRYQ VHPVLLDATL HALILDAVTS |
| 2701 | SADTDQVLLP FSWSGLRVHA PGAEKLRVRI ARTAPDQLAL TAVDGGGGGE |
| 2751 | PVLTLESLTV RPVAANQIAG ARAADRDALF RLVWMEVAAR AEETGGGAPR |
| 2801 | AAVLAPVESG PMGGTSAGAL ADALSDALAA GPVWDTFGAL RDGVAAGGEA |
| 2851 | PDVVLAVCAA PGAGAGAVAD ADGRGGDPAG YARLATVSLL SLLKEWVDDP |
| 2901 | AFAATRLVVV TRGAVAARPG ETAGDLAGAS LWGLVRSAQA ENPGRLTLLD |
| 2951 | VDGLESSPAT LTGVLASGEP ELALRDGRAY VPRLVRDDAS VRLVPPVGSL |
| 3001 | TWRLARCQEA GGGQQLSLVD APEAGRALEP HEVRVAVRAA APGPLTAGQV |
| 3051 | EGAGVVTEVG GEVGSVAVGD RVMGLFDAVG PVAVTDAALL MPVPAGWSWA |
| 3101 | QAAGSLGAYV SAYHVLADVV APRGGETLLV GEETGSVGRA VLRLALAGRW |
| 3151 | RVEAVDGAST ADDSGAERAA DVTLRHEGAL VVHRAGGRPD EGQAVVPPEP |
| 3201 | GRVREILAEL TELTELAEIT ESAEPGLPAE RGDSRALTPL DITVWDIRQA |
| 3251 | PAAMAAPPSA GTTVFSLPPA FDPEGTVLVT GGTGALGSLT ARHLVERYGA |
| 3301 | RHLLLSSRRG ADAPGALELA ADLSALGARV TFAACDPGDR DEAAALLAAV |
| 3351 | PSDHPLTAVF HCAGTVNDAV VQNLTAEQVE EVMRVKADAA WHLHELTRDA |
| 3401 | DLSAFVLYSS VAGLLGGPGQ CSYTAANAFL DALARHRHDG GAAATSLAWG |
| 3451 | YWELASGMSG RLTDADRARH ARAGVVGLGA DEGLALLDAA WAGGLPLYAP |
| 3501 | VRLDLARMRR QAQSHPAPAL LRDLVRGGSK SGGGAVSAGA AALLKSLGAM |
| 3551 | SDPEREEALL DLVCTHIAAV LGYDAATPVN ATQGLRELGF DSLTAVELRN |
| 3601 | RLSAATGLKL PATFVFDHPN PAELAAQLRQ ELAPRAADPL ADVLAEFERI |
| 3651 | EDSLLSVSSK DGSARAELAG RLRATLARLD APQDTAGEVA VATRTRIQDA |
| 3701 | SADEIFAFID RDLGRDGASG QGNGQPTGQG NGHGNGNGNG NGNGHGQAVE |
| 3751 | GQR* |

MonAVII, polyketide synthase multi-enzyme MONS7, housing
extension module 10 Length: 1642 amino acids

| | |
|---|---|
| 1 | MAHTEEKLLE YLKRVTADLR QTERRLQDVE SAGHEPVAVI GMACRLPGGV |
| 51 | RSPEEFWELV STGGDAVAPL PGNRNWDLDS LYDPDPESTG TSYVREGGFV |
| 101 | YDAGDFDPTF FGIGPTEAAA MAPQQRLALE TAWEAIERAG IDPLSLRSSD |
| 151 | TSTFIGCDGL DYALGASEVP EGTAGYFTIG NSGSVTSGRV AYTLGLEGPA |
| 201 | VTVDTACSSS LVSLHLATQA LRTQECSLAL AGGTYVMSSP APLIGFSELR |
| 251 | GLAPDGRCKP FSASSDGMGM AEGTGVVLLE RLSDARRKGH KVLAVIRGSA |
| 301 | INQDGASNGL TAPNGPAQER VIRAALANAR LAPEDIDAVE AHGTGTTLGD |
| 351 | PIEAGALISA YGRERPEDRP LWVGAVKSNI GHTQIAAGVA GVIKMVLALR |
| 401 | HDLLPAILHV DAPSPHVEWD GSGLRLLTDP VKWPRGERPR RAGVSSFGFS |
| 451 | GTNAHLILEE APPEEEDVPG SVAEEPGGVV PWVVSGRTPD ALRAQARRLG |
| 501 | EFAAGPADAS AADVGWSLTT TRSVFEHRAV VVGRDRDALT AGLGALAAGE |
| 551 | ASAGVVAGVA GDVGPGPVLV FPGQGSQWVG MGAQLLDESP VFAARIAECE |
| 601 | RALSAYVDWS LSAVLRGDGS ELSRVEVVQP VLWAVMVSLA AVWADYGVTP |
| 651 | AAVIGHSQGE MAAACVAGAL SLEDAARIVA VRSDALRRLQ GHGDMASLST |
| 701 | GAEQAAELIG DRPGVVVAAV NGPSSTVISG PPEHVAAVVA DAEARGLRAR |
| 751 | VIDVGYASHG PQIDQLHDLL TERLADIRPA NTDVAFYSTV TAERLTDTTA |
| 801 | LDTDYWVTNL RQPVRFADTI EALLADGYRL FIEASAHPVL GLGMEETIEQ |
| 851 | ADIPATVVPT LRRDHGDTTQ LTRAAAHAFT AGAPVDWRRW FPADPTPRTV |
| 901 | DLPTYAFQHQ HYWLERSASA SGAVSGEQSA AEAQLWHAVE ELDLGLLAET |
| 951 | LGSEEGSEEA VRALEPALPV LKGWRRRHQD QATIDSWRYR VTWKQRSDGP |
| 1001 | APELGGDWLL FVPADKAEHP AVRATAEALS EHGAAAVRLH PVETGRAGRQ |
| 1051 | ELAAVDTAGL AGIVNLLALD EEPHPEHPAV PAGLAATTAL LQALGDNGTT |
| 1101 | APLHTVTQGA VSTGATDPLT HPLQAHVWGL GRVAALEHPR LWAGLVDLPA |
| 1151 | RIDRHTLPRL AAALLPQDDE DQTAVRPTGI HHRRLTHAVG SIQNPVHSEA |
| 1201 | TWRPRGTTLI TGGTGGIGAV LARWLARQGA PRLHLTSRRG PDAPGARELA |
| 1251 | AELDGLGTAV TITACDVSDP RQLSGLIDDM PAEHPLTAVI HAAGMTDLTA |
| 1301 | IGDLTTARLG EVLGSKSDAA WNLHELTRDL DLSAFVMFSS GAGVWGSGQQ |
| 1351 | GAYGAANHFL DALAEHRRAQ GLPATSIAWG PWAEAGMSAD PESLTYFKRF |
| 1401 | GLLPIAPDLC VKALHQAVDA GDATLTVANF DWAKFTPTFT AQRPSPFLDD |
| 1451 | LPENQREAEQ TGTAAETSAF REELAKTPAS QRLGFLVQQV RTYAAATLGR |
| 1501 | TVEDIPAAKP FQELGFDSLT AVQLRNQLNT TTGLSLPATV IFDHPTPEAL |
| 1551 | ATHLRGQLGD GAEVAGEGDV LAALDKWDTA FGAAEVDEAA RRRIVGRLQV |
| 1601 | LVSKWSPAQD GPEGTDSAHA DLEAASADDI FDLISSEFGK S* |

MonD, cytochrome P450 hydroxylase Length: 431 amino acids

| | |
|---|---|
| 1 | VGLTVGPDNA KRGIVPITDS KPAATFPDLV DPSFWARPHA ERVALFEEMR |
| 51 | GLPRPAFIRQ NMPGVPWTFG YHALVKYADI VEVSRRPQDF SSNGATTIIG |
| 101 | LPPELDEYYG SMINMDNPEH SRLRRIVSRS FGRNMIPEFE AVATRTARRI |
| 151 | IDELIARGPG DFIRPVAAEM PIAVLSDMMG IPAEDHDFLF DRSNTIVGPL |
| 201 | DPDYVPDRAD SERAVIEASR ELGDYIAGLR AERLAAPGND LITKLVQVQA |
| 251 | DGEQLTRQEL VSFFILLVIA GMETTRNAIS HALVLLTEHP EQKQLLLSDF |
| 301 | DTHAPNAVEE ILRVSTPINW MRRVATRDCD NNGHRFRRGD RIFLFYWSGN |
| 351 | RDESVFPDPY RFDITRGTNA HVTFGAVGPH VCLGAHLARM EITVLYRELL |
| 401 | AALPQIHAVG QPRRLDSSFI EGIKHLHCAF * |

MonRI, probable activator protein Length: 268 amino acids

| | |
|---|---|
| 1 | VRYEMLGPLR IKDGNDYATI NAQKVEIVLT VLLIRADRVV SLEQLMREIW |
| 51 | GEDLPRRATA GLHVYISQLR KFLKVPGSAG NPVETRAPGY VLHKRDDDQI |
| 101 | DAQIFPELVD VGRSLLREKR PDEAASCFGQ ALALWRGPIL GQGGNGPGTN |
| 151 | GPIIDGFSTW LTEIRLECQE MLVECQLQLG RHREAVGMLY ALTAENPMCE |
| 201 | AFYRQLMLAL YRSERQADAL KVYQSVRKTL NDELGLEPGR PLQELQRAIL |
| 251 | AGDMHLMSPP PLALSGR* |

MonAX, thioesterase Length: 278 amino acids

| | |
|---|---|
| 1 | LSAFLAKGKI LSAFPPPDMS DPWIRRFRPR PEAVVRLVCF PHAGGSASYY |
| 51 | HPLAQSPTLP TDSEVLAVQY PGRQDRRRER LLDDIGELAD LITDALGPFD |
| 101 | DRPLAFFGHS MGAVLAYEVA QRLRERTGKQ PCRLFVSGRR APSRFRRGTV |
| 151 | HLLDDTELAA ELRRAGGTDP RFLDDEELLA EIIPVVRNDY RAVELYRWNP |
| 201 | SPPLSCPITA LVGDRDPQAP LDEVEAWQQH TEGPFDLKVF AGGHFYLNTN |
| 251 | QQGVTEVISK ALADSAQQRA TARGNAR* |

ORF29, a homologue of CapK involved in cell wall
biosynthesis Length: 428 amino acids

| | |
|---|---|
| 1 | LADLVAHARS ASPYYRELYH GLPERIEDPT LLPVTDKKQL MDHFDDWPTD |
| 51 | RDITFEKVRA FTDDPELIGR RFLGRYLVAT TSGTSGRRGL FVLDDRYMNV |
| 101 | SSAVSSRVLA SWLGPLGIAR AVVHGGRFAQ LVATEGHYVG FAGYSRLRQD |
| 151 | GEARSKLVRA FSVHEPMSRL VAELNEYRPA FVIGYASTIM LFTAEQEAGR |
| 201 | LHIDPVLVEP AGETMTESDT DRIAAAFGAK VRTMYSATEC TYLSHGCAEG |
| 251 | WYHVNDDWAV LEPVDADHRP TPPGEFSHTT LISNLANRVQ PFLRYDLGDS |
| 301 | VMLRPDPCPC GTPSPAIRVQ GRSGDILTFP SGRGDDVSLA PLAFSSLFDR |
| 351 | MPGVELFQIE QTAPSTLRVR VVQAPGADAD HVWQRAHDGL THLLADNKLD |
| 401 | NVTVERGEEP PRQASGGKYR TIIPLAA* |

LipB, lipase B Length: 338 amino acids

| | |
|---|---|
| 1 | VKVPVEVTVR LSSWLGGLVA AVLAATVLPA SAASAADVSS PPLEIPAAEL |
| 51 | AKALHCGTEL GDLRDAGDKP TVLFVPGTGL KGEENYAWNY MAELKKKGYQ |
| 101 | SCWVDSPGRG LRDMQESVEY VVYATRAIQE ATGRKVDLVG HSQGGLLTAW |
| 151 | ALRFWPDLPG KVDDMVTLGS PFQGTRLASP CRPIAEVAGC PASVLQFARD |
| 201 | SMWSKALGAD GTPMPAGPSY TTIYSYADES VVADGEAPSL PGAHRIGVQD |
| 251 | ICPGRPWPTH IAMVVDQVSY DLVADAIEHP GPADTSRIDR AHCAKPVMPL |
| 301 | NSQEAVDALP GLLNFPIELL THSQPWVDEE PPLRPYAR |

ORF31, putative ion pump Length: 309 amino acids

| | |
|---|---|
| 1 | MGHDHGPSAG AAGGTLSGTY RKRLLWTIGI SGSITVIQVV GALLSGSLAL |
| 51 | LADAAHSLTD AVGVSLALGA ITLAQRAPTP RRTFGFCRVE IFSAVLNALL |
| 101 | LVVIFAWVLW SAIGRFSEPV EVKGGLMFVV ALGGLAANLV GLWLLRDAKE |
| 151 | KSLNLRGAYL EVLGDALGSV AVIVGGLVIL LTGWQAADPI ASIVTGLLIV |
| 201 | PRAYGLLRDS LHVLLEATPQ DVDLGEVRRH LLEERGVVAV HDLHGWTVTS |
| 251 | GMPVLTAHVV VTEEALASGY GELLGRLQRC VGGHFDVAHS TIQLEPEGHV |
| 301 | EEDGALHT* |

ORF32, hypothetical membrane protein Length: 364 amino acids

| | |
|---|---|
| 1 | MTRALTLHDW IVAGIAVVAG VVAGLLLRAL LRWLGERASK TRWSGDDVIV |
| 51 | DALRTLVPCA AITAGLAAAA GALPLTPRTG RNVTMTLTAL LILAATLTAA |
| 101 | RIVTGLVKAV AQSRSGVAGS ATIFVNITRV VVLAMGFLIV LQTLGISIAP |
| 151 | LLTALGVGGL AVALALQDTL ANLFAGVHIL AAKTVQPGDY IQLSSGEEGY |
| 201 | VVDINWRNTT VRQLSNNLVI IPNAKLAGTN MTNYSRPEQE LSIMVQVGVS |
| 251 | YDSDLEQVEK VTTEVVDEVM AEITGAVPDH EAAIRFHTFG DSRISFTVIL |
| 301 | GVGEFSDQYR IKHEFIKRLH QRYRAEGIRV PAPVRTVRVQ QGELPPPLGI |
| 351 | PHQRDTSTQA RLH* |

AmtA, glycine amidinotransferase (partial coding sequence)
Length: 131 amino acids

| | |
|---|---|
| 1 | MSPVNSHNEW DPLEEIIVGR LEGATIPSSH PVVACNIPTW AARLQGLAAG |
| 51 | FEYPQRLIEP AQQELDQFIA LLQSLDVTVR RPAAVDHKHR FGTPDWQSRG |
| 101 | FCNSCPRDSM LVVGDEIIET PMAWPCRCFE T |

1. A DNA sequence which is (a) at least part of the sequence set out in the appended sequence listing; or (b) a variant of a sequence (a) which encodes a polypeptide which is at least 80%, preferably at least 90%, identical with the corresponding peptide as set out in table II; provided that it is not a sequence encoding all or part of the polypeptide consisting of amino acids 1-920 encoded by mon AI as set out in table II.

2. A DNA sequence according to claim 1 comprising the complete monensin gene cluster or a variant thereof.

3. A DNA sequence encoding at least part of at least one polypeptide which is necessary for the biosynthesis of monensin, and which is encoded by DNA included in the appended sequence listing or an allele, mutation or other variant thereof; provided that said polypeptide is not all or part of amino acids 1-920 encoded by mon AI as set out in table II.

4. A DNA sequence according to claim 3 which comprises at least part of one or more of the following genes: mon BI, mon BII, mon CI, mon CII, mon H, mon RI, mon RII, mon T, mon AIX and mon AX.

5. A DNA sequence according to claim 4 comprising all of the genes listed therein or an allele, mutation or other variant thereof.

6. A DNA sequence according to claim 3 encoding at least part of one or more of the polypeptides set out below, said polypeptide having the amino acid sequence as set out in the appended sequence data or being a variant thereof having the specified activity:

| peptide | activity |
|---|---|
| mon CII | epoxyhydrolase/cyclase |
| mon E | S-adenosylmethionine-dependent methyltransferase |
| mon T | monensin resistance gene |
| mon RII | repressor protein |
| mon AIX | thioesterase |
| mon AI | polyketide synthase multienzyme |
| mon AII | polyketide synthase multienzyme |
| mon AIII | polyketide synthase multienzyme |
| mon AIV | polyketide synthase multienzyme |
| mon AV | polyketide synthase multienzyme |
| mon AVI | polyketide synthase multienzyme |
| mon AVII | polyketide synthase multienzyme |
| mon AVIII | polyketide synthase multienzyme |
| mon H | regulatory protein |
| mon CI | flavin-dependent epoxidase |
| mon BII | carbon—carbon double bond isomerase |
| mon BI | carbon—carbon double bond isomerase |
| mon D | cytochrome P450 hydroxylase |
| mon RI | activator protein |
| mon AX | thioesterase |

7. A DNA sequence according to claim 6 encoding a single enzyme activity of a multienzyme encoded by any of mon AI-mon AVIII or a variant or part thereof.

8. A DNA sequence according to any preceding claim encoding any one or more of the domains as set out in Table I or a variant or part thereof.

9. A DNA sequence according to any preceding claim which has a length of at least 30, preferably at least 60, bases.

10. A recombinant cloning or expression vector comprising a DNA sequence according to any preceding claim.

11. A transformant host cell which has been transformed to contain a DNA sequence according to any of claims 1-9 and which is capable of expressing a corresponding polypeptide.

12. A hybridisation probe which is a DNA sequence according to any of claims 1-9.

13. Use of a probe according to claim 12 to detect a PKS cluster, optionally followed by isolation of the detected cluster.

14. Use of a probe according to claim 12 which encodes at least part of a polypeptide having a known function to detect genes encoding polypeptides having analogous function.

15. Use according to claim 14 wherein the polypeptide of known function is AT of module 5 or the regulatory protein encoded by mon RI.

16. A hybridization probe comprising a polynucleotide which binds specifically to a region of the monensin gene cluster selected from mon BI, mon BII, mon CI, mon CII, mon H, mon RI, mon RII, mon T, mon AIX and mon AX.

17. Use of a probe according to claim 16 in a method of detecting the presence of a gene cluster which governs the synthesis of a polyether, and optionally isolating a gene cluster detected thereby.

18. Use of a probe according to claim 12 which comprise a polynucleotide which binds specifically to a gene responsible for levels of activity of the monensin gene cluster, in a method of detecting an analogous gene in a gene cluster for biosynthesis of another polyketide, optionally followed by a step of manipulating the gene detected thereby to alter the level of expression of said other polyketide.

19. Use according to claim 18 wherein the gene is a regulatory gene, resistance gene or thioesterase gene.

20. Use of the mon RI gene or variant and a monensin promoter to control expression of a heterologous gene in S. cinnamonensis.

21. Use of a portion of the monensin gene cluster encoding a polypeptide having chain terminating activity, preferably comprising at least one of mon AIX and mon AX or a mutant, allele or other variant thereof encoding a polypeptide having chain terminating activity, to effect chain release of a peptide other than monensin.

22. Use of a portion of the monensin gene cluster encoding a polypeptide having carbon-carbon double bond isomerase activity, preferably comprising at least one of mon BI and mon BII or a mutant, allele or other variant thereof having isomerase activity to provide a desired stereochemical outcome in the synthesis of a polyketide other than monensin.

23. A polypeptide encoded by a portion of the monensin gene cluster, preferably comprising at least one of mon BI and mon BII or a mutant, allele or other variant thereof, having carbon-carbon double bond isomerase activity, or at least one of mon AIX and mon AX or a mutant, allele or other variant thereof having chain terminating activity.

24. An epoxidase enzyme encoded by mon CI or a derivative or variant thereof having epoxidase activity.

25. A cyclase enzyme encoded by mon CII or a derivative or variant thereof having cyclase activity.

26. Use of a portion of the monensin gene cluster encoding a peptide having epoxidase or cyclase activity, preferably comprising mon CI or mon CII or a mutant, allele or other variant thereof encoding a polypeptide having epoxidase or cyclase activity to provide a said activity in the biosynthesis of a polypeptide other than monensin.

27. A process for producing a polyketide containing a desired starter unit comprising providing a PKS gene having a loading module and a plurality of extension modules, wherein the loading module includes a KS_{q} domain derived from a KS domain of a monensin extension module.

28. A process according to claim 27 wherein the KS_{q} domain is derived from KS of module 5 of monensin.

29. A process according to claim 27 or claim 28 wherein the starter unit also includes an AT_{q} domain derived from an AT domain which is naturally associated with the KS domain.

30. A DNA sequence comprising DNA encoding at least one PKS loading module and a plurality of PKS extension modules, and which can be expressed to produce a polyketide; wherein at least one of said modules or at least one domain thereof is a monensin module or domain or a variant thereof and is contiguous to a further one of said modules or a domain to which it is not naturally contiguous; provided that the sequence is not an ery loading module, the first and second extension modules of the ery PKS and the ery chain-terminating thioesterase in which the DNA encoding AT of the first extension module has been substituted by DNA encoding an ethyl malonyl-CoA AT from the monensin gene cluster.

31. A DNA sequence according to claim 30 wherein said further module or domain is also a monensin module or domain or variant thereof.

32. A DNA sequence according to claim 30 wherein said further module or domain is a module or domain of a PKS of a polyketide other than monensin or a variant thereof.

33. A DNA sequence according to claim 30, 31 or 32 wherein said loading module is adapted to load a starter unit other than a starter unit normally received by the adjacent extension module.

34. A DNA sequence according to claim 33 wherein said loading module is derived from a monensin extension module or variant thereof.

35. A polyketide synthase encoded by the DNA sequence of any of claims 30-34.

36. A polyketide compound as produced by a synthase according to claim 35.

37. A vector containing a DNA sequence of any of claims 30-34.

38. A transformant cell transformed to contain a DNA sequence of any of claims 30-34.

39. A method of producing S. cinnamonensis capable of enhanced levels of production of monensin comprising engineering it to overexpress the mon RI gene.

40. A method according to claim 39 wherein said engineering comprises introducing at least one additional copy of the mon RI gene as shown in the appended sequence data or a variant thereof.

41. S. cinnamonensis containing multiple copies of the mon RI gene as shown in the appended sequence data and/or variant(s) thereof.

42. A method of producing monensin comprising culturing the organism of claim 41 and/or an organism produced by the method of claim 39 or claim 40.

43. A process for expressing a gene heterologous to S. cinnamonensis comprising transforming S. cinnamonensis with DNA encoding a heterologous gene and expressing said gene under control of the activator gene mon RI or actII/orf4.

44. A process according to claim 43 wherein said heterologous gene is a PKS gene.

45. 13-Propyl erythromycin A.


SEQUENCE LISTING (52 sequences)

SEQ ID NO: 1 - DNA, 30000 bp, Streptomyces cinnamonensis
  Nucleotides 1 to 30000 of the monensin biosynthetic gene cluster
gatcagcgcggtggcgtcgtcggcgtccagctcgttctgcgtggcggacggcagcgcgatgtcggcaggcacctcccagacccggcggcccggcacgaagcgggccgaggcgccgcggcgctgggcgtaggtgtccacgcgggcgcgttcgacctccttgacctgcttgaggaggtccaggtcgatgcccttctcgtcgacgacgtaaccggaggagtccgaacacgtcacggcgttggcgcccagggcggcgagcttctggatggtgtagatggcgacgttcccggagccggacacgaccgccgtccggccttcgagggtctcgccgcgctcacgcagcatcgccgccgcgaagaggacgttgccgtagccggtcgcctccggacggatcagggagccgccccagttgcggcccttgccggtgaggacgcccgcctcccagcggttggtgatgcgccggtactgaccgaacagatagccgatctcccggccgccgacgccgatgtcgcccgcgggcacgtccgtgtgttcgccgatgtgccggtacagctccgtcatgaacgactggcagaaacgcatgacttccgcgtcgctgcggccgcgcgggtcgaagtcgctgccgcccttgccgccgccgatgccgaggcccgtcagcgcgttcttgaagatctgctcgaagcccaggaacttgatgacgccgaggttcaccgacgggtggaagcgcaggccgcccttgtacgggccgagggcgctgttgaactccacccggaagccgcggttgacccgcacgcgaccgtggtcgtcctgccacggcacccggaagacgatctggcgctccggttcgcacaggcgctcgatcaggccggcttcggcgtactcggggcgagccgcgatgaccggcgccagggtctcgaggacctcgcgggcggcctggtggaactccggctgggccgggttgcggtgttcgatctcggtgagcagctgggagagtgctgtcttctgcgagagagctgtcttcgtgtcgggtcgcgtggtcaaaggagccctttctggcacggccggcgtaggcgctcggcgccgttgccgtgcgcagggagacgctcgagccgcaagtatgacgcgcatgtaaacacagcgaccagccccccggtccagggagtgaccaccatgcgagaccgggccaccggtagggccaccggtccggcctgcggaccccgtgtcacttccggctcgcggccaggggtgccgcccggcggaccgaatcggcggaggcggccagcagtggcatgcggacggccgggctgggaatgcggttctgggcgtgcagcactcccttgatcaccgtcgggttcggttcggtgaagagggcggcggaaaggcgggcgaggtcggctccgagagcgcgggcgggtgcggcggagccgcgtcgccacagcgcgatcatctcggcgtagtcggcggtacgcagattggccgacgccacgattccgccgtgggcgcccgcagcgaccagcggcgagaggacgatgtcgtcaccgccgagcacggcgaagccgggcaggggcgagtcgagcaactccatggtggtcgggtcgatcgagccggtcgcgtgcttgatgccgacgacctccggcaggcggccgagtgcggtgatcgtgcccgcgccgagcgtctgcccggtgcggtaagggatgtcgtacacgaccagggggaggccgccgtgctcggccagcgcggcgaaatgagccagggtccccgcttccccggggcggatgtagggcggcgcggggaccagcgcggcggcgacgtcaccccgggccgccagctctcgcagggccgtgatggcggtggcggtgtcgttggtgcccaccccgacgatgagcggtgccccgtgtgcccggcacgcggccgagcagacgcggatcaccgtctctctctcctcggcggtcagtgtggcggcctcggcggtcgtaccgagggcgacgagcccggaggcgccggccgacagcgcctcgtcggcgagtcgggccagcgcctcgggggccaggcgcagatcgtcggtgaacggagttaccagggggacgtacaggccgttgaagagcggttcggtggtcggttcgaggctcgatgcgagggtcatgctcttaccctggcccacgccactcggtagatccatttcagattcctgccgtcacacctaagctgaacttatgctcgatgtccgtcgcctccatctgctccgcgaactcgaccggcggggcaccatcgccgccgtggccgaagcgctgaccttcaccgcgtccgccgtctcccagcagctcggcgtgctggagagggaggcgggcgtgccgctgttggaacgcagcggcaggcgcgtggtcctcacgcccgcaggacgctccctcgtcgcacacgccgacgcggtgctgaaccgtctcgaacaggcggtcgccgagctggcgggcgcacgggacggcatcggcgggccgctgcgcatcgggacgttcccttccggcggccacaccatcgtccccggcgcgctggccgaactggcctctcgtcaccccgcgttggagccgatggtgcgggagatcgactccgcgcgcgtctccgacggtctgcgggccggtgagctggacgtggccctcgtacacgactacgacttcgtacccgcgacgccggacacgaccgtggacgaggtgcctctgctcgaagagccgatgtacctcgtcacccatgccgcggacactgccacggactccggctccgggagcacactggcagcgctgctcgggccctgtgccgaggttccgtggatcacggcgcgggacggcacgaccggtcacgcgatggctgtacgcgcctgtcaggccgccgggttccagcccaggatccgccaccaggtcaacgacttccgcacggtgctggctctggtcgccgccgggcagggggccgggttcgtgccgcggatggccgccgagccgagccccgcgggcgtggtgctcacgaagctgccgctgttccgtcgctcgaaggtcgcgttccgtgcgggcggcggtgcccatccggcgatcgccgctttcgtggccgcggcgacgacggcggtcgaacgcatggcgggttcacgaggcccggccggcggctctgagtgaaccggccgaccgtgggaatgtgtgtgccctgggccgcaccattcgtggcctggtgacgtcctggcgacgtcctgacgtcctgatgtccgaacgagaaggcgattttccgcgatggccgatgacgcgtacctgttcctcctccccgaccggcacccccgactgggagcggccctcgccgccgtcggtgccttggaatgcacggaaacccctgcggtgcacgcctggttgcaggctcatgaggcctccgtgtcctcggaacaggtcaggattctgcccgccgatgccgagacactcatcccgaaggacgccgagcggctgccggtgccgttgagcgaggaggaggcgctcaaggtcgagcaggagtgcgcgccccagaccgtcacggacatggagagcgaactgctcgcgttccgggagacgacccaggactggcaggccctcgtgcaccgggccctgaccgcgggcatccccgcgcagcgcatcgcccggctgaccggactcgacccggaggagatcggccgcctgtaggcgctagcggccgcccagtgcggacaccaggatggcgaccgtgacggtgttgaagacgaaggcgatgaccgtgttcgccgccacggtccgtcgcatgtcgcgtgaggtgacgtcgacatcggtggtgccgaacgtcgtcatcgcggccagggcgaaatagacgtagtcggcccaggcgggactccgctccccggggaattccagtgcccgctcgttctccacgaggttgtcggcctggaaggtgacggcgaaggccacgaccacgcagatccaggcggcgacgaccagggcgagcgccaccagggtgcgggggagcgcggagaaggtggtgctgaggtggccgggaagccacagcaccgccaccaccagcgcggcagccgcgatgaagagcgaacccccggggccgggcgcggttccgaggacgtaacgctgcaggaatgtgccgcgggcttcgcgccgcgcccaggagcggacctgctccggagcgacgctcacgaagacggtcatggtgatggcgaggtagggcagcaggtaggcgaagaagacgagcacgccgacatccgctgccgaaatccgcaccacggcgtcgatggggaggaccactgccgcgcacgccgcgacggccaggctcaccgccgaccggcgccgttcggaaagccagcgatgcacggacgagcctctctggtcgggcgtcgggcctcgtgtgatcgtgaccggctccgcgcccgccgaaagcgcggtgcgatctcctgccctcgaacgagcgaaacgcttgcgccggaaagcctccctgctgatgccgacggcggcggcagtggctgcggatgcggatcgtgcgctgtgccctgaccctggatggggggaggaacgcagagaggcaggtgcgcccatgacggtcatggacaagctcaagcagatgctcaaggggcacgaggacaaggccggccagggaatcgacaaggcgggcgacttcgtcgacgggaagacgcagggcaagtacagcggtcaagtcgacacggcccaggacaagctccgggaccagttcggctcggatcagcaggagcctccgcagaggtaggcagcgtcagggcggaatcggtccgggcgaccgctgaccgctgatgcagatgccgcagacgtcggccccgcactcctccgggtaaatcggagcgtaggcggggccgacgtgtgcgcgtgcggcctcgtctctgccgcccctctccgccccgtctctggccccttggtgccagtctgacgggaaaatggcaccacttggtgccacgcatgtgccatgatggcgtcatcgagagcgcgctgccccgactcgcgggcaggaagggcgcgttccgcggagtcggccgtcggaggggttgcatcatggggacagcacagagccaggagcaggccgccgcgcccggtgcctgcgccgccttcgtccgcttcgtgctctgcggtggcggagtgggcctcgcctccagcttcgccgtggtcgccctcgcctcctgggttccctgggcgctggccaacgccctggtcgccgtggtctccaccgtcgtcgccaccgagctccacgcccgcttcaccttcggtgcgggcgggcgcgcgacctggcggcagcacgcgcagtcggccgggtccgcggcggccgcgtacgcggtgacctgcgtggcgatgttcgtcctgcagcagctggtggcggcgcccggcgcggtgctcgagcaggtcgtgtacctgtcggcctccgcgctcgccggtgtcgcgcggttcgtggtgctgcgcctcgtcgtcttcgcccggaaccgctcgctgcccgccgcggccgccgtgcgcaccgcgcgtcccgtgcgtcgcgtgccggcgcccgtgcccgcgaccgtggcccacgccgcatcgcgcccggccggccccgcggcgctctgccccgccgcatgactccgtgcccgcatgtttgtgcccccggtgctccgtgcgtccgggggcggggtgggcgtcgtgcccgggtggtccaggggtcacgcggtggtgtgtgccagttcctggccgaggtggtgggcgagctgtgcgggcgtggggttctcgacgatggcgaccatcgcgatctccatcccggtcagcgtcatcagtgtcttggtgagctcaagggcggtgagggagttgagaccgttctcgaggaagttgctgtcgtcgctgagggtggtgttcagaagggtgccggcctgggtgcggatggtgtcggtgaggagcttctcgcgctcctcgggggtggccgcggcgagctgcttctccagctcggtggcgtcctggccggaggtgtggtcggtgctggtcatgactgctcctgtgtgagtgaggtgttggcgggggtcacaccgcggcgtgcgcggtgtggtcgtgcagccagtaacgcgtggcctggaaggagtacgtcgggaggtcgatggtccgggggtggggggtgcgccggacgagaggggtccagtcgacggtgccgcccgtggtgtgaagccgcgcgagggcggtcaacagggcgcttacggcggaggtttgcgtgccttccggtgagagcgcgcccaggtggagaagcgtgtgggtctcgggggtggggggtgcggtgggggccggcgaggtgaggtggtggtgccagtagtcggcggaggcgatgggggtgtcggccggggcagtgctggtgagcgtgagcgtggcgcgttggaacgtcagctgcttcagcacgggctcgtaggcgtcgggcggagccggttgttcaccctcggcggcctgggcggcagcggcgtgggcggcggccaggcggcacgcgtcgtcgagggtcaggattcccgcggcgtacgcggcggcgatgtggccgacaccgtcgccggtgagggtgtgggggcgtacccccgtttccaggagcagccgcgcgagcgcggtgtggaccgcgaagcgcgccagttcggagtggggagtggggaggggagtcggcagatgggtgtcgaggagcgcgcgcgcttcgtcgaaggcggacgcgaagagcgggaacgccgagtggaactcggcacctccgaaagccgcgccgaatgtagcgccgaatgtcgcgccgggtttggctccgggtgccgcccccgtcgtcaccccgtcggccgggcggccgtcgaagtgccaggcgatcttcttcgggccggccccgggcgtggacctgaccaggtccgggtggtcctctccggcggccagggcgcgggcggcggcgaggagttcggtgtggtcggtgccggtgaggacggcgcggtgttccagggggctgcgggtggcggcgagcgagtaggcgacctcggcgggggagggcgcggggtcggtggccgccaggtgggtgacgagggccttcgcctgtgcccgcagggcctcgggtgtacgagcggacaggctccaggccaccgggagttccggggcaacgggcgacgtctggtcgcgggcggcatccggcaccggagcctcgtccaccggcggctcttcgaggatgaggtgcgcgttcgtgccggacgtggcgaaggcggagatgccgacccggcggggctcctcgcggcggggccagtcgaccgcctcggtgagcagccgtaccgcgcccttcttccaggcggcgaggggcgtcgggcggtcgacgtggagggtcggcggcagggtgccgtgccggaacgcctggaccatcttgatgagcgcggccgcacccgcggccccctgcgtgtgccccgtgttggacttgacggagccgagccacaggggccggtcgggggagcggtcggcgccgtaggtggcgaggagggcctggacctcgatggcgtcgccgatgggggtgcccgtcccgtgcgcctcgacggcgtcgatctggtccggggtgagcccggcgtcggcgagggcggcgcggatcacatgctgctgggaggggccgttgggggcggcgaggccgtatccggcgccgtcctggttgaccgcggagccgcggatgacggcgagcaccgggtggccgttcttcctggcgtcgccgagccgctcaagcaggacgaggccgacgccttcaccgaggcccatgccgtcggccgcggcggcgaacggtttgcaacggccgtcctgcgcgagcgacttctggtgggcgaaggcgtggaaggtgtgcggcgtcgacatgacggtgccgccgccggcgagggcgaggccgcactccccgcggcgcagcgcctggcaggccaggtggagggcgaccagggaggacgagcaggccgtgtccacgctgatggcggggccctcgaggccgagggcgtaggcgatgcggccggagacgaggctgccggacgtgccgccgcccagatagggcagcagctcgtcgggcgcggtctcgagccgtgtcgcgtagtcgtgcccggtggcgccgacgtagacgccggtgagggtggagcgcagggtgtgcggggcgatgtggccgcgttcgacggtctcccacgcgaggtggagcatgaggcgctggaggggttcggtggccacggcctcggtgtcgctgatgtcgaagaagcccgcgtcgaagccggccgcgtcgtccaggaacccgccgagctccgcgtacgggcgttcctcggggagttcccaggcgcggtcgtcggggaagccggtgacggcgtcgcggccctcggacaccagatcccacaggtcgtccggggtgcgggtcttgccgggcagccggcaggccatggagacgacggcgatcggctcgtgctgtgcggccttcagttcgcgcagctgctgctgggcctggtggagctcggccgtcgtccacttgaggtattcgacgagcttctcttcgttcgccacgggaatggtcagccttcctgttctcgcgcgtgaagcctcaggtgggacgaggtcgggcaaggtgggcaggcaggagccgcgcgctgtgggtgccagggtcgccgcggctgcttaagcgggtctaactcccgccttgccgccgggcatcgcctcgcacgagcgggccagcagcaggaggtcggcggcgatctcgtcgggtgcgccggcgtgcagatcgtggtcggagcccgggtaccagcgcacgctcacctgctccagggccgcctcggcggcggccacccaggcccgtacctggtcggacagttgggggatggcggggatgaggggcagcagccgcaccggcacggtgaccttgggataccagtcggccggtgcctcccgttgcaggccggcgacgatcgacatgacctgtgtcgaggtcaggcgggggatgagcaggccgtccggcccgacgcggtagtccgccaggcgtgcctcgatggacgtgggcgaccagtcgggatgggtggcccgcaggtaggcgcgcatgtcggcggcgctggtggtgccctgctgggcgcgccggaccacgtcggcggtgcgctcccagaaggcgcgcatcaccggtccgtcgaactcgtaccagccgccgtcgatcagggcgagaccggccaccaggtccgggtgctcggccgccaggcgcagcgcgaggtgcgcgccccaggagtgcccggccaccagtgcgccggacaggtcgagggcggtgacggccgccaccaggtcggtgacgaccgtcgcgttgtcgtacccgtcgggcggggtgtccgactcgccgtggccgcggtggtcgacggcgtaggccgggtgtccggcggcggcgagacgggcggcgacctcgtcccacatccgggcgttcgacagcatgccgtgcagcagcaggaacggacggcccggggctcccggcccgtccgcgggccggtacctgacattgagggagacggtctgcgacacggggatgcggaggttcttcacaggcgggcccttgtgatcccttgtgctgggggaggaaagcgggggcggcacgctcaggggcgctgcgcggtcgcgaagatgtatccgagctcgggcatcttgccgagggccgcctggttgtgcaggaacagctcgtatccctctacgccgatgatgtcgacgtactcgtcccggtgggcgcggatccactcgacgtaaccgtcgtaggtcttggcggtctcgcgggtgatgtcggtcagttcgaggacggtccagccggcggcgcggaagatgtcggggtagtccccgatgtcggtgagcgcggcgtagatcgtggtgtcgctgacggtggcggtccggggccggctgggatcggggttgaggtagaccatgtcggcgatcggcatccgcgcgccgggcttcacgacgcggtgggcctcggtgagcacctgctgcttgtccggcatgtgcagcatggactccagggcccagcagtggtcgaaggagccgtcgtcgaacggcaggttcatggcgtcgacctgctcgaagcggacccggtcggcgaggccggcctcgcgcgcccggcggttgccgcgctcgacctggcgggcgctgacggagatgccgaccacctcgacgtcgcgggcgcgggccagctgcatggccggggtgccgttgccgcagccgatgtcgaggacgcggtcgccgggggccgggtcgaggcggcggatcatctcgtcggtcatctggaccatggcctcgtcgaacgtggcctgctgctcgccgccgtcgaaccagtagccgtagtgcagattgccgtctccgagctgagtcatcaggtcgaagaccttgtggtcgtagtagtggccgatgtcgctgggctcgggggcgacggtcttgttcaccgtcgggggcttcttggtcgtcgcgttcttcgtcacggcttcagcgtcaccgtgcggcggcaggcgccacaaccccacccccgcccctcaaaagcccctatgggccctcctcgaccgcccctagggagctgctcttgacgcgttccatacggaacgggtggtacccctccgaaaaaaatgagagtacgctcccactagatattgagctctctttaggaggtcgactcccatgtctgctgatctgggtgcgcggcggtggtgggccgtcggtgctctcgtactcgcctcgatggtcgtgggcttcgatgtgacgatcctgagcctggcgttgcccgccatggccgacgacctcggcgcgaacaacgtcgagctgcagtggttcgtgacgtcgtacacgctggtgttcgcggccggcatgatcccggccggcatgctcggtgaccggttcggacgcaagaaggtcctgctcaccgccctggtgatcttcggtatcgcctcgctggcctgtgcctacgcgacgtcctccggcaccttcatcggcgcgcgtgcggtgctcggtctgggcgccgcgctgatcatgccgacgacgctgtcgctgctgccggtcatgttctccgacgaggagcggccgaaggccatcggagcggtggccggtgcggcgatgctcgcctatccgctcggcccgatcctcggcggctacctgctcaaccacttctggtggggctccgtcttcctgatcaacgtgccggtggtgatcctcgccttcctcgcggtctccgcctggctgcccgagtccaaggccaaggaggccaagccgttcgacatcggcggcctggtgttctccagcgtcggtctcgccgcgctgacctacggcgtgatccagggcggcgagaagggctggacggacgtcaccacgctggtgccgtgcatcggcggtctgctcgccctcgtgctgttcgtgatgtgggagaagcgggtggcggacccgctggtcgacctctcgctgttccgctcggcccggttcacctccggcaccatgctcggcaccgtcatcaacttcacgatgttcggcgtgctcttcacgatgccgcagtactaccaggcggtcctcggcaccgacgcgatgggcagcggcttccggctgctcccgatggtcggcggtctgctcgtgggtgtgacggtcgccaacaaggtcgccaaggccctcggcccgaagaccgcggtcggcatcggcttcgccctcctcgccgccgccctgttctacggcgccaccacggacgtcagcagcggcaccggcctggcggccgcctggaccgcggcctacggactcggcctcggcatcgccctgccgaccgccatggacgccgccctcggcgcgctctccgaggactccgccggcgtcggatccggcgtcaaccagtccatccgtaccctcggcggcagcttcggcgcggccatcctcggttccatcctcaactccggctaccgcggcaagctcgacctcgacggcgtgcccgagcaggcacacggcgcggtcaaggactccgtcttcggcggcctcgcggtggcccgggcgatcaagtccaacggactggccgactcggtgcgttccgcgtacgtccacgccctggacgtggtgctcgtggtctccggcggcctcggactgctgggtgtggtgctggcggtggtgtggctgccccgccatgttggtcagagcaccgccaagacagcagaatctgagcatgaagccgcagacgcagtctgaccagggcaaaacagtgcctggtctgagagaacgcaagaaggcccggacgaaggccgcgattcagcgggaggcggtgcgcttgttcagggaacagggctacaccgccacgaccatcgagcagatcgccgaagccgccgaggtcgctcccagcaccgtcttccgctacttcgcgaccaagcaggacctggtcttctcgcacgactacgatctgcccttcgcgatgatggtccaggcccagtcacccgacctgacgccgatccaggccgagcggcaggccatccgctcgatgttgcaggacatcagcgagcaggaactggccctgcagcgcgagcggttcgtcctgattctctccgagccggagctctggggcgccagcctcggcaacatcggccagaccatgcagatcatgagtgagcaggtggccaaacgggccgggcgcgacccgcgggaccccgcggtccgcgcctacaccggagccgtgttcggagtgatgctccaggtctcgatggactgggccaacgatccggacatggacttcgcgaccacgctggacgaggcactccactacctggaagacctgcggccctgaccgaaggggcgggcgcacaccacagagcccgccccggccagacgtcgtacgaggcgccatcggccgtcgcgtacgacccccgcgccccggattcccccgcggggcgcggggtcaagggaaaagagacgaccgcacgcggccactgttcccccggctgccgcgtccggtccaacctggcgtgctccggcttccctcgacggagcacgccaggggtctgtccggccctctcccggcggctcccgtcagacgcccggccccgccgtcagcgcctcggtcacgacggccgccacctgctcctgacagccgtcgaggtagaagtgcccgccgggcagcacccgcagatcgaacgccgcgccggtccgctcccgccacgtggcggcctgctccggcgacgtccgctcgtcggcgtccccgatcagcgccgtgatcgggcagtcgagccggccgggtcccggcgcctcgtaggtggccacggcccggtagtcggcccgcagcgcgggcaggacgagctcctgcagctcgggactgcggaagaaccgctcgtcggtgccgcccatcgcccgcagatgggccaggatgtccgcgtccccgaacgcccccgaacgccccgcgggacggtagggccgcgcgagccccccggaaacgaacaggtgcacgggaaggccgggcccggccggtccccgcagccgccgcgccacctcgaacgccacgatggcgcccaggctgtgcccgaacagcgcgaatggcttcccgtcgcacggcaggtggggcacgacgccgtcggcgagctcggccaccgacgccaggcacggctccgcatgacggtcctgccgccccggatactgcacggcgagcacctcgacgccgggcgcgagcagcccggagagcccgaagtagtaactcgccgaaccgcccgcgaacggaaagcagaccagccgcaccggcgcctctgccgcagcgtggtaccgccgcaaccacaccccgtttccggtggctgcaccgaactcgtcaccgatctgtggtgcccgcgccgccgtgcccctgtccatcgttctccctctcctcgcgtcgctccgcgggcgctgtcctgccccgccccgaaagcccgatgccggccaagccccgatgctggccaaaccccgatgccggccaagccccgatgctggccgcggcccatagcgcccggctaaagccgcaggcggctagccggggtttggttcgcctttagacagcccacccacgatgagcccggtactcgaagcgatctccgatttcggaccgggagcgccgttgatgttttgtggcagccagttgttcagcgcccgaccgcagctgacgtgatggccgcatccgcgtcagcgtccccctcgggaccgagcgcaggacccgacccgatcgccgtcgtcgggatggcctgccgcctgcccggagcacctgaccccgacgcgttctggcggctgctcagcgaggggcgcagcgcggtgagcaccgcaccgcccgagcggcggcgagccgactccggcctccacgggccgggcggctacctggaccggatcgacggcttcgacgcggacttcttccacatcagcccgcgcgaggccgtggcgatggacccccagcagcggctgctcctcgaactgagctgggaggccctcgaagacgcgggcatccggccgcccaccctggcgcgcagccgcaccggcgtcttcgtcggcgcgttctgggacgactacaccgacgtcctgaacctgcgggcgccgggcgccgtcacccgccacaccatgaccggcgtgcaccgcagcattctggccaaccgcatctcgtacgcgtaccacctggccggtccgagcctcaccgtcgacaccgcacagtcctcctcgctcgtcgccgtccacctggcctgcgagagcatccgcagcggcgactccgacatcgccttcgcgggcggcgtcaacctcatctgctcgccgcgcaccaccgagctggccgcggcccgcttcggcggtctctcggccgcaggccgctgccacaccttcgacgcccgcgccgacggtttcgtacgcggcgagggcggcggcctcgtggtgctcaagcccctcgcggcggcacggcgcgacggcgacacggtgtactgcgtgatccgggggagcgccgtcaacagcgacggtacgaccgacggaatcaccctgcccagcgggcaggcgcagcaggacgtggtgcgcctcgcctgccgacgggcgcggatcacgccggaccaggtgcagtacgtcgaactgcacggcaccggcacgcccgtcggggacccgatcgaggccgccgcgctcggcgccgccctcgggcaggacgccgcccgcgccgtgccgctggccgtcggctccgccaagacgaacgtcggccacctcgaagccgccgccggaatcgtcggactgctcaagaccgccctgagcatccaccaccggcggctggcgccgagcctgaacttcaccacccccaatccggccatcccgctcgccgacctcggcctgaccgtccagcaggacctggccgactggccgcgccccgaacagcccctgatcgccggggtgtcgtccttcggcatgggcggcacgaacggtcacgttgtcgtggcggcggcgcccgattcggtggcggtacctgagccggtgggggtgcctgagcgggtggaagtgcctgagccggtggtggtttctgagccggtggtggtgccgacgccatggcccgtgagcgctcacagcgcttccgcgctgcgcgcgcaggccggtcgcctgcggacgcacctcgccgcccaccgccccacccccgacgccgcgcgggtcggccacgcgctcgccaccacccgtgcgcccctcgcccaccgcgcggtcctgctcggcggcgacaccgccgaactgctgggctccctggacgcgctggccgagggcgcggagaccgcgtccatcgtgcgcggcgaggcgtacaccgagggcaggacggccttcctcttcagtgggcagggagcgcaacgcctcggcatggggcgggagttgtatgccgtgttccccgtcttcgccgacgctctcgacgaggcgttcgccgccctggacgtacatctggaccgcccactgcgcgagatcgtcttgggcgagaccgactcgggtgggaacgtctcgggtgagaatgtcatcggcgagggtgccgaccatcaggcactcctcgaccagaccgcctacacccagcccgcgctcttcgcgatcgagacgagcctgtaccggctggcagcctccttcggcctgaagccggactacgtcctcggccactcggtcggcgagatcgccgccgcgcacgtcgccggtgtcctctcgttgccggacgcgagcgctctggtggccacgcggggacggctcatgcaggcggttcgcgcgcccggcgcgatggccgcgtggcaggccacggcggacgaggcggccgaacagctcgccgggcacgagcggcacgtcaccgtggccgccgtcaacggccccgactccgtggtcgtctccggcgaccgcgccaccgtcgacgaactgaccgccgcctggcggggacgcggccgcaaggcccaccacctgaaggtcagccacgccttccactccccgcacatggaccccatcctcgacgagctgcgcgcggtcgccgccggcctgaccttccacgagccggtcattcccgtcgtctccaacgtcaccggtgaactggtgaccgcgaccgcgaccgggagcggcgccgggcaggccgaccccgagtactgggcgcggcatgcgcgcgagcccgtgcggttcctgtccggggtgcgggggctgtgcgagcgcggggtgaccacgttcgtcgagctcggcccggacgcaccgctgtccgcgatggcccgcgactgcttccccgcccccgcggaccggagccgtccgcgccccgccgccatcgccacatgccgccgcgggcgcgacgaggtggccacgttcctgaggtcgctggcccaggcgtacgtccgcggcgccgatgtcgacttcacccgggcctacggcgccaccgccacgcgccgcttccccctccccacgtatcccttccagcgcgagcgccattggcctgccgctgccggggtggggcagcagccggagaccccggaacttccggaatcctcggagtcctcggagcaggcagggcatgagcgggaggagggggcgcgcgcgtggggcgggcctgaagggcggcttgccgggctctccgtgaacgaccaggagcgggtcctcctcggcctggtcaccaagcacgtggccgtcgtgctcggggacgcctcgggcacggtacaagccgcccgcaccttcaagcagttgggcttcgactcgatggccgccgccgagctgagcgaacggctcggcacggagacgggcctgccgttgcccgccaccctcaccttcgactacccgacccctctggccgtcgccgcgcacctgcgcgcggagctcaccggtacgcccgccccggccggctccgcgcccgccacgggcgccctcggcgcgggtgacctcggcacggacgaggacccggtcgccatcgtggccatgagctgccgctatcccggcggcgcaggcacgcccgaggacctgtggcggctggtcgcggacggcgccgacgcgatcggagacttccccaccgaccgcggctgggacctggcgcggctgttccaccccgaccccgaccggtcgggcaccagctgcacgcggcagggcggattcctgtacgacgccgccgacttcgacgccgagttcttcgacatcagcccgcgcgaggccctggccgtcgacccgcagcagcggctgctcctcgagtgcgcctgggaggccttcgaacgggcgggcctggacccgcgggcgctcaagggcagccccaccggcgtgttcgtcggcatgacggggcaggactacggcccccgtctgcacgagccgtcccaggccaccgacggctatctgctgaccggcagcacgccgagcgtggcctcgggccgcctgtcgttcagcttcggccttgaggggcccgccctgacggtggacacggcctgctcgtcgtcgctggtcacgctccatctcgcggcgcaggcgctgcggcgcggcgagtgcgacctggccctcgccggcggcgccaccgtcctggccacgccgggcatgttcaccgagttctcgcggcagcggggcctggcccccgacggccgctgcaagccgttcgcggcgggcgccgacggcacgggctgggccgagggcgtgggcctggtcctcctcgaaaggctctccgaggcccggcgcaaggggcacgccgtcctcgcggtgatccggggttcggcgatcaaccaggacggcgcgagcaacggcctgaccgcgcccaacggcccctcgcagcaacgcgtcatccgtgccgcgctcgcggccgcccggctcaccgcggacgaggtcgacgtagtggaggcgcacggcaccggcaccacgctcggcgacccgatcgaggcgcaggccctgctcgccacgtacggccaagggcgttcggcggagcggccgttgtggctcgggtcggtgaagtcgaacatcggtcacacgcaggccgccgcgggtgtcgcgggcgtcatcaagatggtgatggcgatgcgccacgacctgctccccgccaccctgcacgtcgacgagccgagtggccacgtggactggtccaccggcgcggtgcgactgctcaccgagccggtcgtctggccgcgcggcgaacgtccgcgccgcgccgcggtgtcgtccttcggcatctccggcacgaacgcgcacctggtgctcgaagaggcggggcaggacgagtacgttgcgggagccgccgacgacgccgggccggtggacggtgctgtgctgccgtgggtggtttccggacggaccggagcggcgctgcgcgaacaggcccgccgtttgcgtgagttggtgaccggcggctcggccgatgtctctgtgtccggggtgggccggtcgctggtcaccacgcgggcggtgttcgagcaccgggccgtggtcgtgggccgcgaccgggacacgctgatcggcggcctcgaggcccttgcggcgggtgacgcgtcgccggacgtcgtgtgcggggtcgcgggcgatgtcggccccggcccggtgctggtgttccccgggcagggctcgcagtgggtgggcatgggagcccaactccttggcgagtccgcggtgttcgcggcgcggatcgacgcgtgcgagcaggcgctgtccccgtacgtcgactggtcactgacagaggtcctgcgcggggacgggcgcgaactgtcgcgcgtcgacgtcgtccagcccgtgctgtgggcggtgatggtctcgctcgccgccgtctgggcggaccacggcgtcaccccggccgccgtcgtcgggcactcccagggagagatcgccgctgtggtcgtcgccggcgcgctcaccctggaggacggcgccaagatcgtggccctgcgcagccgggcgctgcgtcagctctcgggcgggggcgccatggcctccctcggggtgggccaggaacaggcagccgaactcgtcgagggccaccccggagtgggcatcgccgccgtcaacggcccgtcatcgaccgtcatttcaggcccgcccgagcaagtcgccgccgtcgtcgccgacgccgaggcgcgcgagctgagaggccgcgtcattgacgtggactacgcctcgcacagcccccaggtcgacgccatcaccgacgaactcacccacaccctgtccggcgtccgccccaccacggccccggtggcgttctactcggccgtgaccggaacccgcatcgacacggcgggcctcgacaccgactactgggtcaccaacctgcgccgcccggtccggttcgccgacgccgtcaccgcgctcctcgccgacggccaccgggtcttcatcgaggccagcagccaccccgtcctcaccctcggcctccaggagaccttcgaggaggccggggtcgacgccgtcaccgtccccaccctgcggcgcgaggacggcggccgggcacgcctggcccgctcgctggcacaggccttcggcgccgggtgcgcggtgaggtgggagaactggtttccggccaccggtacgtccaccgtggagctgccgacgtacgccttccagcgtcgccgttactggctggaggcccccacgggcacccaggacgcggcgggcctgggcctcgccgctgcggggcacccgctcctcggggcggccaccgagatcgcggacggcgacatccgcctgctcaccggccgtatcagcaggcacagccacccctggctcgctcagcacaccctcttcggtgccgcggtcgtgcccgcctccgtcctcgcggaatgggcgctgcgcgccgccgacgaggccggctgcccgcgtgtcgacgacctcacgctgcgcaccccgctggtgctgcccgagaccgcgggcgtgcaggtgcagatcgtggtcggcccggccgacgcgcgggacgggcaccgcgacttccacgtctacgcccgccccgacggcaaggacgcctctgagggcgagggcatcgccgagggcgagggtgcctctgagggcgagggtgcctccggcggcaccgatgcgccgtggacctgccatgccgacggccgactggtcgccgagcccaccggcacggcctcggaggactccccggacacggtgtggccgccgcccggcgccgaacccgtcgacctgggcgacttctacgagcgggccgccgccaccggagtcggctatggaccggtcttcacggggctgcgcgccctgtggcggcgggacggcgagctgttcgccgaggcggtgctgccgcaagaagccccggaaaccgccgggttcggcatgcacccggcgctcctcgacgccgcactgcaccccgcactcctcggcgagcggccggccgaggaggacaaggtgtggctgccgttcacgctgaccggagtgaccctgtgggccaccggtgccacctctgtacgcgtccgtctcaccccgctggacgacgaccccgacgcgtcggcggacgggcgggcctggcgggtcggcgtgagcgacccgaccggcgcggaggtgctgacctgcgaggccctggtcgcggtggcggcgggccgccgcgagctgcgggccgcgggggagcgggtgtccgatctgtacgcggtggagtgggtgccggtgccgggcccggggccggtgggtgagggtgctgacttctcgggctgggccggtctgggggagtgcggggagcgttgggagtgcgtggggcgcgtggagcgctggtacgaggacctggacgctctcggcgcggctgtcgagggtggggcttcggtgccctctgtcgttctcgccaccgcggctgccgcccctggtggagcgggcgacggagccgccgatgcgctgagcgcggtgcggtggaccggcgcgctcctcgatcagtggctcgccgacgcgcggttcgccgacgcccggctggtggtgatcacgtccggcgcggtcgccacgggtgacgatttccttcccgacccggccgccgcggcggtacgaggactggtcgagcaggcgcaggtcaggcaccccggccgcatcctcctcgtcgacacggaagccggggccgggctcggggtcggcgccggagtggatgacgcgctcctggaacaggccgtggccatggctctcggcgccgacgaaccgcaactcgccctgcgcgcggggcgggtcctggcgccccgcctcaccgcaccccaggatgcggccgtcaccgaagcggcgcgaccgctcgacccggacggcaccgtactcatcacagggccggccggtgctccggtggccgacctcgccgaacacctcgtacgcaccgggcagtgcaggcatctgctgctcctgcctggagacggtgaactggaggaaatggccgaggagttgcggggcctcggcgccaccgtggacctgagtaccgccgacccggcggacccgaccgccctcgccgaagtggtcgccgccgtcgagggggaccatcctcttacgggggtcatccacgccaccggagtcgtggacgcgttcgatcccggcgactcggcgagcgacttgatgatcgactcggcgagcgattcgttcgccgaggcatggtcgtcgagggcgggcgtcaccgccgcactgcacaccgcgaccgcccaccttcccctggacctgttcgccgtcctgtccccggcgggcgcggacctgggcattgcccggtcggcggccgccgcgggcgccgacgccttcagcgcggcactcgccctgcgccggcacacgaccgtcacgacggacacgacagccccgccgcgcacgacagccccgccgcgaacgacagcctcgccgcgcacgacagccctgtcgtcgtcgcgcacgacgggcgtggccctcgcctacgggccgcccaccgcgccgaggcccggcatcaaggggacggcgcccggtcggatccccgtgctgctcgacgccgctcgcgctcacgggggcggttcgcccctgctcggggcccgcttggccgcgcgtgccctggccgccgagtccgccgccgagggcgtcgccggcctgcccgcgccgctgcgcgcgctggcagtggccgcagccgcggccggagcaccgacccggcgcaccgccgccgaccgcaagccccccgcggactggccggcccgactggcccccctgtccgcccccgaacaactccgtctgctcatcgacgccgtacgcacccacgccgccgcggtcctcggccgcaccgacccggaagcgctgcgcggggacgccaccttcaagcagctcggccttgactcgctgaccgccgtggagctgcgcaaccggctcgtggaggacaccggtctgcgcctgcccaccgccctcgtctttcgctacccgacccccgcggcgatcgccgcgcacctccgcgagcggctgaccagcccgagcgagacgaccgccacacagaggtccggagggcagacgcccgcagcggggcaggcgtcgtccgcgctcgcccccggcggatcggccgccggaccgcccgccgcagacaccgtgctgagcgacctgacccgcatggagaacaccctctccgtgctcgccgcccagctgccccacaccgagacgggtgagatcaccacccggctcgaagcgctcctcacgcgctggaagaccacgaacgccacggcgaacgacagcggcgacggcaacggcggcgatgacgacgccgccgaacgcctcaaggccgcgtccgccgaccagatcttcgacttcatcgacaacgagcttggtgtcgggcacggcacctcgcgcgtgacccccactccgaaggccgggtgaccgcacatggcgagtgaagagcaactggtcgaatatctgcgcagggtgaccaccgagctccatgacacgcgtcggcgcctggtgcaggaggaggaccgcaggcaggaaccggtggccctggtcggcatggcctgccgcttcccgggcggcgtggcctcaccggaggacctctgggacctggtcgccgcgggcaaggacgccatcgaggactttcccaccgaccggggctgggacctggaggcgctctacgacccggacccggccgcgtacgggaccagctatgtccgccacggcgggttcgtggacgacgcgggctccttcgacgccgacttcttcggcatcagcccgcgagaagccctggcgatggacccgcagcagcggctgatgctggagacgtcctgggagctgttcgagcgcgccggcatcgaacccgtctccctcaagggcagccgtacgggcgtctacgccggggtgtccagcgaggactacatgtcccaactgccccgcatccccgaggggttcgaggggcacgccaccaccggcagcctcaccagcgtcatctcgggccgggtcgcgtacaactacggcctcgaaggcccggccgtcaccgtcgacacagcctgttccgcctcgctcgtcgccatccacctggcgagccaggcgctgcgccagcgtgagtgcgacctcgccctcgcgggcggtgtgctcgtactgtccagcccgctcatgttcaccgagttctgccgccagcggggccttgctcccgacggccgctgcaagccgttcgccgccgcggcggacggcaccggcttctcggagggcatcggtctgctcctcctggagcgcctgtccgacgcgcgccgcaacggccacaaggtgctcgcggtgatccgcggctccgccgtcaaccaggacggcgcgagcaacggcctgaccgcccccaacgacgccgcgcaggaacaggtcatccgcgccgccctcgacaacgcccgcctcaccccgtccgaggtggacgccgtcgaggcgcacggcaccggcaccaaactgggcgaccccatcgaggccggagcgctgctcgccacctacgggcaacaccgcgcccggcccctcctcctcggctccctcaagtccaacatcggccacacccacgccaccgcgggcgtcgccggtgtcatcaagaccgtcatggcgatccgcaacggtctgctccccgccaccctccacgtcgaggaactgagcccgcacgtcgactgggacgcgggcgcggtcgaggtcgtcacggagcccaccccgtggcccgagaccggccacccccggcgcgcgggcgtctccgcgttcgggatctccgggacgaatgcgcacttgatcctggaggaggcgccgccggaggaggatgtgcccgcccccgtggttgtggagtcgggcggggtcgttccgtgggtggtgtccgggcggacgccggaggcgctgcgtgaacaggcccggcgactcggcgagttcgtggcaggcgacacggacgcactgccgaacgaggtcggctggtccttggccacgacccggtcggtgttcgagcaccgggctgtggtcgtggggcgtgaccgggatgcgttgacggctggcctgggggcgttggctgcgggtgaggcttcggcgggtgtggtggccggggtggccggtgatgtgggtcctgggccggtgttggtgtttccggggcagggggcgcagtgggtgggcatgggtgcccagctgttggacgagtctgcggtgttcgcggcgcggatcgcggagtgtgagcgggccctgtcggcgcatgtggactggtcgctgagtgcggtgttgcgcggggacgggagtgagctgtcccgggtggaagtggtgcagccggtgctgtgggcggtgatggtctcgctggctgcggtgtgggcggattacggggtcactccggctgccgtgatcgggcactcgcagggtgagatggctgccgcgtgtgtggcgggggcgctgtcgctggaggatgcggcgcggatcgtagcggtacgcagtgacgcgcttcgtcagctgcaagggcacggcgacatggcctcgctcagcaccggtgccgagcaggccgctgagctgatcggtgaccggccgggcgtggtcgtcgcggcggtcaatgggccgtcgtctacggtgatttcagggccgccggagcatgtggcagccgtggtcgcggatgcggaggcacgtggtctgcgcgcccgtgtcatcgacgtcggctatgcctcgcatggcccccagatcgaccagctccacgatctgctgaccgaacgcctggccgacatccggcccacgaacacggacgtggccttctattcgacggtcaccgccgagcgcctgacggacaccacggccctggacacggattactgggtcaccaacctccgtcagcccgtccggttcgccgacaccatcgaagcccttctcgcggacggctaccgcctgttcatcgaggccagcgcccaccccgtgctgggcctgggcatggaggagaccatcgagcaggcggacatgcccgccaccgtcgtccccaccctccgccgcgaccacggcgacaccacccagctcacccgcgccgccgcccacgccttcaccgccggcgccgatgtcgactggcggcgctggttcccggccgaccccgccccccgcacgatcgatctccccacctacgccttccagcgccgccgctactggctggccgacacagtgaagcgggacagcggatgggacccggccgggtcggggcatgcccagttgccgaccgcggtcgccctcgccgacgggggagtggtgctgaacggccgggtgtccgccgagcgcggtggctggctgggcgggcatgtggtggcggggacggttctggtgccgggtgcggcgttggtggagtgggtgttgcgggccggtgatgaggcgggttgcccctcgcttgaggagttgacgctccaggcgccgttggtgttgcccgagtcgggtgggttgcaggttcaggtggtcgtgggtgcggctgatgagcagggcggccgtcgtgacgtacatgtgtattcgaggtctgagcaggacgcgtcggcggtgtggcagtgccatgccgtcggtgagctcgggcgcgcgtcggtggcgcggccggtgcggcaggccgggcagtggcctccggcgggggccgagccggtggaggtgggcggcttctacgagggggtcgcggccgccggttacgagtacggtccggcgttccgtgggctgcgcgcgatgtggcggcacggtgatgacctccttgcggaggtcgagctgccggaggaggccggttcgccggccggtttcggcatccacccggcgctgctggacgccgccctgcacccgctgctcgcacagcggagccgggacggggccggggcgggggcccacggcgggcaggtgctgctgcctttcagctggagcggtgtttccctgtgggccagcgaggccaccactgtgcgggtgcggctcaccgggctgggaggaggggacgacgagacggtgtccctgacggtaaccgaccccgccggtggccccgtggtggacgtggcagagctgcggttgcggtcgacgagcgcccggcaggtgcggggttcggcaggccccggcgcggacgggctctacgagctgcggtggacaccgttgcccgagccgcttcccgtaccggcccccgcgaacggtcgcgatgtggccgccgacctgtccggatgcgcggtgctcggcgaactggtcgcggaaccgggcccgggcatcgacctggagggctgcccctgctacccgggcgtcggcgcgctcgccgacaacgcctccccgccctccatgatcctcgcccccgtgcacagcgacaccacaggcggcgacggactcgccctgacggaacgggtgttgcgcgtcatccaggacttcctggctgcaccgagtctggaacagaaacagacgcgcctggccttcgtgacccggggcgcggcggacacaggtagcacgacgggaggctcggctgccccggcagaggcagtcgacccggcggtcgcggccgtatggggcctagtacgcagcgcgcagtcggagaaccccggccgcttcgtactgctggacaccgacgcgcccctcgaccaggcgtccgttgcccctctcgtggacgcggtgcggtctgccgtggaggcggacgagccccaagtcgccctgcgcgggggacggttgctcgtgcccaggtgggcgcgggccggcgagcccgtcgagctggccgggccggccggagcgcgggcgtggcggctggtgggcggagactccgggacgctggaggccgtcgtggcggaggcttgcgacgacattgtgctgcgcccgttggcgccgggccaggtccgcgtcgccgtccatacggccggggtcaatttccgtgacgtcctgatcgccctgggcatgtacccggacccggacgcgctgcccggcaccgaggcggccggcgtggtgacggaggtcgggccgggcgtcacccgtctgtcggtgggcgaccgcgtgatgggcatgatggacggcgccttcggcccgtgggccgtcgccgacgcgcgcatgctggccccggtcccgcccggctggggcacccggcaggcggccgccgctcccgccgcgttcctgacggcttggtacgggctggtggagctggccggtctgaaggcgggcgagcgtgtgttgatccatgccgccacgggtggtgtggggatggcggcggtgcagatcgcccggcatgtgggtgccgaggtgttcgccaccgcgagtccgggcaagcacgccgtgctggaggagatgggcatcgacgccgcccaccgcgcctcgtcgcgcgacctcgccttcgaggacgccttccggcaggccaccgacggccgtggcgtggacgtcgtcctcaacagcctcaccggtgaactgctcgacgcgtccctgcgattgctcggcgacggcgggcgcttcgtggagatgggcaagagcgatccgcgcgaccccgagctggtcgcgctggagcaccccggggtgtcgtacgaggccttcgacctcgtcgccgacgccgggcccgagcggctcgggctgatgctcgacaggctcggcgagctcttcgccggcggatcactggtaccgctgccggtcaccgcatggccgctggggcgggcgcgagaggcgctccgccacatgagtcaggcgaggcacaccggcaagctggtgctcgacgtgcccgcgccgctcgaccccgacggcaccgtcctcgtcaccgggggtaccggcaccatcggcgccgccgtggccgaacacctggcgcgtaccggggagagcaagcacctgctcatcgtcagccgcagcgggccggccgcccacggcgccgaggaacttgtctctcgtatagccgagttcggggccgaagccaccttcgtcgctgccgacgtgagtgagcccgacgcggtcgccgccctgatcgaagggatcgatccggcccatccgctgaccggtgtcgtgcatgccgccggagtactcgacaacgctctgatcggctcccagaccaccgaaagcctcacccgcgtatgggcggcgaaggccgccgccgcgcagcaactccacgaggccacgagggagtcgaggctgggactgttcgtgatgttctcctccttcgcctccaccatgggcaccccagggcaggccaactactccgccgccaacgcctattgcgacgcgctggccgctctccgacgcgcggaggggctcgccggcctgtccgtggcgtgggggttgtgggaggccaccagcggcctgaccgggacgttgtcggcggccgaccgggcccgcatcgaccggtacggcatcaggccgaccagcgcggcacgcggctgcgccctgctggcagcggcacgcgcccacgggcgccccgacctgctcgccatggacctggacgcccgcgtacccgccgcgtccgacgctccggtccccgccgtgctgcgcactctggcggccgccggagcgcccgccaccgcccgtcccaccgcggcggcggccgctgacggggcgacggactggtccggcaggctcgccggcctcaccgaggaggcacggctcgaactcctcaccgagttggtgtgcacccacgcggcaggggtgctcgggcacgccgacgcgggcgcggtccaggtggacgcgccgttcaaggaactcggcttcgactcgctgaccgccgtcgaactgcgcaaccggatcgccgccgcgaccggcctgaaactgcccgccgccctcgtcttcgactacccgcaggctcgcgttctcgccgcccacctggccgaacggctcgtcccggagggcgcgggggccatgggcggtgtgagcggtgcggagggcgtgagggacgcgtacggggcaggcggtccgggcggcgacatgaccgcccaggtcttgctggaggtggcccgcgtcgagcacaccctgtccgccgccgtcccgcacggcctggaccgggcggccgtcgcggcccgcctggaggcgctgctcgcccgctgcacggcgacgacggcggccacgggggccgcgggagccgcggtggagggtgacggcgacagcgacggcgacggcgccgtggatcagctggagacggccaccgccgagcaagtactggacttcatcgacaacgaactcggggtgtgagccgcgtgccggccgcacaccaggcgatcacgggcggggagctgcagcgcacatggtgagcgaagagaaactggtcgac

SEQ ID NO: 2 - DNA, 30000 bp, Streptomyces cinnamonensis
  Nucleotides 30001 - 60000 of the monensin biosynthetic gene cluster
tacctcaagcgtgtctccgcggacctgcacgccacccggcagcggctgcgcgaggcggaggagcgcggccaggaacccgtggccgtggtggaggccgcctgccgctaccccggcggcatccgcacccccgaagacctgtgggacctggtcgccgcgggcggcaacgccctgggcgccttccccgacaaccgcggctgggacctgcgacgcctcttccaccccgaccccgaccaccccgggacgacctacgcccgcgagggcggcttcctccacgacgccgacctgttcgacccggagttcttcggcatcagcccccgcgaggccgcggtcctcgacccgcagcagcgactgctcctggagtgcgcctgggaggcactggagcgcgcgggcatcgacccgcggtccctccagggcagccgtaccggcgtgtacgcgggtgccgccctgcccggcttcggcaccccgcacatcgaccccgccgccgagggccacctggtcaccggcagcgccccgagcgtcctctcgggccggctcgcctacaccttcggcctcgaagggcccgcggtgacgatcgacaccgcctgctcgtcgtcgctcgtcgccgtgcacctggcggcccacgcgctgcggcagcgcgagtgcgatctggcgctcgcgggcggtgtcaccgtcatgaccaccccgtacgtgttcaccgagttctcgcgccagcgcggcctggccgccgacggccggtgcaagcccttcgcggccgccgcggacggcacggccttctccgagggcgccggactcctcgtactggaacgcctctccgacgcccgccgggccggccaccgggtgctcgccgtcatccgcggctcggccgtcaaccaggatggcgcgagcaacggcctcaccgcccccaacggccccgcccagcagcgcgtgatccgcgccgccctcgccggggcgcggctctcgcccgcggaggtggacgcggtcgaggcgcacggcaccggcacccggctgggcgaccccatcgaggccgacgcgctcctcgccacctacggtcaggagcgccacgggggccggccgctgtggctcggctcggtgaaatccaacatcggccacacgcagggcgcggccggtgccgcgggcctgatcaagatggtccaggcactgcggcacgagacgctgcccgccacgttgtacgccgacgagcccaccccgcacgccgactgggagtcgggcgcggtgcgcctgctcagcgcgccggtcgcctggccgcgcggggagcacggggagcacacccgcagggccggcatctcctccttcggcatctccggcacgaacgcccacctcatcctggaggaggcgcccgcggccgacgccgaaggagcgggtggcgacggcgatggcgacgggggaggggtgcggccggtggtgcgggtcggcgccacgggcccccgcgaagagcagggccaaggacagggccaagagcagcaccaacagcaacgtcagcagcggcagcggtcgtcgatgatgccgacgccgcacctcccgtggctgctgtccgcccgcagccccgccgcgctccgcgcccaggccgacgcgctggcgaaccatgtcgcccacgcggaccactccatcgccgacatcggcggcacactgctgcgccgcaccctgttcgagcaccgggcggtcgtcctcggaaccgaccgtgatgagcgtgccgcagcgcttgccgccctcgcggcaggacgcgcacaccccgcgctgacccgggccgcagggccggcgaggaacggcggcaccgccttcctgttcaccggccagggaagccaacgcccaggcatgggcaggcagttgtacgacaccttcgacgtcttcgccgagtcgctcgacgagacctgcgcccggctcgaccccctgctcgaacagccgctgaagcccgtcctgttcgcccccgccgacaccgcgcaggccgccgtgctgcacgggaccggcatgacgcaggccgcgctgttcgccctcgaagtcgccctgtaccgccaggtcacctccttcgggatcgcccccagccacctgaccgggcactccgtcggcgagatcgccgccgcccacgtcgccggggtgttctccctggcggacgcctgcacgctggtcgcggcccggggccgcctcatgcaggccctgcccgcaggtggcgccatgctcgccgtccaggcggccgaggacgacgtactgccgctgctcgccgggcaggaggaacgtctctccctcgccgccgtcaacggccccaccgccgtcgtcgtgtccggtgaggccgctgccgtcggggaggtggagaaggcgctgcgcgggcgcggactgaagaccaagcggctcaacgtcagtcacgccttccactcgccgctcatcgagccgatgctcgacgacttccgcgaagtggcccgcgggctgaccttccacgcgccgacgctgcccgtcgtctccaacctcaccggccgcctcgccgacgcggagctgatggccgacgccgagtactgggtgcggcacgtacgccggccggtgcggttccacgacgggctgcgcgctctcagcgagcaaggcgtcgtgcgctacctggagttggggcccgacccggtcctcgccaccatggtccaggacggtctcccggccccggcggagggagaggagcccgagccggtcgtcgccgcggcgctgcgctccaagcacgacgagggacgcaccctgctgggtgccgtcgccgcgctccacaccgacggacagccggccgacctcaccgccctcttccccgccgacgccgggcaagtgccgctccccacctaccggttccagcggcgacggtactggcgcgtcgcgcccgacgccgccgcgccggcccgcgccgccggcctccaggagaccggccacccgctgctgcccgccgtcatccggcaggccgacggcggcatcctgctcgcgggacgcctgtccctgcgtacgcatccatggctcgccgaccacaccatcgcgggcggcgtcccgctgcccgccaccgccttcgtcgaactcgccctgctcgcagggcggcacgccgcctgcgacacgatcgacgatctgacgctggagacgccgctgctgctcgacgacaccggtaccggtgtcggggcggctgtgggcgcgggcgccgatgccctcgtcgatgccatagaagtgcagcttgccctcggcgctcccgacggttccggccgccgtgctctcaccgtccactcccgtcctgccgacgatgcggctgacgacggcgacgcggccgacgcggccgatgcggcaggccggggaggcccgggcggctcgggtgacctgggcgatcctggcgatccgggcgatctgggcgacggcgggggctcccgcggctggcgccgtcacgccaccggcatcctcagcgccggcccggccgccgaaccggccgcccccgacgccgctccctggccgcccgccgacgccaccgccctcgacgtcgacgcgctgtacgcccggctcgacgcgcagggctacagctacgggcccgccttccgggccgtccacgccgcctggcggcacggcgacgacctctacgccgatgtccgcctcgccgacgaacagcgcgctgaagccgacgcgttcgccctccacccggccctgctcgacgccgccctgcatgccgtcgacgagctgtaccgcggcagtgaggggcgggggcaggagcaggggcagggtggtcaggagccggagcagggccgtggcgacgcggacgccccggtacggctgccgttctccttcagcgacatacgccaccacgccaccggggccacacggctgtgggtccgcctcagcccccagggcgacgatcggctgcggctgtccctgaccgacggcgagggcgggcaggtcgcgacagtcgacgccctccaactgcggttgatccccgccgaccggtggcgcgcggcccgccccaccacagccgcccccctgtaccacctggactggcacgagctgccgttgcccgagccggccgagacggacccggccgcccactcctgggctgtgctcggagcgcacgacgcgggcctcgctcccgccgcgcactacccggacctggcggccctgaaagccgccgtcgaggccggcgagcccgtgccggacatcgtcttcgcaccgttccccgcgcaggggacggagaccgatgtcccggctcaggtacgagcccacgcccggcacgccctggagctgctgcgcgactggctcaccacggaagctttcgccgccgcccgcctcgtcgtcctcacgaccggtgcggtcaccgcccgcccagaggacgggcccgccgacctggccaccgcacctgtatggggcctggtccgagccgcccaggccgaacaacccgaccatgtcgtcctggtggacatcgacaaggacatcgataaggacaccgacgaggagaccgaccaggccaccgacgcgggcaccgcatcgcgccacgctctgcccgccgccttggccgcggcggccgcccaagccgagacacagctcgccctgcgcgcgggcaccgtgctcgtgccgcgcctcgccgtcgtcccgccccggaccgacaccccagcgctgcacgccaccgccccggagagcaccacggacactgtggactccacgggcatcgcgggcgctgcggaatccggcggcaccgtcctgatcaccggcggaaccggcggcctcgggcaggccgtcgcccgtcacctcgccgccgcgcatggcgcccgccacctgctcctcgtcagccgcaggggcgacgccgccgagggcgtcgccgagttgcgcgccgacctcgcggacgacggcgtcgacgtacgcgtcgccgcctgcgacatcaccgaccgcgacgcgctggccgggctcctcgcggacatccccgccgcgcacccgctcaccgcggtcgtgcacaccgcgggcgtcatcgacgacagcctcatcacggcgatgacccccgagcggctcgacgccgtcctcgcacccaaggccgacgcggcctggcacctgcacgaactcacccgcgacaaggacctgtcggccttcgtcctcttctcctcgggcgcctccgtcctcggcaacggcggccaggccaactacgcggccgccaacaccttcctcaacaccctcgccgaacaccgccgcgcggccggcctcgccgccacctccgtggcctggggcctgtgggagtccgcgtccggcggcatggccgcccggctcggcgacgccgaccgcgcccgcatccaccgcaccggcgtgacgggcctgaccgacgagcaggccctggccctcttcgacgcggccctgaccgccgagcaccccacggtcctcgccacccgcttcgaccgcgccgtgctgcgcggccaggccgccgcccgcaccctgcagcccgccctgcgcggcctggtacgcactccgcgccccaccgcgtccgccggggccatcgggtccaccgcagccaccgggtccgccacggacgagaacgcgccctcctcgtgggccgcccggctcgcccggctgtccgccgccgaccgcgaccgcgccctcaacgaactcattcgcgagcagatcgcgaccgtcctggcacacccctcacccgacaccatcgaactgggccgcgccttccaggagttgggcttcgactcgctcaccgccctggaactccgcaaccgcctctccacggccaccggcatccggctgcccgccaccctcgtcttcgaccacccgagccccaccgccctcgtacgccatctccacagccatctccccgacgaggcccagcacacgtccccgaccgcccccggcgcctctgcggagggcaccgccgccacggccaccggcatcgacgacgacccgatcgccatcgtcggcatggcgtgccgctacccgggcggcgtgacctcgcccgagcagctgtggcagctcgtggccaccggcaccgacgccatcggcccgttccccgaggaccgcggctgggacacggccggactgttcgatcccgaccccgaccaggtcggccacagctacacccgcgaaggcggcttcctctacgacgccgcccgcttcgacgcgggcttcttcggcatcagcccgcgcgaggccgccgccaccgacccgcagcagcgcctgctcctggaaaccgcctggcaggcgttcgaacacgcgggcatcgaccccgccgccctgcgcggcaccccgtgcggcgtcatcaccggaatcatgtacgacgactacggatcccgcttcctcgcgcgcaaaccggacggcttcgagggccgcatcatgaccggcagcacgccgagcgtggcctccggccgggtcgcgtacaccttcggcctggagggccccgccatcacggtggacaccgcgtgctcctcctcgctggtcgcgatgcacctggcggcgcaggcgctgcggcagggcgagtgcgaactggccctggccgggggtgtgaccgtgatggccaccccgaacaccttcgtggagttctcccgccagcgcggcctggcccccgacggccgctgcaagccgttcgccgccgcggcggacggcaccggctggggcgagggcgccggactcgtcgtcctggagcgcctctccgacgcgcgccgcaagggacaccgcgtcctcgccctgctgcgcggttcggccgtgaaccaggacggcgcgagcaacggcatgaccgccccgaacggtccctcgcaggaacgggtcatccgcaccgccctggccggcgcgggccgtggtcccgaggacatcgacgtggtggaggcgcacggcaccggcaccacgctcggcgacccgatcgaggcgcaggccctgctcgccacgtacgggcaggggcgcccggaggaccgcccgctctggctcggctcggtgaagtcgaacatcggccacacgcaggccgccgccggtgtcgcgggcgtcatcaagatggtcatggcactgcgccacgagcaactgcccacgaccctgcacgccgacgagccgaccccccacgtgcaatgggacggcggcggcgtacgtctcctgaccgaaccggtcccgtggtcgcgcggcgagcgcacgcggcgcgccggggtgtcgtccttcgggatctccgggacgaacgcgcacctgatcctggaggagccgccggaggaggacctgcccgagcccgtggcggcggagccgggtggggtggtgccgtgggtggtgtccgggcggacgccggacgcgttgcgtgaacaggcgcggcggctcggcgagtttgtcgtcggtgccggggatgtgtcggcagccgaggtgggatggtcactggccacgacgcggtcggtgttcgagcaccgggccgtggtggcgggccgggaccgggacgatctggttgccgggatgcaggcgctggcggcaggggagacgccgacagatgtcgtgtccggtgcggcggcttcctccggtgcggggccggtgttggtgttcccggggcaggggtcgcagtgggtgggcatgggtgcccagctccttgacgagtcccccgtcttcgcggcgcggatcgcggagtgtgagcaggcgctgtcggcgtacgtggactggtcgctgagtgatgtcctgcgcggggacgggagtgagctgtcccgggtcgaggtcgtgcagcccgtgttgtgggcggtaatggtctcgctggctgccgtctgggcggattacggggtcactccggccgctgtggtggggcattcgcagggtgagatggctgccgcgtgtgtggcgggggcgctgtcgctggaggatgcggcgcggattgtggcggtacgcagtgacgcgcttcgtcagctgcaagggcacggcgacatggcctcactcggcactggtgccgagcaggccgctgagctgatcggtgatcggccgggagtggtcgtcgcggcagtcaacgggccgtcgtctaccgtgatttcggggccgccggagcatgtggccgctgtggtcgcggaggcggaggcacgtggtctgcgcgcccgtgtgatcgacgtcgggtatgcctcgcacggcccccagatcgaccagctccacgacctcctcaccgagggcctggctgacatccggcccgcgaacacggacgtggccttctattcgacggtcaccgccgagcgcctgacggacaccacagccctggatacggattactgggtgaccaacctccgccagccggtccggttcgccgacaccatcgaagcgcttctcgcggacggctatcgcctgttcatcgaggccagcgcgcacccggtgttgggcctgggcatggaggagaccatcgagcaggcggacatccctgccacggtcgtccccaccctgcgccgcgaccacggcgacaccacccagctcacccgcgccgccgcccacgccttcaccgccggcgccgatgtcgactggcgacgctggttcccggccgaccccaccccccgtaccgtcgacctccccacctacgccttccagcaccagcactactggctggaggagcccagtgggctcaccggagacgccgccgacctcggcatggtggccgccgggcatccgctgctcggtgcctgtgtggaactcgcggagagcgactcgtacttgttcaccgggcggctctcgcgcagggctccgtcctggctggccgaacacgtggtggcggggacggttctggtgccgggtgcggcgttggtggagtgggtgctgcgggccggcgatgaggcgggatgcccgacgattgaggaactgacgctccaggcgccgttggtgctgcccgagtcgggcgggttgcaggttcaggtggtcgtgggtgcgaccgatgagcagagcggccgtcgtgacgtacacgtgtattcgaggtctgagcaggacgcgtcggcggtgtgggtgtgccatgccgtcggtgtggtgagctccgaaatgccagaagcggcagccgagttgagtgggcagtggcctcctgccggggccgaagccgtggatgtcgaggacttctacgcgcgggccgcggaggccggatacgcctacggtccggcgttccaggggctgcgggcgctgtggcggcacgggacggagctgttcgccgaggtggtgctgcccgaacaggcgggtgggcacgacggtttcggcatccacccggcgctgctggacgccgccctgcatccgctgatgctcctcgaccggcccgcggacgggcagatgtggctgccgttcgcgtggagcggggtgtcgctgaacgcggaccgggcgacccacgtccgtgtccggctctccccgcggggggaggcggccgagcgtgacctgcgggtcgtcatcgccgacgcgaccggcgcgcccgtcctgacggtcgacgccctgaccctgcgcgcggccgatcccggccggctgggtgcggcggcccgtggcggtgtcgacggcctctacaccgtcgactggaccccgctgcccctgccccagccccttccgctgccgcggacggatgcaggggggagtgccgactgggtcatactctcggacaactccagtgcagctctggctgatgccgtgtcgtccgcgacggcggcaggtggcggagcgccgtgggcattgctcgctcccgtgggtggcggctctgccgatgacgggctgccggtggtgcggcggaccctctccctcgtacaggagttcctggccgccccggagctgaccgagtcccgtctcgtcatcgtgacacgcggtgccgtggccaccgacgccgatggtgacgtcgcggcgtccgcggcagcggtatggggcctgatccgcagcgcccagtcggagaacccgggccgcttcgtcctgctcgacgtcgaggaggagcacctccacccggacggcggggaactgccgtacgccgccctgcgccacgccgtagaggagctcgacgagcctcaacttgccctccgcagcggcaaattcctcgtaccgcgcatgacgcccgccgccgcccccgaggagctcgtcccgccggtcggtacgtccggctggcgcctcggcacctccggtacggccaccctggagaatctgtcggtgatcgacgctcccgaggcgttcgcgccgctggagcccgggcaggtgcggatctccgtacgggcggcgggcatgaacttccgtgacgtgctgatcgcgttgggcatgtatcccgacaagggcacgttcgcgggaagcgagggcgccggacatgtgacggaggtgggaccgggcgtcactcatctgtcggtcggtgaccgggtgatgggtctgttcgagggcgcgttcgctccgctggccgtcgcggacgcccggatggtcgtcccgattccggagggctggagcttccaggaggccgcggcggtgcccgtggtgttcctcacggcctggtacggcctcgtggacctcggccgcctccgggcgggcgaatcgctgctcatccacgcgggcaccggcggagtgggcatggccgccacccagatcgcccgccacctgggcgccgaggtgttcgccaccgcgagccccgccaagcacggcgtgctcgacggcatgggcatcgacgcggcccaccgcgcctcctcccgtgacctcgacttcgaggagaccttgcgggcggcgacgggcgggcgcggcatggacgtcgtactcaacagtctggccggggagttcaccgacgcctcgctgcggctgctcgccgagggcgggcgcatggtggacatgggcaagaccgacaagcgcgaccccgaccgggtcgcggccgagcacgcgggcgcgtggtaccgggccttcgacctcgtgccgcacgcggggcccgaccggatcggggaaatgctggcggagctgggcgagttgttcgcctccggcgccctggcgccgctgcccgtccagacctggccgctgggccgggcgcgtgaggcgttccggttcatgagccaggcgaagcacaccggcaagctggtgctggagatcccgcccgccctcgatccggacggcacggtgctcatcaccggcggcaccggggtcctcgccgccgcggtggccgagcatctggtgagggagtggggcgtacgacacctgctgctggccgggaggcgcggttccgaggcgcccgggagcagtgaactcgccgaggaactgaccgagttgggggccgaggtgacctttgccgcggccgatgtcagtgatccggacgccgtggcggagctcgtcggcaagaccgatccggcgcacccgctgaccggtgtgatccacgcggccggtgtgctggacgacgccgtggtcaccgcacagaccccggagagcctcgcgcgggtgtgggcggcgaaggcgacggccgcacacctgctgcacgaggcgacccgggaggcgcgcctcggtctcttcctggtgttctcctcggcggcggcgacactcggcagtccgggacaggccaactacgcggcggccaacgcctattgcgacgccctcgtccggcaacggcgtgccgagggcctggccggtctctcgatcggctggggtctgtggcagacggcgagcggcatgaccggacacctcggcgagacggacctggcacgcatgaagcgcaccgggttcaccccgctgaccaccgaaggtggcttggccctcctcgacgccgcccgcgcccacggccgcccgcacgtggtcgcggtggacctcgacgcgcgcgccgtcgccgcgcagcccgccccgtcccggcccgcgctcctgcgcgccctggccgcgggtgcgaccccgggggcccgcaccgcccggcgcaccgcggccgcgggcagcgtcgccccggcgggcggtctcgccgaccggctcgccggcctgccgcatcccgaacggcgccggctgctgctcgacctcgtacgtggcaacgtcgccggcgtcctcgggcacagcgaccacgacgccgtccgcccggacacgtcgttcaaggagctcggcttcgactccctgaccgccgtggaactgcgcaaccggctggccgccgccaccggcctgaagctgcccgcggcgctcgtcttcgactaccccgagtcggccaccctcgtcgaccacctcctggagcgtctgtcgcccgacggcgcgccgccgcccgtcaaggacgccgcggaccccgttctcaacgacctcggcaggatcgagtcctccctggacgcgctcgccctcgacgcggacgcgcgcagccgggtcaccaggcgtctgaacaccctgctgtcgaagctgaacggagccgccaccgccggctccccggcggacgtcacggacctggacgcgctggacgcgctggacgacgtgtccgacgacgagatgttcgagttcatcgaccgagagctgtgacccccctgcccgccccgtcccccttccccgcccccacgttccccgtgcccttcgctgatggagaagtgacgttcgatgtcgagtgctgaagagtcgagtcctgatgtgtccggcacgggtgtgtccggtacgggagagtccgctacgggtacgtcgagtacggaagccaagcttcggcagtatctgaagcgggtcacggtggacctcggccaggcccgccggcggctgcgcgaggtggaggagcgggcccaggagccgatcgccatcgtctccatggcgtgccgcttccccggcgacacccgcacgcccgaggccctgtgggacctggtcgccgagggcggcgacgccatcgacgacttccccaccaatcgcggctgggacctggagagcctctaccaccccgaccccgaccaccccggcaccagctacgtccgacgcggcgggttcctgtacgacgcccccgccttcgacgcgtcgttcttcgggatcagcccgcgcgaagccctggccatggacccgcagcagcgggtgctcatggagacggcctggcagctcctggagcgggccggcatcgacccggcctcgctgaagctgagcgccaccggcgtctacatcggcgcgggcgtgctcgggttcggcggcgcgcagcccgacaagacggtagagggccacctcctgaccggcagcgcgctgagtgtcctgtccggccgcatctccttcacgctcggcctcgagggcccgtcggtcagtgtcgacacggcgtgctcctcctcgctggtctccatgcacctggcggcccaggcgctgcggcagggggagtgcgatctcgcgctggccggcggtgtcaccgtgatgtcgacgcccggcgcgttcaccgagttctcccgccagggcgcgctgtctccggacggccgctcgaaggctttcgcggcctcggccgacggcaccggtttctcggagggcgcgggactgctcctcctggagcggctctccgacgcgcgccgcaacggccacaaggtgctcgcggtgatccgcggctcggccgtcaaccaggacggcgcgagcaacggtctcaccgcccccaacggcccctcccaggaacgcgtgatccgcgccgccctcgccaacgcgggcctgggcgccgccgaggtcgacgcggtcgaggcacacggcaccggcacgaagctcggcgaccccatcgaggccggtgcgctgctcgccacctacggccgcgacagggacgaggaccggccgctgtggctgggctcggtcaagtcgaacatcggtcacccgcagggcgcagcaggcgtcgcgggcgtcatcaagatggtgatggcgctgcagcgcgaactgctccccgccaccctgtacgtcgacgagcccaccccgcacgtcgactggtcctcgggctccgtcaggctcctcaccgaaccggtcccgtggacccgcggcgagcgcccgcgccgcgcgggcgtgtccgccttcggcatgtccgggacgaacgcccacgtgatcctggaggaggcaccgcccgaggaggcagcggccgcggagacaccggcggaagggacaggcgcagtcgtcccgtgggtcgtctccggccggggcgaggaagcgctgcgggcccaggccgcacagctcgccgagcacgtgcgcgacgacgaccagcggccggcgtcaccgctggaggtggggtggtcgctcgccacgacacggtcggtgttcgagaaccgggccgtcgtcgtcggggacgaccgcgacgcgctcctcgacggcctccggtcgctggcggcaggtgaggcgtcgccggacgtggtgtccggggcggtcggccccacggggcccgggccggtcatggtgttccccggccagggcggccagtgggtgggcatgggggcccggctcctcgacgagtccccggtgttcgcggcccggatcgccgagtgcgagcaggccctgtcggcgtacgtggactggtccctgaccgacgtgctgcgcggggacgggtcggagctggcccggatcgacgtcgtccagcccgtgctgtgggccgtcatggtcgcgctcgccgccgtctgggcggaccagggaatcgaacccgccgccgtcgtcggccactcgcagggcgagatagccgcggcgtgcgtcgtgggcgccatctccctggacgaggcggcccgcatcgtcgccgtacgcagtgtgctgctgcggcagctgtccggacgcggcggcatggcgtccctggggatgggccaggagcaggccgccgacctgatcgacggacacccgggtgtggtcgtcgcggccgtcaacgggccgtcgtccaccgtcatctcgggcccgcccgagggcatcgccgccgtcgtcgccgacgcccaggagcggggccttcgcgccagggccgtcgcctccgacgtcgcgggccacggcccgcagctggacgcgatcctggaccagctcacggagggcctggccggcatccggcccgccgcgaccgacgtcgcgttctactccaccgtcaccgccgggcacctcaccgacaccaccgaactcgacaccgcgtactgggtgcggaacgtgcgccggacggtgcgtttcgccgacacgatcgacgcgctgctcgcggacgggtaccgcctgttcatcgaggtgagcccccaccccgtcctcaacctcgcgctggaaggcctcatcgaacgggcggccgtgcccgccacggtcgtgcccaccctgcgccgcgaccacggcgacaccacccagctcgcccgcgccgcggcccacgccttcgccgccggcgcggacgtcgactggcggcgctggttcccggccgaccccgccccccgtaccgtcgacctgcccacctacgccttccagcgccaggacttctggccggcccccgccggcgggcggtccggcgaccctgccgggctcggcctcgccgcctccggacacccgctcctgggcgcctccgtgggcctcgcgagcggggacgtacacctgctgagcgggcgggtgtcccggcagtccgccgcgtggctggacgaccacgtcgtggcgggccaggccctggtgcccggcgcggcgcaggtggagtgggtgctgcgggccggcgacgacgcgggctgctccgccctggaggagctgacgctccagacgccgctcgtgctgcccgacaccggcggcctgcggatccaggtcgtcgtcgaagcggccgacgcacacggccggcgcgacgtccggctgttctcccgccccgatgacgacgacgccttcgcgtcgacgcacccctggacctgccacgccacgggcgtgctcgcccccgccccgacggacggcaccaacggaacgcgggacgccgccgacaccctggacggcgcatggcccccggccgacgccgaacccgtccccgccgacgacctctacgcgcaggccgaccgcaccggatacggctacggccccgccttccggggcgtacgggcgctgtggcgccacggcaaggacgtcctggccgaggtgacgctgcccaaggaggccggcgacccggacggcttcggtatccacccggccctcctcgacgccgtcctgcaacccgccgcactgctgctgcccccgaccgacgccgaacaggtctggctgccgttcgcctggaacgacgtggcgctgcacgccgtacgggccaccacggtccgggtgcgcctcaccccgctcggcgagcggatcgaccaggggctgcgcatcaccgtggccgacgccgtgggcgcgcccgtgctcaccgtccgcgacctgcgctcgcgcccgaccgacacaggccgcctcgccgcggccgcgacccgcgaccggcacgggctgttcgacctggagtggatcgcgccggagaacgcggcggagaacgcggcgggtccggcccgggacgcgtccgaagggtgggtgacactcggcgaggacgccgcgagcctcgcggacctgctggcgtccgtcgaggcgggcgctccggcgccgcagctcgtggccgcccccgtcgaacccgaccggaccgacgacggcctggcactcgccacccacgtcctcgacctcgtacagacctggctcgcctcgcccctgcacgactcccgcctggtcctggtgacgcgaggggcagtgacggatgcggatgtggatgtggctgccgcggccgtttggggtctggtacgcagcgcccagtcggagcaccccggccgcttcacgctgatcgacctcggccccgacgacacgcttgccgcagccatgcaggcggcgcacctggaagagccgcaactggcggtgcacggcggcgagatacgagtgccgcgactggtccgcgccacgaccgacccgaccgccccgaacgggacaccggaggccgaccggacggcggacccgtccgaaggactccaccggaacggtacggttctcatcaccggcggcaccggcgtactcggccgactggtggccgaacacctggtcacggagtggggcgtacgccacctgctgctcgcgagccgacgcggcgaccaggcgccgggtagcgccgaactccgcgcccgcctgagcgaattgggagcatcggtcgagatcgccccggccgatgtcggcgacgcggaagcggtcgccgcactgatcgcgtcggtcgacccggcgcacccgctcaccggtgtgatccacgcggccggtgtcctggacgacgccgtgatcaccgcccagacccccgagagcctcgcgcgggtgtgggcgacgaaggcgacggcggcccgccatctgcacgaggcgacacgggagacacccctcgacttcttcgtggtgttctcctcggcggccgcctcgctcggcagccccggccaggccaactacgcggcggccaacgcctattgcgacgccctcgtccagcaccgccgcgcccaagggctcgcgggcctctcgatcgcctggggcctgtggcaggcgaccagcggcatgaccgggcagctgagcgagaccgacctggcgcgcatgaagcgcaccgggttcgccgcgctgaccgacgagggcggcctggccctgctcgacgccgcccgtgcccacgaccgggcctacgtggtcgcggccgacctcgacccgcgcgccgtgaccgatggcctgtccccgctcctgcgcgccctcacggcgcccgccacgcggcggcgcgtggcctccgaaggcctcgccgacggggcgctcgcgacccgcctggccggcctcgacgcggacggccgcctaaggctcctcaccgatgtcgtacgcgagtacgtcgcggccgtcctcggccatggttccgccgcccgggtgggcgtcgacatcgccttcaaggacctgggtttcgactcgctgaccgcggtggagctgcgcaaccggctgtcggccgcctgtgacgtgcggctgcccgccacactgatcttcgaccaccccaccccgcaggctctcgccacccacctggtggaccgcttggcgggcagcacctccgcgaccacgacggtgaatgcgacggcgccggcagccgcccacgtcgccgcaggggccgacgtcgacgcagacaccgacgacccggtcgccatcgtcgccatgacgtgccggttcccgggcggcgtcgcgtccccggacgacctgtgggacctgctcgacgcacgcaaggacgcgatgggcgccttccccaccgaccgcggctgggacctggaacgcctcttccaccccgacccggaccaccccggcaccagctacaccgaccagggcggatttcttcccgacgcgggtgatttcgatgcggcgttcttcgggatcaatccgcgggaggcgctggcgatggatccgcagcagcggttgttgctggaggcgtcgtgggaggtgttggagcgtgcgggtatcgatccgacgacgctcaagggcaccccgaccggcacctacgtgggcctcatgtaccacgactacgccaagtccttccccacggccgacgcccagttggagggctactcctacttggcgagcaccggcagcatggtctccggccgcgtcgcctacaccctgggccttgaaggtccggcggtgacggtcgacaccgcgtgctcctcctccctggtctccatccacctggcgacgcaggcactccggcacggcgagtgcgacctcgccctggcaggcggtgtgaccgtcatggccgacccggacatgttcgcgggcttctcgcgccagcgcggcctctcacctgacggccgctgcaaggcctacgccgccgcggccgacggagtcggattctccgagggagtgggcgtattgctccttgagcggttgtcggatgcgcggcgtcatgggcgtcgggtgttgggtgtggtgcggggttcggcggtgaatcaggacggtgcgagtaatgggttgacggcgccgaatggtccgtcgcaggagcgggtgattcgtcaggcgttggccagtggtgggttgtcgtcggtggatgttgatgtggtggaggggcatgggacggggaccacgttgggtgatccgatcgaggcgcaggctctgctggccacatatgggcaggggcgtccggaggaccgtccgttgtggttggggtcggtgaagtcgaacattggtcatacgcaggcggctgcgggtgttgcgggtgtcatcaagatggtgatggcgatgcggcatggtgtggtgccggcgagtttgcatgtggatgtgccgtcgccgcatgtggagtgggattcgggtgcggtgcggttggcggttgagtcggtgccatggccgcaggtggagggtcgtccgcgtcgggcgggtgtgtcgtcgttcggcgcttcggggacgaatgcgcacgtgatcgtggagtctgttcccgatgggctggaggaggactcggtatcggtcggcggtgaggctcttgagacggagactgacgggcgcttggtgccgtgggtggtgtcggcccgcagcccgcaggccctgcgcgaccaggcactacgcctgcgtgactttgccagtgacgcgtcgttccgcgcgccgctcgccgacgtgggctggtcgctgctgaagacgcgtgcgctgcatgagcatcgcgccgttgtggtgggcgcggagcgggcagagctgatcgccgctctggaggcgctggcgacgggtgagccgcatgcggcgctggtcggcccggcttgctcgcaggctcgggtgggtggcgatgacgtggtgtggctgttcagtggtcagggcagtcagttggtcggtatgggtgctggtttgtatgagcggttcccggtgtttgcggctgcgtttgatgaggtgtgcggcctgttggaggggccgttgggcgtggaggcgggtgggttgcgggaggtggtgttccgtggcccgcgggagcggttggatcacacggtgtgggcgcaggcggggttgtttgcgctgcaggtggggttggcccggttgtgggagtcggtcggggtgcggccggatgtggtgctcgggcattcgatcggtgagatcgcggccgcgcatgtggcgggggtttttgatctggcggatgcgtgtcgggtggtgggtgcgcgggcgcgtttgatgggtgggctgcctgagggtggggcgatgtgcgcggtgcaggccacgcccgccgagctggccgccgacgtggacggatcggctgtaagtgtggcggcagtcaacacccccgactccacggtgatttcgggcccgtcggacgaggtggaccggattgctggggtgtggcgggagcgtgggcgcaagacgaaggcgctgagcgtcagtcatgccttccattcggcgttgatggagccgatgctcgcggagttcaccgaagcgatacgaggggtcaagttcaggcagccgtcgatcccgctcatgagcaatgtctccggagagcgggccggcgaggagatcacggatccggagtactgggcgaggcatgtacgtaatgcggtgctcttccagcccgccatcgcccaagtagcggattcagcgggcgtgtttgtggagctcggccccgcgcctgtgctgaccacggccgcccagcacaccctggacgagtcggacagccaggagtcggtgctggtcgcgtctctcgccggtgagcgtcctgaggagtcggcgtttgtggaggcgatggctcgtctgcataccgctggtgttgctgtggactggtcggtgttgttcgcgggtgatcgtgtgcctgggctggtggagttgccgacgtatgcgttccagcgggagcggttctggttgagtggccgttctgggggtggggatgcggcgactttggggttggtggcggcggggcatccgttgttgggtgcggcggtggagttcgcggaccggggtgggtgtctgctgaccggtcgtctgtcgcggtctggggtgtcgtggcttgctgatcatgtggtggcgggtgcggttttggtgccgggtgctgcgttggtggagtgggcgttgcgggccggtgatgaggtcggttgtgtgacggtggaggagttgatgttgcaggcgcctttggtggtgcctgaggcgtcgggtctgcgggttcaggtggtggttgaggaggcgggtgaggacgggcggcgcggtgttcagatctacagccggcccgacgcggacgccgtgggcggcgatgactcgtggatctgccacgcgaccggcgtactgtcacccgaaagcgctcgtctggacacggagttgggtggcgtctggccaccggccggtgccgaaccgctggatgtcgacggcttctacgcgcaggccggtgaggccgggtacggatacggtccggcgttccgggggctgcgtgccgtgtggcggcacggccaggacctgctggccgaggtcgtcctgcccgaagccgccggtgcccatgacggctacgggatccaccccgccctcctcgacgccaccctccatccgctgctcgccgcccgcttcatggacggttccgaggacgatcagctctacgtaccgttcgggtgggccggagtgtctctgcgggcggtgggagccacgactgtgcgcgtgcgcctccgtccggtcggggagagcgtcgaccaagggctgagcgtgacggtcaccgatgcgaccggcggtcccgttctgagcgtcgactccctccagacccgccccgtgaagccgagccaattggctgcggcccaacagccggacgtacgcggtctgttcactgtggagtggacgccgctgccgcagacggatgccgacggggaggccgactgggttgtgctctcggacggtgttggccgtctggctgatgtggtgtcggcggcgggtggtgaagcgccgtgggcagtggtcgctcctgtcgatgcgtctgtgggcgacggccgtgagggtcttgacggtcggctggtcgtggagcgggtgctgtcactcgtacaggagttcctggccctgccggagctggccgagtcccgtctcctcgtggtgacgcgcggtgcggtggccaccggcgtcgacggtgacggtgacgtggacgcgtccgccgcagctgtatggggcctggtccgcagtgctcagtccgagaatccgggccgcttcatcctgctcgacgtggacggcgacggcgacgaccagggcccggacctgaacggccggcatctgccccacgccaccctgcgtcacgccgccgaggaactcgacgagccccaactcgccctgcgggaagggacgctctacgtcccccgactgacccaggcgcgccagtccgccgaactcgtcgtgccgcccggtgaaccggcgtggcgcctgcggatggtgcacgacggctcgctggacgccctggcggcagtggcctgcccggaggccctggagcccttggcgccggggcaggtgcgtatcgccgtacacgccgcgggcatcaacttccgtgacgtactggtggccttgggtatggtccccgcgtacggggccatgggtggcgaaggtgccggtgtcgtgacggaggtcggtcccgaggtcacccatgtctcggtgggcgaccgcgtgatgggcgtgttcgagggcgcgttcggccctgtggtgatcgccgaggcgcggatggtcacacctgtcccgcagggctgggacatgcgggaggcggccggtattccggcggccttcctgacggcttggtacgggttggtggagctggccggtctgaaggcgggcgagcgggtgctggtccatgccgcgacgggtggtgtggggatggcggcggtgcagatcgcccggcatgtgggtgccgaggtgttcgccaccgcgagtccgggcaagcacgccgtgctggaggagatgggcatcgacgccgcccaccgcgcctcctcccgggacctcgccttcgagggcacgttcagggaagcaacgggcggccgcggcatggacgtcgtgctcaacagccttgccggcgagttcatcgacgcctctctgcggttgctcggcgacggcggccggttcctggagatgggcaagaccgatgtgcgggccgccgaagaggtggctgcggagcacgcggacgtctcgtacacggcgtacgacctcgtcggtgatgccggacccgaccgcatcagcaacatgctggacaagctcgtcgaattgttcgcctcagaacggcttaagccgctgccggtacgttcctggccgctggacaaggcgcaggaggcgttccggttcatgagtcaggcgaagcacaccggcaagctggtgcttgagatcccgcctgccctcgaccccgagggcacggttctggtcaccgggggcaccggtgcgctggggcaggtcgtggccgagcatctggtccgggagtggggcgtacggcacctgctgctggccagccgtcgcggtccggaggcgccgggcagcgacgaactggcctcgaagctcaccgggttgggtgccgaggtcaccattgtcgcggccgatgtcagcgacccggcctcggtggtggagctggtcggcaagacggatccctcgcatccgttgacgggtgtcgtgcacgcggcgggcgtgttggaggacggtgtcgtgaccgctcagacgcctgaggggctggcgcgggtgtgggcggccaaggctgctgcggcggcgaatctccatgaggcgacccgggagatgcgtctcggcctgttcgtggtgttctcctcggcggccgccacgctcggcagtccgggccaggccaactacgcggccgccaatgcctattgcgacgcgctgatgcagcaccgacgggcggtgggccaggtcggcctgtcggtcggctggggtctctgggaggcgccggacgccaagccgggtgttgccgccgacgccaaggcgagtgctgccaccgtcggcaaggcgagtgctctatccgacggcacgaacggcagcgctccccaggacacgaccggcaccgccccccagggcatgaccggcggactcaccgacaccgacgtagcccgcatggcacgtatcggcgtcaagggcatgagcaacgcccacggtctcgccctgttcgacgccgcgcaccgccacggccgcccccacctggtcggcttcaacctcgacctgcgcaccctggccacgcaccccctgcacacccggcccgcccttctgcgcggcctggccacccccaccgccggcggggcgagcaggccgaccgcgacggcgggcggacagcccgccgacctggcgggccggctggccgcgctgtcgccgtcggaccggcaccacacgctggtccggctcatcagggaacaggccgccaccgtgctcgggcaccacccggacagtctcaccacgggcagcaccttcaaggaactcggattcgactccctgaccgcggtcgaactgcgcaacaggctgtccgccgccaccggtctccggctccccgccggcctggtcttcgaccacccggacgccgacatcctggccgaacacctcggcgcgcaactcgcccccgacggggacacccccgccggtgcggaagccaccgacccggtcctccgcgacctggcgaaactcgagaacgccctctcctccaccctcgtcgagcacctcgacgccgacgcggtcacggcccgactggaagcactcctgtcgaactggaaggcggcgagcgcggcgcccggctcgggcagcacgaaggagcagctccaggttgccacgaccgaccaggtcctcgacttcatcgacaaagaactgggtgtgtgaaacgaccgtgcacggcgcgacaaccacgctgaaggctgggtgaactctcatggcgagtgaagaggaactggtcgactacctcaagcgggtcgccgccgaactgcacgacacccggcagcgcctgcgcgaggtcgaggaccggcggcaggagccggtggccgtcgtcggcatggcctgccgtttccccggcggcatcgagacgcccgagggactgtgggagctggtcgcggccggcgacgacgccattgagcccttccccaccgaccggggctgggacctggaaggcatctaccacccggaccccgaccacccgggtacctgctacgtccgggagggcgggttcctagccgcccctgaccggttcgactccgacttcttcggcttcagcccgcgcgaggccctggccagcagcccgcaactgcgactgctcctggagacgtcctgggaggccctcgaacgggcgggcatcaaccccgcctcgctcaagggcagccccaccggcgtctacgtcggcgccgcgaccaccggcaaccagacgcagggcgaccccggcggcaaggcgaccgagggttacgcgggcaccgcgcccagcgtcctctcgggccgcctctcgttcacgctcggcctggagggcccggcggtgaccgtcgagacagcgtgctcctcctcgctggtggcgatgcacctggcggccaacgccctgcgccagggcgagtgcgacctcgccctcgcgggcggcgtcaccgtcatgtccacccccgaggtgttcacaggcttctcgcgtcagcggggactggcccccgacggccgctgcaagccgttcgccgccgcggccgacggcacgggctggggcgagggcgcgggcctgatcctcctggagcgcctctccgacgcccgcaggaagggccacaaggtcctcgcggtgatccggggctcggcgatcaaccaggacggcgcgagcaacggcttcaccgcgcccaacggcccctcgcagcgccgcgtcatccgccaggcactctccagcgcccacctctccacgtcggagatcgacgtcgtcgaggcgcacggcaccggcaccaggctcggcgaccccatcgaggccgaggcgctcatcgccacctacggcaaggagcgcgaggacgaccgtcccctgtggctcggctcggtcaagtccaacatcggccacacgcaggccgccgcgggcgtcgccggagtcatcaagatggtgatggcgctacagcgcgaactgcttcccgccaccctgaacgtcgacgagccgaccccgcacgtccagtgggagggcggcggcgtacgcctcctgaccgaaccggtcccgtggtcgcgcggcgaacgcccgcgccgcgccggaatctcctccttcggcatatcgggcacgaacgcgcacgtggtcctggaggaggcgccgccggaggaggacgtgccgggccccgtggctgcggagccggaaggggtggtgccgtgggtggtctccgcgcggaccgaggaggcgttgagcgaacaggcgcggcgcctgggcgagttcgtggccgacacggacccgtcgaccgctgacgtcgggtggtcactgaccacgagcagggcgatccttgaacaccgcgctgtggtggtggggcgtgatcgggatgcgctgacggccggcctggcggcgttggccgcgggtgaggagtcggcggatgtggtggctggggtggccggtgatgtgggtcctgggccggtgttggtgtttccggggcaggggtcgcagtgggtgggcatgggcgcccagctccttgacgagtcgcccgtcttcgcggcgcggatcgcggagtgtgagcaggcgctgtcggcgtacgtggactggtcgctgagtgcggtgttgcgcggggatgggagtgaactgtcccgggtcgaggtcgtgcagccggtgttgtgggcggtgatggtctcgctggctgccgtctgggcggattacggggtcaccccggccgctgtgatcgggcactcgcagggcgagatggccgccgcgtgcgtggcgggggcgctgtctttggaggatgcggcgcgcgtcgtggccgtacgcagtgacgcgcttcgtcagctgatggggcagggcgacatggcgtcgttgggcgccagctcggagcaggcggctgagctcatcggtgatcggccgggcgtatgcatcgcagcggtcaacgggccgtcctcgacagtcatttcaggaccgccggagcatgtggcagccgtggtcgcggatgcggaggaacgtggtctgcgcgcccgtgtcatcgatgtcggctatgcctcgcacggtccccagatcgatcagctccacgacctcctcaccgaccggctcgccgacatccggcccgcgaccacggacgtggccttctattcgacggtcaccgccgagcgcctgacggacaccacggccctggatacggattactgggttaccaacctccgccagccggtccgtttcgccgacaccatcgatgcgcttctcgcggacggctatcgcctgttcatcgaggccagcgcgcacccggtgctgggtctgggcatggaggagaccatcgagcaggcggacatccccgccacggtcgtccccaccctgcgccgcgatcacggtgacaccacccagctcacccgtgccgcagcgcacgccttcaccgccggcgccaccgtcgactggcggcgctggttcccggccgaccccaccccccgcacgatcgacctgcccacctacgccttccagcgccgcagctactggttgccggtggacggtgtcggagatgtgcggtcggccgggctgcggcgggtggaacactcgctgttgcccgcggcgctcggtctcgccgatggtgcgctcgtgctgaccggacggctcgcggcgtccggtggtggtggcggttggctcgcggatcacgcggtggcgggcacgacgctcgtccccggtgccgcgctggtcgagtgggcgttgcgggccgccgacgaggcgggctgcccctcccttgaggagctgacgctccaggcacctctggtgctgcccggctccgggggcctccaggtccaagtggtcgtgggtccggccgacggacagggcggccggcgtgaggtgcgcgtcttctcgcgtgtcgactcggacgacgaggcagcggggcaggacgaggggtggtcgtgtcacgcgaccggtgtgctgagccccgagcccggtgcggtaccggacgggctcagcggacagtggcccccgacgggcgccgagccgctggagatcagtgatctctacgagcaggcggcatcggcgggatacgagtacgggccgtcgttccggggcctgcgctccgtgtggcggcacgggcataacctgctggcagaggtggagctgcccgaacaggcaggtgcgcacgacgacttcggcatccaccccgtactgctggacgccgcgctgcacccggcgctgctgctcgaccagaacgcgcccggcgaagagcaagagccagcccagcccgctcttcgcctgccgttcgtgtggaacggcgtctccctgtgggccaccggcgccgcgaccgtgcgggtacggctggccccgcacgggggaggggagacggacgatagcgccgggctgcgcgtgacggtcgccgacgccaccggagcaccggtgctgagcgtggactccctcgctctgcgccccgctgaccccgaactgctgcgcacggccggtcgggcgggcagcggcaccaacggcttgttcacggtggagtggaccgctctgcccccggcggacgtggccgaccacgccgcaggcgacggctgggcggtgctcggtcaggacgtacccgactgggccggagcggacatgccccggcatcccgacatggcctccctgtcggccgcgctggacgagggaacgcaggcccctgcggccgtcttcgtggagaccacagccacatcgcacgccacaccgaacaccgcagcggacgtgacgctcgacgcgtccggccgggcggtcgccgagcgcaccctgcacctgctgcgggactggctcgccgaaccgcgcctcgccgagacccggctcgtcctcatcacccaccacgcggtgacgaccccggcggacgacgacgtgaacgccgcacccctcgacgtcccggccgccgccctgtggggactgatccgcagcgcacaggccgaacacccggaccgcttcgttctgttggacaccgacgcgaaggccaacaccgaccccggccccgacaccagtactgaccacagcaccgcatcgggtacgtaccgaaccgtcatcgcgcgggccctcgccaccggggagccacagctggccgtgcgcgcgggagaactgctggctccccgcctcgcccgagccgccacccccacacccgagacccccacacccgagacacagcccgacaccggatccgggtccgaggccggggccgggtccggatctggacccggcgcgacactggaccccgacggcaccgtcctcatcgcgggcggcaccggcatgatgggtggtctcgtcgccgaacacctggtccgcgcctggtcggtgcggcacctcctgctcgtcagccggcaagggcccgacgcgccggacgcccgcgacctcgccgaccggctggtcggcctgggcgcgacggtacggatcgtcgcggccgacctgacggacgggcgggccaccgcggacctcgtcgcgtcggtcgacccggcgcacccgctcaccggtgtgatccacgcggccggcgtcctggacgacgccgtggtcaccgcgcagacctccgaccagctggccagggtgtgggcggccaaggcgtccgtcgccgccaacctggacgcggccacgtcggagctgccgctcggcttgttcctgatgttctcgtccgccgccggtgtcctcggcaacgcgggccaggccggttacgcggccgccaacgccttcgtcgacgccctggtcggccgccgtcgcgccaccggcctgcccggcctgtcgatcgcctggggcctgtgggcgcgcggcagcgccatgacccggcacctggacgacgccgacctcgcgcggctgcgtgccggcggggtcaagcccctgctggacgagcagggcctcgccctcctcgacgcggcgcgcgccaccgccgcgcacacctcgctggtggtcgcggccggtatcgacgtacgcggactgaacagggacgacgtccccgcgatcctccgcgacctggcgggccggacccgccgcagggcggccgccgactccaccgtcgaccaggccgcgctggagcggcgcctcacgggcctggacgaggccgagcgccgggctgtcgtcaccgacgtcgtacgcgaatgcgtggcggccgtgctcggccaccggtcggcggccgacgtacgcaccgaggccaacttcaaggacctcggcttcga

SEQ ID NO: 3 - DNA, 30000 bp, Streptomyces cinnamonensis
  Nucleotides 60001 - 90000 of the monensin biosynthetic gene cluster
ctcgctcactgcggtgcagctgcgcaaccgcctctcggcggcgagcggcctccgcctgcccgccaccctggccttcgaccaccccaccccccaggcgctggcggcgtacctcggcacgcgcctgagcggccggaccgccacccccgtcgcacccgtggcgccttccgcggccgcgacggacgagccggtggcgatcgtcgcgatggcctgcaagtacccgggtggagcgacctcgccggaaggcctctgggacctggtcgcggagggcgtggacgcggtcggcgccttcccgacgggccgcggctgggacctcgaacggctcttccaccccgacccggaccaccccggcacgagttacgccgacgaaggggccttccttcctgacgcgggcgatttcgatgcggcgttcttcgggatcaatccgcgggaggcgctggcgatggatccgcagcagcggctgttgctggaggcgtcgtgggaggtgttggagcgtgcgggtatcgacccgacgacgctcaagggcaccccgaccggcacgtacgtcggcgtgatgtaccacgactacgcggcaggcctcgcccaggacgcccaactggagggctactccatgctcgccggctccggcagcgtggtgtccggccgcgtcgcctacaccctggggcttgagggtcctgcggtgacggtcgacaccgcgtgctcctcgtccctggtctccatccacctggccgcgcaagcactgcgacagggcgagtgcactctcgccctcgcgggcggcgtgaccgtcatggccacgcccgaggtgttcaccggattctcgcgccagcgcggcctggcccccgacggccgctgcaagccgttcgccgccgccgccgacggcaccggctggggcgagggtgtcggtgtgttgttgctcgagcggttgtcggatgcgcggcgtcatgggcgtcgggtgttgggtgtggtgcggggttcggcggtgaatcaggacggtgcgagtaatgggttgacggcgccgaatggtccgtcgcaggagcgggtgattcgtcaggcgttggccagtggtgggttgtcgtcggtggatgttgatgtggtggaggggcatgggacggggaccacgttgggtgatccgatcgaggcgcaggctctgctggccacgtatgggcaggggcgtccggtggatcgtccgttgtggttggggtcggtgaagtcgaatattggtcatacgcaggcggctgcgggtgttgcgggtgtcatcaagatggtgatggcgatgcggcatggtgtggtgccggcgagtttgcatgtggatgtgccgtcgccgcatgtggagtgggattcgggtgcggtgcggttggcggttgagtcggtgccatggccggaggtggagggtcgtccgcgtcgggcgggtgtgtcgtcgttcggggcttcgggaacgaatgcgcacgtgatcgtggagtctgtgcccgatgggctgggggaggactcggtatcggtcagtggtgaggctcccgagactgagactgacgggcgcttggtgccgtgggtggtatcggcccgcagcccgcaggccctgcgcgaccaggcactacgcctgcgtgatgcggtggcggccgactcaacggtgtcggtgcaggatgtgggctggtcgctgctgaagacgcgtgcgctgttcgagcagcgggcggtggtggtggggcgtgagagggctgaactcctgtcggggcttgctgtgttggccgctggcgaggagcacccggctgtgacgcggtcccgtgaggacggggttgctgcgagcggtgctgtggtgtggctgttcagtggtcagggcagtcagttggtcggtatgggtgctggtttgtatgagcggttcccggtgtttgcggctgcgtttgatgaggtgtgcggcctgttggaggggccgttgggcgtggaggcgggtgggttgcgggaggtggtgttccgtggcccgagggagcggttggatcacacgatgtgggcgcaggcggggttgtttgcgctgcaggtggggttggcccggttgtgggagtcggtcggggtgcggccggatgtggtgctcgggcattcgatcggtgagatcgcggccgcgcatgtggcgggggtctttgatctggcggatgcctgtcgggtggtgggggcgcgggcccgtttgatgggtgggctgcctgagggcggggcgatgtgcgcggtgcaggccacgcccgccgagctggccgccgacgtggacgactctggtgtgagtgtggcggcggtcaacacacctgattcgacggtgatttcagggccgtctggtgaggtggatcggattgctggggtgtggcgggagcgtgggcgtaagacgaaggcgctgagcgtcagtcatgccttccactcggcgttgatggagccgatgctcgcggagttcaccgaagcgatacgagaggtcaagttcacgcggccgaaggtgtcgttgatcagcaacgtctctggtctggaggcgggtgaggagatcgcgtccccggagtactgggcacgccatgtacgccagacagtgctcttccagcccggcatcgcccaagtggcttccacggcaggcgtgtttgtcgagctcggccccggccccgtactgactactgccgcccagcacaccctggacgacgtaaccgataggcatggccccgaaccggtactggtgtcctcgctggccggtgagcgtcctgaggagtcggcgttcgtggaggcgatggctcgtctgcataccgctggtgttgctgtggactggtcggtgttgttcgcgggtgatcgtgtgcctgggctggtggagttgccgacgtatgcgttccagcgggagcggttctggttgagcggccgttctgggggtggggatgcggcgactttgggtctggtggcggcggggcatccgttgttgggtgcggcggtggagttcgcggaccggggtgggtgtctgctgaccggtcggctgtcgcggtctggggtgtcgtggcttgctgatcatgtggtggcgggtgcggttttggtgccgggtgctgcgttggtggagtgggcgttgcgggccggtgatgaggtcggttgtgtgacggtggaggagttgatgttgcaggcgcctttggtggtgcctgaggcgtcgggtctgcgggttcaggtggtggtcgaggaggcgggtgaggacgggcggcgcggtgtccagatctatagccggcctgacgcggacgccgtgagcggcgacgactcgtggatctgccacgcgaccggcaccctcaccccccagcacaccgacgctccgaacgacggactggccggcgcgtggcccgcggcgggcgccgtgccggtggacctggcgggcttctacgagcgcgtggcggacgcgggctatgcgtacggcccggggttccaggggctgcgtgccgtgtggcggcacggtcaggacctgctggccgaggtcgtcctgcccgaagccgcgggtgcccatgacggctacggcatccaccccgccctcctcgacgccaccctccacccggccctgctcctcgactggcccggggaggtgcaggacgacgacgggaaggtctggctgcctttcacctggaaccaggtctccttgcgggctgcgggagccgccaccgtacgcgtacgtctctcgcccggcgagcacgacgaggcggaacgggaagtacaggtactggtggccgacgccaccgggaccgacgtcctgagcgtggggtcggtgacgttgcgtcccgccgacatccggcaactgcaggccgtgccgggtcacgacgacggtctgttctcggtggactggacgccgctgccgctgtcgcggacggatgtgtcgcagacggatgccgacggggatgccgactgggttgtgctctcggacggtgtcggcagcctggctgatgtggtgtcggcggcgggtggtgaagcgccgtgggcagtggtcgctcccgtcggtgcatccgcgggcggcggccttgccggctttgaccgccgtgagggtcttgacggtcggctggtcgtggagcgggtgttgtcactcgtacaggagttcctggccgcgccggagctggccgagtcccggctcctcgtgctgacccgcggcgccgtggcgaccggcggcgacggcgacggtgatgtggacgcgtccgccgcagccgtatggggcctggtccgcagtgctcagtccgagaacccgggccgcttcatcctgctcgacgtggacatggacgtggacgtcgacgtggacatggacgtggacgtcgacgtggacgtcgacgtggacgtggacggagacggcaatggcagcgacctggacccggacctgaacggccgacgacttccccacgccaccctgcgtcacgccgccgaggaactcgacgagccccaactcgccctgcgcgacggacaactgctcgttccgcggctggtccgcgccaccggcggcggactcgtcgtggcgcccaccgaccgtgcctggcgcctggacaagggaagcgccgagacgctggagagcgtcgcgccggtcgcgtaccccggagtcatggaacccctgggccccggccaggtccgcctcggcatccacgccgcgggcatcaacttccgcgacgtcctggtcagcctcggcatggtgcccggccaggtcggcctgggcggcgaaggcgccggtgtcgtgacggagacaggccccgatgtcacccacctgtcggtcggcgaccgcgtgatgggcgtcctccacggctccttcggcccgacggccgtggcggacacccgcatggtcgcgccggttccgcagggctgggacatgcggcaggcggccgcgatgcccgtcgcgtatctgacggcttggtacgggttggtggagctggccggtctgaaggcgggcgagcgcgtgctgatccacgcagccacgggtggtgtgggaatggcggcggtgcagatcgcccgtcacctgggtgccgaggtgttcgccaccgccagtgcagccaagcacgtcgtactggaagagatgggcatcgacgccgcccaccgcgcctcctcccgggacctcgccttcgaggacaccttccggcaggccaccgacgggcgcggcatggacgtcgtcctcaacagcctgaccggcgagttcatcgacgcatctctgcggttgctcggcgacggcggccggttcctggagatgggcaagaccgatgtgcgcacgccggaggaggtggccgcggagtacccgggtgtcacctacaccgtgtacgacctcgtcaccgacgcggggccggatcgcatcgcggtcatgatgagtgagctgggcgagaggttcgcttccggtgcccttgaccctctgccggtgcgttcctggccgctggacaaggcgcgtgaggcgttccggttcatgagtcaggccaagcacaccggcaaactcgtactcgacgtgcccgcaccgctcgaccccgacgggaccgtcctgatcaccggaggcacgggggcgctggggcaggtcgtggccgagcatctggtgcgggagtggggcgtacggcacctgctgctggccagccgccgtggactggacgcccccggcagcggtgaactcgccgacaggctgtcggacttgggcgccgaggtgaccgtcgcggcggccgatgtgagcgacccggcctcggtggtggagctggtcggcaagacggatccctcgcatccgttgacgggtgtcgtgcacgcggcgggcgtgcttgaggacgggatcgtgacggctcagacgcctgaggggctggcgcgggtgtgggcggccaaggccgctgcggcggcgaatctccatgaggcgacccgggagatgcgtctcggtctgttcgtggtgttctcctcggcggccgccacgctcggcagtccgggccaggccaactacgcggctgccaatgcctattgtgacgcgctgatgcagcgccgacgggcggcgggccaggtcggcctgtcggtcggctggggtctctgggaggcaccggacgccaagccgggtgttgccgccgacgccaaaccggatgttgccgccgacgccaagacgggagttgccgccgacggcactccccagggcatgaccggcaccctgagcggcaccgacgtggcccgcatggcacgcatcggcgtcaaggcgatgaccagcgcacacggtctcgccctgctcgacgccgcacaccgccacggccgcccccacctcgtcgccgtcgacctcgacacccgcgtcctggcgcacaaacccgccccggccctccccgccctcctgcgcgccttcgccggagaccagggaggccagggaggcggccgaggcggcggtcggggcggcggcccggcacgaccggcggccgccaccacccggcagaacgtcgactgggccgcgaagctctccgtcctgacagccgaggaacagcaccgcaccctcctcgacctggtacggacgcacgcggcagccgtcctcgggcacgcgggcaccgacgccgtacgcgccgacgccgccttccaggatctcggcttcgactccctcaccgcggtcgaactgcgcaaccgcctctccgcctccaccggcctgcgcctgcccgccacgttcatcttccggcacccgaccccgtcggccatcgccgacgaactgcgcgcacagctggcccccgcgggggccgacccggccgcgccgctcttcggtgaactggacaagctggagacggtgatcacggggcacgcgcacgacgagagcacccggacccgcctggcggcacgcctgcagaacctgctgtggcgcctggacgacacttcggcccgctcggaccacgcggccggcgcgagcgacgccgacggcgacgccgtcgagaaccgagacctcgagtccgcgtcggacgacgagctcttcgagctgatcgaccgagaactgccttcttgatcaggagtggagaagacatgccgggtacgaacgacatgccgggtaccgaggacaagctccgccactacctgaagcgagtgaccgcggatctcggacagacccgtcagcgcctgcgcgacgtggaggagcgccagcgggaaccgatcgccatcgtcgcgatggcctgccgctacccgggcggggtggcctcccccgagcagctgtgggacctggtcgcctcacgcggcgacgccatcgaggagttccccgccgaccgcggctgggacgtggcgggcctctaccaccccgacccggaccaccccggcacgacctatgtacgagaggccggattcctgcgggacgccgcccgcttcgacgccgacttcttcggcatcaacccgcgcgaggcgctcgccgccgacccgcagcaacgggtgctcctcgaagtgtcgtgggaactgttcgagcgggcgggcatcgaccccgccacgctcaaggacaccctcaccggcgtgtacgcgggggtgtccagccaggaccacatgtccgggagccgggtcccgccggaggtcgagggctacgccaccacgggaaccctctccagcgtcatctccggccgcatcgcctacaccttcggcctggagggcccggcggtgacgctcgacacggcgtgctcggcatcgctggtcgcgatccacctcgcctgccaggccctgcgccagggcgactgcggcctggcggtggcgggaggcgtgaccgtactgtccacgccgacggcgttcgtggagttctcacgccagcgcggactcgcaccggacggccgctgcaagccgttcgccgaggccgccgacggcaccggattctccgagggcgtcggcctgatcctcctggaacgcctctccgacgcccgccgcaacggacatcaagtactcggcgtcgtacgcggatcggccgtcaaccaggacggcgcgagcaacggcctgaccgccccgaacgacgtcgcccaggaacgcgtgatccgccaggccctgaccaacgcccgcgtcaccccggacgccgtcgacgccgtggaggcacacggcaccggcaccacgctcggcgacccgatcgaggggaacgcactcctcgcgacgtacggaaaggaccgccccgccgaccggccgctgtggctcggctctgtgaagtcgaacatcggccacacgcaggcggctgcgggcgtcgcaggcgtcatcaagatggtgatggcgatgcgccacggcgagctgcccgcctccctgcacatcgaccggcccacgccccacgtggactgggagggcgggggagtgcggttgctcaccgatcccgtgccgtggccacgggccgaccgcccccgccgcgcgggggtctcctccttcggcatcagcggcaccaacgcccacctgatcgtggaacaggcccccgccccgcccgacacggccgacgacgccccggaaggcgccgcaacccccggcgcttccgacggcctcgtggtgccgtgggtggtgtcggcccgtagtccgcaggccctgcgtgatcaggccctgcgtctgcgcgactttgccggtgacgcgtcccgagcgccgctcaccgacgtgggctggtctttgctgcggtcgcgtgcgctgttcgagcagcgggcggtggtggcggggcgtgagagggctgaactgctggcggggctggctgcgttggccgctggtgaggagcacccggctgtgacgcggtcccgtgaggaagcggcggttgctgcgagcggtgatgtggtgtggctgttcagtggtcagggcagtcagttggtcggtatgggtgctggtttgtatgagcggttcccggtgtttgcggctgcgtttgatgaggtgtgcggcttgctggagggggagctgggggttggttcgggtgggttgcgggaggtggtgttctggggcccgcgggagcggttggatcacacggtgtgggcgcaggcggggttgtttgcgttgcaggtggggttggcccggttgtgggagtcggtcggggtgcggccggatgtggtgctcgggcattcgatcggtgagatcgcggccgcgcatgtggcgggggtctttgatctggcggatgcgtgtcgggtggtgggggcgcgggcgcgtttgatgggtgggttgcctgagggtggggcgatgtgtgcggtgcaggccacgcccgccgagctggccgcggatgtggatggctcgtccgtgagtgtggcggcggtcaacacacctgactcgacggtgatttcaggtccgtcgggtgaggtggatcggattgctggggtgtggcgggagcgtgggcgtaagacgaaggcgctgagcgtgagtcatgctttccattcggcgttgatggagccgatgctcggggagttcacggaagcgatacgaggggtcaagttcaggcagccgtcgatcccgctcatgagcaatgtctccggagagcgggccggcgaggagatcacatccccggagtactgggcgaggcatgtacgccagacagtgctcttccagcccggcgtcgcccaagtggccgctgaggcacgcgcgttcgtcgaactcggccccggccccgtactgaccgccgccgcccagcacaccctcgaccacatcaccgagccggaaggccccgagccggtcgtcaccgcgtccctccaccccgaccggccggacgacgtggccttcgcgcacgccatggccgacctccacgtcgccggtatcagcgtggactggtcggcgtacttccctgacgaccccgccccccgcaccgtcgacctgcccacctacgccttccaggggcggcgcttctggctggcggacatcgcggcgcccgaggccgtgtcctcgacggacggtgaggaggccgggttctgggccgccgtcgaaggtgcggacttccaggcgctctgcgacaccctgcacctcaaggacgacgagcaccgcgcggctctggagacggtgttccccgcgctgtccgcgtggcggcgcgaacgacgtgagcggtcgatcgtcgatgcctggcggtaccgggtcgactggcggcgcgtcgagctgccgacacccgttccgggcgccggtaccggtcccgacgccgacacgggcctcggggcgtggctgatcgtggctcccacgcacgggtcgggtacttggccgcaagcctgtgcccgggcgttggaggaggcgggcgcgccggtacgtatcgtcgaggccggcccgcacgccgaccgggcggacatggcggacctggtccaggcatggcgggcaagctgtgcggacgacaccacccagctcggaggagtgctctccctgctggctctcgccgaggcaccggccaccagttccgacaccacttcccacaccagtaccagttgcggtaccggctctctcgcgtcccacggcctcaccggcaccttgacgctgctgcacggtctgctggatgcgggcgtcgaagcgcctctctggtgtgccacgcgcggcgccgtgtcgtgcggcgacgccgatccgctcgtctccccgtcgcaggccccggtctggggactcggacgcgtggccgccctggagcatccggagttgtggggcggcctggtcgacctgcccgccgacccggagtcgctcgacgcgagcgcgttgtatgcggttctgcgcggagacggcggcgaggatcaggtcgcgctgcgccggggcgcggtcctcggccgtcgcctggtgcccgacgcaaccccggacgtggcccccggctcgtccccggacgtgtccggaggcgcagcccatgccgacgcgacctccggggagtggcagccgcatggtgccgtcctcgtcaccggaggcgtcggccacctggccgatcaggtcgtacggtggctcgccgcgtccggcgccgaacacgtcgtactcctggacacgggccccgccaacagccgtggtcccggccggaacgacgacctcgccgcggaagccgccgaacacggcaccgagctgacggtcctgcggtccctgagcgagctgacagacgtatccgtacgtcccatacggaccgtcatccacacatcgctgcccggcgagctcgcgccgctggccgaggtcacccccgacgcgctcggcgcggccgtgtccgccgccgcgcggctgagcgaactccccggcatcgggtcagtggagaccgtgctgttcttctcctccgtgacggcttcgctcggcagtagggagcacggcgcgtacgccgccgccaacgcctacctcgacgccctggcgcaacgggccggtgccgatgctgcgagcccccggacggtctcggtcgggtggggcatctgggatctgccggacgacggtgacgtggcacgcggcgccgccgggctgtcccggaggcagggactcccgccgctggaaccgcagttggcgctcggcgccctgcgcgcggcgctcgacgggggcaaggggcacacgctggtcgccgacatcgagtgggagcggttcgcgccgctgttcacgctggccaggcccacccggctgctcgacgggatccccgcggcccagcgggtcctcgacgcctcctcggagagcgccgaggcctcggagaacgcctcggccctccgtcgcgaactgacggccctgcccgtgcgggagcggaccggggcacttctcgacctggtccgcaaacaggtggccgccgtcctgcgctacgagccgggccaagacgtggcgcccgagaaggccttcaaggacctgggcttcgactcgctcgtggtcgtggagctgcgcaaccggctgcgcgccgccaccgggctccggctgcccgccaccctggtctacgactaccccacaccccgcaccctcgccgcacacctgctggacagggtgctgcccgacggcggcgcggcagagctccccgtggccgcccacctggacgacctggaggcggccctcaccgacctgccggccgacgacccccggcgcaagggcctggtccggcgtctacagacgctgctgtggaagcagcccgacgccatgggggcggcgggccccgccgacgaggaggagcaagccgcgcccgaggacctgtcgaccgcgagcgccgacgacatgttcgccctgatcgaccgggagtggggcacgcggtgagcggggtggagcggggtgtggggtcggcgggccctgtggaacagggtgacggactcgcgggcctggtcgagcgggccgaggcgctggccgctctgcggggcgccttcgacggctccccgggcaccggcggcagcctcgtcgtgctcagcggcgcggtgggcaccggcaagaccgcgctgctacgggcgtgggccgaccgcatcggcgccgatgccgacgccctggtcctgaccgccaccgcctgccgcgccgagcgcgacctgccgcttggcgtcctggaacagctggtacgcagccccggcctgcccccggccagcgccgagcgcgcgctggcgtggtgggacgaggaggcctcggccacccccggaaagacggacgcgaacgggacgagtgccaacgggacggacgccaacgggacgggcgcgggacagacgggcgcggggcaggcgggcgtgggacagacgggcgtgggcggagagcccgtcctggccgcctccgccctgcgaggcctgtgcgaggtgctgcgggacctgctcgccgagcggcccgtcgtggtcgccgtcgacgacgcgcaccatgccgacgcggcgtcgctccagtgcctgctctccgtggtgcgccggctgcggtcggcacgactccatgtgctgttcaccgagtacgcccatcagaaggcgcagaacgccctgctgagcagcgagttcctgcacgagcccgccctgcggcggatccgcctggaaccgctgtcgaaggcgggcgtggaggccttgctcgcccggcacctcgacgagcggacggcacaagacctcacccccgtcgtccacggcatgagcgcgggccacccgctcctcgtacgggcgctggccgaggaccaccgtgcggcgggcggcgccggggaggcgtacggtcgtgccgtcctcagctttctgtaccggcacgagactccggtcacccaagtcgcccgcgccatcgctgcgttgggcgcgcacgccggacccggtcaggtcgggcggctgctcgatgtcgacgcggcgtccgtcgagcgggccgtgcggcagctgaccgtcgcggaggtgctgcacgagggccgcctgtgccacccggcgttcgcggcggcggtcctggacggcatgccgcccgaggaacgccgcgccctgcacggacgggtcgccgacctcctgcacgaggagggggcgccggccaccgaagtggccgcccacctcgtcgccgccgaccggtccgacgccccgtgggcggtacccgtcttccaggaagcggcccaactcgccctggacgaggaccaggtggagaccggcgtcgactatctgcgcgcggcccaccagcggtgccggggcgccgcgcagcgtgccgcggtcgtcggtgcgctcgccgacgccgagtggcggctcgacccagcaaaggtcctgcgccacctgcccgaccctgcagccatggccccacaaacggaccctgccgccctggccccacacacggaccccgcacccacagccgcacccacagccgcccccacccccacccccatcccgaccaccccacccctccccacccacctgctctggcacgggcgggtcgaggaaggcctggacgccatcggcacgctcaccgggcccggacccaacccggcgggtgcgccgccgatgaaccccgcggacctggacaccccatggctgtggggcgcctacctctatcccgggcacgtcaaggagcgcctgggatccggcgccctgtccccgcagcgctcgaccccgccggcggtcacgccggagctccaaggcgcgggcacgctgatgaacgacctgctgcacggcggcgaacgcgacgccaccgaggccgccgagcgcgccctcaaccgctaccggctcggcccccgcaccatcgcggtccagacggccgcgctggccgccctcacctaccgcgaccggccgcaccgcgcggccgcctggtgcgacggcctcgtcgcccaggccgacgagcgcaacagccccacctggcgggccctgttcaccgcgtggcgtgccctgctccacctgcggcagggcgacccggccgcagcggaacagcgcgccgaaaccgccctcgccctgctcggatcgaagggctggggcgccgcgatcggcctgccgctggcagccgccgtacaggccaaggcggccctcggcgatgtcgacggggcggcggccctcctggaacggcccgtgccccaggcggtcttccagacccgcaccggactgcactacctggcggcccggggccgctatcacctcgccaccggctgccactacgccgcactgtgcgacttctacgcctgcgggacccgcatgagcagctggggagtggacctgcccgcgctggagccgtggcgcctcggcgcggcggaagcgtacctggccctcggcgaaggactcctggcacgccaactcgtcgacggccagctgccgttgcccacgcctgacgacggccgcacctggggcatgacgttgcgcctgcgggcggccacgtcccccgcgccggcccgggccgaactcctcgacgaggccgtggcggtgctccgggagagcggcgacaccttcgagctggcgcgggccgtcgccgaccaggctgttgccgtacgcgaagggggcgaggcggaacgcgcccggctgctggcccgcaaggcggagctgctggcccggcgctggggcagcgcccccgcgcccgccaccgtccccgaaccgccggagcggccaggaccggccactccggacgccgaactgaccagtgcggagcggagggtggccgagctggccgccgaagggttcaccaaccgggagatctcccggaagctgtgcgtcacggtcagcaccgtggaacagcacctgacccggatctaccggaagctcgacgtcaggcgactggacctccaggcagccctcggctgaccttcaggcggccctcggctgaccgcaggccacgcgcctacggtcagccttcctgagtcaggaccgtacagccgccgtaggtgtaggtgtaggcgtgggcgagatcgtcgccgcgtccagacccaccacggccagctcctccggaaggaacgggggagcggtcagctccgggaggcgttcgtcggcgcgcatcgccatcaggaaacggttggagcccagttcggcctgcggcgcgttgaggctcatcacgtccgtgacgatctcggacgccttcggggaacggatcgacgccgcggtgatggcctcggcgaaccgcagacgctgctcggtgtccacaccgatgagccgcggatccgtcgccgagacacggcagttgacgtagtcgatgtccttggtcgcggcgaggatccacgggtcgtccacggccgcgccgatcgccttctgcagggcgcgggtgccggcgcgggcggaccccgtaccctcctgcacgctccgctcgaactcgcggtcgatcgtggtggcgcagcgcgcggccgagctcatgccgtggccgtagatcgggttgaaagcggtcagcgagtcgccgatgacgagcagaccgtcgggccactgttcgaggcgctccggatagaggcggcggttggcgccggagcgggaaccgaagacgggggtgagtggttcggcgtcccggagcaggtcggcgaggatcgggtggttcaggttctcggcgaaggggatgaactcgtcctcgtgtgtgggcagttgcgcgccccgcgtgcaggagagcgtcgcgagccagcggccgccctcgatggggtagaccacgccgaagcggccgggttcgcgcacccggtcgtcggcggcgatgttcacggcggggaagtgcgtcgtagcgcccggcggggccttgaagagccgggtggcgtaggcgacgcccgcgtccacgacgtcttcctccagtgccggcacgccgagggcggcgagccactgcttgaggcgggagccgcgcccggtggcgtcgatcaccaggtcggcctccagctgctcctgccgaccgctgtcgaggtcgcggacgacgacaccggtgacccggccgccactgccaccaccacttcccgtcagctcgacggcctcggtgcgctgccggacggtgatgttgtcggctcccaaggcctgctgacgtaccgtcaagtccagcagcgggcggctggcgaccagcgcgaactgggtggcggggaagcggtgctgccacccctgaccggtcagcgtcaccaggtcctcggggaagccgaggcggcgggcgccggccgcgaggaggcggtcggtggtgccgggcagcatctcctcgatgaggcgggcgccgttggaccacaggaggtgcgcgtggcgggcctgcgggacccccttgcggtgctggggctcctcgggcagcgcgtcacgttccacgacggtgacggcgtcgacgtgccgggccaggacgtgggccgccagggtgcctgccatgctggcacccaggacgacggcatgtgcgggtcgggtggtggtcacgcgcgtatcccttcggggtgggtggtgtcggcgggcccggccggatcgtccatggtcacgtccgtgacgccccagaacgcctggacccggcggccgagcccgtgctcgtcgagttcgacgatgccgacgatgcggaaggtcatcggccgcggccgctgcacggtgaccgtggtcggcgtcaccacgaaacggtcgtccatcgacgtcatcggcgggtccggcacctcgtgcgtaccgcaggagacggccagttcgagatggcggcggagatcgtccttgcccaccatcgggggccgccccacggggtcctcgaagacgatgtcgtccgtgaacaggtcgaggacgccttcgatgtcaccggcgttgatgcgctcggcgtagtcgacggccatctgcttgcgcgcggcctcgtcgggcatggcacctccaggaagggtgggcagaccttgtgaaagtcatcgagggccgttcggttcagccgaggaccgtgagatcggatgtgccccagtacgacttcagatgccggatgaggccggacgcgtccatgcggatcacgagcatcgccgtgcggtgtatgcgggccgtccccggggcgtcgggggccttgagccagccccgctccgcgtagagcgggcccacgggcaggtagtccatgacggaggaaatctggatcagcgcgtgcgtggcgtcctgcccggcgacgggctcggccgcctcctcgcgcaggtgcgcggcgagcagcggttcgtagtgggcgcgcagcgcgtcgtgcccggtgacgggcgggaggccgaccgggtcctcgaggaccgcgtcgggcgcgtacagatcgatgatcgcgtccaggtccccggcgttgatccgccggctgtgctccagggcccgcttcttgcgggcgaactcgttcatcgctgcccctccactgcctgaccgtgtccgttgccgttgccgttgccgttgccgttgccgtgtccgttgccctgcccggtgggctgtccgttgccctgtccgctcgcgccgtccctgccgaggtcccggtcgatgaacgcgaagatctcgtccgccgacgcgtcctggatacgtgtacgagtggccaccgcgacctcgccggccgtgtcctgcggcgcgtcgagcctggccagcgtcgcgcgcagccgccccgccagttcggcccgcgccgagccgtccttcgaggagaccgagagcagcgagtcctcgatgcgctcgaactccgccaggacgtcggcgagcggatccgccgcgcgcggggccagctcctgccgcagctgcgcggcgagctccgccgggttgggatggtcgaagacgaacgtggcgggcagcttcagccccgtcgcggccgagagccggttgcgcagctccaccgcggtcagggagtcgaagccgagttcccgcagcccctgcgtcgcgttgacgggcgtggccgcgtcgtagccgaggacggccgcgatatgggtgcacaccaggtcgagcagcgcctcctcccgctcggggtcggacatcgcgccgagcgacttgagcagcgcggccgcccccgccgacacggcaccgccgccgctcttgctccccccgcgcaccaggtcgcgcagcagcgccggtgcggggtggctctgggcctgccggcgcatccgggccaggtccagacggaccggcgcgtacaggggcagtccgccggcccacgccgcgtcgaggagggcgagtccctcgtcggcgccgagcccgaccacgccggcgcgggcatggcgcgcccggtcggcgtcggtgagccgtcccgacatgccgctcgccagctcccagtagccccacgccagggaggtcgccgccgcaccgccgtcgtgccggtgccgggccagcgcgtccaagaaggcgttggcggccgtgtagctgccctggccggggccgccgagcagcccggcgaccgaggagtacaggacgaacgcggacaggtccgcgtcccgcgtcagctcgtgcaggtgccacgcggcgtccgccttcacgcgcatcacctcctcgacctgctcggccgtgaggttctgcaccacggcgtcgttcacggtgcccgcgcagtggaagacggcggtcagcgggtggtccgagggcaccgccgcgaggagggcggcggcttcgtcccggtcgcccgggtcgcacgcggcgaaggtgactcgcgcgccgagcgcggagaggtcggcggccagttcgagtgcgcccggcgcgtcggctccccgcctgctggacagcaacaggtgcctggctccgtaccgttccaccaggtgacgggccgtcagcgagccgagtgctccggtgccgccggtgaccagcacggtgccctcggggtcgaaggcgggaggcagcgagaacacggtcgtgcccgccgagggcggggccgccatcgcggcgggcgcctgccggatgtcccacacggtgatgtcgagcggcgtcagagcacggctgtcaccccgttccgcgggcagcccgggctccgccgactccgtgatctcggcaagctcggtcagctcggtcagctccgcgaggatttcccgtacgcgcccgggctcgggcggcacgacagcctgtccctcgtccggacgaccgcccgcacggtggaccaccagggccccctcgtggcggagggtgacgtcggccgcgcgctcggcgcccgaatcatccgccgtcgacgcaccgtccacggcctcgacccgccaccgcccggcaagagcaagacgcagcacggcacggccgacggaaccggtttcctccccgacgagcagagtctcgccgccgcgcggcgccacgacatccgccagcacgtgatacgcggacacataggcccccaaggacccggccgcctgcgcccaactccagcccgccggaaccggcataagcagcgcggcatcggtgacggccaccgggcccaccgcgtcgaacaaccccatcacccggtcgcccacggccaccgaaccgacctcgccgccgacttccgtcaccacaccggcaccctcgacctggcccgccgtgagcggccccggcgccgcggcccgcaccgccacccgcacctcgtgcggctccagcgcccgtccggcctcgggagcgtcgacaagggacaactgctgtccgccgcccgcctcttggcaccgagccagccgccacgtgagcgatccgaccggcggcaccagccgcaccgacgcgtcgtcgcgcacgagccgtggcacgtaggcgcgcccgtcacgcagcgccaattccggttcgccggaggccagtacgccggtcagcgtggccggagaagactccagtccgtccacgtcgagcagcgtgaggcgaccgggattctcggcctgcgcgctgcgcaccagaccccacagcgacgcgcccgccagatcaccggcggtctcacccggccgcgcggcgaccgcgcctcgggtgacgacgacgagacgggtcgccgcgaacgccgggtcgtccacccactccttgagcagcgacagaagggacacggtggccagccgcgcgtacccggccgggtcgccgcccctgccatcggcatccgcaacggccccggcacctgcgccgggcgcggcgcacacggcgagcacgacatcgggcgcttcgcccccagccgccactccgtcccggagcgcaccgaacgtgtcccacacggggccggcggccagcgcatcggacaaggcgtcggccagcgcaccggccgacgtaccgcccatcgggccactctcgaccggcgcgaggaccgcggcacgcggggcgccgccgcccgtctcctcggcccgcgcggcgacctccatccacacgagccggaacagcgcgtcacggtccgccgcacgggcgcccgcgatctggtgggcggccaccggccgtaccgtgagcgactccagcgtgagaaccggctccccgcctccgcccccgtccacggccgtgagggccagctggtcgggcgcggtgcgtgcgatacgtacccgcaacttctcagcgcccggcgcgtgcacccgcaacccgctccaggagaacggcagcagcacttggtcggtgtcggcggacgacgtgaccgcgtccaggatcagcgcgtgcagcgtggcgtcgagcaacaccgggtgcacctggtagcggtcggccctgccgctctccgcctcgggcagcgccacctcggcgaaaaggtcgtccccgagccgccacgcgctcaccagtccctgtgagccgggcccgaagtcatagccgtacgaagcgagttccccgtacggatcctgctcgccgaccggtgtggcgcccgggggcggccacgtcccgccgaacgaggcgtccccggcgtcgggccccgggggagcgaccacgcccgcggcatgccgggtccacacggcctcctcgccctcacccgtgggccgcgaatggacggtcacgggacgccgcccgtcctcggccacggaaccgaccaccacctgcacgtcgaccgcgcccgcaccctcgtccccgaaggcgagcggagtgtgcagcgtcagctccgccaactccgcgcagccggcccgcaccgcggcctgcagcgcgagctccacgaacgccgaaccgggcagcagcaccgtgtccatgacccggtgctcggccagccacgcctggtcccgcggagagatccggccggtcagcaggtgactgccgccgtccgcgagttccacggcggctccgagcagcggatgccccgcggacgcgagcccgagccccgccgggtccccggcgagccccctgcgcccctccagccagaaccgctcccgctggaaggcgtacgtcggcagatccaccacccgaggcagcggcacggccgggaaccagcccgtccagtcgacctccgcccccgcgccgaaggcctgggcggccgcgcgggtgagctgcgcggcgtcgccgtggtcgcggcgcagggtgggcacgacggtggcgggcatgtcggcccgctcgatggtctcctccatgccgaggttgaggacggggtgggggctggcctcgatgaacaggcggtagccgtcggccagcagcgcttcgatggtgtcggcgaagcggacgggctggcggaggttggtgacccagtaatccgtgtcgagggtggtggtgtcgtcgaggcgttcggcggtgaccgtggagtagaaggcgacgtccgtggtcgtgggccggatgtcggccaggcgctcggtgaggaggtcgtggagctggtcgatctgggggccgtgggaggcgtatccgacgtcgatgacgcgggcgcgcaggcctcgcgcctccgcatccgcgaccacggctgccacatgctccggcggccctgaaatgaccgtagaggagggcccgttgacggcagcgacacacacgccgggccggtcgccgatgagctcagcaacctgctccgagccggcccccaacgacgccatgtcgccctgccccatgagctgacggagcgcgtcactgcgtacggccacgatccgcgccgcatcctccagtgacagtgcccccgccacacacgcggcggccatctcgccctgcgagtgcccgatgacggcagccggggtgatgccgtaatcggcccacaccgaagccagcgagaccatcaccgcccacaacacgggctgcacgacctcgacccgggacagctcactcccgtccccgcgcaacaccgcactcagcgaccagtccacatgcgccgacagggcccgctcacactccgcgatccgcgccgcgaagacgggggactcgtcaaggagctgggcacccatgcccacccactgcgacccctgccccggaaacaccaacaccggacccgcgccggaggcgccctgtacggcgccctcgacgacgtccggtgacggctcgcccgccgccagggaccgtagcccggcgaggagagtctggcggtccttgcccacgacgacggctcggttctcgaacaccgaccgggtcttgaccagggaccagcccacgtccagcggcgacgcgagccgcgggtcggcggtggcgcggtcggccagcaggcgggcctgggcccgcagcgcctcctcgccgcgcgccgacaccacccagggcaccactccggccggcgccgcggcgtcctccgccggagcggtcacgggctccggcgcgtccggggcctgttccaggatgaggtgcgcgttggtgccggagatgccgaaggcggacaccccggcgcggcgcgggcgttcgccgcgcggccaggagaccggttcggacagcaggcggacgccactgccgtcccagtccacgtgcggcgtgggcgcgtcgatgtgcagggaggcgggcagctgttcgttgcgcagcgccatgaccatcttgatcacaccggcgacaccggccgacgcctgcgcgtgcccgatgttcgacttgatcgagccgagccacagcggccggtccgcgggccgctccttgccgtaggtggcgacgagcgcgctggcttcgatggggtcgcccagcatggtgccggtgccgtgcgcctccaccgcgtcgacgtcctcggcggagagccgcgcgttggcgagtgcctgccggatcacccgctgctgcgcctgcccgttgggtgccgtgagcccgttgctcgtgccgtcctggttgatggccgaaccccggatcaccgccaggacgttgtggccgttgcgccgggcctccgagagccgttcgagtacgaccaggccgactccctcggcccagccggtgccgtcggcggcggccgcgaacggcttgcaccggccgtccttggcgagcccgcgctgcagcgagaactcgacgaacgagcccggcgtggccatcaccgtcgcgccgcccgcgagagcgagcgagcactcgccctggcgcagcgcgtgcgccgcctggtggatcgccaccagggacgaagagcagccggtgtcgatcgtcatggcggggccttctaggccgagtacgtacgacaccctgccggaggcgacacagccgaggttgccggtgccgatgtagccctcgacctcggtgggctgttcaccgacgagcgcgaggtagtcgaagatggtcaggcccgtgaacaccccggcgtcgctgcccttgagggtctcccggtcgaggcccgcgcgttcgatcgcctcccacgcggtctccaggagcagccgctgctgcgggtccatcgcgacggcctcgcgggggctgatgccgaagaatccggcgtcgaagtcgcccgcgtcgtagaggaacccgccttcgcgcacatagctggtgccgcggctctccgggtccgggtcgtacagcgtctccaggtcccagccccggtcgtcggggaaggcccccatggcgtccttgccggccgcgaccagatcccacagctcctcggcggagcggacgtcgcccggatagcggcaggccatgccgacgatcgcgatcggctcgtcgtcggcggcgcccctggaggccccggccgcccgcaccgggtcggcggaggccgccgcgtcaccggacagctcggcccgcaggacgtcggtgagcgcgtcgggggtggggtggtcgaagacgaccgtggtcggcagtgtcaggccggtgctcttgttcagcctgttgcgcagctccaccgcggtcagcgagtcgaagcccagctcctggaacggcttggtggcgggcaccgcgtcgacgtccgagtgccccagcgtggccgccgcctgggagcgcacgtgctgcagcagcaactgccgctgctgcgccggcttcgcctccgtcagctcctgctggagcgacgatgcctccgtggcgtcttcctgctgtgccgcgggtgcgctggcccgccggttctcgggcagatcggcgaggagcgggctgggccgctgcgcggtgaacgtcgacgtgaactgcgcccagtcgaagttcgccacggtcagcgtcgtctcacccgcgtccagggcctgctgcagcgccttgacgcacagctccgggctgagcgggtgcaggccgaagcggctgaagaacgtcaacgcggcctggtccgccgccatgcccgcctcggcccagggcccccaggcgatggaggtggcgggcaggccctcggcgcggcggtgctcggcgagggcgtcgaggaagtggttggccgcaccataggcgccctgctggccactgccccacacgcctgcgcccgacgagaacatcacgaacgccgagagcggcaactcccgggtcagttcatgcagatggtgagcggcgagcgccttcggacgcagcacctcgtccagctcggcacccgacacgtcgccgagaccgatgtagttcggcacgccggccgcgtggatgacggcggtcagcgggtgctcggcggggacatcgtcgatgaggcgtcgcacctgctcgcggtcgccgacgtcgcaggcggtgacggtgacggcggcccccaactccgtcagttccgcggcgagttcctgtgctcccggggcgtcggggccgcggcggctggtcaggaggaggtgcggggcgcccgcacgggcgagccaccgcgcgaggacggcgccgatgccgccggtcccgccggtgatgagagtggtgccgtcgggccgccaaccaagcccgctgccgaccgtgttggcgggcgcgtgtgcaaggcgacgggcatggacgccggacggccggatggagatctggtcctcgtcctgcggaaccagcgcggcggccagccgggccagcgtctgatggtcgatacgagcgggcagatcgaccagcccgccccacagccgcggatactccagcgcagcgacgcgccccagcccccacacctgagcctgcaccgggtgggtgagggcgtcgccggcgctcgtggaaacagccccctgcgtgagagtgcgtacggcgatgtcggcgccgttgtccgcgagggcctggacgagagcggtcgtcgcggcgagtccggcgggcacggccgagtgctcgggatgcggctcctcgtccagggccagcagattgacgactccggcaaacgcggccccgtccatcaggacacgcagctcctgcgccaactccgtacgctccatggcacgtgcgtcgaccacgtggcgtcgcacctcgccaccatgggcggtcagcgtctgcgcggtcgcgaggacggccgggtggtcggcgtgcgcggcgggcacgagcagcagccaggccccgctgagctccggcgccggcacgtcgggcagatgcttccaagtgacctgataacgccaggagtcgacggtggactgctcgcggtgccgacgccgccaggccgagaggacgggcagcgcggactccagcgctccgacgctctccgcctgcccctcgatctccagactgccggcgagggcgtcgatgtccaggtcctcgatcgcctgccacacccgggcctcgaccggatcgtgcccaccacccacggctgcgaccgccgcgggcggctccacccagtagtgcttgtgctggaaggcgtaggtggggaggtcgacggtacggggggtggggtcggccgggaaccagcgccgccagtcgacgggggcgccggcggtgaaggcgtgggcggccgcgcgggtgagctgggtggtgtcaccgtggtcgcgacgcagggtggggatggtgacggccgtccccgcagcaccggcctgctgctcgatggtctcctggatgccgaggttgaggacggggtgggggctggcctcgatgaacaggcggtagccgtcggccagcagcgcttcgatggtgtcggcgaagcggacgggctggcggaggttggtgacccagtaggcggtgtctagggcggtggtgtcgtcgaggcgctctgcggtgaccgtcgagtagaacgccacgtcggtggtggtcggctggatgtcggcgagccggtcggtgaggaggtcgtggagctggtcgatctggggaccgtgggaggcgtacctgacgtcgatgacgcgggccctgagtccctgcgcctccgcatcggcgacgacggctgccacatgctccggcgggcccgaaatcacggtcgacgacggtccgttgacggccgcgacgactacgcccggccggtcgccgatcagctctgcggcctgctcggcaccggtgctgagcgaggccatgtcgccgtgcccttgcagctgacgaagcgcgtcgctgcgtacggctacgatccgtgccgcatcctccagtgacagtgcccccgccacacacgcggcagccatctcgccctgcgagtgcccgatgacggcagccggggtgatgccgtaatcggcccacaccgcagccagcgagaccatcaccgcccacagcacgggctgcacgacctcgacccgggacagctcgctcccgtccccgcgcaagacatcactcagcgaccagtccacatgcgccgacagcgcctgctcacactccgcgatccgcgccgcgaagacgggcgactcgtcaaggagctgggcgcccatgcccacccactgcgacccctgccccggaaacaccaacaccggcccaggacccacatcaccggccaccccggccaccacatccgccgacgcctcacccgcggccaatgcctccaggctggcaccagcctgagccaagtcccgccccacgaccacggcccgctgatcgaacaacgcgcgtgtcgtggccagcgaccagcccacctcggagaccgacgcatccgccagcccggccgcgaactcgcccagccgccgcgcctgttcacgcaacgcgtccggcgtccgcccggacaccacccacggcacgaccccacccggctcagccgccacggggcccggcgcgtcctcttccggcggcgcctcctccagaatcaggtgcgcgttcgtcccggagatcccgaacgccgagatgcctgcccgccgcgtgcgctccgccggccagtccacgggctcggagagcagtcgtacgctgccctgttcccactggacgtgcggtgacggggcgtcgatgtgcagggaggtcgggaggagaccgttgcgcatcgccatgaccatcttgatgacgcccgcgacaccggcggcggcctgcgtgtggccgatgttggatttcaccgagccgagccagagcggacggtcctccgggcgcccctggccgtacgtggcgatcagggcctgcgcctcgatggggtcgccgagcgtggtgccggtgccgtgcgcctctacggcgtcgatgtcctcggcggagaggcgggcgttggcgagggcggcgcggatgacgcgttcctgggaggggccgttgggggcggcgagcccgttgctcgtaccgtcctggttggtggccgaaccccgtatcaccgcaaggaccttgtggccgcggcgccgcgcttcggagagcagctccagcgccaccaccccggcgccctcgccccagccggtgccgtcggcggcggccgcgaacggcttgcaccgcccgtcgggcgcgagcccccgctgccgggagaactcggtgaacgaacccggcgtcgccatcaccgtcgaaccgcccgccagcgcgagcgagcactcgccctgccgcagcgcctgacttgccagatggatcgccaccagggacgacgagcacgccgtgtcgacggtgaccgcgggaccttcgagccccaccgtgtaggagatccggcccgacaccacactgccgaggttgccggtgccgatgtacccctcgacgtcgctggccgtctggctgatcagcgtcaggtagtcgtgggcgctcactccggtgaagacgccggtgtcgctgcccttcagcgcgtgcgggttcatgcccgcgtgctcgatcgcctcccacgcggtctccaggagcagccgctgctgcggatccatcgccgtggcctcgcgcgggctgatgccgaagaactcggcgtcgaaatggccggcgtcgtacaggaaggcgccgtcccgcacatagctggtggccggatgctccggatccgggtgatacagcgactccaggtcccagccccggtcgtcggggaaccccgcgaccgcgtcacccccgtcgcgcacgagttcccagaggtcctccgccgaccgggcgccgcccgggtagcggcaggccatgccgacgatggcgaccggctcggtcgattccttgtcgtggagccgttgccgggcctggcgcagctccgcggtgacccacttgaggtgatcgagaagcttctcctcgttcgacatctgacccaggctccttggcgctacgtggtgatcggggcgtatgaggttgggggagggcaagggggccggtgtggccggggctcatcgcgctcaggactgatcgctgctcaggacttcccgaactcactggagatgaggtcgaagatgtcgtccgcgctcgccgcctccagatcggcatgggccgaatcagtgccttccggcccgtcctgcgccggactccacttcgacacaaggacctgcagccggcccacgatgcggcgccgggccgcctcgtccacctcggccgctccgaacgccgtgtcccacttgtcgagcgccgcgagcacgtcgccctcacctgcgacctcggcgccgtcgccgagctgtccgcgcaagtgcgtggcgagggcctcgggcgtgggatggtcgaagatcacggtggcggggagcgagagtccggtcgtggtgttgagctggttgcgcagctggaccgcggtgagcgagtcgaagcccagctcctggaacggcttcgcggcgggaatgtcctccaccgtgcggccgagcgtcgcggccgcgtatgtccggacctgctggaccaggaagccgagccgctgtgatgcgggcgtcttcgccagctcctcgcggaacgcgctcgtctcggcggcggtccccgtctgctcggcctcccgctggttctccggaaggtcgtcgaggaacggactgggccgctgcgcggtgaacgtcggcgtgaacttcgcccagtcgaagttcgccacggtcagcgtggcgtcgcccgcgtcgaccgcctggtgcagcgccttgacgcacagatccggagcgatcgggagcagaccgaagcgcttgaagtacgtcagtgactccgggtcggcggacatgcccgcctcggcccagggcccccaggcgatggaggtggcgggcaggccctgggcgcggcggtgctcggcgagggcgtcgaggaagtggttggccgcaccataggcgccctgctggccactgccccacacgccggcgcccgacgagaacatcacgaacgccgagaggtccaggtcgcgcgtcaactcgtgcaggttccaggccgcgtcggacttcgaccccagcacctcgccgaggcgcgcggtcgtcagatcaccgatcgcggtcagatcggtcatgcccgccgcgtggatgacggctgtgaggggatgctcggccggcatgtcgtcgatgaggccgctcagttggcggggatcgctgacgtcgcaggcggtgatggtgacggcggtgccgagcccgtcgagctcggcggcgagttc

SEQ ID NO: 4 - DNA, 13600 bp, Streptomyces cinnamonensis
  Nucleotides 90001 - 103600 of the monensin biosynthetic gene cluster
ccgggcgccgggcgcgtcgggcccgcgacggctggtgaggtgaagacggggggcgccctgccgggccagccaacgggcgaggacggcaccgatgccgccggtcccgccggtgatcagggtggtgccccgaggccgccaggtggcctcgctgtgcacgggattctgaatgcttccgacggcgtgcgtgaggcgccggtggtggattccggtggggcggacggcggtctggtcctcgtcgtcctgggggaggagagcggcggcgaggcgggggagggtgtggcggtcgatacgagcggggaggtcgacgagtccggcccagaggcgcgggtgttcgagggctgcgacgcggccgagcccccagacgtgagcctggagggggtgggtgagtgggtcggtggcgcccgtggacacggcaccctgcgtgacggtgtgcaggggtgcggtcgtgccgttgtcgccgagggcctggaggagagcggtcgtcgcggcgagccccgcgggcacggcggggtgctcggggtgcggctcctcgtccagcgccagcagattgacgattccggcaagaccggccgtgtccaccgcggccagctcctgacgtcccgcccggccggtctcgaccggatgcagccggacggcggccgccccgtgctcgctcaacgcctcggcggtggctcgtacggcggggtgctccgccttgtcggcagggacgaacagcagccagtcgccgccgagttccggtgcgggcccgtcggaccgctgtttccacgtgacgcggtaccgccaggagtcgatggtcgcctggtcctggtgccgacgccgccagcccttgagcaccggcaacgcgggctccagcgcccggaccgcctcctcgctgccctcctccgaccccagcgtctcggccagcagaccgagatcgagctcctcgacggcgtgccacagctgggcctcggccgcactctgctcaccgctgacggcgcccgaggcggacgcggaacgttcgagccagtagtgctggtgttggaaggcgtaggtggggaggtcgacggtgcggggggtggggtcggccgggaaccagcgccgccagtcgacgggggcgccggcggtgaaggcgtgggcggcggcacgggtgagctgggtggtgtcgccgtggtcgcggcggagggtggggacgacggtggcggggatgtccgcctgctcgatggtctcctccatgcccaggcccagcacggggtgggcgctggcctcgatgaacaggcggtagccgtccgcgagaagggcttcgatggtgtcggcgaaccggaccggctggcggaggttggtcacccagtaatccgtatccagggctgtggtgtccgtcagacgctcggcggtgaccgtcgaatagaaggccacgtccgtgttcgcgggccggatgtcagccaggcgttcggtcagcagatcgtggagctggtcgatctgggggccatgcgaggcgtacccgacgtcgatgacacgggcgcgcagaccacgtgcctccgcatcggcgaccacggcagccacatgctccggcggccctgaaatcaccgtagacgacggcccattgaccgccgcgacgaccacgcccggccggtcaccgatcagctcagcggcctgctcggcaccggtgctcagcgaggccatgtcaccgtgcccttgcagccgacgaagcgcgtcactgcgtacggctacgatgcgcgccgcatcctccagcgacagcgcccccgcgacgcacgcggcagccatctcaccctgcgagtgcccgatcacagcagccggagtgaccccgtaatcagcccacaccgcagccagcgagaccatcaccgcccacaacaccggctgcacgacctcgacccgggacagctcactcccatccccgcgcaacaccgcactcagcgaccagtccacatacgccgacagcgcccgctcacactccgcaatccgcgccgcgaagacgggggactcgtccagcagctgggcacccatgcccacccactgcgacccctgccccggaaacaccaacaccggcccaggacccacatcaccagcaaccccggccaccacacccgccgaagcctcacccgcagccaacgcccccaggccagccgtcaacgcatcgcggtcacgccccaccaccacagcccggtgctcgaacaccgaccgggtcgtggtcaacgaccagcccacatcagccgccgacgcatccgccggcccggccgcgaactcgcccagccgccgcgcctgtgcacgcagcgcgtccggcgtccgcccggacaccacccacggaacgaccccacccggctcctcggccacggagcccggcacgtcctcctcctccggtggtgcctcctccaggatcagatgcgcgttcgtccccgagaagccgaacgaggacacccccgcccggcgcgggcgctcgccccggggccacttcaccgggtcggtgagcaggcgcagcccgctgccgtcccactccacgtggggcgagggggcgtcgacgtgcaggatggcgggcagcaggtcgtgccgcagggccaggaccatcttgatgacaccggccacaccggcggcgatctgcgtgtggccgatgttggacttcaccgctcccacccacagcggccggtcctccggccgttcccggccgtaggcggagatgagagccccggcctcgatggggtcgccgagcgtggtgccggtgccgtgcgcctccacggcgtcgatgtcctcgggggcgaggcgggcgttggcgagggcggcgcggatgacgcgttcctgggcggggccgttgggggcggtcaggccattgctcgcgccgtcctggttgatcgccgaaccccggatcaccgcgaggaccttgtggcccttcctgcgggcgtcggagagacgctcaaggagaaccacccccgtaccctccgccatgcccatgccgtcgctgctcgccgagaacggcttgcaccgtccgtcgggggccaggccgcgcagttcgctgaagccgatcagcggggcgggcgacgacatcacgtacgtgccgcccgccagcgccagcgagcactcctgtgtgcgcagggcctgggtggcgaggtgaagggagaccagcgacgaggagcacgccgtgtcgaccgtcaccgcggggccttcgaggcccagggtgtaggcgacgcggccggaggtgacgctgccggagttgccgatggtgaagtatccggcggtgccctcggggacctcggacgcgccgagggcgtagtcgagtccgtcacagccgatgaaggtgctggtgtcgctggagcggaggctgagggggtcgatgccggcccgttcgatcgcctcccacgccgtctccagggcgagccgctgctgcggcgccatggccgcggcctcggtgggtccgatgccgaagaaggtggggtcgaagtcaccggcgtcgtagacgaagccgccttcccggacgtaactggtgccggtgctctcggggtccgggtcgtagagggaatcgaggtcccagttgcggttgccgggcaggggcgcgacggcgtcgccgccggtggagaccagctcccagaactcttcgggagaccggactccgccgggcagccggcaggccatgccgatgaccgcgaccggttcgtggcccgccgactcgacgtcctgcagccggcgttccgtctgacgcaggtccgcggtgacacgcttgaggtattccagaagtttctcttcggtgtgcgccatcccggtgacaaccgcccctctccgcgagaacagaccgcagactcgtcgacggcgctaaagccctcctaatactcggctgtgtaccgctcgctgccacgggtgtccgcactggtcggaggctccggcccagggaacaggggctttcttaggggcgcttaagcggtgcctgccagggtgtgccggtgtcaggccgtcacgccctgatcagcggcgtcgcccgtgccgtgcccgtgcggtcggtgggcctgaccgtcggtccggacaacgcgaagcgaggcatcgtgcccatcacggatagcaagccggccgccacattccccgacctggtcgacccgtcgttctgggcgcggccgcacgcggaacgcgtggcgctgttcgaggagatgcgcgggctgccgcggccggcgttcatccggcagaacatgcccggcgtgccctggacgttcggctaccacgcgctggtcaagtacgcggacatcgtggaggtgagccgccgcccgcaggacttctcctcgaacggcgcgaccaccatcatcggtctgccgcccgagctggacgagtactacggctcgatgatcaacatggacaacccggaacactcgcggctgcggcgcatcgtctcgcgttccttcggccgcaacatgatccccgagttcgaggccgtggcgacccgcaccgcccgccgcatcatcgacgagctcatcgcgcggggacccggcgacttcatcaggcccgtcgccgcggagatgcccatcgccgtgctcagcgacatgatgggcatcccggcggaggaccacgacttcctcttcgaccggtccaacacgatcgtcggccccctcgacccggactacgtgccggaccgggcggactccgaacgcgcggtgatcgaggcgtcacgcgaactcggcgactacatcgctggccttcgtgcggaacggctcgccgcccccggcaacgacctcatcaccaagctcgtgcaagtccaggcggacggcgagcagttgacgcggcaggaactcgtctccttcttcatcctgctcgtcatcgccgggatggagaccacccgcaacgccatctcgcacgcgctggtactgctgaccgagcatcccgagcagaagcagctgctgctctcggacttcgacacgcacgcgccgaacgcggtcgaggagatcctcagggtctccacgcccatcaactggatgcggcgcgtcgccacccgcgactgcgacatgaacggccacaggttccgcaggggcgaccggatcttcctgttctactggtcgggcaaccgggacgaatccgtcttccctgacccgtaccggttcgacatcacgcgcgggacgaacgcgcacgtcacgttcggcgcggtgggcccgcacgtctgcctcggggcccacctcgcccgtatggagatcaccgtcctgtaccgggagctgctcgcggcgctgccccagatccatgccgtggggcagccccgcaggctggactccagcttcatcgaagggatcaagcacctgcactgcgccttctgagcacatacgcttccctctgcgcatgtgcgctcacgacgctccgatcagcgactgccaacgactgtcagcgaccggacagggccaagggcggtggggacatcaggtgcatgtcacccgcgagtatggcccgctgcagctcctggagcgggcgcccgggttcgagccccagctcgtcgttgagcgtcttgcgcaccgactggtacaccttcagcgcgtccgcctgccgctcggagcggtagagcgccagcatcagctggcggtagaacgcctcgcacatcgggttctccgcggtgagggcgtacagcatgcccacggcctcgcggtgccggccgagctggagctggcactcgacgagcatctcctgacactccaggcggatctcggtcagccaggtcgagaagccgtcgatgatcgggccgttggtgccgggaccgttcccgccctgcccgaggatcgggccgcgccacagcgcgagcgcctgcccgaaacaggaggccgcctcgtcgaaccgcttctccctgagcaacgaccgccccacgtccaccagttcggggaagatctgggcatcgatctggtcgtcgtcccgcttgtgcaggacgtaccccggcgcacgggtctcgacggggttgcccgccgaaccgggcaccttgaggaacttgcggagctgggagatgtacacatgcagtcccgccgtggcgcgccgcggcaggtcctcgccccagatctcccgcatcagctgctccagggagaccacccggtcggcgcggatgaggagcacggtgaggacgatctccaccttctgggcgttgatggtggcgtagtcgtttccgtccttgatgcggagcgggcccagcatttcgtatctcaccgagcgttcccccttgctgtcgcacgctgctgcgcactgtcggccagggccttggagatgacttccgtgacgccctgctggtgcgtgttcagatagaagtggcccccggcgaagaccttgaggtcgaaggggccttccgtgtgctgctgccaggcctcgacctcgtccagcggcgcctgcgggtcccggtcgcccaccagggcggtgatggggcaggacagcggcggcgacgggttccaccggtacagctcgaccgcccggtagtcgttgcggacgaccgggatgatctccgcgagcagttcctcgtcgtccaggaaccgcgggtcagtgccaccggcccggcgcagctcggcggccaactcggtgtcgtcgaggaggtgtacggtgccgcgccggaagcgggacggcgcgcggcgtcccgagacgaacagccggcagggctgcttccccgtgcgctcgcggagccgctgggcgacttcgtaggcgaggacggcgcccatgctgtggccgaagaacgccaacgggcggtcgtcgaacgggccgagcgcatcggtgatgaggtcggcgagttccccgatgtcgtccaggagccgctctctgcggcggtcctgtcgcccggggtactgcaccgcgaggacctcgctgtcggtcgggagagtgggggattgcgcaagggggtggtagtaggaggccgagccgcccgcgtgggggaagcagaccaggcgaacgacggcttccggtcggggccggaagcgacgtatccaagggtccgacatatcgggtggggggaaggcagacaagatctttcccttcgccaggaacgctgacaacggtgtgtcgccacatcacatagccgctcctgatcatgcgcagctcaaagtttaaacggcaacgtcgctaacgggggagcagggcggaatcagacattccccatcctttattccgcgattcttacgtgatcgaatcccggcggccaagatggagtaaatttcaatatgaatgcttaacgccgcacagcttgtacggcgggccgcccgggcggtgactggcgtccctgccagccgtgatggcctgacgaggcctccgggatccatcccccgcccgctgtcgccgagttctttgcgggattattacgttgcattggtttgcttcgtggcccgggccgttggcctgcgctatttggcagccttccgtcatgggtggtaaaagatcgcctttcccctctggggtgccggtcgagctggcctcgaccgcgattgtggcttgttgttttcttgtggcgccgcgtgtgaaacagcggcagttggccactcgctctgacaggctccggggacggggttgtcaccttttggggtgactggcctcgttcaaggcgtcctggcccgtggtgcatccgcgatcgtcgtgccatgggtgaagtgggaaggagcacagaacgatgagcgagagcatggcgtggctgacgcgggacgtccgcaaggcccgcaaggagggcagtgcggggaccgcgcggcgccgagccgaccggctggcggacctggtcgcccacgcccgctcggcgtcgccgtactaccgggagctctaccacggcctgcccgagcggatcgaggacccgacgctgctgccggtgacggacaagaagcagctgatggaccacttcgacgactggccgacggaccgcgacatcaccttcgagaaggtccgcgcgttcaccgacgaccccgagctgatcgggcggcgcttcctcggccgctatctggtggccaccacgtcgggcaccagcggcaggcgcggcctgttcgtgctcgacgaccggtacatgaacgtgtcctccgccgtctcctcccgggtgctcgcctcctggctcggccccctcggcatcgcccgggccgtcgtccacggcggccgcttcgcccaactcgtcgccaccgagggacattacgtcggcttcgccggatactcccgcctgcgccaggacggcggagcgcgcagcaagctcgtccgcgccttctctgtgcacgagccgatgtcacgtctggtcgccgaactcaacgagtaccggcccgcgttcgtcatcggctacgccagtacgatcatgctgctcaccgccgaacaggaagcgggccggctgcacatcgacccggtgctggtcgagcccgcgggcgagacgatgaccgagagcgacaccgaccgcatcgctgcggcgttcggcgccaaggtgcgcacgatgtacagcgcgaccgagtgcacctacctcagccacggctgcgccgagggctggtaccacgtcaacgacgactgggccgtgctcgaaccggtcgacgccgaccaccggcccaccccgccgggggagttctcgcacaccaccctgatcagcaacctcgccaaccgcgtccagccgttcctccgctacgacctgggcgacagcgtcatgctccgccccgacccctgcccctgcggcaccccctcgcccgcgatccgggtccagggcaggtcgggcgacatcctcaccttcccctcgggccggggcgacgacgtcagcctcgccccgctcgccttcagcagcctcttcgaccgcatgcccggagtcgagctcttccagatcgagcagaccgcgccgtcgaccctgcgcgtccgcgtggtccaggcgcccggcgccgacgcggaccacgtgtggcagcgggcccacgacgggctgacccacctcctcgccgacaacaagctcgacaacgtaaccgtcgaacggggcgaggagccgccgcggcaggcatccggcggcaagtaccggacgatcatcccgctcgccgcctgaacgctcgccgactagccgcgcgccgcctgagctgctctcaccgcgcgtacgggcgcagcggaggctcctcgtcgacccacggctggctgtggatcagcagctcgatcgggaagttcagcaggccgggcagggcgtcgacggcctcctggctgttgagcggcatgaccggcttggcgcagtgcgcgcggtcgatgcggctcgtgtcggcgggaccggggtgctcgatcgcatcggcgaccaggtcgtagctgacctggtcgacgaccatggcgatgtgggtcggccacggccgacccggacagatgtcctggacgccgatgcggtgggcgcccggcagcgacggcgcctccccgtcggccaccacggactcgtcggcgtatgaatagatcgtggtgtacgacggtcctgcgggcatgggcgtgccgtcggcgcccagagccttcgaccagttcgagtcgcgggcgaactgcaggaccgacgccgggcagcccgccacctcggcgatcgggcggcagggcgaggccagccgggtcccctggaacggggagcccagggtcaccatgtcgtcgaccttccccggcaggtccggccagaagcgcagggcccacgccgtgaggaggccgccctggctgtgcccgacgagatcgaccttccggccggtggcctcctggatcgcgcgggtcgcgtacaccacgtactcgacggactcctgcatgtcacggagcccgcgaccgggagaatccacccaacaggactggtagcccttcttcttcaactcggccatgtagttccaggcgtagttctcctcgcccttgaggccggtcccgggcacgaagaggacggtcggcttgtcaccggcgtcacgcaggtcccccagctccgtcccgcagtgcagcgccttggcgagctcggccgccggtatctccaacgggggagaggaaacatccgccgccgaagcggcggaggccggaagcacggtggcggccagcacggccgccacgagtccgccgagccatgaggacaagcgcacggtgacctccacaggaaccttcacgagtgagcggaaactccctccggagggagcacctcatcgtgcggcggcgccacagtagccgtcaactgccccacggggctgagtagttgacagttggccgggctcggccggcgaagcgcccgggccccgccgccccgcgccgtgcgcgaggggtccgtgacctgggtggacggtccggttggacatcccgggggagcctctggcatggtcgcccgtccgtccccctcaagaaccgaagggagcgtcacgatcacgatgatcgaagtcagcacgcgcagcatgaaggaagcggctgccgccgagcagctccgcgcggagaccacgacactggacattccaaagggtttcgacctgtggacggccgacgagatcgcggagtggctcgacggcgtcgaggacgacccggcagtctccgacgccgacttctacgcggcccagcagcggtgcgacgggtcctcggcaccgagggcacctgacccgccggcggccctgcgcggccctacgtgtgcagcgccccgtcctcctccacatgcccctccggctccagctggatcgtcgagtgggccacgtcgaagtggcccccgacacaccgctgaaggcgccccaggagctccccgtacccgctcgcgagagcctcctccgtgaccaccacgtgcgcggtgagcaccggcatccccgaggtgaccgtccagccgtgcagatcgtgcacggcgaccacgccccgctcctccagcaggtgccggcgcacctcgccgaggtcgacgtcctgcggggtcgcctccagcaggacgtgcaaggagtcccgcagcaggccgtacgcgcgcggcacgatcagcaggccgatgacgatcgacgcgatcgggtcggcggcctgccaccccgtgagcaggatgaccaggccgcccacgatcaccgcgaccgagccgagcgcgtcgcccagcacctccaggtacgcgccccgcagattgaggctcttctccttggcgtcccgcagcagccacaggcccaccaggttggcggcgagcccgcccagcgcgaccacgaacatcaggccgcccttcacctccaccggctcgctgaaccggccgatcgccgaccacaggacccaggcgaagatgacgaccaggagcagcgcgttcaggaccgcggagaagatctccacgcggtagaacccaaaggtgcgccgcggcgtcggcgcccgctgggcgagggtgatggcaccgagggccagcgagacgccgaccgcgtcggtcaggctgtgcgcggcgtcggcgagcagcgcgaggctgccggacaggagcgcgccgaccacctggatgacggtgatcgagccgctgatgccgatggtccacagcaggcgcttgcggtacgtgccgctgagagtgccgcccgccgccccggcggacggaccgtggtcgtgccccatgcccgcgagtggaccacggcggcgcggcacccgccaccgagcggccgccggtcggctcagtgcagccgggcctgggtggaggtgtcgcgctggtgcgggatgccgagcggcggcggcagctcgccctgctgcaccctgaccgtgcgcacgggggcggggacccggatgccctcggcgcggtaacgctggtgcaggcgcttgatgaactcgtgcttgatgcggtactggtcgctgaactcgccgacgccgaggatcaccgtgaagctgatccgcgagtcgccgaaggtgtggaagcggatcgccgcctcgtggtcggggaccgcgccggtgatctcggccatcacctcgtccaccacctcggtcgtgaccttctcgacctgctccaggtcgctgtcgtagctgaccccgacctgcaccatgatcgacagctcctgctcggggcggctgtagttggtcatgttggtgccggcgagcttcgcgttggggatgatgacgaggttgttggagagctggcggaccgtggtgttgcgccagttgatgtcgacgacgtagccctcctccccgctgctgagctggatgtagtcgccgggctgcacggtcttcgcggcgaggatgtgcacgcccgcgaagagattggcgagcgtgtcctgcagtgcgagggcgaccgcgagacctcccacgccgagggcggtgagcagcggtgcgatggagatgccgagggtctgaaggacgatgaggaagcccatcgcgagcaccacgacgcgggtgatgttcacgaagatggtggccgatccggccactccggagcgggactgtgccacggccttcaccaggccggtgacgatccgggccgccgtgagcgtggcggccaggatgagcagcgcggtcagcgtcatggtgacgttgcgtccggtgcgcggcgtgagcggcagcgcgcccgccgcggcggcgagcccggcggtgatggccgcgcagggcacgagggtgcgcagggcgtcgacgatgacgtcgtcaccgctccaccgggttttgctcgcccgttcgccgagccacctcagaagtgcgcggagcagcagcccggcgacgacgccggcgacgaccgcgataccggccacgatccagtcgtgcagtgtgagggcacgggtcatcagttcgctcccgtcgtacggggggagtgcgcctgtgtggggcgtatgtgatgtgacgtcaccttgtgatacctgctcgattccggggagtgcggtcacgccgggacgagagctcggttccggcgcggacgtcatcctgccccatccgcccacggcaggcgtgcatacccccacctggatcttcacagaccggccacgtctgtccatgcgccgatgagcgcgctgcccgtggtaaagcattgagtcaggcgatttggccactcggcactcggcggaccggtcgagccggtcgatctacgtgagcggaggcggttgagcatggcgtccatgtgcagacccggaatgtcacccgtcaattcgcacaacgagtgggatccgctggaggagatcatcgtcgggcggctggagggcgcgaccattccctccagccatccggtcgtggcgtgcaacatcccgacctgggcggcacggctgcagggtctcgccgccgggttcgagtatccgcagcggctgatcgagccggcgcagcaggagctcgaccagttcatcgctctcctgcaatccctcgacgtcacagtgagacggccggcggccgtcgaccacaagcaccgcttcgggacccccgactggcagtcgcgcggcttctgcaattcctgtccgcgggacagcatgctcgtcgtcggcgacgagatcatcgagaccccgatggcgtggccgtgccgctgtttcgagacgcactcgtaccgcgaactcctcaaggactacttccggcgcggcgcgcgctggacggcggcgccgcgcccccagctcaccgaggccctgtacgagaaggacttccgccctcccgaggagggcgaacgatgcgctacatcctcaccgagttcgagccggtgttcgacgcggcggatttcgtgcgggcgggccgcgacctgttcgtgacgcggagcaacgtcgccaacctgctgggcatcgagtggctgcgccgccaccttcgggccggagtaccgcgtgccacgaga

SEQ ID NO: 5 - protein, 346 aa, Streptomyces cinnamonensis
LTTRPDTKTALSQKTALSQLLTEIEHRNPAQPEFHQAAREVLETLAPVIAARPEYAEAGLIERLCEPERQIVFRVPWQDDHGRVRVNRGFRVEFNSALGPYKGGLRFHPSVNLGVIKFLGFEQIFKNALTGLGIGGGKGGSDFDPRGRSDAEVMRFCQSFMTELYRHIGEHTDVPAGDIGVGGREIGYLFGQYRRITNRWEAGVLTGKGRNWGGSLIRPEATGYGNVLFAAAMLRERGETLEGRTAVVSGSGNVAIYTIQKLAALGANAVTCSDSSGYVVDEKGIDLDLLKQVKEVERARVDTYAQRRGASARFVPGRRVWEVPADIALPSATQNELDADDATALI

SEQ ID NO: 6 - protein, 306 aa, Streptomyces cinnamonensis
MTLASSLEPTTEPLFNGLYVPLVTPFTDDLRLAPEALARLADEALSAGASGLVALGTTAEAATLTAEERETVIRVCSAACRAHGAPLIVGVGTNDTATAITALRELAARGDVAAALVPAPPYIRPGEAGTLAHFAALAEHGGLPLVVYDIPYRTGQTLGAGTITALGRLPEVVGIKHATGSIDPTTMELLDSPLPGFAVLGGDDIVLSPLVAAGAHGGIVASANLRTADYAEMIALWRRGSAAPARALGADLARLSAALFTEPNPTVIKGVLHAQNRIPSPAVRMPLLAASADSVRRAAPLAASRK

SEQ ID NO: 7 - protein, 313 aa, Streptomyces cinnamonensis
MLDVRRLHLLRELDRRGTIAAVAEALTFTASAVSQQLGVLEREAGVPLLERSGRRVVLTPAGRSLVAHADAVLNRLEQAVAELAGARDGIGGPLRIGTFPSGGHTIVPGALAELASRHPALEPMVREIDSARVSDGLRAGELDVALVHDYDFVPATPDTTVDEVPLLEEPMYLVTHAADTATDSGSGSTLAALLGPCAEVPWITARDGTTGHAMAVRACQAAGFQPRIRHQVNDFRTVLALVAAGQGAGFVPRMAAEPSPAGVVLTKLPLFRRSKVAFRAGGGAHPAIAAFVAAATTAVERMAGSRGPAGGSE

SEQ ID NO: 8 - protein, 138 aa, Streptomyces cinnamonensis
MADDAYLFLLPDRHPRLGAALAAVGALECTETPAVHAWLQAHEASVSSEQVRILPADAETLIPKDAERLPVPLSEEEALKVEQECAPQTVTDMESELLAFRETTQDWQALVHRALTAGIPAQRIARLTGLDPEEIGRL

SEQ ID NO: 9 - protein, 207 aa, Streptomyces cinnamonensis
LAVAACAAVVLPIDAVVRISAADVGVLVFFAYLLPYLAITMTVFVSVAPEQVRSWARREARGTFLQRYVLGTAPGPGGSLFIAAAALVVAVLWLPGHLSTTFSALPRTLVALALVVAAWICVVVAFAVTFQADNLVENERALEFPGERSPAWADYVYFALAAMTTFGTTDVDVTSRDMRRTVAANTVIAFVFNTVTVAILVSALGGR

SEQ ID NO: 10 - protein, 62 aa, Streptomyces cinnamonensis
MTVMDKLKQMLKGHEDKAGQGIDKAGDFVDGKTQGKYSGQVDTAQDKLRDQFGSDQQEPPQR

SEQ ID NO: 11 - protein, 184 aa, Streptomyces cinnamonensis
MGTAQSQEQAAAPGACAAFVRFVLCGGGVGLASSFAVVALASWVPWALANALVAVVSTVVATELHARFTFGAGGRATWRQHAQSAGSAAAAYAVTCVAMFVLQQLVAAPGAVLEQVVYLSASALAGVARFVVLRLVVFARNRSLPAAAAVRTARPVRRVPAPVPATVAHAASRPAGPAALCPAA

SEQ ID NO: 12 - protein, 105 aa, Streptomyces cinnamonensis
MTSTDHTSGQDATELEKQLAAATPEEREKLLTDTIRTQAGTLLNTTLSDDSNFLENGLNSLTALELTKTLMTLTGMEIAMVAIVENPTPAQLAHHLGQELAHTTA

SEQ ID NO: 13 - protein, 828 aa, Streptomyces cinnamonensis
VANEEKLVEYLKWTTAELHQAQQQLRELKAAQHEPIAVVSMACRLPGKTRTPDDLWDLVSEGRDAVTGFPDDRAWELPEERPYAELGGFLDDAAGFDAGFFDISDTEAVATEPLQRLMLHLAWETVERGHIAPHTLRSTLTGVYVGATGHDYATRLETAPDELLPYLGGGTSGSLVSGRIAYALGLEGPAISVDTACSSSLVALHLACQALRRGECGLALAGGGTVMSTPHTFHAFAHQKSLAQDGRCKPFAAAADGMGLGEGVGLVLLERLGDARKNGHPVLAVIRGSAVNQDGAGYGLAAPNGPSQQHVIRAALADAGLTPDQIDAVEAHGTGTPIGDAIEVQALLATYGADRSPDRPLWLGSVKSNTGHTQGAAGAAALIKMVQAFRHGTLPPTLHVDRPTPLAAWKKGAVRLLTEAVDWPRREEPRRVGISAFATSGTNAHLILEEPPVDEAPVPDAARDQTSPVAPELPVAWSLSARTPEALRAQAKALVTHLAATDPAPSPAEVAYSLAATRSPLEHRAVLTGTDHTELLAAARALAAGEDHPDLVRSTPGAGPKKIAWHFDGRPADGVTTGAAPGAKPGATFGATFGAAFGGAEFHSAFPLFASAFDEARALLDTHLPTPLPTPHSELARFAVHTALARLLLETGVRPHTLTGDGVGHIAAAYAAGILTLDDACRLAAAHAAAAQAAEGEQPAPPDAYEPVLKQLTFQRATLTLTSTAPADTPIASADYWHHHLTSPAPTAPPTPETHTLLHLGALSPEGTQTSAVSALLTALARLHTTGGTVDWTPLVRRTPHPRTIDLPTYSFQATRYWLHDHTAHAAV

SEQ ID NO: 14 - protein, 299 aa, Streptomyces cinnamonensis
VKNLRIPVSQTVSLNVRYRPADGPGAPGRPFLLLHGMLSNARMWDEVAARLAAAGHPAYAVDHRGHGESDTPPDGYDNATVVTDLVAAVTALDLSGALVAGHSWGAHLALRLAAEHPDLVAGLALIDGGWYEFDGPVMRAFWERTADVVRRAQQGTTSAADMRAYLRATHPDWSPTSIEARLADYRVGPDGLLIPRLTSTQVMSIVAGLQREAPADWYPKVTVPVRLLPLIPAIPQLSDQVRAWVAAAEAALEQVSVRWYPGSDHDLHAGAPDEIAADLLLLARSCEAMPGGKAGVRPA

SEQ ID NO: 15 - protein, 276 aa, Streptomyces cinnamonensis
VNKTVAPEPSDIGHYYDHKVFDLMTQLGDGNLHYGYWFDGGEQQATFDEAMVQMTDEMIRRLDPAPGDRVLDIGCGNGTPAMQLARARDVEVVGISVSARQVERGNRRAREAGLADRVRFEQVDAMNLPFDDGSFDHCWALESMLHMPDKQQVLTEAHRVVKPGARMPIADMVYLNPDPSRPRTATVSDTTIYAALTDIGDYPDIFRAAGWTVLELTDITRETAKTYDGYVEWIRAHRDEYVDIIGVEGYELFLHNQAALGKMPELGYIFATAQRP

SEQ ID NO: 16 - protein, 511 aa, Streptomyces cinnamonensis
MSADLGARRWWAVGALVLASMVVGFDVTILSLALPAMADDLGANNVELQWFVTSYTLVFAAGMIPAGMLGDRFGRKKVLLTALVIFGIASLACAYATSSGTFIGARAVLGLGAALIMPTTLSLLPVMFSDEERPKAIGAVAGAAMLAYPLGPILGGYLLNHFWWGSVFLINVPVVILAFLAVSAWLPESKAKEAKPFDIGGLVFSSVGLAALTYGVIQGGEKGWTDVTTLVPCIGGLLALVLFVMWEKRVADPLVDLSLFRSARFTSGTMLGTVINFTMFGVLFTMPQYYQAVLGTDAMGSGFRLLPMVGGLLVGVTVANKVAKALGPKTAVGIGFALLAAALFYGATTDVSSGTGLAAAWTAAYGLGLGIALPTAMDAALGALSEDSAGVGSGVNQSIRTLGGSFGAAILGSILNSGYRGKLDLDGVPEQAHGAVKDSVFGGLAVARAIKSNGLADSVRSAYVHALDVVLVVSGGLGLLGVVLAVVWLPRHVGQSTAKTAESEHEAADAV

SEQ ID NO: 17 - protein, 191 aa, Streptomyces cinnamonensis
VPGLRERKKARTKAAIQREAVRLFREQGYTATTIEQIAEAAEVAPSTVFRYFATKQDLVFSHDYDLPFAMMVQAQSPDLTPIQAERQAIRSMLQDISEQELALQRERFVLILSEPELWGASLGNIGQTMQIMSEQVAKRAGRDPRDPAVRAYTGAVFGVMLQVSMDWANDPDMDFATTLDEALHYLEDLRP

SEQ ID NO: 18 - protein, 268 aa, Streptomyces cinnamonensis
MDRGTAARAPQIGDEFGAATGNGVWLRRYHAAAEAPVRLVCFPFAGGSASYYFGLSGLLAPGVEVLAVQYPGRQDRHAEPCLASVAELADGVVPHLPCDGKPFALFGHSLGAIVAFEVARRLRGPAGPGLPVHLFVSGGLARPYRPAGRSGAFGDADILAHLRAMGGTDERFFRSPELQELVLPALRADYRAVATYEAPGPGRLDCPITALIGDADERTSPEQAATWRERTGAAFDLRVLPGGHFYLDGCQEQVAAVVTEALTAGPGV

SEQ ID NO: 19 - protein, 3025 aa, Streptomyces cinnamonensis
MAASASASPSGPSAGPDPIAVVGMACRLPGAPDPDAFWRLLSEGRSAVSTAPPERRRADSGLHGPGGYLDRIDGFDADFFHISPREAVAMDPQQRLLLELSWEALEDAGIRPPTLARSRTGVFVGAFWDDYTDVLNLRAPGAVTRHTMTGVHRSILANRISYAYHLAGPSLTVDTAQSSSLVAVHLACESIRSGDSDIAFAGGVNLICSPRTTELAAARFGGLSAAGRCHTFDARADGFVRGEGGGLVVLKPLAAARRDGDTVYCVIRGSAVNSDGTTDGITLPSGQAQQDVVRLACRRARITPDQVQYVELHGTGTPVGDPIEAAALGAALGQDAARAVPLAVGSAKTNVGHLEAAAGIVGLLKTALSIHHRRLAPSLNFTTPNPAIPLADLGLTVQQDLADWPRPEQPLIAGVSSFGMGGTNGHVVVAAAPDSVAVPEPVGVPERVEVPEPVVVSEPVVVPTPWPVSAHSASALRAQAGRLRTHLAAHRPTPDAARVGHALATTRAPLAHRAVLLGGDTAELLGSLDALAEGAETASIVRGEAYTEGRTAFLFSGQGAQRLGMGRELYAVFPVFADALDEAFAALDVHLDRPLREIVLGETDSGGNVSGENVIGEGADHQALLDQTAYTQPALFAIETSLYRLAASFGLKPDYVLGHSVGEIAAAHVAGVLSLPDASALVATRGRLMQAVRAPGAMAAWQATADEAAEQLAGHERHVTVAAVNGPDSVVVSGDRATVDELTAAWRGRGRKAHHLKVSHAFHSPHMDPILDELRAVAAGLTFHEPVIPVVSNVTGELVTATATGSGAGQADPEYWARHAREPVRFLSGVRGLCERGVTTFVELGPDAPLSAMARDCFPAPADRSRPRPAAIATCRRGRDEVATFLRSLAQAYVRGADVDFTRAYGATATRRFPLPTYPFQRERHWPAAAGVGQQPETPELPESSESSEQAGHEREEGARAWGGPEGRLAGLSVNDQERVLLGLVTKHVAVVLGDASGTVQAARTFKQLGFDSMAAAELSERLGTETGLPLPATLTFDYPTPLAVAAHLRAELTGTPAPAGSAPATGALGAGDLGTDEDPVAIVAMSCRYPGGAGTPEDLWRLVADGADAIGDFPTDRGWDLARLFHPDPDRSGTSCTRQGGFLYDAADFDAEFFDISPREALAVDPQQRLLLECAWEAFERAGLDPRALKGSPTGVFVGMTGQDYGPRLHEPSQATDGYLLTGSTPSVASGRLSFSFGLEGPALTVDTACSSSLVTLHLAAQALRRGECDLALAGGATVLATPGMFTEFSRQRGLAPDGRCKPFAAGADGTGWAEGVGLVLLERLSEARRKGHAVLAVIRGSAINQDGASNGLTAPNGPSQQRVIRAALAAARLTADEVDVVEAHGTGTTLGDPIEAQALLATYGQGRSAERPLWLGSVKSNIGHTQAAAGVAGVIKMVMAMRHDLLPATLHVDEPSGHVDWSTGAVRLLTEPVVWPRGERPRRAAVSSFGISGTNAHLVLEEAGQDEYVAGAADDAGPVDGAVLPWVVSGRTGAALREQARRLRELVTGGSADVSVSGVGRSLVTTRAVFEHRAVVVGRDRDTLIGGLEALAAGDASPDVVCGVAGDVGPGPVLVFPGQGSQWVGMGAQLLGESAVFAARIDACEQALSPYVDWSLTEVLRGDGRELSRVDVVQPVLWAVMVSLAAVWADHGVTPAAVVGHSQGEIAAVVVAGALTLEDGAKIVALRSRALRQLSGGGAMASLGVGQEQAAELVEGHPGVGIAAVNGPSSTVISGPPEQVAAVVADAEARELRGRVIDVDYASHSPQVDAITDELTHTLSGVRPTTAPVAFYSAVTGTRIDTAGLDTDYWVTNLRRPVRFADAVTALLADGHRVFIEASSHPVLTLGLQETFEEAGVDAVTVPTLRREDGGRARLARSLAQAFGAGCAVRWENWFPATGTSTVELPTYAFQRRRYWLEAPTGTQDAAGLGLAAAGHPLLGAATEIADGDIRLLTGRISRHSHPWLAQHTLFGAAVVPASVLAEWALRAADEAGCPRVDDLTLRTPLVLPETAGVQVQIVVGPADARDGHRDFHVYARPDGKDASEGEGIAEGEGASEGEGASGGTDAPWTCHADGRLVAEPTGTASEDSPDTVWPPPGAEPVDLGDFYERAAATGVGYGPVFTGLRALWRRDGELFAEAVLPQEAPETAGFGMHPALLDAALHPALLGERPAEEDKVWLPFTLTGVTLWATGATSVRVRLTPLDDDPDASADGRAWRVGVSDPTGAEVLTCEALVAVAAGRRELRAAGERVSDLYAVEWVPVPGPGPVGEGADFSGWAGLGECGERWECVGRVERWYEDLDALGAAVEGGASVPSVVLATAAAAPGGAGDGAADALSAVRWTGALLDQWLADARFADARLVVITSGAVATGDDFLPDPAAAAVRGLVEQAQVRHPGRILLVDTEAGAGLGVGAGVDDALLEQAVAMALGADEPQLALRAGRVLAPRLTAPQDAAVTEAARPLDPDGTVLITGPAGAPVADLAEHLVRTGQCRHLLLLPGDGELEEMAEELRGLGATVDLSTADPADPTALAEVVAAVEGDHPLTGVIHATGVVDAFDPGDSASDLMIDSASDSFAEAWSSRAGVTAALHTATAHLPLDLFAVLSPAGADLGIARSAAAAGADAFSAALALRRHTTVTTDTTAPPRTTAPPRTTASPRTTALSSSRTTGVALAYGPPTAPRPGIKGTAPGRIPVLLDAARAHGGGSPLLGARLAARALAAESAAEGVAGLPAPLRALAVAAAAAGAPTRRTAADRKPPADWPARLAPLSAPEQLRLLIDAVRTHAAAVLGRTDPEALRGDATFKQLGLDSLTAVELRNRLVEDTGLRLPTALVFRYPTPAAIAAHLRERLTSPSETTATQRSGGQTPAAGQASSALAPGGSAAGPPAADTVLSDLTRMENTLSVLAAQLPHTETGEITTRLEALLTRWKTTNATANDSGDGNGGDDDAAERLKAASADQIFDFIDNELGVGHGTSRVTPTPKAG

SEQ ID NO: 20 - protein, 2238 aa, Streptomyces cinnamonensis
MASEEQLVEYLRRVTTELHDTRRRLVQEEDRRQEPVALVGMACRFPGGVASPEDLWDLVAAGKDAIEDFPTDRGWDLEALYDPDPAAYGTSYVRHGGFVDDAGSFDADFFGISPREALAMDPQQRLMLETSWELFERAGIEPVSLKGSRTGVYAGVSSEDYMSQLPRIPEGFEGHATTGSLTSVISGRVAYNYGLEGPAVTVDTACSASLVAIHLASQALRQRECDLALAGGVLVLSSPLMFTEFCRQRGLAPDGRCKPFAAAADGTGFSEGIGLLLLERLSDARRNGHKVLAVIRGSAVNQDGASNGLTAPNDAAQEQVIRAALDNARLTPSEVDAVEAHGTGTKLGDPIEAGALLATYGQHRARPLLLGSLKSNIGHTHATAGVAGVIKTVMAIRNGLLPATLHVEELSPHVDWDAGAVEVVTEPTPWPETGHPRRAGVSAFGISGTNAHLILEEAPPEEDVPAPVVVESGGVVPWVVSGRTPEALREQARRLGEFVAGDTDALPNEVGWSLATTRSVFEHRAVVVGRDRDALTAGLGALAAGEASAGVVAGVAGDVGPGPVLVFPGQGAQWVGMGAQLLDESAVFAARIAECERALSAHVDWSLSAVLRGDGSELSRVEVVQPVLWAVMVSLAAVWADYGVTPAAVIGHSQGEMAAACVAGALSLEDAARIVAVRSDALRQLQGHGDMASLSTGAEQAAELIGDRPGVVVAAVNGPSSTVISGPPEHVAAVVADAEARGLRARVIDVGYASHGPQIDQLHDLLTERLADIRPTNTDVAFYSTVTAERLTDTTALDTDYWVTNLRQPVRFADTIEALLADGYRLFIEASAHPVLGLGMEETIEQADMPATVVPTLRRDHGDTTQLTRAAAHAFTAGADVDWRRWFPADPAPRTIDLPTYAFQRRRYWLADTVKRDSGWDPAGSGHAQLPTAVALADGGVVLNGRVSAERGGWLGGHVVAGTVLVPGAALVEWVLRAGDEAGCPSLEELTLQAPLVLPESGGLQVQVVVGAADEQGGRRDVHVYSRSEQDASAVWQCHAVGELGRASVARPVRQAGQWPPAGAEPVEVGGFYEGVAAAGYEYGPAFRGLRAMWRHGDDLLAEVELPEEAGSPAGFGIHPALLDAALHPLLAQRSRDGAGAGAHGGQVLLPFSWSGVSLWASEATTVRVRLTGLGGGDDETVSLTVTDPAGGPVVDVAELRLRSTSARQVRGSAGPGADGLYELRWTPLPEPLPVPAPANGRDVAADLSGCAVLGELVAEPGPGIDLEGCPCYPGVGALADNASPPSMILAPVHSDTTGGDGLALTERVLRVIQDFLAAPSLEQKQTRLAFVTRGAADTGSTTGGSAAPAEAVDPAVAAVWGLVRSAQSENPGRFVLLDTDAPLDQASVAPLVDAVRSAVEADEPQVALRGGRLLVPRWARAGEPVELAGPAGARAWRLVGGDSGTLEAVVAEACDDIVLRPLAPGQVRVAVHTAGVNFRDVLIALGMYPDPDALPGTEAAGVVTEVGPGVTRLSVGDRVMGMMDGAFGPWAVADARMLAPVPPGWGTRQAAAAPAAFLTAWYGLVELAGLKAGERVLIHAATGGVGMAAVQIARHVGAEVFATASPGKHAVLEEMGIDAAHRASSRDLAFEDAFRQATDGRGVDVVLNSLTGELLDASLRLLGDGGRFVEMGKSDPRDPELVALEHPGVSYEAFDLVADAGPERLGLMLDRLGELFAGGSLVPLPVTAWPLGRAREALRHMSQARHTGKLVLDVPAPLDPDGTVLVTGGTGTIGAAVAEHLARTGESKHLLIVSRSGPAAHGAEELVSRIAEFGAEATFVAADVSEPDAVAALIEGIDPAHPLTGVVHAAGVLDNALIGSQTTESLTRVWAAKAAAAQQLHEATRESRLGLFVMFSSFASTMGTPGQANYSAANAYCDALAALRRAEGLAGLSVAWGLWEATSGLTGTLSAADRARIDRYGIRPTSAARGCALLAAARAHGRPDLLAMDLDARVPAASDAPVPAVLRTLAAAGAPATARPTAAAAADGATDWSGRLAGLTEEARLELLTELVCTHAAGVLGHADAGAVQVDAPFKELGFDSLTAVELRNRIAAATGLKLPAALVFDYPQARVLAAHLAERLVPEGAGAMGGVSGAEGVRDAYGAGGPGGDMTAQVLLEVARVEHTLSAAVPHGLDRAAVAARLEALLARCTATTAATGAAGAAVEGDGDSDGDGAVDQLETATAEQVLDFIDNELGV

SEQ ID NO: 21 - protein, 4132 aa, Streptomyces cinnamonensis
MVSEEKLVDYLKRVSADLHATRQRLREAEERGQEPVAVVEAACRYPGGIRTPEDLWDLVAAGGNALGAFPDNRGWDLRRLFHPDPDHPGTTYAREGGFLHDADLFDPEFFGISPREAAVLDPQQRLLLECAWEALERAGIDPRSLQGSRTGVYAGAALPGFGTPHIDPAAEGHLVTGSAPSVLSGRLAYTFGLEGPAVTIDTACSSSLVAVHLAAHALRQRECDLALAGGVTVMTTPYVFTEFSRQRGLAADGRCKPFAAAADGTAFSEGAGLLVLERLSDARRAGHRVLAVIRGSAVNQDGASNGLTAPNGPAQQRVIRAALAGARLSPAEVDAVEAHGTGTRLGDPIEADALLATYGQERHGGRPLWLGSVKSNIGHTQGAAGAAGLIKMVQALRHETLPATLYADEPTPHADWESGAVRLLSAPVAWPRGEHGEHTRRAGISSFGISGTNAHLILEEAPAADAEGAGGDGDGDGGGVRPVVRVGATGPREEQGQGQGQEQHQQQRQQRQRSSMMPTPHLPWLLSARSPAALRAQADALANHVAHADHSIADIGGTLLRRTLFEHRAVVLGTDRDERAAALAALAAGRAHPALTRAAGPARNGGTAFLFTGQGSQRPGMGRQLYDTFDVFAESLDETCARLDPLLEQPLKPVLFAPADTAQAAVLHGTGMTQAALFALEVALYRQVTSFGIAPSHLTGHSVGEIAAAHVAGVFSLADACTLVAARGRLMQALPAGGAMLAVQAAEDDVLPLLAGQEERLSLAAVNGPTAVVVSGEAAAVGEVEKALRGRGLKTKRLNVSHAFHSPLIEPMLDDFREVARGLTFHAPTLPVVSNLTGRLADAELMADAEYWVRHVRRPVRFHDGLRALSEQGVVRYLELGPDPVLATMVQDGLPAPAEGEEPEPVVAAALRSKHDEGRTLLGAVAALHTDGQPADLTALFPADAGQVPLPTYRFQRRRYWRVAPDAAAPARAAGLQETGHPLLPAVIRQADGGILLAGRLSLRTHPWLADHTIAGGVPLPATAFVELALLAGRHAACDTIDDLTLETPLLLDDTGTGVGAAVGAGADALVDAIEVQLALGAPDGSGRRALTVHSRPADDAADDGDAADAADAAGRGGPGGSGDLGDPGDPGDLGDGGGSRGWRRHATGILSAGPAAEPAAPDAAPWPPADATALDVDALYARLDAQGYSYGPAFRAVHAAWRHGDDLYADVRLADEQRAEADAFALHPALLDAALHAVDELYRGSEGRGQEQGQGGQEPEQGRGDADAPVRLPFSFSDIRHHATGATRLWVRLSPQGDDRLRLSLTDGEGGQVATVDALQLRLIPADRWRAARPTTAAPLYHLDWHELPLPEPAETDPAAHSWAVLGAHDAGLAPAAHYPDLAALKAAVEAGEPVPDIVFAPFPAQGTETDVPAQVRAHARHALELLRDWLTTEAFAAARLVVLTTGAVTARPEDGPADLATAPVWGLVRAAQAEQPDHVVLVDIDKDIDKDTDEETDQATDAGTASRHALPAALAAAAAQAETQLALRAGTVLVPRLAVVPPRTDTPALHATAPESTTDTVDSTGIAGAAESGGTVLITGGTGGLGQAVARHLAAAHGARHLLLVSRRGDAAEGVAELRADLADDGVDVRVAACDITDRDALAGLLADIPAAHPLTAVVHTAGVIDDSLITAMTPERLDAVLAPKADAAWHLHELTRDKDLSAFVLFSSGASVLGNGGQANYAAANTFLNTLAEHRRAAGLAATSVAWGLWESASGGMAARLGDADRARIHRTGVTGLTDEQALALFDAALTAEHPTVLATRFDRAVLRGQAAARTLQPALRGLVRTPRPTASAGAIGSTAATGSATDENAPSSWAARLARLSAADRDRALNELIREQIATVLAHPSPDTIELGRAFQELGFDSLTALELRNRLSTATGIRLPATLVFDHPSPTALVRHLHSHLPDEAQHTSPTAPGASAEGTAATATGIDDDPIAIVGMACRYPGGVTSPEQLWQLVATGTDAIGPFPEDRGWDTAGLFDPDPDQVGHSYTREGGFLYDAARFDAGFFGISPREAAATDPQQRLLLETAWQAFEHAGIDPAALRGTPCGVITGIMYDDYGSRFLARKPDGFEGRIMTGSTPSVASGRVAYTFGLEGPAITVDTACSSSLVAMHLAAQALRQGECELALAGGVTVMATPNTFVEFSRQRGLAPDGRCKPFAAAADGTGWGEGAGLVVLERLSDARRKGHRVLALLRGSAVNQDGASNGMTAPNGPSQERVIRTALAGAGRGPEDIDVVEAHGTGTTLGDPIEAQALLATYGQGRPEDRPLWLGSVKSNIGHTQAAAGVAGVIKMVMALRHEQLPTTLHADEPTPHVQWDGGGVRLLTEPVPWSRGERTRRAGVSSFGISGTNAHLILEEPPEEDLPEPVAAEPGGVVPWVVSGRTPDALREQARRLGEFVVGAGDVSAAEVGWSLATTRSVFEHRAVVAGRDRDDLVAGMQALAAGETPTDVVSGAAASSGAGPVLVFPGQGSQWVGMGAQLLDESPVFAARIAECEQALSAYVDWSLSDVLRGDGSELSRVEVVQPVLWAVMVSLAAVWADYGVTPAAVVGHSQGEMAAACVAGALSLEDAARIVAVRSDALRQLQGHGDMASLGTGAEQAAELIGDRPGVVVAAVNGPSSTVISGPPEHVAAVVAEAEARGLRARVIDVGYASHGPQIDQLHDLLTEGLADIRPANTDVAFYSTVTAERLTDTTALDTDYWVTNLRQPVRFADTIEALLADGYRLFIEASAHPVLGLGMEETIEQADIPATVVPTLRRDHGDTTQLTRAAAHAFTAGADVDWRRWFPADPTPRTVDLPTYAFQHQHYWLEEPSGLTGDAADLGMVAAGHPLLGACVELAESDSYLFTGRLSRRAPSWLAEHVVAGTVLVPGAALVEWVLRAGDEAGCPTIEELTLQAPLVLPESGGLQVQVVVGATDEQSGRRDVHVYSRSEQDASAVWVCHAVGVVSSEMPEAAAELSGQWPPAGAEAVDVEDFYARAAEAGYAYGPAFQGLRALWRHGTELFAEVVLPEQAGGHDGFGIHPALLDAALHPLMLLDRPADGQMWLPFAWSGVSLNADRATHVRVRLSPRGEAAERDLRVVIADATGAPVLTVDALTLRAADPGRLGAAARGGVDGLYTVDWTPLPLPQPLPLPRTDAGGSADWVILSDNSSAALADAVSSATAAGGGAPWALLAPVGGGSADDGLPVVRRTLSLVQEFLAAPELTESRLVIVTRGAVATDADGDVAASAAAVWGLIRSAQSENPGRFVLLDVEEEHLHPDGGELPYAALRHAVEELDEPQLALRSGKFLVPRMTPAAAPEELVPPVGTSGWRLGTSGTATLENLSVIDAPEAFAPLEPGQVRISVRAAGMNFRDVLIALGMYPDKGTFAGSEGAGHVTEVGPGVTHLSVGDRVMGLFEGAFAPLAVADARMVVPIPEGWSFQEAAAVPVVFLTAWYGLVDLGRLRAGESLLIHAGTGGVGMAATQIARHLGAEVFATASPAKHGVLDGMGIDAAHRASSRDLDFEETLRAATGGRGMDVVLNSLAGEFTDASLRLLAEGGRMVDMGKTDKRDPDRVAAEHAGAWYRAFDLVPHAGPDRIGEMLAELGELFASGALAPLPVQTWPLGRAREAFRFMSQAKHTGKLVLEIPPALDPDGTVLITGGTGVLAAAVAEHLVREWGVRHLLLAGRRGSEAPGSSELAEELTELGAEVTFAAADVSDPDAVAELVGKTDPAHPLTGVIHAAGVLDDAVVTAQTPESLARVWAAKATAAHLLHEATREARLGLFLVFSSAAATLGSPGQANYAAANAYCDALVRQRRAEGLAGLSIGWGLWQTASGMTGHLGETDLARMKRTGFTPLTTEGGLALLDAARAHGRPHVVAVDLDARAVAAQPAPSRPALLRALAAGATPGARTARRTAAAGSVAPAGGLADRLAGLPHPERRRLLLDLVRGNVAGVLGHSDHDAVRPDTSFKELGFDSLTAVELRNRLAAATGLKLPAALVFDYPESATLVDHLLERLSPDGAPPPVKDAADPVLNDLGRIESSLDALALDADARSRVTRRLNTLLSKLNGAATAGSPADVTDLDALDALDDVSDDEMFEFIDREL

SEQ ID NO: 22 - protein, 4038 aa, Streptomyces cinnamonensis
MSSAEESSPDVSGTGVSGTGESATGTSSTEAKLRQYLKRVTVDLGQARRRLREVEERAQEPIAIVSMACRFPGDTRTPEALWDLVAEGGDAIDDFPTNRGWDLESLYHPDPDHPGTSYVRRGGFLYDAPAFDASFFGISPREALAMDPQQRVLMETAWQLLERAGIDPASLKLSATGVYIGAGVLGFGGAQPDKTVEGHLLTGSALSVLSGRISFTLGLEGPSVSVDTACSSSLVSMHLAAQALRQGECDLALAGGVTVMSTPGAFTEFSRQGALSPDGRSKAFAASADGTGFSEGAGLLLLERLSDARRNGHKVLAVIRGSAVNQDGASNGLTAPNGPSQERVIRAALANAGLGAAEVDAVEAHGTGTKLGDPIEAGALLATYGRDRDEDRPLWLGSVKSNIGHPQGAAGVAGVIKMVMALQRELLPATLYVDEPTPHVDWSSGSVRLLTEPVPWTRGERPRRAGVSAFGMSGTNAHVILEEAPPEEAAAAETPAEGTGAVVPWVVSGRGEEALRAQAAQLAEHVRDDDQRPASPLEVGWSLATTRSVFENRAVVVGDDRDALLDGLRSLAAGEASPDVVSGAVGPTGPGPVMVFPGQGGQWVGMGARLLDESPVFAARIAECEQALSAYVDWSLTDVLRGDGSELARIDVVQPVLWAVMVALAAVWADQGIEPAAVVGHSQGEIAAACVVGAISLDEAARIVAVRSVLLRQLSGRGGMASLGMGQEQAADLIDGHPGVVVAAVNGPSSTVISGPPEGIAAVVADAQERGLRARAVASDVAGHGPQLDAILDQLTEGLAGIRPAATDVAFYSTVTAGHLTDTTELDTAYWVRNVRRTVRFADTIDALLADGYRLFIEVSPHPVLNLALEGLIERAAVPATVVPTLRRDHGDTTQLARAAAHAFAAGADVDWRRWFPADPAPRTVDLPTYAFQRQDFWPAPAGGRSGDPAGLGLAASGHPLLGASVGLASGDVHLLSGRVSRQSAAWLDDHVVAGQALVPGAAQVEWVLRAGDDAGCSALEELTLQTPLVLPDTGGLRIQVVVEAADAHGRRDVRLFSRPDDDDAFASTHPWTCHATGVLAPAPTDGTNGTRDAADTLDGAWPPADAEPVPADDLYAQADRTGYGYGPAFRGVRALWRHGKDVLAEVTLPKEAGDPDGFGIHPALLDAVLQPAALLLPPTDAEQVWLPFAWNDVALHAVRATTVRVRLTPLGERIDQGLRITVADAVGAPVLTVRDLRSRPTDTGRLAAAATRDRHGLFDLEWIAPENAAENAAGPARDASEGWVTLGEDAASLADLLASVEAGAPAPQLVAAPVEPDRTDDGLALATHVLDLVQTWLASPLHDSRLVLVTRGAVTDADVDVAAAAVWGLVRSAQSEHPGRFTLIDLGPDDTLAAAMQAAHLEEPQLAVHGGEIRVPRLVRATTDPTAPNGTPEADRTADPSEGLHRNGTVLITGGTGVLGRLVAEHLVTEWGVRHLLLASRRGDQAPGSAELRARLSELGASVEIAPADVGDAEAVAALIASVDPAHPLTGVIHAAGVLDDAVITAQTPESLARVWATKATAARHLHEATRETPLDFFVVFSSAAASLGSPGQANYAAANAYCDALVQHRRAQGLAGLSIAWGLWQATSGMTGQLSETDLARMKRTGFAALTDEGGLALLDAARAHDRAYVVAADLDPRAVTDGLSPLLRALTAPATRRRVASEGLADGALATRLAGLDADGRLRLLTDVVREYVAAVLGHGSAARVGVDIAFKDLGFDSLTAVELRNRLSAACDVRLPATLIFDHPTPQALATHLVDRLAGSTSATTTVNATAPAAAHVAAGADVDADTDDPVAIVAMTCRFPGGVASPDDLWDLLDARKDAMGAFPTDRGWDLERLFHPDPDHPGTSYTDQGGFLPDAGDFDAAFFGINPREALAMDPQQRLLLEASWEVLERAGIDPTTLKGTPTGTYVGLMYHDYAKSFPTADAQLEGYSYLASTGSMVSGRVAYTLGLEGPAVTVDTACSSSLVSIHLATQALRHGECDLALAGGVTVMADPDMFAGFSRQRGLSPDGRCKAYAAAADGVGFSEGVGVLLLERLSDARRHGRRVLGVVRGSAVNQDGASNGLTAPNGPSQERVIRQALASGGLSSVDVDVVEGHGTGTTLGDPIEAQALLATYGQGRPEDRPLWLGSVKSNIGHTQAAAGVAGVIKMVMAMRHGVVPASLHVDVPSPHVEWDSGAVRLAVESVPWPQVEGRPRRAGVSSFGASGTNAHVIVESVPDGLEEDSVSVGGEALETETDGRLVPWVVSARSPQALRDQALRLRDFASDASFRAPLADVGWSLLKTRALHEHRAVVVGAERAELIAALEALATGEPHAALVGPACSQARVGGDDVVWLFSGQGSQLVGMGAGLYERFPVFAAAFDEVCGLLEGPLGVEAGGLREVVFRGPRERLDHTVWAQAGLFALQVGLARLWESVGVRPDVVLGHSIGEIAAAHVAGVFDLADACRVVGARARLMGGLPEGGAMCAVQATPAELAADVDGSAVSVAAVNTPDSTVISGPSDEVDRIAGVWRERGRKTKALSVSHAFHSALMEPMLAEFTEAIRGVKFRQPSIPLMSNVSGERAGEEITDPEYWARHVRNAVLFQPAIAQVADSAGVFVELGPAPVLTTAAQHTLDESDSQESVLVASLAGERPEESAFVEAMARLHTAGVAVDWSVLFAGDRVPGLVELPTYAFQRERFWLSGRSGGGDAATLGLVAAGHPLLGAAVEFADRGGCLLTGRLSRSGVSWLADHVVAGAVLVPGAALVEWALRAGDEVGCVTVEELMLQAPLVVPEASGLRVQVVVEEAGEDGRRGVQIYSRPDADAVGGDDSWICHATGVLSPESARLDTELGGVWPPAGAEPLDVDGFYAQAGEAGYGYGPAFRGLRAVWRHGQDLLAEVVLPEAAGAHDGYGIHPALLDATLHPLLAARFMDGSEDDQLYVPFGWAGVSLRAVGATTVRVRLRPVGESVDQGLSVTVTDATGGPVLSVDSLQTRPVKPSQLAAAQQPDVRGLFTVEWTPLPQTDADGEADWVVLSDGVGRLADVVSAAGGEAPWAVVAPVDASVGDGREGLDGRLVVERVLSLVQEFLALPELAESRLLVVTRGAVATGVDGDGDVDASAAAVWGLVRSAQSENPGRFILLDVDGDGDDQGPDLNGRHLPHATLRHAAEELDEPQLALREGTLYVPRLTQARQSAELVVPPGEPAWRLRMVHDGSLDALAAVACPEALEPLAPGQVRIAVHAAGINFRDVLVALGMVPAYGAMGGEGAGVVTEVGPEVTHVSVGDRVMGVFEGAFGPVVIAEARMVTPVPQGWDMREAAGIPAAFLTAWYGLVELAGLKAGERVLVHAATGGVGMAAVQIARHVGAEVFATASPGKHAVLEEMGIDAAHRASSRDLAFEGTFREATGGRGMDVVLNSLAGEFIDASLRLLGDGGRFLEMGKTDVRAAEEVAAEHADVSYTAYDLVGDAGPDRISNMLDKLVELFASERLKPLPVRSWPLDKAQEAFRFMSQAKHTGKLVLEIPPALDPEGTVLVTGGTGALGQVVAEHLVREWGVRHLLLASRRGPEAPGSDELASKLTGLGAEVTIVAADVSDPASVVELVGKTDPSHPLTGVVHAAGVLEDGVVTAQTPEGLARVWAAKAAAAANLHEATREMRLGLFVVFSSAAATLGSPGQANYAAANAYCDALMQHRRAVGQVGLSVGWGLWEAPDAKPGVAADAKASAATVGKASALSDGTNGSAPQDTTGTAPQGMTGGLTDTDVARMARIGVKGMSNAHGLALFDAAHRHGRPHLVGFNLDLRTLATHPLHTRPALLRGLATPTAGGASRPTATAGGQPADLAGRLAALSPSDRHHTLVRLIREQAATVLGHHPDSLTTGSTFKELGFDSLTAVELRNRLSAATGLRLPAGLVFDHPDADILAEHLGAQLAPDGDTPAGAEATDPVLRDLAKLENALSSTLVEHLDADAVTARLEALLSNWKAASAAPGSGSTKEQLQVATTDQVLDFIDKELGV

SEQ ID NO: 23 - protein, 4106 aa, Streptomyces cinnamonensis
MASEEELVDYLKRVAAELHDTRQRLREVEDRRQEPVAVVGMACRFPGGIETPEGLWELVAAGDDAIEPFPTDRGWDLEGIYHPDPDHPGTCYVREGGFLAAPDRFDSDFFGFSPREALASSPQLRLLLETSWEALERAGINPASLKGSPTGVYVGAATTGNQTQGDPGGKATEGYAGTAPSVLSGRLSFTLGLEGPAVTVETACSSSLVAMHLAANALRQGECDLALAGGVTVMSTPEVFTGFSRQRGLAPDGRCKPFAAAADGTGWGEGAGLILLERLSDARRKGHKVLAVIRGSAINQDGASNGFTAPNGPSQRRVIRQALSSAHLSTSEIDVVEAHGTGTRLGDPIEAEALIATYGKEREDDRPLWLGSVKSNIGHTQAAAGVAGVIKMVMALQRELLPATLNVDEPTPHVQWEGGGVRLLTEPVPWSRGERPRRAGISSFGISGTNAHVVLEEAPPEEDVPGPVAAEPEGVVPWVVSARTEEALSEQARRLGEFVADTDPSTADVGWSLTTSRAILEHRAVVVGRDRDALTAGLAALAAGEESADVVAGVAGDVGPGPVLVFPGQGSQWVGMGAQLLDESPVFAARIAECEQALSAYVDWSLSAVLRGDGSELSRVEVVQPVLWAVMVSLAAVWADYGVTPAAVIGHSQGEMAAACVAGALSLEDAARVVAVRSDALRQLMGQGDMASLGASSEQAAELIGDRPGVCIAAVNGPSSTVISGPPEHVAAVVADAEERGLRARVIDVGYASHGPQIDQLHDLLTDRLADIRPATTDVAFYSTVTAERLTDTTALDTDYWVTNLRQPVRFADTIDALLADGYRLFIEASAHPVLGLGMEETIEQADIPATVVPTLRRDHGDTTQLTRAAAHAFTAGATVDWRRWFPADPTPRTIDLPTYAFQRRSYWLPVDGVGDVRSAGLRRVEHSLLPAALGLADGALVLTGRLAASGGGGGWLADHAVAGTTLVPGAALVEWALRAADEAGCPSLEELTLQAPLVLPGSGGLQVQVVVGPADGQGGRREVRVFSRVDSDDEAAGQDEGWSCHATGVLSPEPGAVPDGLSGQWPPTGAEPLEISDLYEQAASAGYEYGPSFRGLRSVWRHGHNLLAEVELPEQAGAHDDFGIHPVLLDAALHPALLLDQNAPGEEQEPAQPALRLPFVWNGVSLWATGAATVRVRLAPHGGGETDDSAGLRVTVADATGAPVLSVDSLALRPADPELLRTAGRAGSGTNGLFTVEWTALPPADVADHAAGDGWAVLGQDVPDWAGADMPRHPDMASLSAALDEGTQAPAAVFVETTATSHATPNTAADVTLDASGRAVAERTLHLLRDWLAEPRLAETRLVLITHHAVTTPADDDVNAAPLDVPAAALWGLIRSAQAEHPDRFVLLDTDAKANTDPGPDTSTDHSTASGTYRTVIARALATGEPQLAVRAGELLAPRLARAATPTPETPTPETQPDTGSGSEAGAGSGSGPGATLDPDGTVLIAGGTGMMGGLVAEHLVRAWSVRHLLLVSRQGPDAPDARDLADRLVGLGATVRIVAADLTDGRATADLVASVDPAHPLTGVIHAAGVLDDAVVTAQTSDQLARVWAAKASVAANLDAATSELPLGLFLMFSSAAGVLGNAGQAGYAAANAFVDALVGRRRATGLPGLSIAWGLWARGSAMTRHLDDADLARLRAGGVKPLLDEQGLALLDAARATAAHTSLVVAAGIDVRGLNRDDVPAILRDLAGRTRRRAAADSTVDQAALERRLTGLDEAERRAVVTDVVRECVAAVLGHRSAADVRTEANFKDLGFDSLTAVQLRNRLSAASGLRLPATLAFDHPTPQALAAYLGTRLSGRTATPVAPVAPSAAATDEPVAIVAMACKYPGGATSPEGLWDLVAEGVDAVGAFPTGRGWDLERLFHPDPDHPGTSYADEGAFLPDAGDFDAAFFGINPREALAMDPQQRLLLEASWEVLERAGIDPTTLKGTPTGTYVGVMYHDYAAGLAQDAQLEGYSMLAGSGSVVSGRVAYTLGLEGPAVTVDTACSSSLVSIHLAAQALRQGECTLALAGGVTVMATPEVFTGFSRQRGLAPDGRCKPFAAAADGTGWGEGVGVLLLERLSDARRHGRRVLGVVRGSAVNQDGASNGLTAPNGPSQERVIRQALASGGLSSVDVDVVEGHGTGTTLGDPIEAQALLATYGQGRPVDRPLWLGSVKSNIGHTQAAAGVAGVIKMVMAMRHGVVPASLHVDVPSPHVEWDSGAVRLAVESVPWPEVEGRPRRAGVSSFGASGTNAHVIVESVPDGLGEDSVSVSGEAPETETDGRLVPWVVSARSPQALRDQALRLRDAVAADSTVSVQDVGWSLLKTRALFEQRAVVVGRERAELLSGLAVLAAGEEHPAVTRSREDGVAASGAVVWLFSGQGSQLVGMGAGLYERFPVFAAAFDEVCGLLEGPLGVEAGGLREVVFRGPRERLDHTMWAQAGLFALQVGLARLWESVGVRPDVVLGHSIGEIAAAHVAGVFDLADACRVVGARARLMGGLPEGGAMCAVQATPAELAADVDDSGVSVAAVNTPDSTVISGPSGEVDRIAGVWRERGRKTKALSVSHAFHSALMEPMLAEFTEAIREVKFTRPKVSLISNVSGLEAGEEIASPEYWARHVRQTVLFQPGIAQVASTAGVFVELGPGPVLTTAAQHTLDDVTDRHGPEPVLVSSLAGERPEESAFVEAMARLHTAGVAVDWSVLFAGDRVPGLVELPTYAFQRERFWLSGRSGGGDAATLGLVAAGHPLLGAAVEFADRGGCLLTGRLSRSGVSWLADHVVAGAVLVPGAALVEWALRAGDEVGCVTVEELMLQAPLVVPEASGLRVQVVVEEAGEDGRRGVQIYSRPDADAVSGDDSWICHATGTLTPQHTDAPNDGLAGAWPAAGAVPVDLAGFYERVADAGYAYGPGFQGLRAVWRHGQDLLAEVVLPEAAGAHDGYGIHPALLDATLHPALLLDWPGEVQDDDGKVWLPFTWNQVSLRAAGAATVRVRLSPGEHDEAEREVQVLVADATGTDVLSVGSVTLRPADIRQLQAVPGHDDGLFSVDWTPLPLSRTDVSQTDADGDADWVVLSDGVGSLADVVSAAGGEAPWAVVAPVGASAGGGLAGFDRREGLDGRLVVERVLSLVQEFLAAPELAESRLLVLTRGAVATGGDGDGDVDASAAAVWGLVRSAQSENPGRFILLDVDMDVDVDVDMDVDVDVDVDVDVDGDGNGSDLDPDLNGRRLPHATLRHAAEELDEPQLALRDGQLLVPRLVRATGGGLVVAPTDRAWRLDKGSAETLESVAPVAYPGVMEPLGPGQVRLGIHAAGINFRDVLVSLGMVPGQVGLGGEGAGVVTETGPDVTHLSVGDRVMGVLHGSFGPTAVADTRMVAPVPQGWDMRQAAAMPVAYLTAWYGLVELAGLKAGERVLIHAATGGVGMAAVQIARHLGAEVFATASAAKHVVLEEMGIDAAHRASSRDLAFEDTFRQATDGRGMDVVLNSLTGEFIDASLRLLGDGGRFLEMGKTDVRTPEEVAAEYPGVTYTVYDLVTDAGPDRIAVMMSELGERFASGALDPLPVRSWPLDKAREAFRFMSQAKHTGKLVLDVPAPLDPDGTVLITGGTGALGQVVAEHLVREWGVRHLLLASRRGLDAPGSGELADRLSDLGAEVTVAAADVSDPASVVELVGKTDPSHPLTGVVHAAGVLEDGIVTAQTPEGLARVWAAKAAAAANLHEATREMRLGLFVVFSSAAATLGSPGQANYAAANAYCDALMQRRRAAGQVGLSVGWGLWEAPDAKPGVAADAKPDVAADAKTGVAADGTPQGMTGTLSGTDVARMARIGVKAMTSAHGLALLDAAHRHGRPHLVAVDLDTRVLAHKPAPALPALLRAFAGDQGGQGGGRGGGRGGGPARPAAATTRQNVDWAAKLSVLTAEEQHRTLLDLVRTHAAAVLGHAGTDAVRADAAFQDLGFDSLTAVELRNRLSASTGLRLPATFIFRHPTPSAIADELRAQLAPAGADPAAPLFGELDKLETVITGHAHDESTRTRLAARLQNLLWRLDDTSARSDHAAGASDADGDAVENRDLESASDDELFELIDRELPS

SEQ ID NO: 24 - protein, 1700 aa, Streptomyces cinnamonensis
MPGTNDMPGTEDKLRHYLKRVTADLGQTRQRLRDVEERQREPIAIVAMACRYPGGVASPEQLWDLVASRGDAIEEFPADRGWDVAGLYHPDPDHPGTTYVREAGFLRDAARFDADFFGINPREALAADPQQRVLLEVSWELFERAGIDPATLKDTLTGVYAGVSSQDHMSGSRVPPEVEGYATTGTLSSVISGRIAYTFGLEGPAVTLDTACSASLVAIHLACQALRQGDCGLAVAGGVTVLSTPTAFVEFSRQRGLAPDGRCKPFAEAADGTGFSEGVGLILLERLSDARRNGHQVLGVVRGSAVNQDGASNGLTAPNDVAQERVIRQALTNARVTPDAVDAVEAHGTGTTLGDPIEGNALLATYGKDRPADRPLWLGSVKSNIGHTQAAAGVAGVIKMVMAMRHGELPASLHIDRPTPHVDWEGGGVRLLTDPVPWPRADRPRRAGVSSFGISGTNAHLIVEQAPAPPDTADDAPEGAATPGASDGLVVPWVVSARSPQALRDQALRLRDFAGDASRAPLTDVGWSLLRSRALFEQRAVVAGRERAELLAGLAALAAGEEHPAVTRSREEAAVAASGDVVWLFSGQGSQLVGMGAGLYERFPVFAAAFDEVCGLLEGELGVGSGGLREVVFWGPRERLDHTVWAQAGLFALQVGLARLWESVGVRPDVVLGHSIGEIAAAHVAGVFDLADACRVVGARARLMGGLPEGGAMCAVQATPAELAADVDGSSVSVAAVNTPDSTVISGPSGEVDRIAGVWRERGRKTKALSVSHAFHSALMEPMLGEFTEAIRGVKFRQPSIPLMSNVSGERAGEEITSPEYWARHVRQTVLFQPGVAQVAAEARAFVELGPGPVLTAAAQHTLDHITEPEGPEPVVTASLHPDRPDDVAFAHAMADLHVAGISVDWSAYFPDDPAPRTVDLPTYAFQGRRFWLADIAAPEAVSSTDGEEAGFWAAVEGADFQALCDTLHLKDDEHRAALETVFPALSAWRRERRERSIVDAWRYRVDWRRVELPTPVPGAGTGPDADTGLGAWLIVAPTHGSGTWPQACARALEEAGAPVRIVEAGPHADRADMADLVQAWRASCADDTTQLGGVLSLLALAEAPATSSDTTSHTSTSCGTGSLASHGLTGTLTLLHGLLDAGVEAPLWCATRGAVSCGDADPLVSPSQAPVWGLGRVAALEHPELWGGLVDLPADPESLDASALYAVLRGDGGEDQVALRRGAVLGRRLVPDATPDVAPGSSPDVSGGAAHADATSGEWQPHGAVLVTGGVGHLADQVVRWLAASGAEHVVLLDTGPANSRGPGRNDDLAAEAAEHGTELTVLRSLSELTDVSVRPIRTVIHTSLPGELAPLAEVTPDALGAAVSAAARLSELPGIGSVETVLFFSSVTASLGSREHGAYAAANAYLDALAQRAGADAASPRTVSVGWGIWDLPDDGDVARGAAGLSRRQGLPPLEPQLALGALRAALDGGKGHTLVADIEWERFAPLFTLARPTRLLDGIPAAQRVLDASSESAEASENASALRRELTALPVRERTGALLDLVRKQVAAVLRYEPGQDVAPEKAFKDLGFDSLVVVELRNRLRAATGLRLPATLVYDYPTPRTLAAHLLDRVLPDGGAAELPVAAHLDDLEAALTDLPADDPRRKGLVRRLQTLLWKQPDAMGAAGPADEEEQAAPEDLSTASADDMFALIDREWGTR

SEQ ID NO: 25 - protein, 980 aa, Streptomyces cinnamonensis
VSGVERGVGSAGPVEQGDGLAGLVERAEALAALRGAFDGSPGTGGSLVVLSGAVGTGKTALLRAWADRIGADADALVLTATACRAERDLPLGVLEQLVRSPGLPPASAERALAWWDEEASATPGKTDANGTSANGTDANGTGAGQTGAGQAGVGQTGVGGEPVLAASALRGLCEVLRDLLAERPVVVAVDDAHHADAASLQCLLSVVRRLRSARLHVLFTEYAHQKAQNALLSSEFLHEPALRRIRLEPLSKAGVEALLARHLDERTAQDLTPVVHGMSAGHPLLVRALAEDHRAAGGAGEAYGRAVLSFLYRHETPVTQVARAIAALGAHAGPGQVGRLLDVDAASVERAVRQLTVAEVLHEGRLCHPAFAAAVLDGMPPEERRALHGRVADLLHEEGAPATEVAAHLVAADRSDAPWAVPVFQEAAQLALDEDQVETGVDYLRAAHQRCRGAAQRAAVVGALADAEWRLDPAKVLRHLPDPAAMAPQTDPAALAPHTDPAPTAAPTAAPTPTPIPTTPPLPTHLLWHGRVEEGLDAIGTLTGPGPNPAGAPPMNPADLDTPWLWGAYLYPGHVKERLGSGALSPQRSTPPAVTPELQGAGTLMNDLLHGGERDATEAAERALNRYRLGPRTIAVQTAALAALTYRDRPHRAAAWCDGLVAQADERNSPTWRALFTAWRALLHLRQGDPAAAEQRAETALALLGSKGWGAAIGLPLAAAVQAKAALGDVDGAAALLERPVPQAVFQTRTGLHYLAARGRYHLATGCHYAALCDFYACGTRMSSWGVDLPALEPWRLGAAEAYLALGEGLLARQLVDGQLPLPTPDDGRTWGMTLRLRAATSPAPARAELLDEAVAVLRESGDTFELARAVADQAVAVREGGEAERARLLARKAELLARRWGSAPAPATVPEPPERPGPATPDAELTSAERRVAELAAEGFTNREISRKLCVTVSTVEQHLTRIYRKLDVRRLDLQAALG

SEQ ID NO: 26 - protein, 496 aa, Streptomyces cinnamonensis
VTTTRPAHAVVLGASMAGTLAAHVLARHVDAVTVVERDALPEEPQHRKGVPQARHAHLLWSNGARLIEEMLPGTTDRLLAAGARRLGFPEDLVTLTGQGWQHRFPATQFALVASRPLLDLTVRQQALGADNITVRQRTEAVELTGSGGGSGGRVTGVVVRDLDSGRQEQLEADLVIDATGRGSRLKQWLAALGVPALEEDVVDAGVAYATRLFKAPPGATTHFPAVNIAADDRVREPGRFGVVYPIEGGRWLATLSCTRGAQLPTHEDEFIPFAENLNHPILADLLRDAEPLTPVFGSRSGANRRLYPERLEQWPDGLLVIGDSLTAFNPIYGHGMSSAARCATTIDREFERSVQEGTGSARAGTRALQKAIGAAVDDPWILAATKDIDYVNCRVSATDPRLIGVDTEQRLRFAEAITAASIRSPKASEIVTDVMSLNAPQAELGSNRFLMAMRADERLPELTAPPFLPEELAVVGLDAATISPTPTPTPTAAVRS

SEQ ID NO: 27 - protein, 140 aa, Streptomyces cinnamonensis
MPDEAARKQMAVDYAERINAGDIEGVLDLFTDDIVFEDPVGRPPMVGKDDLRRHLELAVSCGTHEVPDPPMTSMDDRFVVTPTTVTVQRPRPMTFRIVGIVELDEHGLGRRVQAFWGVTDVTMDDPAGPADTTHPEGIRA

SEQ ID NO: 28 - protein, 144 aa, Streptomyces cinnamonensis
MNEFARKKRALEHSRRINAGDLDAIIDLYAPDAVLEDPVGLPPVTGHDALRAHYEPLLAAHLREEAAEPVAGQDATHALIQISSVMDYLPVGPLYAERGWLKAPDAPGTARIHRTAMLVIRMDASGLIRHLKSYWGTSDLTVLG

SEQ ID NO: 29 - protein, 3753 aa, Streptomyces cinnamonensis
MSNEEKLLDHLKWVTAELRQARQRLHDKESTEPVAIVGMACRYPGGARSAEDLWELVRDGGDAVAGFPDDRGWDLESLYHPDPEHPATSYVRDGAFLYDAGHFDAEFFGISPREATAMDPQQRLLLETAWEAIEHAGMNPHALKGSDTGVFTGVSAHDYLTLISQTASDVEGYIGTGNLGSVVSGRISYTVGLEGPAVTVDTACSSSLVAIHLASQALRQGECSLALAGGSTVMATPGSFTEFSRQRGLAPDGRCKPFAAAADGTGWGEGAGVVALELLSEARRRGHKVLAVIRGSATNQDGTSNGLAAPNGPSQERVIRAALANARLSAEDIDAVEAHGTGTTLGDPIEAQALIATYGQGRPEDRPLWLGSVKSNIGHTQAAAGVAGVIKMVMAMRNGLLPTSLHIDAPSPHVQWEQGSVRLLSEPVDWPAERTRRAGISAFGISGTNAHLILEEAPPEEDAPGPVAAEPGGVVPWVVSGRTPDALREQARRLGEFAAGLADASVSEVGWSLATTRALFDQRAVVVGRDLAQAGASLEALAAGEASADVVAGVAGDVGPGPVLVFPGQGSQWVGMGAQLLDESPVFAARIAECEQALSAHVDWSLSDVLRGDGSELSRVEVVQPVLWAVMVSLAAVWADYGITPAAVIGHSQGEMAAACVAGALSLEDAARIVAVRSDALRQLQGHGDMASLSTGAEQAAELIGDRPGVVVAAVNGPSSTVISGPPEHVAAVVADAEAQGLRARVIDVRYASHGPQIDQLHDLLTDRLADIQPTTTDVAFYSTVTAERLDDTTALDTAYWVTNLRQPVRFADTIEALLADGYRLFIEASPHPVLNLGIQETIEQQAGAAGTAVTIPTLRRDHGDTTQLTRAAAHAFTAGAPVDWRRWFPADPTPRTVDLPTYAFQHKHYWVEPPAAVAAVGGGHDPVEARVWQAIEDLDIDALAGSLEIEGQAESVGALESALPVLSAWRRRHREQSTVDSWRYQVTWKHLPDVPAPELSGAWLLLVPAAHADHPAVLATAQTLTAHGGEVRRHVVDARAMERTELAQELRVLMDGAAFAGVVNLLALDEEPHPEHSAVPAGLAATTALVQALADNGADIAVRTLTQGAVSTSAGDALTHPVQAQVWGLGRVAALEYPRLWGGLVDLPARIDHQTLARLAAALVPQDEDQISIRPSGVHARRLAHAPANTVGSGLGWRPDGTTLITGGTGGIGAVLARWLARAGAPHLLLTSRRGPDAPGAQELAAELTELGAAVTVTACDVGDREQVRRLIDDVPAEHPLTAVIHAAGVPNYIGLGDVSGAELDEVLRPKALAAHHLHELTRELPLSAFVMFSSGAGVWGSGQQGAYGAANHFLDALAEHRRAEGLPATSIAWGPWAEAGMAADQAALTFFSRFGLHPLSPELCVKALQQALDAGETTLTVANFDWAQFTSTFTAQRPSPLLADLPENRRASAPAAQQEDATEASSLQQELTEAKPAQQRQLLLQHVRSQAAATLGHSDVDAVPATKPFQELGFDSLTAVELRNRLNKSTGLTLPTTVVFDHPTPDALTDVLRAELSGDAAASADPVRAAGASRGAADDEPIAIVGMACRYPGDVRSAEELWDLVAAGKDAMGAFPDDRGWDLETLYDPDPESRGTSYVREGGFLYDAGDFDAGFFGISPREAVAMDPQQRLLLETAWEAIERAGLDRETLKGSDAGVFTGLTIFDYLALVGEQPTEVEGYIGTGNLGCVASGRVSYVLGLEGPAMTIDTGCSSSLVAIHQAAHALRQGECSLALAGGATVMATPGSFVEFSLQRGLAKDGRCKPFAAAADGTGWAEGVGLVVLERLSEARRNGHNVLAVIRGSAINQDGTSNGLTAPNGQAQQRVIRQALANARLSAEDVDAVEAHGTGTMLGDPIEASALVATYGKERPADRPLWLGSIKSNIGHAQASAGVAGVIKMVMALRNEQLPASLHIDAPTPHVDWDGSGVRLLSEPVSWPRGERPRRAGVSAFGISGTNAHLILEQAPDAPEPVTAPAEDAAAPAGVVPWVVSARGEEALRAQARLLADRATADPRLASPLDVGWSLVKTRSVFENRAVVVGKDRQTLLAGLRSLAAGEPSPDVVEGAVQGASGAGPVLVFPGQGSQWVGMGAQLLDESPVFAARIAECERALSAHVDWSLSAVLRGDGSELSRVEVVQPVLWAVMVSLASVWADYGITPAAVIGHSQGEMAAACVAGALSLEDAARIVAVRSDALRQLMGQGDMASLGAGSEQVAELIGDRPGVCVAAVNGPSSTVISGPPEHVAAVVADAEARGLRARVIDVGYASHGPQIDQLHDLLTERLADIRPTTTDVAFYSTVTAERLDDTTTLDTDYWVTNLRQPVRFADTIEALLADGYRLFIEASPHPVLNLGMEETIERADMPATVVPTLRRDHGDAAQLTRAAAQAFGAGAEVDWTGWFPAVPLPRVVDLPTYAFQRERFWLEGRRGLAGDPAGLGLASAGHPLLGAAVELADGGSHLLTGRISPRDQAWLAEHRVMDTVLLPGSAFVELALQAAVRAGCAELAELTLHTPLAFGDEGAGAVDVQVVVGSVAEDGRRPVTVHSRPTGEGEEAVWTRHAAGVVAPPGPDAGDASFGGTWPPPGATPVGEQDPYGELASYGYDFGPGSQGLVSAWRLGDDLFAEVALPEAESGRADRYQVHPVLLDATLHALILDAVTSSADTDQVLLPFSWSGLRVHAPGAEKLRVRIARTAPDQLALTAVDGGGGGEPVLTLESLTVRPVAAHQIAGARAADRDALFRLVWMEVAARAEETGGGAPRAAVLAPVESGPMGGTSAGALADALSDALAAGPVWDTFGALRDGVAAGGEAPDVVLAVCAAPGAGAGAVADADGRGGDPAGYARLATVSLLSLLKEWVDDPAFAATRLVVVTRGAVAARPGETAGDLAGASLWGLVRSAQAENPGRLTLLDVDGLESSPATLTGVLASGEPELALRDGRAYVPRLVRDDASVRLVPPVGSLTWRLARCQEAGGGQQLSLVDAPEAGRALEPHEVRVAVRAAAPGPLTAGQVEGAGVVTEVGGEVGSVAVGDRVMGLFDAVGPVAVTDAALLMPVPAGWSWAQAAGSLGAYVSAYHVLADVVAPRGGETLLVGEETGSVGRAVLRLALAGRWRVEAVDGASTADDSGAERAADVTLRHEGALVVHRAGGRPDEGQAVVPPEPGRVREILAELTELTELAEITESAEPGLPAERGDSRALTPLDITVWDIRQAPAAMAAPPSAGTTVFSLPPAFDPEGTVLVTGGTGALGSLTARHLVERYGARHLLLSSRRGADAPGALELAADLSALGARVTFAACDPGDRDEAAALLAAVPSDHPLTAVFHCAGTVNDAVVQNLTAEQVEEVMRVKADAAWHLHELTRDADLSAFVLYSSVAGLLGGPGQGSYTAANAFLDALARHRHDGGAAATSLAWGYWELASGMSGRLTDADRARHARAGVVGLGADEGLALLDAAWAGGLPLYAPVRLDLARMRRQAQSHPAPALLRDLVRGGSKSGGGAVSAGAAALLKSLGAMSDPEREEALLDLVCTHIAAVLGYDAATPVNATQGLRELGFDSLTAVELRNRLSAATGLKLPATFVFDHPNPAELAAQLRQELAPRAADPLADVLAEFERIEDSLLSVSSKDGSARAELAGRLRATLARLDAPQDTAGEVAVATRTRIQDASADEIFAFIDRDLGRDGASGQGNGQPTGQGNGHGNGNGNGNGNGHGQAVEGQR

SEQ ID NO: 30 - protein, 1641 aa, Streptomyces cinnamonensis
MAHTEEKLLEYLKRVTADLRQTERRLQDVESAGHEPVAVIGMACRLPGGVRSPEEFWELVSTGGDAVAPLPGNRNWDLDSLYDPDPESTGTSYVREGGFVYDAGDFDPTFFGIGPTEAAAMAPQQRLALETAWEAIERAGIDPLSLRSSDTSTFIGCDGLDYALGASEVPEGTAGYFTIGNSGSVTSGRVAYTLGLEGPAVTVDTACSSSLVSLHLATQALRTQECSLALAGGTYVMSSPAPLIGFSELRGLAPDGRCKPFSASSDGMGMAEGTGVVLLERLSDARRKGHKVLAVIRGSAINQDGASNGLTAPNGPAQERVIRAALANARLAPEDIDAVEAHGTGTTLGDPIEAGALISAYGRERPEDRPLWVGAVKSNIGHTQIAAGVAGVIKMVLALRHDLLPAILHVDAPSPHVEWDGSGLRLLTDPVKWPRGERPRRAGVSSFGFSGTNAHLILEEAPPEEEDVPGSVAEEPGGVVPWVVSGRTPDALRAQARRLGEFAAGPADASAADVGWSLTTTRSVFEHRAVVVGRDRDALTAGLGALAAGEASAGVVAGVAGDVGPGPVLVFPGQGSQWVGMGAQLLDESPVFAARIAECERALSAYVDWSLSAVLRGDGSELSRVEVVQPVLWAVMVSLAAVWADYGVTPAAVIGHSQGEMAAACVAGALSLEDAARIVAVRSDALRRLQGHGDMASLSTGAEQAAELIGDRPGVVVAAVNGPSSTVISGPPEHVAAVVADAEARGLRARVIDVGYASHGPQIDQLHDLLTERLADIRPANTDVAFYSTVTAERLTDTTALDTDYWVTNLRQPVRFADTIEALLADGYRLFIEASAHPVLGLGMEETIEQADIPATVVPTLRRDHGDTTQLTRAAAHAFTAGAPVDWRRWFPADPTPRTVDLPTYAFQHQHYWLERSASASGAVSGEQSAAEAQLWHAVEELDLGLLAETLGSEEGSEEAVRALEPALPVLKGWRRRHQDQATIDSWRYRVTWKQRSDGPAPELGGDWLLFVPADKAEHPAVRATAEALSEHGAAAVRLHPVETGRAGRQELAAVDTAGLAGIVNLLALDEEPHPEHPAVPAGLAATTALLQALGDNGTTAPLHTVTQGAVSTGATDPLTHPLQAHVWGLGRVAALEHPRLWAGLVDLPARIDRHTLPRLAAALLPQDDEDQTAVRPTGIHHRRLTHAVGSIQNPVHSEATWRPRGTTLITGGTGGIGAVLARWLARQGAPRLHLTSRRGPDAPGARELAAELDGLGTAVTITACDVSDPRQLSGLIDDMPAEHPLTAVIHAAGMTDLTAIGDLTTARLGEVLGSKSDAAWNLHELTRDLDLSAFVMFSSGAGVWGSGQQGAYGAANHFLDALAEHRRAQGLPATSIAWGPWAEAGMSADPESLTYFKRFGLLPIAPDLCVKALHQAVDAGDATLTVANFDWAKFTPTFTAQRPSPFLDDLPENQREAEQTGTAAETSAFREELAKTPASQRLGFLVQQVRTYAAATLGRTVEDIPAAKPFQELGFDSLTAVQLRNQLNTTTGLSLPATVIFDHPTPEALATHLRGQLGDGAEVAGEGDVLAALDKWDTAFGAAEVDEAARRRIVGRLQVLVSKWSPAQDGPEGTDSAHADLEAASADDIFDLISSEFGKS

SEQ ID NO: 31 - protein, 430 aa, Streptomyces cinnamonensis
VGLTVGPDNAKRGIVPITDSKPAATFPDLVDPSFWARPHAERVALFEEMRGLPRPAFIRQNMPGVPWTFGYHALVKYADIVEVSRRPQDFSSNGATTIIGLPPELDEYYGSMINMDNPEHSRLRRIVSRSFGRNMIPEFEAVATRTARRIIDELIARGPGDFIRPVAAEMPIAVLSDMMGIPAEDHDFLFDRSNTIVGPLDPDYVPDRADSERAVIEASRELGDYIAGLRAERLAAPGNDLITKLVQVQADGEQLTRQELVSFFILLVIAGMETTRNAISHALVLLTEHPEQKQLLLSDFDTHAPNAVEEILRVSTPINWMRRVATRDCDMNGHRFRRGDRIFLFYWSGNRDESVFPDPYRFDITRGTNAHVTFGAVGPHVCLGAHLARMEITVLYRELLAALPQIHAVGQPRRLDSSFIEGIKHLHCAF

SEQ ID NO: 32 - protein, 267 aa, Streptomyces cinnamonensis
VRYEMLGPLRIKDGNDYATINAQKVEIVLTVLLIRADRVVSLEQLMREIWGEDLPRRATAGLHVYISQLRKFLKVPGSAGNPVETRAPGYVLHKRDDDQIDAQIFPELVDVGRSLLREKRFDEAASCFGQALALWRGPILGQGGNGPGTNGPIIDGFSTWLTEIRLECQEMLVECQLQLGRHREAVGMLYALTAENPMCEAFYRQLMLALYRSERQADALKVYQSVRKTLNDELGLEPGRPLQELQRAILAGDMHLMSPPPLALSGR

SEQ ID NO: 33 - protein, 277 aa, Streptomyces cinnamonensis
LSAFLAKGKILSAFPPPDMSDPWIRRFRPRPEAVVRLVCFPHAGGSASYYHPLAQSPTLPTDSEVLAVQYPGRQDRRRERLLDDIGELADLITDALGPFDDRPLAFFGHSMGAVLAYEVAQRLRERTGKQPCRLFVSGRRAPSRFRRGTVHLLDDTELAAELRRAGGTDPRFLDDEELLAEIIPVVRNDYRAVELYRWNPSPPLSCPITALVGDRDPQAPLDEVEAWQQHTEGPFDLKVFAGGHFYLNTHQQGVTEVISKALADSAQQRATARGNAR

SEQ ID NO: 34 - protein, 427 aa, Streptomyces cinnamonensis
LADLVAHARSASPYYRELYHGLPERIEDPTLLPVTDKKQLMDHFDDWPTDRDITFEKVRAFTDDPELIGRRFLGRYLVATTSGTSGRRGLFVLDDRYMNVSSAVSSRVLASWLGPLGIARAVVHGGRFAQLVATEGHYVGFAGYSRLRQDGEARSKLVRAFSVHEPMSRLVAELNEYRPAFVIGYASTIMLFTAEQEAGRLHIDPVLVEPAGETMTESDTDRIAAAFGAKVRTMYSATECTYLSHGCAEGWYHVNDDWAVLEPVDADHRPTPPGEFSHTTLISNLANRVQPFLRYDLGDSVMLRPDPCPCGTPSPAIRVQGRSGDILTFPSGRGDDVSLAPLAFSSLFDRMPGVELFQIEQTAPSTLRVRVVQAPGADADHVWQRAHDGLTHLLADNKLDNVTVERGEEPPRQASGGKYRTIIPLAA

SEQ ID NO: 35 - protein, 338 aa, Streptomyces cinnamonensis
VKVPVEVTVRLSSWLGGLVAAVLAATVLPASAASAADVSSPPLEIPAAELAKALHCGTELGDLRDAGDKPTVLFVPGTGLKGEENYAWNYMAELKKKGYQSCWVDSPGRGLRDMQESVEYVVYATRAIQEATGRKVDLVGHSQGGLLTAWALRFWPDLPGKVDDMVTLGSPFQGTRLASPCRPIAEVAGCPASVLQFARDSNWSKALGADGTPMPAGPSYTTIYSYADESVVADGEAPSLPGAHRIGVQDICPGRPWPTHIAMVVDQVSYDLVADAIEHPGPADTSRIDRAHCAKPVMPLNSQEAVDALPGLLNFPIELLIHSQPWVDEEPPLRPYAR

SEQ ID NO: 36 - protein, 308 aa, Streptomyces cinnamonensis
MGHDHGPSAGAAGGTLSGTYRKRLLWTIGISGSITVIQVVGALLSGSLALLADAAHSLTDAVGVSLALGAITLAQRAPTPRRTFGFCRVEIFSAVLNALLLVVIFAWVLWSAIGRFSEPVEVKGGLMFVVALGGLAANLVGLWLLRDAKEKSLNLRGAYLEVLGDALGSVAVIVGGLVILLTGWQAADPIASIVIGLLIVPRAYGLLRDSLHVLLEATPQDVDLGEVRRHLLEERGVVAVHDLHGWTVTSGMPVLTAHVVVTEEALASGYGELLGRLQRCVGGHFDVAHSTIQLEPEGHVEEDGALHT

SEQ ID NO: 37 - protein, 363 aa, Streptomyces cinnamonensis
MTRALTLHDWIVAGIAVVAGVVAGLLLRALLRWLGERASKTRWSGDDVIVDALRTLVPCAAITAGLAAAAGALPLTPRTGRNVTMTLTALLILAATLTAARIVTGLVKAVAQSRSGVAGSATIFVNITRVVVLAMGFLIVLQTLGISIAPLLTALGVGGLAVALALQDTLANLFAGVHILAAKTVQPGDYIQLSSGEEGYVVDINWRNTTVRQLSNNLVIIPNAKLAGTNMTNYSRPEQELSIMVQVGVSYDSDLEQVEKVTTEVVDEVMAEITGAVPDHEAAIRFHTFGDSRISFTVILGVGEFSDQYRIKHEFIKRLHQRYRAEGIRVPAPVRTVRVQQGELPPPLGIPHQRDTSTQARLH

SEQ ID NO: 38 - protein, 131 aa, Streptomyces cinnamonensis
MSPVNSHNEWDPLEEIIVGRLEGATIPSSHPVVACNIPTWAARLQGLAAGFEYPQRLIEPAQQELDQFIALLQSLDVTVRRPAAVDHKHRFGTPDWQSRGFCNSCPRDSMLVVGDEIIETPMAWPCRCFET

SEQ ID NO: 39 - DNA, 32 bp, Artificial Sequence
  Description of Artificial Sequence Oligonucleotide
ccactagtatgcatgcgagtgtccgttcgagt

SEQ ID NO: 40 - DNA, 29 bp, Artificial Sequence
  Description of Artificial Sequence Oligonucleotide
ttgtatacacctaggatggttggccgtgc

SEQ ID NO: 41 - DNA, 29 bp, Artificial Sequence
  Description of Artificial Sequence Primer
agataccatatgctgggcccgctccgcat

SEQ ID NO: 42 - DNA, 35 bp, Artificial Sequence
  Description of Artificial Sequence Primer
aatgctctagactgtcagcgaccggacagggccaa

SEQ ID NO: 43 - DNA, 35 bp, Artificial Sequence
  Description of Artificial Sequence Primer
ggtggccacggaaacaccaacaccggacccgcgcc

SEQ ID NO: 44 - DNA, 29 bp, Artificial Sequence
  Description of Artificial Sequence Primer
ctctcggaggcccggcgcaacggccacaa

SEQ ID NO: 45 - DNA, 34 bp, Artificial Sequence
  Description of Artificial Sequence Primer
ggcctagggctgcctcgggtggtggatctgccga

SEQ ID NO: 46 - DNA, 27 bp, Artificial Sequence
  Description of Artificial Sequence Primer
tggtcgggcgcggtgcgtgcgatacgt

SEQ ID NO: 47 - DNA, 30 bp, Artificial Sequence
  Description of Artificial Sequence Primer
cctggccagggcggccagtgggtgggcatg

SEQ ID NO: 48 - DNA, 35 bp, Artificial Sequence
  Description of Artificial Sequence Primer
ggcctaggggtcggccgggaaccagcgccgccagt

SEQ ID NO: 49 - DNA, 31 bp, Artificial Sequence
  Description of Artificial Sequence Primer
gtgacgtcatatgtcgagtgctgaagagtcg

SEQ ID NO: 50 - DNA, 32 bp, Artificial Sequence
  Description of Artificial Sequence Primer
ggggtcgcctaggaaccagcgccgccagtcga

SEQ ID NO: 51 - DNA, 50 bp, Artificial Sequence
  Description of Artificial Sequence Primer
cggcctcgagggcccgtcggtcagtgtcgacacggcgcagtcctcctcgc

SEQ ID NO: 52 - DNA, 32 bp, Artificial Sequence; identical to SEQ ID NO: 50
  Description of Artificial Sequence Primer